(19) United States
(12) Patent Application Publication
(10) Pub. No.: US 2026/0325622 A1
(43) Pub. Date: Oct. 22, 2026

(54) HIV VACCINE IMMUNOGENS FOR THE INDUCTION OF V3-GLYCAN TARGETING ANTIBODIES

(71) Applicants: Duke University, Durham, NC (US); Triad National Security, LLC,, Los Alamos, NM (US)

(72) Inventors: Rory Henderson, Durham, NC (US); Priyamvada Acharya, Durham, NC (US); Victoria Stalls, Durham, NC (US); Barton F. Haynes, Durham, NC (US); Kshitij G. Wagh, Los Alamos, NM (US); Bette T. Korber, Los Alamos, NM (US)

(21) Appl. No.: 19/107,742

(22) PCT Filed: Aug. 31, 2023
(86) PCT No.: PCT/US2023/073257

§ 371 Date: Feb. 28, 2025

Related U.S. Application Data
(60) Provisional application No. 63/402,877, filed on Aug. 31, 2022.

(51) Int. Cl. A61K 39/21; A61K 9/16; A61K 39/00; C07K 14/005

(52) U.S. Cl. CPC A61K 39/21; A61K 9/1658; C07K 14/005; A61K 2039/575

(57) ABSTRACT

The invention is directed to modified HIV-1 envelopes, compositions comprising these modified envelopes, nucleic acids encoding these modified envelopes, compositions comprising these nucleic acids, and methods of using these modified HIV-1 envelopes and/or these nucleic acids to induce immune responses.

This application claims the benefit of and priority to U.S. Provisional Patent Application No. 63/402,877, filed on Aug. 31, 2022, the contents of which is hereby incorporated by reference in its entirety.

This invention was made with government support under Center for HIV/AIDS Vaccine Immunology-Immunogen Design grant UM1AI100645 from the NIH, NIAID, Division of AIDS. The government has certain rights in the invention.

### SEQUENCE LISTING

The instant application contains a Sequence Listing which has been submitted electronically in XML format and is hereby incorporated by reference in its entirety. Said XML copy, created on Aug. 31, 2023, is named 1234300_00423WO1_SL.xml and is 833,000 bytes in size.

### TECHNICAL FIELD

The present invention relates in general, to a composition suitable for use in inducing anti-HIV-1 antibodies, and, in particular, to immunogenic compositions comprising envelope proteins and nucleic acids to induce cross-reactive neutralizing antibodies and increase their breadth of coverage. The invention also relates to methods of inducing such broadly neutralizing anti-HIV-1 antibodies using such compositions.

### BACKGROUND

The development of a safe and effective HIV-1 vaccine is one of the highest priorities of the scientific community working on the HIV-1 epidemic. While anti-retroviral treatment (ART) has dramatically prolonged the lives of HIV-1 infected patients, ART is not routinely available in developing countries.

### SUMMARY OF THE INVENTION

In certain embodiments, the invention provides compositions and methods for induction of an immune response, for example cross-reactive (broadly) neutralizing (bn) Ab induction.

In certain aspects the invention provides a recombinant protein or nucleic acid encoding a recombinant protein as described in Table 1. In certain aspects the invention provides a selection of HIV-1 envelopes for use as prime and boost immunogens in methods to induce HIV-1 neutralizing antibodies. In certain aspects the invention provides a selection of HIV-1 envelopes for use a boost immunogen in methods to induce HIV-1 neutralizing antibodies.

In certain aspects, one or more of the mutations as described in Table 1 can be incorporated into any HIV-1 envelope sequences from the CH848 infected individual and variants thereof. See e.g., US2020/0113997 incorporated herein by reference in its entirety including FIGS. 40A-C, 41A-41C, 44A-D, 45, 46, 47A, 49A-B, 50A-D, 51, 52A-B, 53A, 53D, 54A-F, 77A-L, and 78A-B and SEQ ID NOs disclosed therein. In some embodiments, one or more of the mutations as described in Table 1 can be incorporated into envelope CH848.3.D0949.10.17 (also referred to as CH848.d0949.10.17WT) and variants thereof, including, but not limited to, CH848.d0949.10.17 DT (also referred to as CH848.d0949.10.17.N133D.N138T). In some embodiments, one or more of the mutations as described in Table 1 can be incorporated into envelope >CH848.3.D0949.10.17chim.6R.DS.SOSIP.664 as provided in FIG. 52. In some embodiments, one or more of the mutations as described in Table 1 can be incorporated into envelope CH848.d0808.15.15 and variants thereof. In some embodiments, one or more of the mutations as described in Table 1 can be incorporated into envelope CH848.d0358.80.06 and variants thereof. In some embodiments, one or more of the mutations as described in Table 1 can be incorporated into envelope CH848.d1432.5.41 and variants thereof. In some embodiments, one or more of the mutations as described in Table 1 can be incorporated into envelope CH848.d1621.4.44 and variants thereof. In some embodiments, one or more of the mutations as described in Table 1 can be incorporated into envelope CH848.d1305.10.35 and variants thereof.

In certain aspects the invention provides a selection of a series of immunogens and immunogen designs for induction of neutralizing HIV-1 antibodies, e.g. but not limited to V3 glycan epitope targeting antibodies, the selection comprising envelopes as follows: 1) CH848.d0949.10.17 DT (also referred to as CH848.d0949.10.17.N133D.N138T), 2) CH848.d0949.10.17 (also referred to as CH848.d0949.10.17WT), 3) CH848.d0808.15.15, 4) CH848.d0358.80.06, 5) CH848.d1432.5.41, 6) CH848.d1621.4.44 and 7) CH848.d1305.10.35 (see Tables 3 and 4), wherein one or more of the mutations as described in Table 1 or Table 2B can be incorporated into one or more of the envelopes of the selection. In some embodiments, the selection further comprises any HIV-1 envelope sequence from the CH848 infected individual and variants thereof comprising one or more of the mutations as described in Table 1 or Table 2B. In some embodiments the selection comprises additional HIV-1 Envs, P0402.c2.11 and ZM246F.

In certain aspects the invention provides a selection of a series of immunogens and immunogen designs for induction of neutralizing HIV-1 antibodies, e.g. but not limited to V3 glycan epitope targeting antibodies, the selection comprising envelopes as follows: 1) CH848.d0949.10.17 DT (also referred to as CH848.d0949.10.17.N133D.N138T), 2) CH848.d0949.10.17 (also referred to as CH848.d0949.10.17WT), 3) CH848.d0808.15.15, 4) CH848.d0358.80.06, 5) CH848.d1432.5.41, 6) CH848.d1621.4.44, 7) CH848.d1305.10.35, (see Tables 3 and 4) and 8) any HIV-1 envelope sequence from the CH848 infected individual and variants thereof comprising one or more of the mutations as described in Table 1 or Table 2B. In some embodiments the selection comprises additional HIV-1 Envs, P0402.c2.11 and ZM246F.

In certain embodiments, the methods use compositions comprising HIV-1 envelope immunogens designed to bind to precursors, and/or unmutated common ancestors (UCAs) of different HIV-1 bnAbs. In certain embodiments, these are UCAs of VIV2 glycan and V3 glycan binding antibodies. Thus, in certain embodiments the invention provides HIV-1 envelope immunogen designs with multimerization and variable region sequence optimization for enhanced UCA-targeting. In certain embodiments the invention provides HIV-1 envelope immunogen designs with multimerization and variable region sequence optimization for enhanced targeting and inductions of multiple antibody lineages, e.g. but not limited to V3 lineage, VIV2 lineages of antibodies.

In certain aspects the invention provides compositions comprising a selection of HIV-1 envelopes and/or nucleic acids encoding these envelopes as described herein for example but not limited to designs as described herein. Without limitations, these selected combinations comprise envelopes which provide representation of the sequence (genetic) and antigenic diversity of the HIV-1 envelope variants which lead to the induction of V1V2 glycan and V3 glycan antibody lineages.

In certain aspects the invention provides compositions comprising recombinant HIV-1 envelopes and/or nucleic acids encoding these envelopes with modifications at position 300 (HXB2 numbering).

The autologous envelope sequence changed during infection in the CH848 individual leading to mutation of G300 to N300 at a timepoint coinciding with the appearance of the DH270 clonal lineage. The G300N substitution stabilizes the GDIR/K motif in turn stabilizing the DH270 antibody interaction. Later, as the antibody matured, envelope sequences acquired mutations at this site that likely eliminated this stabilization, resulting in subsequent mutations in the DH270 clone to accommodate this lack of stability. The G300N mutation also plays a role in stabilizing the loop in which it resides, through hydrogen bonding of its side chain with the Pro299 main chain and the Asn302 side chain. Thus, it likely has an impact on the presentation of glycan 301. By disrupting or bolstering these interactions, glycan 301 presentation can be modulated. See e.g., Example 1.

In certain embodiments, the invention provides a recombinant HIV-1 envelope sequence or nucleic acid encoding a recombinant protein HIV-1 envelope sequence comprising a mutation of amino acid residue N300. In certain embodiments the mutation is N300G, N300A, N300R, N300K or N300D. In certain embodiments, the envelope further comprises a mutation at position N302. In certain embodiments, the mutation is N302D. In certain embodiments, the envelope comprises mutations N300G and N302D. In certain embodiments, the envelope comprises mutations N300A and N302D. In certain embodiments, the envelope comprises mutations N300R and N302D. In certain embodiments, the envelope comprises mutations N300K and N302D. In certain embodiments, the envelope comprises mutations N300D and N302D. The amino acid numbering position is with respect to HXB2 envelope sequence.

In certain aspects the invention provides compositions comprising recombinant HIV-1 envelopes and/or nucleic acids encoding these envelopes with modifications at position 417 (HXB2 numbering).

HIV-1 envelope position Q417 interacts with CDRH3 Y105. In the improved cryo-EM structure with DH270.6, this interaction is relatively clear. See Example 1. Therefore, modifying the envelope at position 417 with bulkier substitutions can enhance the interaction between Q417 and CDRH3 Y105 and simultaneously modulate the CDRH3 interaction with N332 glycan.

In certain embodiments, the invention provides a recombinant HIV-1 envelope sequence or nucleic acid encoding a recombinant protein HIV-1 envelope sequence comprising a mutation of amino acid residue Q417. In certain embodiments the mutation is Q417Y, Q417R or Q417K. The amino acid numbering position is with respect to HXB2 envelope sequence.

In certain aspects the invention provides compositions comprising recombinant HIV-1 envelopes and/or nucleic acids encoding these envelopes with modifications at position 330 (HXB2 numbering).

H330 interacts with CDRH3 Y105 and also with glycan 332 and Q328. Therefore, the envelope with H330A can alter the geometry of the N332 bearing loop and can modulate antibody (Ab) recognition. The bulkier substitution at position 330 can alter interactions to varying degrees.

In certain embodiments, the invention provides a recombinant HIV-1 envelope sequence or nucleic acid encoding a recombinant protein HIV-1 envelope sequence comprising a mutation of amino acid residue H330. In certain embodiments, the mutation is H330A, H330W, H330Y, H330N, H330Q or H330R. The amino acid numbering position is with respect to HXB2 envelope sequence.

In certain aspects the invention provides compositions comprising recombinant HIV-1 envelopes and/or nucleic acids encoding these envelopes with modifications at position 415 (HXB2 numbering).

HIV-1 envelope position 415 can modulate glycan 332 interaction.

In certain embodiments, the invention provides a recombinant HIV-1 envelope sequence or nucleic acid encoding a recombinant protein HIV-1 envelope sequence comprising a mutation of amino acid residue T415. In certain embodiments, the mutation is T415S, T415N or T415A. The amino acid numbering position is with respect to HXB2 envelope sequence.

In certain aspects the invention provides compositions comprising recombinant HIV-1 envelopes and/or nucleic acids encoding these envelopes with modifications at position 323 (HXB2 numbering).

To optimize around glycan 301, in certain embodiments position 323 of an HIV-1 envelope is modified.

In certain embodiments, the invention provides a recombinant HIV-1 envelope sequence or nucleic acid encoding a recombinant protein HIV-1 envelope sequence comprising a mutation of amino acid residue I323. In certain embodiments, the mutation is I323N, I323Q, I323R or I323K. In certain embodiments, the envelope further comprises mutation at amino acid residue T303. In certain embodiment, the mutation is T303N. In certain embodiment, the envelope comprises mutations I323N and T303N. In certain embodiment, the envelope comprises mutations I323Q and T303N. In certain embodiment, the envelope comprises mutations I323R and T303N. In certain embodiment, the envelope comprises mutations I323K and T303N. The amino acid numbering position is with respect to HXB2 envelope sequence.

In certain aspects the invention provides compositions comprising recombinant HIV-1 envelopes and/or nucleic acids encoding these envelopes with modifications at position 444 (HXB2 numbering).

To optimize around glycan 442, in certain embodiments position 444 of an HIV-1 envelope is be modified.

In certain embodiments, the invention provides a recombinant HIV-1 envelope sequence or nucleic acid encoding a recombinant protein HIV-1 envelope sequence comprising a mutation of amino acid residue T444. In certain embodiments, the mutation is T444H or T444G. Mutation with histidine (H) can strengthen glycan 442 configuration. Mutation with glycine (G) can loosen glycan 442 configuration. The amino acid numbering position is with respect to HXB2 envelope sequence.

In certain aspects the invention provides compositions comprising recombinant HIV-1 envelopes and/or nucleic acids encoding these envelopes with modifications at positions 439 and 443 (HXB2 numbering).

An HIV-1 envelope with I439 and I444 mutations can alter conformation of loop that holds glycan 442. To optimize around glycan 442, in certain embodiments an HIV-1 envelope at positions 439 and 443 is be modified.

In certain embodiments, the invention provides a recombinant HIV-1 envelope sequence or nucleic acid encoding a recombinant protein HIV-1 envelope sequence comprising mutations of amino acid residue I439 and I443. In certain embodiments, the mutations are I439A or I439N and I443A or I443N. In certain embodiments, the envelope comprises mutations I439A and I443A. In certain embodiments, the envelope comprises mutations I439N and I443N. In certain embodiments, the envelope comprises mutations I439A and I443N. In certain embodiments, the envelope comprises mutations I439N and I443A. The amino acid numbering position is with respect to HXB2 envelope sequence.

In certain aspects the invention provides compositions comprising recombinant HIV-1 envelopes and/or nucleic acids encoding these envelopes with modifications at position 137 (HXB2 numbering).

To optimize around GDIK motif (SEQ ID NO: 1), in certain embodiments an HIV-1 envelope is modified at position 137. Changing charges around GDIK motif (SEQ ID NO: 1) might introduce a bond with G324 of the GDIK (SEQ ID NO: 1).

In certain embodiments, the invention provides a recombinant HIV-1 envelope sequence or nucleic acid encoding a recombinant protein HIV-1 envelope sequence comprising a mutation of amino acid residue K137. In certain embodiments, the mutation is K137E, K137D or K137A. The amino acid numbering position is with respect to HXB2 envelope sequence.

In certain aspects the invention provides compositions comprising recombinant HIV-1 envelopes and/or nucleic acids encoding these envelopes with modifications at position 327 (HXB2 numbering).

In certain embodiments, the invention provides a recombinant HIV-1 envelope sequence or nucleic acid encoding a recombinant protein HIV-1 envelope sequence comprising a mutation of amino acid residue K327. In certain embodiments, the mutation is K327R. The amino acid numbering position is with respect to HXB2 envelope sequence.

In certain aspects the invention provides compositions comprising recombinant HIV-1 envelopes and/or nucleic acids encoding these envelopes with modifications at any one or more of position 300, position 302, position 417, position 330, position 415, position 323, position 303, position 444, position 439, position 443, position 137, and position 327. In some embodiments, the recombinant HIV-1 envelope comprises modifications to one or more of these positions. The amino acid numbering position is with respect to HXB2 envelope sequence. In some embodiments, the recombinant HIV-1 envelope comprises modifications to all of these positions. In some embodiments, the recombinant HIV-1 envelope comprises modifications to a subset of these positions.

In certain aspects the recombinant HIV-1 envelope optionally comprises any combinations of additional modifications, such as the modifications described in Tables 2A-B. In certain aspects the invention provides a recombinant HIV-1 envelope comprising a 17 amino acid (17aa) V1 region, lacking glycosylation at position N133 and N138 (HXB2 numbering), comprising glycosylation at N301 (HXB2 numbering) and N332 (HXB2 numbering), comprising modifications wherein glycan holes are filled (D230N_H289N_P291S (HXB2 numbering)), comprising the “GDIR” (SEQ ID NO: 2) or “GDIK” (SEQ ID NO: 1), or any trimer stabilization modifications, UCA targeting modification, immunogenicity modification, d526 V1 loop, or combinations thereof, for example but not limited to those described in Tables 2A-B.

In certain aspects the invention provides compositions comprising recombinant HIV-1 envelopes and/or nucleic acids encoding these envelopes comprising the sequence of d526 V1 loop as described in Table 2B. In certain aspects, the d526 V1 loop as described in Table 2B can be incorporated into any HIV-1 envelope sequences from the CH848 infected individual and variants thereof. See e.g., US2020/0113997 incorporated herein by reference in its entirety including FIGS. 40A-C, 41A-41C, 44A-D, 45, 46, 47A, 49A-B, 50A-D, 51, 52A-B, 53A, 53D, 54A-F, 77A-L, and 78A-B and SEQ ID NOs disclosed therein. In some embodiments, the d526 V1 loop as described in Table 2B can be incorporated into envelope CH848.3.D0949.10.17 (also referred to as CH848.d0949.10.17WT) and variants thereof, including, but not limited to, CH848.d0949.10.17 DT (also referred to as CH848.d0949.10.17.N133D.N138T). In some embodiments, the d526 V1 loop as described in Table 2B can be incorporated into envelope >CH848.3.D0949.10.17chim.6R.DS.SOSIP.664 as provided in FIG. 52. In some embodiments, the d526 V1 loop as described in Table 2B can be incorporated into envelope CH848.d0808.15.15 and variants thereof. In some embodiments, the d526 V1 loop as described in Table 2B can be incorporated into envelope CH848.d0358.80.06 and variants thereof. In some embodiments, the d526 V1 loop as described in Table 2B can be incorporated into envelope CH848.d1432.5.41 and variants thereof. In some embodiments, the d526 V1 loop as described in Table 2B can be incorporated into envelope CH848.d1621.4.44 and variants thereof. In some embodiments, the d526 V1 loop as described in Table 2B can be incorporated into envelope CH848.d1305.10.35 and variants thereof. In certain aspects the invention provides compositions comprising recombinant HIV-1 envelopes and/or nucleic acids encoding these envelopes with modifications at any one or more of position 300, position 302, position 417, position 330, position 415, position 323, position 303, position 444, position 439, position 443, position 137, and position 327 (see e.g., Table 1) and comprising the sequence of d526 V1 loop as described in Table 2B. In some embodiments, the recombinant HIV-1 envelope comprises modifications to one or more of these positions. The amino acid numbering position is with respect to HXB2 envelope sequence. In some embodiments, the recombinant HIV-1 envelope comprises modifications to all of these positions and comprises the sequence of d526 V1 loop as described in Table 2B. In some embodiments, the recombinant HIV-1 envelope comprises modifications to a subset of these positions and comprises the sequence of d526 V1 loop as described in Table 2B.

In certain embodiments, the recombinant HIV-1 envelope binds to precursors, and/or UCAs of different HIV-1 bnAbs. In certain embodiments, these are UCAs of V1V2 glycan and V3 glycan antibodies. In certain embodiments the recombinant HIV-1 envelope is 19CV3. In certain embodiments the recombinant HIV-1 envelope is any one of the recombinant HIV-1 envelopes listed in Table 1 (and optionally comprising any combinations of additional modifications, such as the modifications described in Tables 2A-B) or Table 2B. In certain embodiments, the envelope is not CH848 10.17 DT variant described previously in US2020/0113997.

In certain embodiments the recombinant HIV-1 envelope is a protomer which could be comprised in a stable trimer.

In certain embodiments the envelope comprises additional mutations stabilizing the recombinant HIV-1 envelope trimer. In certain embodiments these include, but are not limited to, SOSIP mutations. In certain embodiments mutations are selected from sets F1-F14, VT1-VT8 mutations described herein, or any combination or subcombination within a set. In certain embodiments, the selected mutations are F14. In other embodiments, the selected mutations are VT8. In certain embodiments, the selected mutations are F14 and VT8 combined.

In certain embodiments, the invention provides a recombinant HIV-1 envelope of Table 1 (and optionally comprising any combinations of additional modifications, such as the modifications described in Tables 2A-B). In certain embodiments, the invention provides a nucleic acid encoding any of the recombinant HIV-1 envelopes. In certain embodiments, the nucleic acids comprise an mRNA formulated for use as a pharmaceutical composition.

In certain embodiments, the invention provides a recombinant HIV-1 envelope of Table 2B. In certain embodiments, the invention provides a nucleic acid encoding any of the recombinant HIV-1 envelopes. In certain embodiments, the nucleic acids comprise an mRNA formulated for use as a pharmaceutical composition.

In certain embodiments the inventive designs comprise specific changes (D230N_H289N_P291S (HXB2 numbering)) which fill glycan holes with the introduction of new glycosylation sites to prevent the binding of strain-specific antibodies that could hinder broad neutralizing antibody development. See Wagh, Kshitij et al. “Completeness of HIV-1 Envelope Glycan Shield at Transmission Determines Neutralization Breadth.” Cell reports vol. 25, 4 (2018): 893-908.e7. doi:10.1016/j.celrep.2018.09.087; Crooks, Ema T et al. “Vaccine-Elicited Tier 2 HIV-1 Neutralizing Antibodies Bind to Quaternary Epitopes Involving Glycan-Deficient Patches Proximal to the CD4 Binding Site.” PLoS pathogens vol. 11, 5 e1004932. 29 May 2015, doi:10.1371/journal.ppat.1004932.

In certain embodiments, CH848.d0949.10.17DT envelope comprises additional modifications D230N.H289N.P291S.E169K and is referred to as CH848.d0949.10.17 DTe. In certain embodiments, CH848.d0949.10.17 envelope comprises additional modifications D230N.H289N.P291S.E169K and is referred to as CH848.d0949.10.17WTe. In certain embodiments, CH848.d0949.10.17DT envelope comprises additional modifications referred to as CH848.0949.10.17DT.GS designs. See Table 2A. In certain embodiments, CH848.d0949.10.17DT.GS envelopes comprise additional modifications D230N.H289N.P291S.E169K. See Table 2A.

In non-limiting embodiments, the envelope in the selections for immunization are included as trimers, protein and/or mRNA. In non-limiting embodiments, the envelope in the selections for immunization are included as nanoparticles, protein and/or mRNA. The designation scNP refers to a non-limiting embodiment of a protein nanoparticle formed by sortase conjugation reaction. In non-limiting embodiments, nanoparticles comprise fusion proteins, for example ferritin-envelope fusion proteins.

In certain embodiments, the inventive designs comprise modifications, including without limitation fusion of the HIV-1 envelope with ferritin using linkers between the HIV-1 envelope and ferritin designed to optimize ferritin nanoparticle assembly.

In certain embodiments, the invention provides recombinant HIV-1 envelopes comprising Lys327 (HXB2 numbering) optimized for administration as a prime to initiate V3 glycan antibody lineage, e.g. DH270 antibody lineage.

In certain embodiments, the invention provides recombinant HIV-1 envelopes comprising Lys169 (HXB2 numbering).

In certain embodiments, the invention provides a composition comprising any one of the inventive recombinant HIV-1 envelopes, e.g., as disclosed in Table 1 (and optionally comprising any combinations of additional modifications, such as the modifications described in Tables 2A-B) or Table 2B or nucleic acid sequences encoding the same. In certain embodiments, the nucleic acid is mRNA. In certain embodiments, the mRNA is comprised in a lipid nano-particle (LNP).

In certain embodiments, the invention provides compositions comprising a nanoparticle which comprises any one of the recombinant HIV-1 envelopes of the invention, e.g., as disclosed in Table 1 (and optionally comprising any combinations of additional modifications, such as the modifications described in Tables 2A-B) or Table 2B.

In certain embodiments, the invention provides compositions comprising a nanoparticle which comprises any one of the recombinant HIV-1 envelopes of the invention, e.g., as disclosed in Table 1 (and optionally comprising any combinations of additional modifications, such as the modifications described in Tables 2A-B) or Table 2B, wherein the nanoparticle is a ferritin self-assembling nanoparticle.

In certain aspects, the invention provides a composition comprising a nanoparticle and a carrier, wherein the nanoparticle comprises trimers of any of the recombinant HIV-1 envelopes, e.g. as disclosed in Table 1 (and optionally comprising any combinations of additional modifications, such as the modifications described in Tables 2A-B) or Table 2B. In certain embodiments, the nanoparticle is a ferritin self-assembling nanoparticle. In certain embodiments, the nanoparticle comprises multimers of trimers. Provided also are method for using these compositions comprising nanoparticles.

In certain embodiments, the invention provides a method of inducing an immune response in a subject comprising administering an immunogenic composition comprising any one of the recombinant HIV-1 envelopes of the invention e.g., as disclosed in Table 1 (and optionally comprising any combinations of additional modifications, such as the modifications described in Tables 2A-B) or Table 2B, or compositions comprising these recombinant HIV-1 envelopes, in an amount sufficient to induce an immune response. In certain embodiments, the composition is administered as a prime and/or a boost. In certain embodiments, the composition is administered as a prime. In certain embodiments, the composition is administered as a boost. In certain embodiments, the composition comprises nanoparticles. In certain embodiments, methods of the invention further comprise administering an adjuvant.

In certain embodiments, the invention provides a composition comprising a plurality of nanoparticles comprising a plurality of the recombinant HIV-1 envelopes or trimers of recombinant HIV-1 envelopes of the invention, e.g., as disclosed in Table 1 (and optionally comprising any combinations of additional modifications, such as the modifications described in Tables 2A-B) or Table 2B. In non-limiting embodiments, the envelopes/trimers of the invention are multimeric when comprised in a nanoparticle. The nanoparticle size is suitable for delivery. In non-liming embodiments the nanoparticles are ferritin based nanoparticles.

In certain aspects, the invention provides nucleic acids comprising sequences encoding HIV-1 envelopes of the invention, e.g., as disclosed in Table 1 (and optionally comprising any combinations of additional modifications, such as the modifications described in Tables 2A-B) or Table 2B. In certain embodiments, the nucleic acids are DNAs. In certain embodiments, the nucleic acids are mRNAs, modified or unmodified, suitable for use any use, e.g. but not limited to use as pharmaceutical compositions. In certain aspects, the invention provides expression vectors comprising the nucleic acids of the invention.

In certain aspects, the invention provides a pharmaceutical composition comprising mRNAs encoding the inventive HIV-1 envelopes of the invention, e.g., as disclosed in Table 1 (and optionally comprising any combinations of additional modifications, such as the modifications described in Tables 2A-B) or Table 2B. In certain embodiments, these are optionally formulated in lipid nanoparticles (LNPs). In certain embodiments, the mRNAs are modified. Modifications include without limitations modified ribonucleotides, poly-A tail, 5′cap.

In certain embodiments, the nucleic acids are formulated in lipid, such as but not limited to LNPs. Non-limiting embodiments include LNPs without polyethylene glycol.

In certain aspects, the invention provides a nucleic acid encoding any of the recombinant HIV-1 envelopes and methods for their use to induce immune response in a subject in need thereof.

In certain aspects the invention provides a method of inducing an immune response comprising administering an immunogenic composition comprising a prime immunogen followed by at least one boost immunogen from Table 1 (and optionally comprising any combinations of additional modifications, such as the modifications described in Tables 2A-B) or Table 2B, wherein the boost immunogens are administered in an amount sufficient to induce an immune response. In certain embodiments, the prime is one of the CH848.0949.10.17DT, CH848.0949.10.17DTe, CH848.d0949.10.17DT.GS, or CH848.d0949.10.17DT.GS comprising additional modifications D230N.H289N.P291S.E169K designs. See Table 2A and WO2022/087031 which content is herein incorporated by reference in its entirety. In certain embodiments, the first boost is one of the CH848.0949.10.17WT, CH848.0949.10.17WTe designs. See Table 2A and WO2022/087031 which content is herein incorporated by reference in its entirety. In certain embodiments, the first boost is one of the CH848.0949.10.17DT or CH848.0949.10.17DTe designs. See Table 2A. In some embodiments, one or more of the mutations as described in Table 1 (and optionally comprising any combinations of additional modifications, such as the modifications described in Tables 2A-B) or Table 2B can be incorporated into the envelope used as the prime and/or boost. In some embodiments, the method further comprises administering an immunogenic composition comprising any HIV-1 envelope sequence from the CH848 infected individual and variants thereof comprising one or more of the mutations as described in Table 1 (and optionally comprising any combinations of additional modifications, such as the modifications described in Tables 2A-B) or Table 2B. In some embodiments, the method comprises administering an immunogenic composition comprising any HIV-1 envelope sequence from the CH848 infected individual and variants thereof comprising one or more of the mutations as described in Table 1 (and optionally comprising any combinations of additional modifications, such as the modifications described in Tables 2A-B) or Table 2B as a prime.

In certain embodiments, the methods further comprise administering a boost from Table 4, wherein the boost is CH848.0808.15.15 in any suitable form.

In certain embodiments, the methods further comprise administering a boost from Table 4, wherein the boost is CH848.0358.80.06 in any suitable form.

In certain embodiments, the methods further comprise administering a boost from Table 4, wherein the boost is CH848.1432.5.41 in any suitable form.

In certain embodiments, the methods further comprise administering a boost from Table 4, wherein the boost is CH848.1621.4.44 in any suitable form.

In certain embodiments, the methods further comprise administering a boost from Table 4, wherein the boost is CH848.1305.10.35 in any suitable form.

In certain embodiments, the methods further comprise comprising administering a boost from Table 4, wherein the boost is P0402.c2.11 (G) in any suitable form.

In certain embodiments, the methods further comprise administering a boost from Table 4, wherein the boost is ZM246F (C) in any suitable form.

In certain embodiments, the methods further comprise administering a boost from Table 1 (and optionally comprising any combinations of additional modifications, such as the modifications described in Tables 2A-B) or Table 2B, wherein the boost is an envelope from Table 1 (and optionally comprising any combinations of additional modifications, such as the modifications described in Tables 2A-B) or Table 2B in any suitable form. In certain embodiments, the envelope possesses a substitution at position 300. In certain embodiments, the substitution is an N300G mutation. In certain embodiments, the boost comprises envelope CH848.3.D0949.10.17chim.6R.DS.SOSIP.664_N300G.

In certain embodiments, the prime and/or boost immunogen are administered as a nanoparticle. In certain embodiments, the nanoparticle is a ferritin nanoparticle. In certain embodiments, the methods further comprise administering the prime and/or boost immunogen as a mRNA-LNP formulation.

In certain embodiments, the methods further comprise administering any suitable adjuvant.

### BRIEF DESCRIPTION OF THE DRAWINGS

The patent or application file contains at least one drawing executed in color. To conform to the requirements for PCT patent applications, many of the figures presented herein are black and white representations of images originally created in color.

FIGS. 1A-B. The V3-glycan targeting DH270 broadly neutralizing antibody lineage. A) (upper left) A DH270 antibody Fab bound HIV-1 Envelope ectodomain highlighting the gp120 (blue) and gp41 (orange) domains. Antibody Fab heavy and light chains (red and grey, respectively) depicted with epitope glycans (dark blue). (right) Close views of the paratope-epitope contacts from a top and side view. Dashed circles indicate locations of distinct contact regions. (lower left) Number indicators identify contact sites highlighted in the panels to the right. B) The inferred DH270 lineage phylogenetic tree depicting cryo-EM maps determined for each lineage member Fab bound to the CH848 day 949 trimer (white). The lineage member names are colored according to the Fab color of each map. Black numbering indicates locations of consequential mutations in each antibody according to sites listed in (A). Affinities for antibodies on the path the most broad and potent mature form, M6, indicated in pink. Intermediate lineage members are denoted with an I while mature antibodies are denoted with an M.

FIGS. 2A-F. Sequence alignments for the DH270 lineage. Sequence alignments at each branch point with secondary structure and mutations highlighted in bold. FIGS. 2A-F disclose SEQ ID NOS 13-46, respectively, in order of appearance.

FIG. 3. Fourier shell correlation (FSC) plots for DH270 clonal clone antibody Fab bound maps of DH270.I1, DH270.I5, DH270.UCA3+G57R, DH270.6 vs.526.25, DH270.3, DH270.4, DH270.5 and DH270.1. Gold-standard FSC curves calculated from two independently refined half-maps. The dotted line indicates FSC=0.143.

FIG. 4. Fourier shell correlation (FSC) plots for DH270 clonal clone antibody Fab bound maps of DH270.I3, DH270.I2, DH270.6, DH270.I4 and DH270.2. Gold-standard FSC curves calculated from two independently refined half-maps. The dotted line indicates FSC=0.143.

FIG. 5. Local resolution for the DH270 clonal clone antibody Fab bound structures, set two. Refined cryo-EM maps for each complex colored by local resolution.

FIG. 6. Table of Cryo-EM Data Collection and Refinement Statistics. $, Resolutions are reported according to the FSC 0.143 gold-standard criterion. @, A highly heterogeneous dataset with many 3D classes. The top two classes were refined with C1 symmetry. #, Statistics are reported for the protein residues within the complex excluding the antibody constant domains.

FIGS. 7A-E. The UCA to I5 transition. A) Lineage tree highlighting the UCA and I5 intermediate antibodies. B) Top and side views of the I5 contact site. Heavy and light chain mutation alpha-carbons are presented as spheres. C) Top view of the UCA and UCA+G57R heavy chain contacts with gp120 highlighting the shift in the antibody position and the Env gp120 V1 loop. D) Filtered map of the UCA+G57R Fab bound trimer complex (grey) overlayed with a filtered and subtracted I5 Fab bound trimer complex map (green). The location of the S27Y mutation on the maps is indicated in orange. E) (upper left) Contact angle theta and dihedral phi with positions of the centroids used for calculations indicated with spheres. (middle left) Alignment (gp120 only) of UCA and UCA+G57R bound state structures highlighting the shift in antibody disposition about the theta angle. (lower left) Alignment (gp120 only) of UCA+G57R and I5 bound state structures highlighting the shift in antibody disposition about the phi dihedral. (upper right) Antibody Fab V_{H}/V_{L} centroid distance from the gp120 epitope centroid. (lower right) Theta vs. phi plot for the UCA, UCA+G57R, and I5 structures. Point colors are according to structure heavy chain coloring. FIG. 7E discloses “GDIK” as SEQ ID NO: 1.

FIGS. 8A-C. UCA to I5 structure differences. A) Aligned gp120/gp41 domains of the DH270 UCA and VRC01 bound CH848 day 949 structures highlighting similarity between the liganded (UCA bound structure) and unliganded (VRC01 bound structure) epitope V1 loop conformations. B) The UCA+G57R map (grey) overlayed with a UCA difference map (green) highlighting V1 loop differences. FIG. 8A discloses “GDIK” as SEQ ID NO: 1. C) The UCA+G57R map (grey) overlayed with a UCA difference map (green) highlighting the shift in antibody position.

FIGS. 9A-F. The I5 to I3 and I4 branch point. A) Lineage tree highlighting the I5, I3, and I4 intermediate antibodies. B) Top views of the I3 and I4 contact sites. Heavy and light chain mutation alpha-carbons are presented as spheres. C) (left) The N332-glycan D1 arm contact with the I5 V_{H}/V_{L} cleft. (right) The N332-glycan D1 arm contact with the I3 V_{H}/V_{L} cleft. D) Aligned I5 and I3 V_{H} domains highlighting the change in elbow angle (magenta). E) Structure of the I3 intermediate at the R87T site near potential N156-glycan contacts. F) (left) Map and fit coordinates of the new N332-glycan contact formed at the I4 HCDR3 S108Y mutation site. (right) Aligned I5 and I4 V_{H} domains showing HCDR3 loop arrangements.

FIGS. 10A-L. The I5 branch point structures: I3 and I4. A) R57 displaced gp120 V1 loops of the I5, I4, and I3 bound state structures. B) Map and fit for the I3 intermediate at the R57 position. C) Alignment of the I5, I4, and I3 bound gp120/gp41 domains. D) (left) Map and fit for the I5 intermediate at the R98 and D115 position showing interactions with the N332-glycan D1 arm. (right) Map and fit for the I3 intermediate at the R98T and D115 position showing improved interactions with the N332-glycan D1 arm. E) Map and fit of the I3 Fab V_{H} showing interaction between Y27 and T98. F) Comparison of the I5 and I4 intermediate antibody bound state from alignment of each structure's gp120 domain. G) Structure of the I3 V_{L} highlighting hydrogen bonds formed by the N54 mutations. H) Aligned I5 and I4 V_{H} domains showing little difference in elbow angle. I) Map and fit for the I4 intermediate at the R57 position. R57 sidechain adjacent density is consistent with a closed V1 loop. J) Gaussian filtered I4 intermediate bound state map showing the extent of the N156-glycan density. K) Map and fit for the I4 intermediate at the R84 position showing limited density informing possible interactions with R84. L) Alignment of the I5 and I4 structures with the I4 map showing slight rearrangement in the N332-glycan adjacent loop.

FIGS. 11A-H. The I4 to mature DH270.2 and DH270.3 transition. A) Lineage tree highlighting the I4 intermediate antibody and the mature DH270.2 (M2) and DH270.3 (M3) antibodies. B) Top views of the DH270.2 and DH270.3 contact sites. Heavy and light chain mutation alpha-carbons are presented as spheres. C) (left) Alignment (gp120 only) of I4 and DH270.2 bound state structures highlighting the shift in antibody disposition (right) Alignment (gp120 only) of I4 and DH270.3 bound state structures highlighting the shift in antibody disposition. D) Theta vs. phi plot for the I4, DH270.2, and DH270.3 structures. Point colors are according to structure heavy chain coloring. E) Structure overlay of the I4 and DH270.3 N301-glycans showing their change in disposition and the location of the S27 or Y27 residues on the antibody light chain. F) Alignment (gp120 only) of UCA+G57R and DH270.3 bound state structures indicating similar bound state arrangements. G) Comparison of the LCDR3 contact site with the gp120 protein and N301-glycan between the I4 (upper) and DH270.3 (lower) structures. H) Comparison of the N332-glycan D1 arm antibody contacts between the I4 (upper) and DH270.2 (lower) structures.

FIGS. 12A-C. The I4 branch point structures: Mature antibodies DH270.2 and DH270.3. A) Map and backbone structure fits for the I4, DH270.2 (M2) and DH270.3 (M3) heavy chains V_{H} domains. B) Gaussian filtered map alignments of the I4 and DH270.3 bound maps highlighting the shift in antibody and N301-glycan positions. C) Map and fit of the DH270.3 structure in the HCDR3 region highlighting hydrogen bonding between N332-glycan GlcNac-2 and S103.

FIGS. 13A-H. The I3 to I2 and DH270.1 branch point. A) Lineage tree highlighting the I3 and I2 intermediate antibodies and the mature DH270.1 (M1) antibody. B) Top views of the I2 and DH270.1 contact sites. Heavy and light chain mutation alpha-carbons are presented as spheres. C) Alignment (gp120 only) of I3 and I2 bound state structures highlighting the shift in gp120 V1 loop arrangement and contact between the I2 R84 sidechain and the N137-glycan. D) Alignment (gp120 only) of UCA and I2 bound structures highlighting conformational similarity between the gp120 V1 loops. E) (right) Antibody Fab V_{H}/V_{L} centroid distance from the gp120 epitope centroid. (left) Theta vs. phi plot for the I3, I2, and DH270.1 structures. Point colors are according to structure heavy chain coloring. F) Side view comparison of the LCDR3 region epitope contact between I3 and I2 highlighting the shift in LCDR3 conformation (magenta arrow). G) Top view comparison of the LCDR3 region epitope contact between I3 and I2 highlighting the shift in the aromatic residue 93 rotamer (magenta arrow). H) Alignment (gp120 only) of I3 and DH270.1 bound state structures with DH270.1 map highlighting the Y106S mutation

FIGS. 14A-F. The I3 branch point: Intermediate I2 and mature DH270.1. A) Gaussian filtered maps of the 13, 12, and DH270.1 (M1) bound maps showing differences in the V1 loop conformations. B) Map and fit of the I2 bound structure showing the shift in the I2 R57 sidechain toward gp120 residue D325 of the GDIK motif (SEQ ID NO: 1). C) Map and fit of the I2 bound structure showing the position of Y110 relative to F93 and the N301-glycan base. D) Map and fit of the I2 bound structure showing the newly formed disulfide bond between C91 and the F101C site. E) DH270.1 structure near the LCDR3 mutations. F) Map and fit of the DH270.1 bound structures showing the position of the acquired hydrophobic mutations near LCDR3

FIGS. 15A-H. The I2 to I1, DH270.4, DH270.5, and DH270.6 transition. A) Lineage tree highlighting the I2 and I1 intermediate antibodies and the mature DH270.4 (M4), DH270.5 (M5), and DH270.6 (M6) antibodies. B) Top views of the I1, DH270.4, DH270.5, and DH270.6 contact sites. Heavy and light chain mutation alpha-carbons are presented as spheres. C) Structure of the I1 V_{H}/V_{L} contacts with the N332-glycan D1 arm showing the positions of the V_{L} N62H and R56W mutations. D) Structure of the DH270.4 heavy chain showing the position of possible contact between the S85F mutation and the N156-glycan. E) Structure of the DH270.5 V_{H}/V_{L} mutations proximal to the N301-glycan. F) Structure of the DH270.6 V_{H}/V_{L} mutations proximal to the N301-glycan. G) Structure of the DH270.6 V_{L} mutations proximal to the N442-glycan H) Structure of the DH270.6 V_{H} showing two different states of the gp120 V1 loop

FIGS. 16A-F. The I2 branch point: Intermediate I1 and mature antibodies DH270.4, DH270.5, and DH270.6. A) Map and fit of the I1 bound structure at the N332-glycan adjacent light chain loop showing limited density for the mutation sites. B) Map and fit of the DH270.4 (M4) bound structure showing map densities at R85 limit inspection of possible N156-glycan interactions. C) Map and fit of the DH270.5 (M5) bound structure in the region of the light chain N-terminus highlighting nearby mutations. D) Map and fit of the DH270.6 (M6) bound structure in the region of the light chain N-terminus highlighting nearby mutations. E) Map and fit of the DH270.6 (M6) bound structure showing the closed gp120 V1 loop and the shifted R57 sidechain. F) Gaussian filtered map and closed and open V1 loops of the DH270.6 bound structure showing evidence of both a closed and an open state V1

FIG. 17. Longitudinal Env evolution in CH848 at key Env sites. The left panels show the distribution of hypervariable V1 loop lengths for longitudinally sampled CH848 Envs at each time point indicated to left of each plot (TF=transmitted founder; d0134=134 days post infection). The right panels show frequencies of amino acid and glycan mutations that arise over time at structurally key Env sites. For each plot, the height of the amino acid is proportional to its frequency at that time point, and the TF amino acid, shown only in the top LOGO, is blanked out to accentuate the mutations. Charge-based color-coding of amino acids is used (red: Asp, Glu; blue: His, Arg, Lys; black: all other amino acids), and potential N-linked glycan sites are indicated by “O” and colored cyan. Env sequences are from Bonsignori, M. et al. Staged induction of HIV-1 glycan-dependent broadly neutralizing antibodies. Sci Transl Med 9 (2017). The webtool AnalyzeAlign at the Los Alamos HIV Database was used for generating sequence logos.

FIGS. 18A-E. Envelope-DH270 lineage structural coevolution. A) Timeline of antibody and Env coevolution. The upper boxes indicate whether Envs at each time point are sensitive (green) or resistant to early intermediates (upper) and later intermediates/mature lineage members. Periods of infection at which long V1 loops are dominant are shown in grey with key mutation time points are highlighted along the timeline axis. Env structures were determined for sequences isolated at days 358, 526, 836, and 949 post infection highlighted on the timeline axis with period at which the D270 lineage appeared highlighted. B) (left) Side view of a single gp120/gp41 protomer highlighting the V1 and GDIR/K motif. (right) V1 loop structures of each Env isolate. C) (left) Top view of a single gp120/gp4l protomer highlighting the V1 and GDIR/K motif. Maps and structure fits for the unliganded day 358 (middle left), unliganded day 949 (middle right), and I3 bound day 949 (right) structures. D) The I3 (left) and I2 (right) bound structures showing similarity in epitope conformation. E) The DH270.6 bound day 526 structure (left) and DH270.6 bound day 949 structure (right) showing similarity in epitope conformation.

FIGS. 19A-D. Unliganded V3-glycan epitopes of CH848 Env with various V1 loop lengths. A-D) Map and fits of the CH848 day 358, 526, 836, and 949 structures at the V3-glycan epitope. FIGS. 19A-C disclose “GDIR” as SEQ ID NO: 2 and FIG. 19D discloses “GDIK” as SEQ ID NO: 1.

FIGS. 20A-B. Env features of autologous viruses neutralized by intermediate and mature DH270 Abs. (A) Histograms of hypervariable V1 loop lengths for the group of viruses that could be neutralized by I5, I3, I2 and DH270.4, arranged from top to bottom. The bottom-most plot shows histogram of hypervariable V1 loop lengths for autologous viruses that are resistant to all above antibodies. These V1 length distributions across antibodies were statistically compared using Wilcoxon Rank Sum Test and the bottom panel shows comparisons that yielded p-values<0.05. (B) Similar to panel (A) top, except amino acid frequency are shown using logos. To highlight diversity recognition, the amino acids and N-linked glycosylation sites of the consensus form are blanked out of the LOGOs; the consensus form is written along the bottom. The TF form matched the consensus except at position 300, which was Glycine. Variants are color-coded according to which intermediate could first recognize the variant, or if the variants were predominantly found in the resistant virus group. The table below summarizes the variants recognized by each antibody at the variable Env sites. Autologous neutralization data are from Bonsignori, M. et al. Staged induction of HIV-1 glycan-dependent broadly neutralizing antibodies. Sci Transl Med 9 (2017) measured on 90 autologous Env pseudoviruses including the TF and representative longitudinal Envs sampled between 78 to 1720 days post infection.

FIGS. 21A-B. Variability in V1 loop length and sequence retains vulnerable Env motif protection. A) Structures of unliganded V3-glycan epitope Env ectodomains for Env sequences isolated from the CH848 infected individual at days 358, 526, 836, and 949 post infection. Upper panels depict a single Env gp120 (blue) protomer with the V1 loop (green) and the GDIR/K motif (red) highlighted. The middle panels present zoomed in, side view images of the V3-glycan epitope showing the conserved hydrophobic core residues in stick representation. The lower panel presents a top view of the epitope. FIG. 21A discloses “GDIR” as SEQ ID NO: 2 and “GDIK” as SEQ ID NO: 1. B) Comparison between the DH270.6 bound day 526 and day 949 trimer ectodomains showing the fit structure and maps. Green spheres in the structures identify the residue positions at which the gp120 V1 loops are poorly resolved in the maps. The magenta circle over the maps indicates the position of missing gp120 V1 loop densities. FIG. 21B discloses “GDIR” as SEQ ID NO: 2.

FIGS. 22-28 show non-limiting embodiments of nucleic acid sequences and amino acid (“aa”) sequences of Table 1. Signal peptide is underlined—the signal peptide is MGSLQPLATLYLLGMLVASVLA (SEQ ID NO: 407). The mutated codons are highlighted. FIG. 22 discloses SEQ ID NOS 47-88, respectively, in order of appearance. FIG. 23 discloses SEQ ID NOS 89-106, respectively, in order of appearance. FIG. 24 discloses SEQ ID NOS 107-142, respectively, in order of appearance. FIG. 25 discloses SEQ ID NOS 143-162, respectively, in order of appearance. FIG. 26 discloses SEQ ID NOS 163-190, respectively, in order of appearance. FIG. 27 discloses SEQ ID NOS 191-202, respectively, in order of appearance. FIG. 28 discloses SEQ ID NOS 203-214, respectively, in order of appearance.

FIGS. 29-37 show non-limiting embodiments of immunogen design strategies.

FIG. 38 shows one embodiment of a design for the production of trimeric HIV-1 Env on ferritin nanoparticles.

FIG. 39 shows non-limiting embodiments of nucleic acid and amino acid sequences of envelopes of the invention. FIG. 39 discloses SEQ ID NOS 215-216, respectively, in order of appearance.

FIG. 40 shows non-limiting embodiments of nucleic acid and amino acid sequences of envelopes of the invention. FIG. 40 discloses SEQ ID NOS 217-222, respectively, in order of appearance.

FIG. 41 shows non-limiting embodiments of the sortase design of an envelope of the invention. FIG. 41 discloses SEQ ID NOS 223-224, respectively, in order of appearance.

FIGS. 42A-B show non-limiting embodiments for sequences of the invention comprising amino acid Arg327 (K327R). In the amino acid sequences (FIG. 42B), underlined is the signal peptide and the preceding four amino acids indicate the cloning site/kozak sequence (VDTA (SEQ ID NO: 3)) neither of which that would not be part of the final recombinant protein. FIG. 42B discloses SEQ ID NOS 225-238, respectively, in order of appearance.

FIGS. 43A-B show non-limiting embodiments of sequences of the invention comprising varying linkers between the envelope and ferritin proteins. In the amino acid sequences (FIG. 43B), underlined is the signal peptide and the preceding four amino acids indicate the cloning site/kozak sequence (VDTA (SEQ ID NO: 3)) neither of which that would not be part of the final recombinant protein. FIG. 43 discloses SEQ ID NOS 239-256, respectively, in order of appearance.

FIGS. 44A-B show non-limited embodiments of designs of 19CV3 sequences. In the amino acid sequences (FIG. 44B), underlined is the signal peptide and the preceding four amino acids indicate the cloning site/kozak sequence (VDTA (SEQ ID NO: 3)) neither of which that would not be part of the final recombinant protein. FIG. 44A discloses SEQ ID NOS 257-260, respectively, in order of appearance. FIG. 44B discloses SEQ ID NOS 261-264, respectively, in order of appearance.

FIGS. 45A-B show non-limited embodiments of designs of 19CV3 sequences. Amino acids H66A_A582T_L587A are referred to JS2 or “joe2” mutations. In the amino acid sequences (FIG. 45B), underlined is the signal peptide and the preceding four amino acids indicate the cloning site/kozak sequence (VDTA (SEQ ID NO: 3)) neither of which that would not be part of the final recombinant protein. FIG. 45A discloses SEQ ID NOS 265-274, respectively, in order of appearance. FIG. 45B discloses SEQ ID NOS 275-284, respectively, in order of appearance.

FIG. 46 shows non-limiting examples of envelopes designs and sequences of 10.17 DT.GS envelope designs. FIG. 46 discloses SEQ ID NOS 285-310, respectively, in order of appearance.

FIG. 47 shows non-limiting examples of envelopes designs and sequences described in Table 3. FIG. 47 discloses SEQ ID NOS 311-318, respectively, in order of appearance.

FIG. 48 shows non-limiting examples of envelope designs and sequences described in Table 4-envelopes CH848.0808.15.15, CH848.1621.4.44, CH848.1305.10.35, P0402.c2.11 (G), ZM246F (C). FIG. 48 discloses SEQ ID NOS 319-380, respectively, in order of appearance.

FIG. 49 shows non-limiting examples of designs and sequences based on envelope CH848.0358.80.06 and CH848.1432.5.41. FIG. 49 discloses SEQ ID NOS 381-400, respectively, in order of appearance.

FIGS. 50A-C show comparison of V1-V3 sites in d358 (FIG. 50A), d949 (FIG. 50B), d1432 (FIG. 50C) Envs. V1 loop residues and GDIR/K motifs are colored light pink and orange, respectively. The distance between d358 Arg and glycan is 2.9 Å.

FIGS. 51A-B show interactions observed in the V1-V3 sites of d358 and d526 Envs. FIG. 51A shows models with zoom-in views around the V1-V3 sites with a focus on the N332-glycan. FIG. 51B show a schematic diagram of the common high-mannose sugar in HIV-1 Env and the interaction leverage against N332-glycan.

FIG. 52 shows non-limiting examples of amino acid sequences of envelopes of the invention including d926 and d1432 Envs with V1 loop substitution of d526 Env. FIG. 52 discloses SEQ ID NOS: 401-403.

FIG. 53 shows non-limiting examples of amino acid sequences of antibodies of the invention. FIG. 53 discloses SEQ ID NOS: 404-406.

### DETAILED DESCRIPTION OF THE INVENTION

The development of a safe, highly efficacious prophylactic HIV-1 vaccine is of paramount importance for the control and prevention of HIV-1 infection. A major goal of HIV-1 vaccine development is the induction of broadly neutralizing antibodies (bnAbs) (Immunol. Rev. 254: 225-244, 2013). BnAbs are protective in rhesus macaques against SHIV challenge, but as yet, are not induced by current vaccines.

The invention provides methods of using these pan bnAb envelope immunogens.

In certain aspect, the invention provides compositions for immunizations to induce lineages of broad neutralizing antibodies. In certain embodiments, there is some variance in the immunization regimen; in some embodiments, the selection of HIV-1 envelopes may be grouped in various combinations of primes and boosts, either as nucleic acids, proteins, or combinations thereof. In certain embodiments the compositions are pharmaceutical compositions which are therapeutic and/or immunogenic. In certain embodiments, the compositions comprise amounts of envelopes which are therapeutic and/or immunogenic.

In one aspect the invention provides a composition for a prime boost immunization regimen comprising any one of the envelopes described herein, or any combination thereof wherein the envelope is a prime or boost immunogen. In certain embodiments the composition for a prime boost immunization regimen comprises one or more envelopes described herein.

In certain embodiments, the compositions contemplate nucleic acid, as DNA and/or RNA, or recombinant protein immunogens either alone or in any combination. In certain embodiments, the methods contemplate genetic, as DNA and/or RNA, immunization either alone or in combination with recombinant envelope protein(s).

In some embodiments the antigens are nucleic acids, including but not limited to mRNAs which could be modified and/or unmodified. See US Pub 20180028645A1, US Pub 20170369532, US Pub 20090286852, US Pub 20130111615, US Pub 20130197068, US Pub 20130261172, US Pub 20150038558, US Pub 20160032316, US Pub 20170043037, US Pub 20170327842, each content is incorporated by reference in its entirety. mRNAs delivered in LNP formulations have advantages over non-LNPs formulations. See US Pub 20180028645A1.

In certain embodiments the nucleic acid encoding an envelope is operably linked to a promoter inserted an expression vector. In certain aspects the compositions comprise a suitable carrier. In certain aspects the compositions comprise a suitable adjuvant.

In certain embodiments the induced immune response includes induction of antibodies, including but not limited to autologous and/or cross-reactive (broadly) neutralizing antibodies against HIV-1 envelope. Various assays that analyze whether an immunogenic composition induces an immune response, and the type of antibodies induced are known in the art and are also described herein.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In certain embodiments, the envelope used in the compositions and methods of the invention is a gp160, gp150, gp145, gp140, gp120, gp4l, N-terminal deletion variants as described herein, cleavage resistant variants as described herein, or codon optimized sequences thereof. In certain embodiments the composition comprises envelopes as trimers. In certain embodiments, envelope proteins are multimerized, for example trimers are attached to a particle such that multiple copies of the trimer are attached and the multimerized envelope is prepared and formulated for immunization in a human. In certain embodiments, the compositions comprise envelopes, including but not limited to trimers as a particulate, high-density array on liposomes or other particles, for example but not limited to nanoparticles. In some embodiments, the trimers are in a well ordered, near native like or closed conformation. In some embodiments the trimer compositions comprise a homogenous mix of native like trimers. In some embodiments the trimer compositions comprise at least 85%, 90%, 95% native like trimers.

In certain embodiments the envelope is any of the forms of HIV-1 envelope. In certain embodiments the envelope is gp120, gp140, gp145 (i.e. with a transmembrane domain), or gp150. In certain embodiments, gp140 is designed to form a stable trimer. See Table 1 (and optionally comprising any combinations of additional modifications, such as the modifications described in Tables 2A-B) and Table 2B for non-limiting examples of sequence designs. In certain embodiments envelope protomers form a trimer which is not a SOSIP timer. In certain embodiment the trimer is a SOSIP based trimer wherein each protomer comprises additional modifications. In certain embodiments, envelope trimers are recombinantly produced. In certain embodiments, envelope trimers are purified from cellular recombinant fractions by antibody binding and reconstituted in lipid comprising formulations. See for example WO2015/127108 titled “Trimeric HIV-1 envelopes and uses thereof” and US2020/0002383 which content is herein incorporated by reference in its entirety. In certain embodiments the envelopes of the invention are engineered and comprise non-naturally occurring modifications.

In certain embodiments, the envelope is in a liposome. In certain embodiments the envelope comprises a transmembrane domain with a cytoplasmic tail, wherein the transmembrane domain is embedded in a liposome. In certain embodiments, the nucleic acid comprises a nucleic acid sequence which encodes a gp120, gp140, gp145, gp150, or gp160.

In certain embodiments, where the nucleic acids are operably linked to a promoter and inserted in a vector, the vector is any suitable vector. Non-limiting examples include, VSV, replicating rAdenovirus type 4, MVA, Chimp adenovirus vectors, pox vectors, and the like. In certain embodiments, the nucleic acids are administered in NanoTaxi block polymer nanospheres. In certain embodiments, the composition and methods comprise an adjuvant. Non-limiting examples include, 3M052, AS01 B, AS01 E, gla/SE, alum, Poly I poly C (poly IC), polyIC/long chain (LC) TLR agonists, TLR7/8 and 9 agonists, or a combination of TLR7/8 and TLR9 agonists (see Moody et al. (2014) J. Virol. March 2014 vol. 88 no. 6 3329-3339), or any other adjuvant. Non-limiting examples of TLR7/8 agonist include TLR7/8 ligands, Gardiquimod, Imiquimod and R848 (resiquimod). A non-limiting embodiment of a combination of TLR7/8 and TLR9 agonist comprises R848 and oCpG in STS (see Moody et al. (2014) J. Virol. March 2014 vol. 88 no. 6 3329-3339). In non-limiting embodiments, the adjuvant is an LNP. See e.g., without limitation Shirai et al. “Lipid Nanoparticle Acts as a Potential Adjuvant for Influenza Split Vaccine without Inducing Inflammatory Responses” Vaccines 2020, 8, 433; doi:10.3390/vaccines8030433, published 3 Aug. 2020.

In non-limiting embodiments, LNPs used as adjuvants for proteins or mRNA compositions are composed of an ionizable lipid, cholesterol, lipid conjugated with polyethylene glycol, and a helper lipid. Non-limiting embodiments include LNPs without polyethylene glycol.

In certain aspects the invention provides a cell comprising a nucleic acid encoding any one of the envelopes of the invention suitable for recombinant expression. In certain aspects, the invention provides a clonally derived population of cells encoding any one of the envelopes of the invention suitable for recombinant expression. In certain aspects, the invention provides a stable pool of cells encoding any one of the envelopes of the invention suitable for recombinant expression.

In certain aspects, the invention provides a recombinant HIV-1 envelope polypeptide as described here, wherein the polypeptide is a non-naturally occurring protomer designed to form an envelope trimer. The invention also provides nucleic acids encoding these recombinant polypeptides. Non-limiting examples of amino acids and nucleic acid of such protomers are disclosed herein.

In certain aspects the invention provides a recombinant trimer comprising three identical protomers of an envelope. In certain aspects the invention provides an immunogenic composition comprising the recombinant trimer and a carrier, wherein the trimer comprises three identical protomers of an HIV-1 envelope as described herein. In certain aspects the invention provides an immunogenic composition comprising nucleic acid encoding these recombinant HIV-1 envelope and a carrier.

Described herein are nucleic and amino acids sequences of HIV-1 envelopes. The sequences for use as immunogens are in any suitable form. In certain embodiments, the described HIV-1 envelope sequences are gp160s. In certain embodiments, the described HIV-1 envelope sequences are gp120s. Other sequences, for example but not limited to stable SOSIP trimer designs, gp145s, gp140s, both cleaved and uncleaved, gp140 Envs with the deletion of the cleavage (C) site, fusion (F) and immunodominant (I) region in gp41-named as gp140ΔCFI (gp140CFI), gp140 Envs with the deletion of only the cleavage (C) site and fusion (F) domain—named as gp140ΔCF (gp140CF), gp140 Envs with the deletion of only the cleavage (C)—named gp140ΔC (gp140C) (See e.g. Liao et al. Virology 2006, 353, 268-282), gp150s, gp41s, are readily derived from the nucleic acid and amino acid gp160 sequences. In certain embodiments the nucleic acid sequences are codon optimized for optimal expression in a host cell, for example a mammalian cell, a rBCG cell or any other suitable expression system.

An HIV-1 envelope has various structurally defined fragments/forms: gp160; gp140—including cleaved gp140 and uncleaved gp140 (gp140C), gp140CF, or gp140CFI; gp120 and gp41. A skilled artisan appreciates that these fragments/forms are defined not necessarily by their crystal structure, but by their design and bounds within the full length of the gp160 envelope. While the specific consecutive amino acid sequences of envelopes from different strains are different, the bounds and design of these forms are well known and characterized in the art.

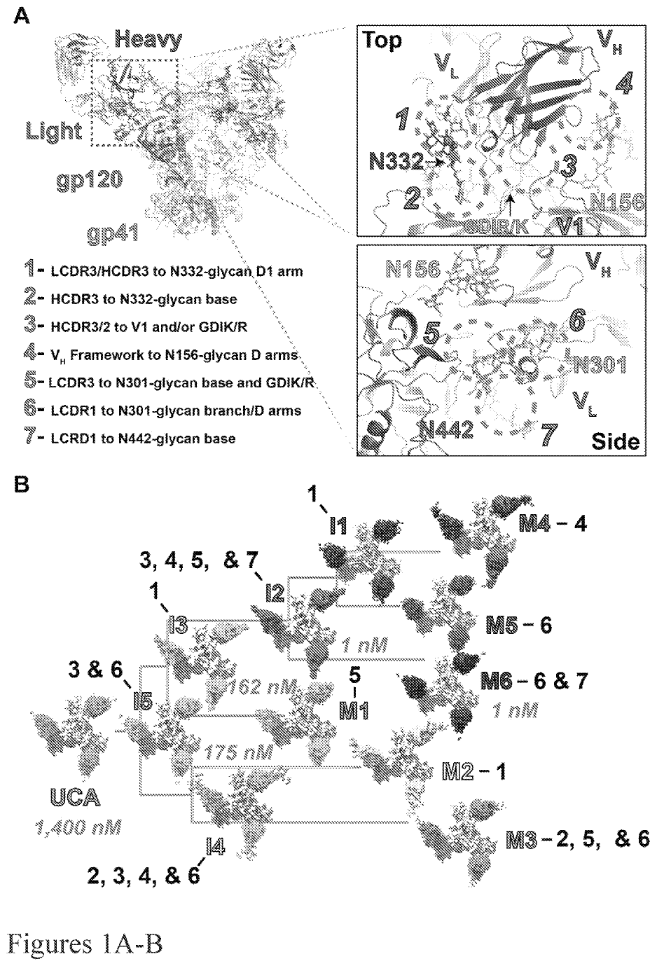

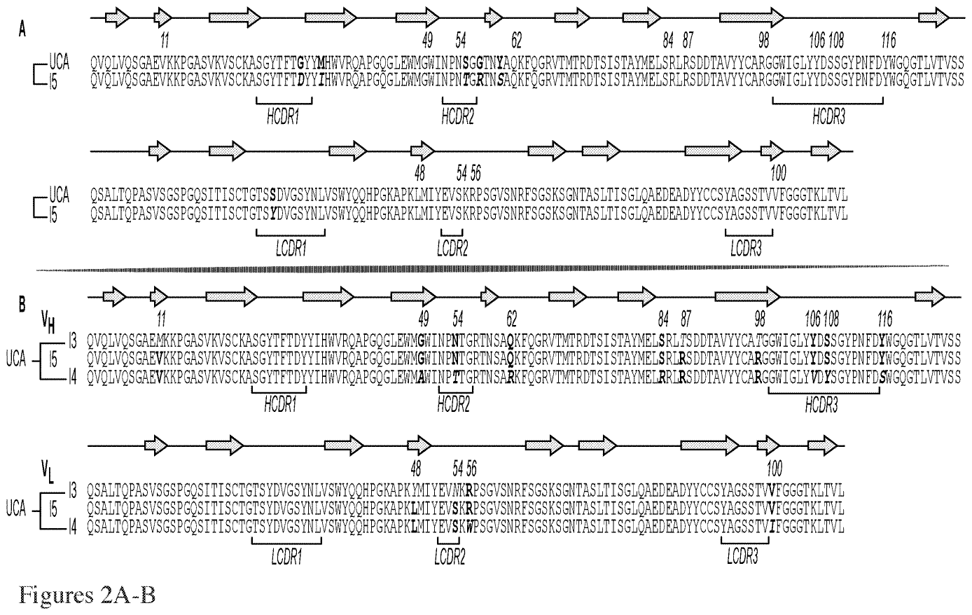

For example, it is well known in the art that during its transport to the cell surface, the gp160 polypeptide is processed and proteolytically cleaved to gp120 and gp41 proteins. Cleavages of gp160 to gp120 and gp41 occurs at a conserved cleavage site “REKR.” (SEQ ID NO: 4), See Chakrabarti et al. Journal of Virology vol. 76, pp. 5357-5368 (2002); see, e.g., FIG. 1, and second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353(2): 268-282 (2006).

The role of the furin cleavage site was well understood both in terms of improving cleavage efficiency, see Binley et al. supra, and eliminating cleavage, see Bosch and Pawlita, Virology 64 (5):2337-2344 (1990); Guo et al. Virology 174: 217-224 (1990): McCune et al. Cell 53:55-67 (1988); Liao et al. J Virol. April; 87(8):4185-201 (2013).

Likewise, the design of gp140 envelope forms is also well known in the art, along with the various specific changes which give rise to the gp140C (uncleaved envelope), gp140CF and gp140CFI forms. Envelope gp140 forms are designed by introducing a stop codon within the gp41 sequence. See Chakrabarti et al. at FIG. 1.

Envelope gp140C refers to a gp140 HIV-1 envelope design with a functional deletion of the cleavage (C) site, so that the gp140 envelope is not cleaved at the furin cleavage site. The specification describes cleaved and uncleaved forms, and various furin cleavage site modifications that prevent envelope cleavage are known in the art. In some embodiments of the gp140C form, two of the R residues in and near the furin cleavage site are changed to E, e.g., RRVVEREKR (SEQ ID NO: 5) is changed to ERVVEREKE (SEQ ID NO: 6), and is one example of an uncleaved gp140 form. Another example is the gp140C form which has the REKR (SEQ ID NO: 4) site changed to SEKS (SEQ ID NO: 7). See supra for references.

Envelope gp140CF refers to a gp140 HIV-1 envelope design with a deletion of the cleavage (C) site and fusion (F) region. Envelope gp140CFI refers to a gp140 HIV-1 envelope design with a deletion of the cleavage (C) site, fusion (F) and immunodominant (I) region in gp41. See Chakrabarti et al. Journal of Virology vol. 76, pp. 5357-5368 (2002) at for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353(2): 268-282 (2006).

In certain embodiments, the envelope design in accordance with the present invention involves deletion of residues (e.g., 5-11, 5, 6, 7, 8, 9, 10, or 11 amino acids) at the N-terminus. For delta N-terminal design, amino acid residues ranging from 4 residues or even fewer to 14 residues or even more are deleted. These residues are between the maturation (signal peptide, usually ending with CXX, wherein X is any amino acid) and “VPVXXXX . . . ”. In case of CH505 T/F Env as an example, 8 amino acids (italicized and underlined in the below sequence) were deleted: MRVMGIQRNYPQWWIWSMLGFWMLMICNGMWVTVYYGVPVWKEAKTTLFCASDA KAYEKEVHNVWATHACVPTDPNPQE . . . (rest of envelope sequence is indicated as “ . . . ”) (SEQ ID NO: 8). In other embodiments, the delta N-design described for CH505 T/F envelope is used to make delta N-designs of other envelopes. In certain embodiments, the invention relates generally to an HIV-1 envelope immunogen, gp160, gp120, or gp140, without an N-terminal Herpes Simplex gD tag substituted for amino acids of the N-terminus of gp120, with an HIV leader sequence (or other leader sequence), and without the original about 4 to about 25, for example 4, 5, 6, 7, 8, 9, 10, 11, 12, 13, 14, 15, 16, 17, 18, 19, 20, 21, 22, 23, 24, 25 amino acids of the N-terminus of the envelope (e.g. gp120). See US2014/0248311, e.g. at paragraphs [0043]-[0050], the contents of which publication is hereby incorporated by reference in its entirety.

The general strategy of deletion of N-terminal amino acids of envelopes results in proteins, for example gp120s, expressed in mammalian cells that are primarily monomeric, as opposed to dimeric, and, therefore, solves the production and scalability problem of commercial gp120 Env vaccine production. In other embodiments, the amino acid deletions at the N-terminus result in increased immunogenicity of the envelopes.

In certain aspects, the invention provides composition and methods which use a selection of Envs, as gp120s, gp140s cleaved and uncleaved, gp145s, gp150s and gp160s, stabilized and/or multimerized trimers, as proteins, DNAs, RNAs, or any combination thereof, administered as primes and boosts to elicit an immune response. Envs as proteins could be co-administered with nucleic acid vectors containing Envs to amplify antibody induction. In certain embodiments, the compositions and methods include any immunogenic HIV-1 sequences to give the best coverage for T cell help and cytotoxic T cell induction. In certain embodiments, the compositions and methods include mosaic and/or consensus HIV-1 genes to give the best coverage for T cell help and cytotoxic T cell induction. In certain embodiments, the compositions and methods include mosaic group M and/or consensus genes to give the best coverage for T cell help and cytotoxic T cell induction. In some embodiments, the mosaic genes are any suitable gene from the HIV-1 genome. In some embodiments, the mosaic genes are Env genes, Gag genes, Pol genes, Nef genes, or any combination thereof. See e.g. U.S. Pat. No. 7,951,377. In some embodiments the mosaic genes are bivalent mosaics. In some embodiments the mosaic genes are trivalent. In some embodiments, the mosaic genes are administered in a suitable vector with each immunization with Env gene inserts in a suitable vector and/or as a protein. In some embodiments, the mosaic genes, for example as bivalent mosaic Gag group M consensus genes, are administered in a suitable vector, for example but not limited to HSV2, would be administered with each immunization with Env gene inserts in a suitable vector, for example but not limited to HSV-2.

In certain aspects the invention provides compositions and methods of Env genetic immunization either alone or with Env proteins to recreate the swarms of evolved viruses that have led to bnAb induction. Nucleotide-based vaccines offer a flexible vector format to immunize against virtually any protein antigen. Currently, two types of genetic vaccination are available for testing-DNAs and mRNAs.

In certain aspects the invention contemplates using immunogenic compositions wherein immunogens are delivered as DNA. See Graham B S, Enama M E, Nason M C, Gordon I J, Peel S A, et al. (2013) DNA Vaccine Delivered by a Needle-Free Injection Device Improves Potency of Priming for Antibody and CD8+ T-Cell Responses after rAd5 Boost in a Randomized Clinical Trial. PLoS ONE 8(4): e59340, page 9. Various technologies for delivery of nucleic acids, as DNA and/or RNA, so as to elicit immune response, both T-cell and humoral responses, are known in the art and are under developments. In certain embodiments, DNA is delivered as naked DNA. In certain embodiments, DNA is formulated for delivery by a gene gun. In certain embodiments, DNA is administered by electroporation, or by a needle-free injection technology, for example but not limited to Biojector device. In certain embodiments, the DNA is inserted in vectors. The DNA is delivered using a suitable vector for expression in mammalian cells. In certain embodiments the nucleic acids encoding the envelopes are optimized for expression. In certain embodiments DNA is optimized, e.g. codon optimized, for expression. In certain embodiments the nucleic acids are optimized for expression in vectors and/or in mammalian cells. In non-limiting embodiments these are bacterially derived vectors, adenovirus based vectors, rAdenovirus (e.g. Barouch D H, et al. Nature Med. 16: 319-23, 2010), recombinant mycobacteria (e.g. rBCG or M. smegmatis) (Yu, J S et al. Clinical Vaccine Immunol. 14: 886-093, 2007; ibid 13: 1204-11, 2006), and recombinant vaccinia type of vectors (Santra S. Nature Med. 16: 324-8, 2010), for example but not limited to ALVAC, replicating (Kibler K V et al., PLoS One 6: e25674, 2011 nov 9.) and non-replicating (Perreau M et al. J. virology 85: 9854-62, 2011) NYVAC, modified vaccinia Ankara (MVA)), adeno-associated virus, Venezuelan equine encephalitis (VEE) replicons, Herpes Simplex Virus vectors, and other suitable vectors.

In certain aspects the invention contemplates using immunogenic compositions wherein immunogens are delivered as DNA or RNA in suitable formulations. Various technologies which contemplate using DNA or RNA, or may use complexes of nucleic acid molecules and other entities to be used in immunization. In certain embodiments, DNA or RNA is administered as nanoparticles consisting of low dose antigen-encoding DNA formulated with a block copolymer (amphiphilic block copolymer 704). See Cany et al., Journal of Hepatology 2011 vol. 54 j 115-121; Arnaoty et al., Chapter 17 in Yves Bigot (ed.), Mobile Genetic Elements: Protocols and Genomic Applications, Methods in Molecular Biology, vol. 859, pp 293-305 (2012); Arnaoty et al. (2013) Mol Genet Genomics. 2013 August; 288(7-8):347-63. Nanocarrier technologies called Nanotaxi® for immunogenic macromolecules (DNA, RNA, Protein) delivery are under development. See for example technologies developed by In-Cell-Art.

In certain aspects, the invention provides nucleic acids comprising sequences encoding envelopes of the invention. In certain embodiments, the nucleic acids are DNAs. In certain embodiments, the nucleic acids are mRNAs. In certain aspects, the invention provides expression vectors comprising the nucleic acids of the invention.

In certain aspects, the invention provides a pharmaceutical composition comprising mRNAs encoding the inventive antibodies. In certain embodiments, these are optionally formulated in lipid nanoparticles (LNPs). In certain embodiments, the mRNAs are modified. Modifications include without limitations modified ribonucleotides, poly-A tail, 5′cap.

In certain aspects the invention provides nucleic acids encoding the inventive envelopes. In non-limiting embodiments, the nucleic acids are mRNA, modified or unmodified, suitable for use any use, e.g. but not limited to use as pharmaceutical compositions. In certain embodiments, the nucleic acids are formulated in lipid, such as but not limited to LNPs.

In some embodiments the immunogens are administered as nucleic acids, including but not limited to mRNAs which could be modified and/or unmodified. See US Pub 20180028645A1, US Pub 20090286852, US Pub 20130111615, US Pub 20130197068, US Pub 20130261172, US Pub 20150038558, US Pub 20160032316, US Pub 20170043037, US Pub 20170327842, U.S. Pat. Nos. 10,006,007, 9,371,511, 9,012,219, US Pub 20180265848, US Pub 20170327842, US Pub 20180344838A1 at least at paragraphs [0260]-[0281], US Pub 20190153425 for non-limiting embodiments of chemical modifications, wherein each content is incorporated by reference in its entirety.

mRNAs delivered in LNP formulations have advantages over non-LNPs formulations. See US Pub 20180028645A1, US Pub 20190274968, US Pub 20180303925, wherein each content is incorporated by reference in its entirety.

In certain embodiments the nucleic acid encoding an envelope is operably linked to a promoter inserted an expression vector. In certain aspects the compositions comprise a suitable carrier. In certain aspects the compositions comprise a suitable adjuvant.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In one embodiment, the nucleic acid is an RNA molecule. In one embodiment, the RNA molecule is transcribed from a DNA sequence described herein. In some embodiments, the RNA molecule is encoded by one of the inventive sequences. In another embodiment, the nucleotide sequence comprises an RNA sequence transcribed by a DNA sequence encoding any one of the polypeptide sequence of the sequences of the invention, or a variant thereof or a fragment thereof. Accordingly, in one embodiment, the invention provides an RNA molecule encoding one or more of inventive envelopes. The RNA may be plus-stranded. Accordingly, in some embodiments, the RNA molecule is translated by cells without needing any intervening replication steps such as reverse transcription.

In some embodiments, a RNA molecule of the invention may have a 5′ cap (e.g. but not limited to a 7-methylguanosine, 7mG(5′)ppp(5′)NlmpNp). This cap can enhance in vivo translation of the RNA. The 5′ nucleotide of an RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. In some embodiments, a RNA molecule useful with the invention may be single-stranded. In some embodiments, a RNA molecule useful with the invention may comprise synthetic RNA.

In certain embodiments, the recombinant nucleic acid sequence is an optimized nucleic acid sequence. Such optimization can increase or alter the immunogenicity of the envelope. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

In certain aspects the invention contemplates using immunogenic compositions wherein immunogens are delivered as recombinant proteins. Various methods for production and purification of recombinant proteins, including trimers such as but not limited to SOSIP based trimers, suitable for use in immunization are known in the art. In certain embodiments recombinant proteins are produced in CHO cells.

It is readily understood that the envelope glycoproteins referenced in various examples and figures comprise a signal/leader sequence. It is well known in the art that HIV-1 envelope glycoprotein is a secretory protein with a signal or leader peptide sequence that is removed during processing and recombinant expression (without removal of the signal peptide, the protein is not secreted). See for example Li et al. Control of expression, glycosylation, and secretion of HIV-1 gp120 by homologous and heterologous signal sequences. Virology 204(1):266-78 (1994) (“Li et al. 1994”), at first paragraph, and Li et al. Effects of inefficient cleavage of the signal sequence of HIV-1 gp120 on its association with calnexin, folding, and intracellular transport. PNAS 93:9606-9611 (1996) (“Li et al. 1996”), at 9609. Any suitable signal sequence could be used. In some embodiments the leader sequence is the endogenous leader sequence. Most of the gp120 and gp160 amino acid sequences include the endogenous leader sequence. In other non-limiting examples, the leader sequence is human Tissue Plasminogen Activator (TPA) sequence, human CD5 leader sequence (e.g. MPMGSLQPLATLYLLGMLVASVLA (SEQ ID NO: 9)). Most of the chimeric designs include CD5 leader sequence. A skilled artisan appreciates that when used as immunogens, and for example when recombinantly produced, the amino acid sequences of these proteins do not comprise the leader peptide sequences.

The immunogenic envelopes can also be administered as a protein prime and/or boost alone or in combination with a variety of nucleic acid envelope primes (e.g., HIV-1 Envs delivered as DNA expressed in viral or bacterial vectors).

Dosing of proteins and nucleic acids are readily determined by a skilled artisan. A single dose of nucleic acid can range from a few nanograms (ng) to a few micrograms (μg) or milligram of a single immunogenic nucleic acid. Recombinant protein dose can range from a few μg micrograms to a few hundred micrograms, or milligrams of a single immunogenic polypeptide.

Administration: In certain embodiments, the compositions are formulated with appropriate carriers using known techniques to yield compositions suitable for various routes of administration. In certain embodiments the compositions are delivered via intramuscular (IM), via subcutaneous, via intravenous, via nasal, via mucosal routes, or any other suitable route of immunization.

In certain embodiments, the compositions are formulated with appropriate carriers and adjuvants using techniques to yield compositions suitable for immunization. The compositions can include an adjuvant, such as, for example but not limited to 3M052, alum, poly IC, MF-59 or other squalene-based adjuvant, ASOIB, or other liposomal based adjuvant suitable for protein or nucleic acid immunization. In certain embodiments, the adjuvant is GSK ASOIE adjuvant containing MPL and QS21. This adjuvant has been shown by GSK to be as potent as the similar adjuvant ASOIB but to be less reactogenic using HBsAg as vaccine antigen (Leroux-Roels et al., IABS Conference, April 2013). In certain embodiments, TLR agonists are used as adjuvants. In other embodiment, adjuvants which break immune tolerance are included in the immunogenic compositions.

In certain embodiments, the compositions and methods comprise any suitable agent or immune modulation which could modulate mechanisms of host immune tolerance and release of the induced antibodies. In non-limiting embodiments modulation includes PD-1 blockade; T regulatory cell depletion; CD40L hyperstimulation; soluble antigen administration, wherein the soluble antigen is designed such that the soluble agent eliminates B cells targeting dominant epitopes, or a combination thereof. In certain embodiments, an immunomodulatory agent is administered in at time and in an amount sufficient for transient modulation of the subject's immune response so as to induce an immune response which comprises broad neutralizing antibodies against HIV-1 envelope. Non-limiting examples of such agents is any one of the agents described herein: e.g. chloroquine (CQ), PTP1B Inhibitor—CAS 765317-72-4—Calbiochem or MSI 1436 clodronate or any other bisphosphonate; a Foxo1 inhibitor, e.g. 344355 Foxo1 Inhibitor, AS1842856—Calbiochem; Gleevac, anti-CD25 antibody, anti-CCR4 Ab, an agent which binds to a B cell receptor for a dominant HIV-1 envelope epitope, or any combination thereof. In non-limiting embodiments, the modulation includes administering an anti-CTLA4 antibody, OX-40 agonists, or a combination thereof. Non-limiting examples are of CTLA-1 antibody are ipilimumab and tremelimumab. In certain embodiments, the methods comprise administering a second immunomodulatory agent, wherein the second and first immunomodulatory agents are different.

### Multimeric Envelopes

Presentation of antigens as particulates reduces the B cell receptor affinity necessary for signal transduction and expansion (see Baptista et al. EMBO J. 2000 Feb. 15; 19(4): 513-520). Displaying multiple copies of the antigen on a particle provides an avidity effect that can overcome the low affinity between the antigen and B cell receptor. The initial B cell receptor specific for pathogens can be low affinity, which precludes vaccines from being able to stimulate and expand B cells of interest. In particular, very few naïve B cells from which HIV-1 broadly neutralizing antibodies arise can bind to soluble HIV-1 Envelope. Provided are envelopes, including but not limited to trimers as particulate, high-density array on liposomes or other particles, for example but not limited to nanoparticles. See, e.g. He et al. Nature Communications 7, Article number: 12041 (2016), doi:10.1038/ncomms12041; Bamrungsap et al. Nanomedicine, 2012, 7 (8), 1253-1271.

For development as a vaccine immunogen, we have also created multimeric nanoparticles that comprise and/or display HIV envelope protein or fragments on their surface.

The nanoparticle immunogens are composed of various forms of HIV-envelope protein, e.g. without limitation envelope trimer, and self-assembling protein, e.g. without limitation ferritin protein. Any suitable ferritin could be used in the immunogens of the invention. In non-limiting embodiments, the ferritin is derived from Helicobacter pylori. In non-limiting embodiments, the ferritin is insect ferritin. In non-limiting embodiments, each nanoparticle displays 24 copies of the envelope protein on its surface.

Presenting multiple copies of antigens to B cells has been a longstanding approach to improving B cell receptor recognition and antigen uptake (See Batista et al. EMBO J. 2000 Feb. 15; 19(4): 513-520). The improved recognition of antigen is due to the avid interaction of multiple antigens with multiple B cell receptors on a single B cells, which results in clustering of B cells and stronger cell signaling. Furthermore, multimeric presentation improves antigen binding to mannose binding lectin which promotes antigen trafficking to B cell follicles. Self-assembling complexes comprising multiple copies of an antigen are one strategy of immunogen design approach for arraying multiple copies of an antigen for recognition by the B cell receptors on B cells (Kanekiyo, M., Wei, C. J., Yassine, H. M., McTamney, P. M., Boyington, J. C., Whittle, J. R., Rao, S. S., Kong, W. P., Wang, L., and Nabel, G. J. (2013). Self-assembling influenza nanoparticle vaccines elicit broadly neutralizing H1N1 antibodies. Nature 499, 102-106; Ueda, G., Antanasijevic, A., Fallas, J. A., Sheffler, W., Copps, J., Ellis, D., Hutchinson, G. B., Moyer, A., Yasmeen, A., Tsybovsky, Y., et al. (2020). Tailored design of protein nanoparticle scaffolds for multivalent presentation of viral glycoprotein antigens. Elife).

In some instances, the gene of an antigen is fused via a linker/spacer to a gene of a protein which could self-assemble. Upon translation, a fusion protein is made that can self-assemble into a multimeric complex—also referred to as a nanoparticle displaying multiple copies of the antigen. In other instances, the protein antigen could be conjugated to the self-assembling protein via an enzymatic reaction, thereby forming a nanoparticle displaying multiple copies of the antigen. Non-limiting embodiments of enzymatic conjugation include without limitation sortase mediated conjugation. In some embodiments, linkers for use in any of the designs of the invention could be 2-50 amino acids long, e.g. 2, 3, 4, 5, 6, 7, 8, 9, 10, 11, 12, 13, 14, 15, 16, 17, 18, 19, 20, 25, 30, 35, 40, 45, or 50 amino acids long. In certain embodiments, these linkers comprise glycine and serine amino acid in any suitable combination, and/or repeating units of combinations of glycine, serine and/or alanine.

Ferritin is a well-known protein that self-assembles into a hollow particle composed of repeating subunits. In some species ferritin nanoparticles are composed of 24 copies of a single subunit, whereas in other species it is composed of 12 copies each of two subunits.

Non-limiting embodiments of sortase linkers could be used so long as their position allows multimerization of the envelopes. In a non-limiting embodiment, a C-terminal tag is LPXTG, where X signifies any amino acid but most commonly Ala, Ser, Glu (SEQ ID NO: 10), or a N-terminal pentaglycine repeat tag is added to the envelope trimer gene. In a non-limiting embodiment, a C-terminal tag is LPXTGG, where X signifies any amino acid but most commonly Ala, Ser, Glu (SEQ ID NO: 11).

To improve the interaction between the naïve B cell receptor and immunogens, in some embodiments, the envelope design is created so the envelope is presented on particles, e.g. but not limited to nanoparticle. In some embodiments, the HIV-1 Envelope trimer could be fused to ferritin. Ferritin protein self assembles into a small nanoparticle with three fold axis of symmetry. At these axes the envelope protein is fused. Therefore, the assembly of the three-fold axis also clusters three HIV-1 envelope protomers together to form an envelope trimer. Each ferritin particle has 8 axes which equates to 8 trimers being displayed per particle. See e.g. Sliepen et al. Retrovirology 2015 12:82, DOI: 10.1186/s12977-015-0210-4.

Any suitable ferritin sequence could be used. In non-limiting embodiments, ferritin sequences are disclosed in US2019/0330279, the content of which is hereby incorporated by reference in its entirety.

Ferritin nanoparticle linkers: The ability to form HIV-1 envelope ferritin nanoparticles relies self-assembly of 24 ferritin subunits into a single ferritin nanoparticle. The addition of a ferritin subunit to the c-terminus of HIV-1 envelope may interfere with the ability of the ferritin subunit to fold properly and or associate with other ferritin subunits. When expressed alone ferritin readily forms 24-subunit nanoparticles, however appending it to envelope only yields nanoparticles for certain envelopes. Since the ferritin nanoparticle forms in the absence of envelope, the envelope could be sterically hindering the association of ferritin subunits. Thus, ferritin can be designed with elongated glycine-serine linkers to further distance the envelope from the ferritin subunit. To make sure that the glycine linker is attached to ferritin at the correct position, constructs can be created that attach at second amino acid position or the fifth amino acid position. The first four n-terminal amino acids of natural Helicobacter pylori ferritin are not needed for nanoparticle formation but may be critical for proper folding and oligomerization when appended to envelope. Thus, in some embodiments, constructs are designed with and without the leucine, serine, and lysine amino acids following the glycine-serine linker. The goal will be to find a linker length that is suitable for formation of envelope nanoparticles when ferritin is appended to most envelopes. Any suitable linker between the envelope and ferritin could be uses, so long as the fusion protein is expressed and the trimer is formed.

Another approach to multimerize expression constructs uses staphylococcus sortase A transpeptidase ligation to conjugate inventive envelope trimers to cholesterol. The trimers can then be embedded into liposomes via the conjugated cholesterol. To conjugate the trimer to cholesterol either a C-terminal LPXTG tag (SEQ ID NO: 10) or a N-terminal pentaglycine repeat tag (SEQ ID NO: 12) is added to the envelope trimer gene. Cholesterol is also synthesized with these two tags. Sortase A is then used to covalently bond the tagged envelope to the cholesterol. The sortase A-tagged trimer protein can also be used to conjugate the trimer to other peptides, proteins, or fluorescent labels. In non-limiting embodiments, the sortase A tagged trimers are conjugated to ferritin to form nanoparticles. See FIG. 38.

The invention provides design of envelopes and trimer designs wherein the envelope comprises a linker which permits addition of a lipid, such as but not limited to cholesterol, via a sortase A reaction. See e.g. Tsukiji, S. and Nagamune, T. (2009), Sortase-Mediated Ligation: A Gift from Gram-Positive Bacteria to Protein Engineering. ChemBioChem, 10: 787-798. doi:10.1002/cbic.200800724; Proft, T. Sortase-mediated protein ligation: an emerging biotechnology tool for protein modification and immobilisation. Biotechnol Lett (2010) 32: 1. doi:10.1007/s10529-009-0116-0; Lena Schmohl, Dirk Schwarzer, Sortase-mediated ligations for the site-specific modification ofproteins, Current Opinion in Chemical Biology, Volume 22, October 2014, Pages 122-128, ISSN 1367-5931, dx.doi.org/l0.1016/j.cbpa.2014.09.020; Tabata et at. Anticancer Res. 2015 August; 35(8):4411-7; Pritz et al. J. Org. Chem. 2007, 72, 3909-3912.

The lipid modified envelopes and trimers could be formulated as liposomes. Any suitable liposome composition is contemplated.

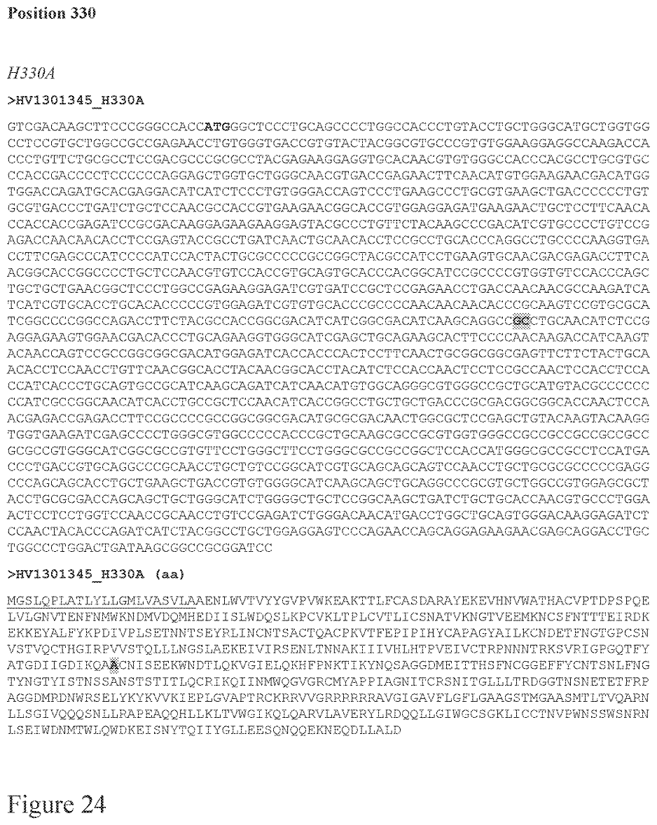

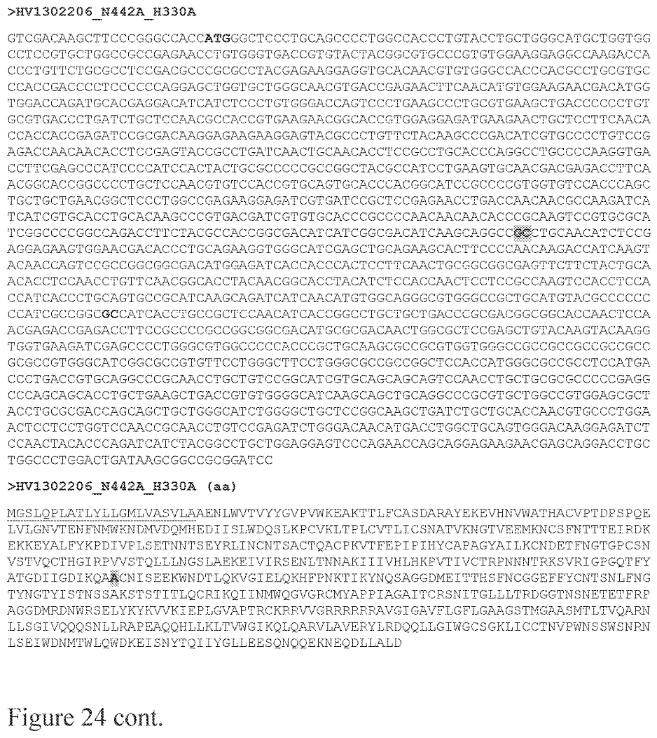

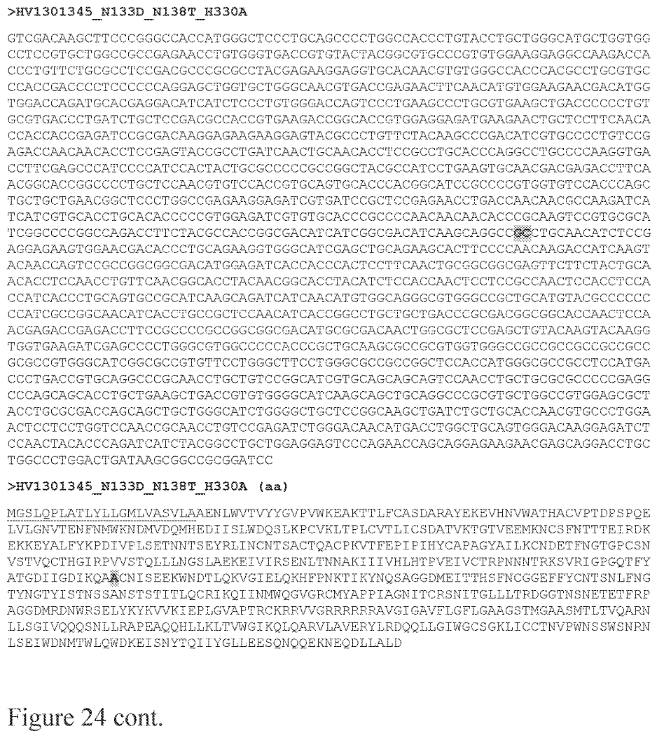

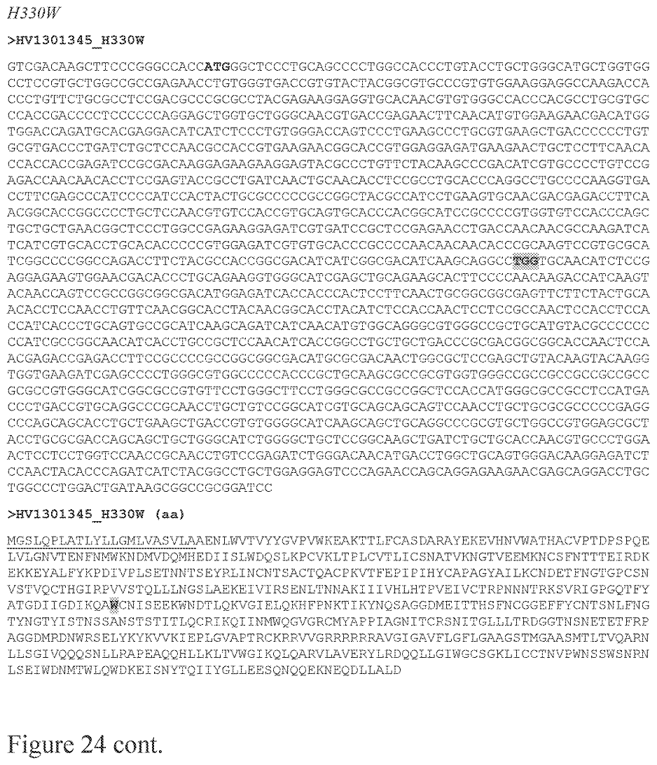

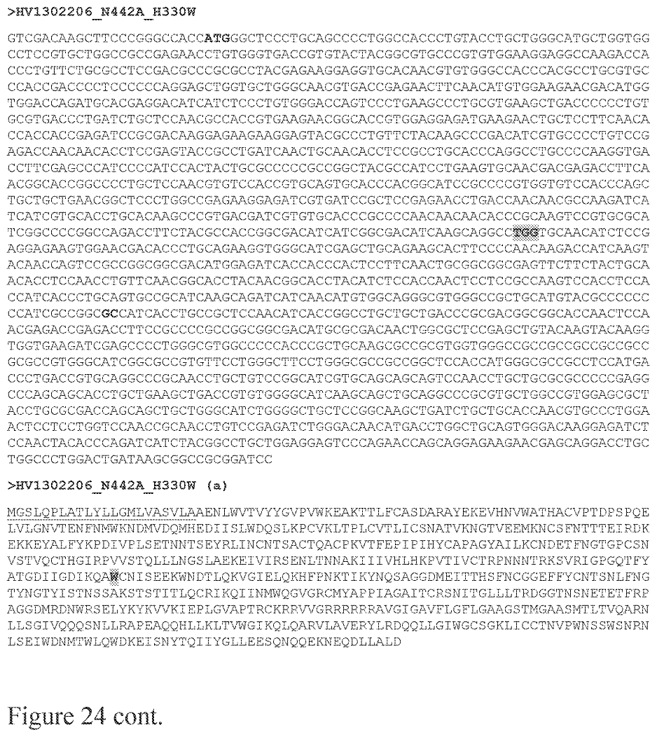

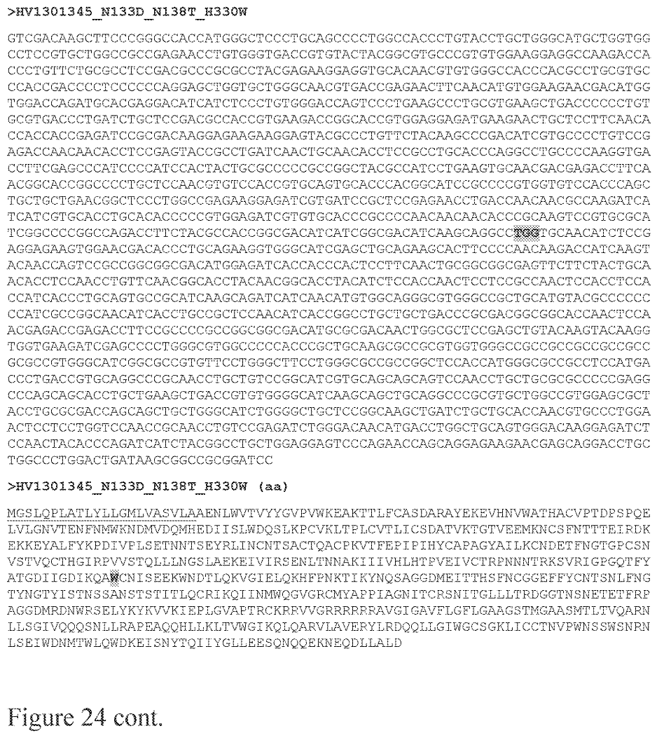

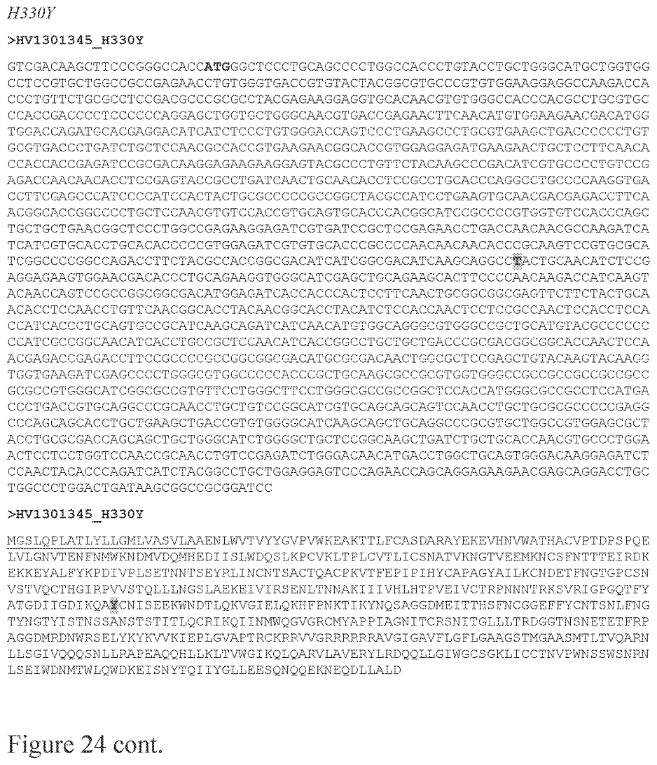

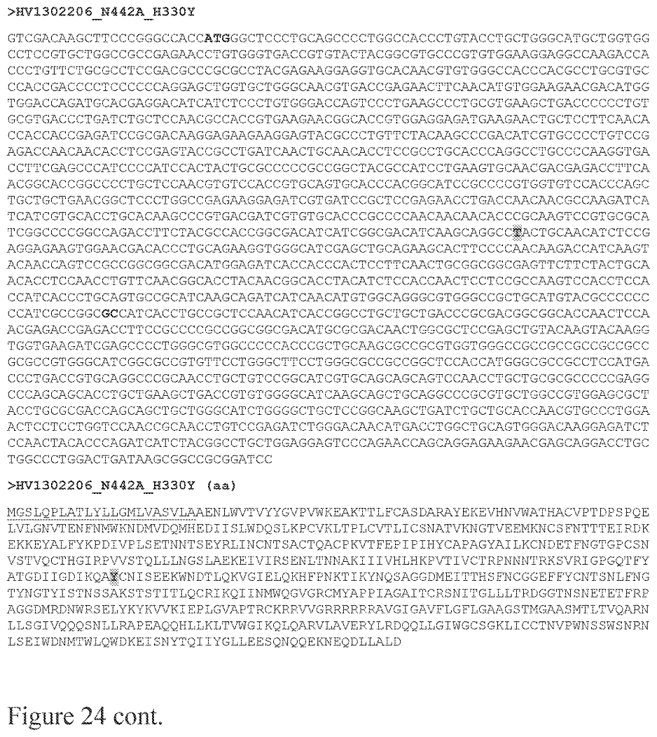

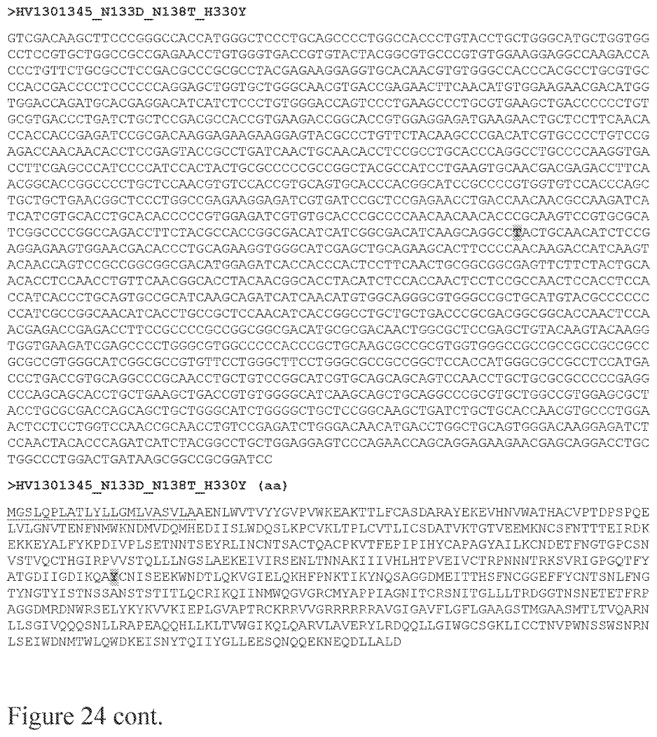

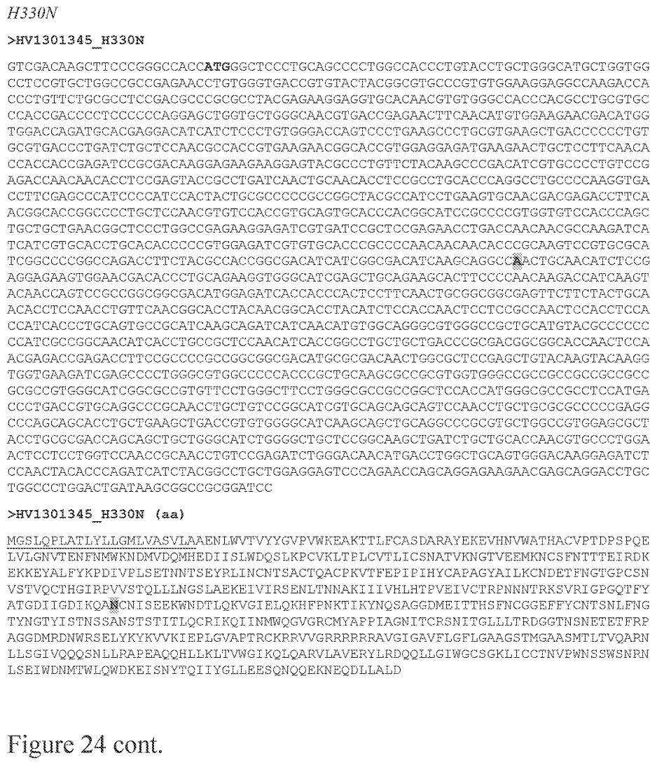

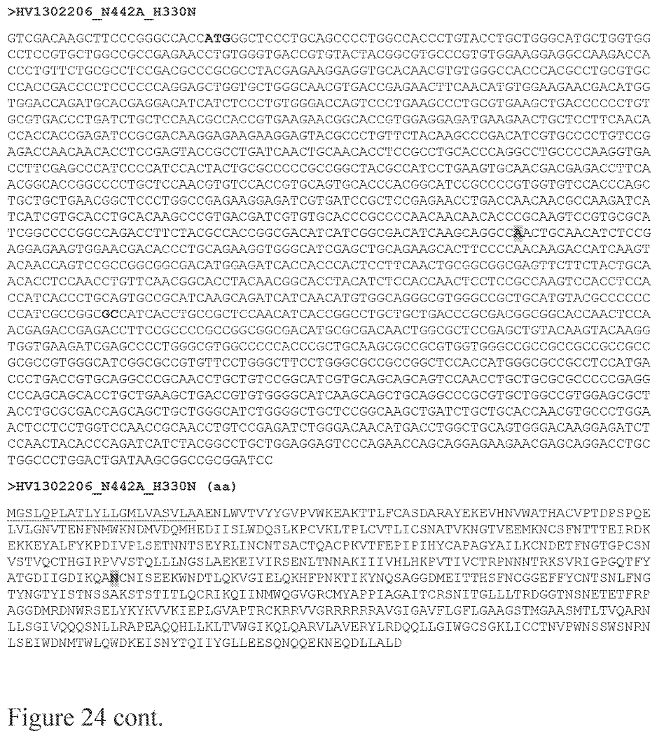

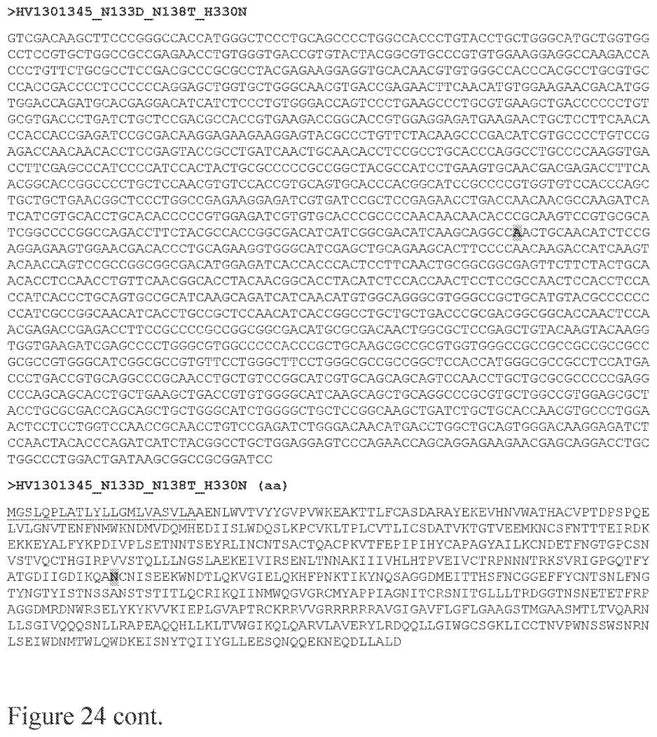

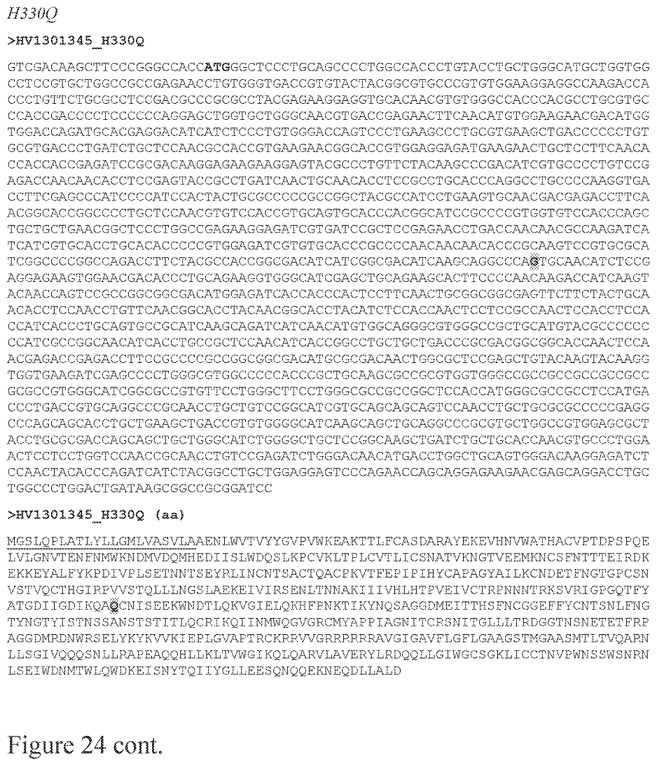

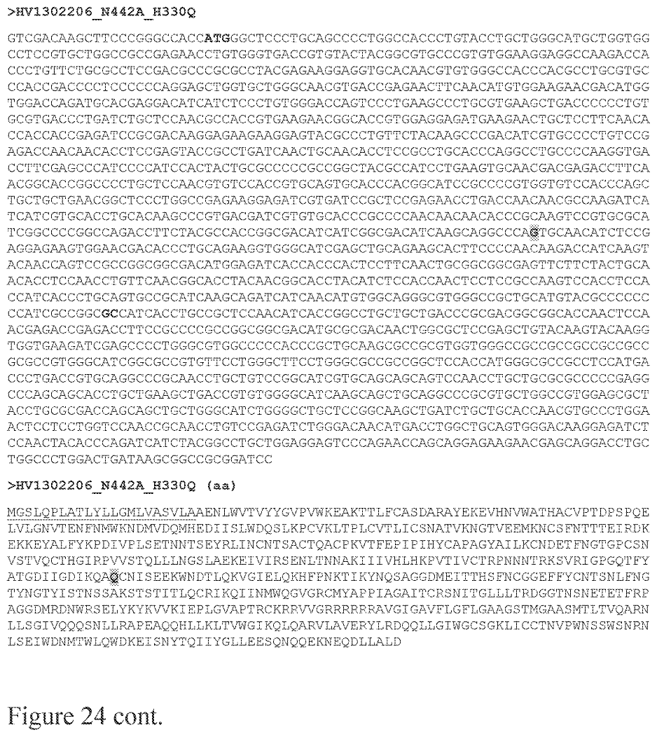

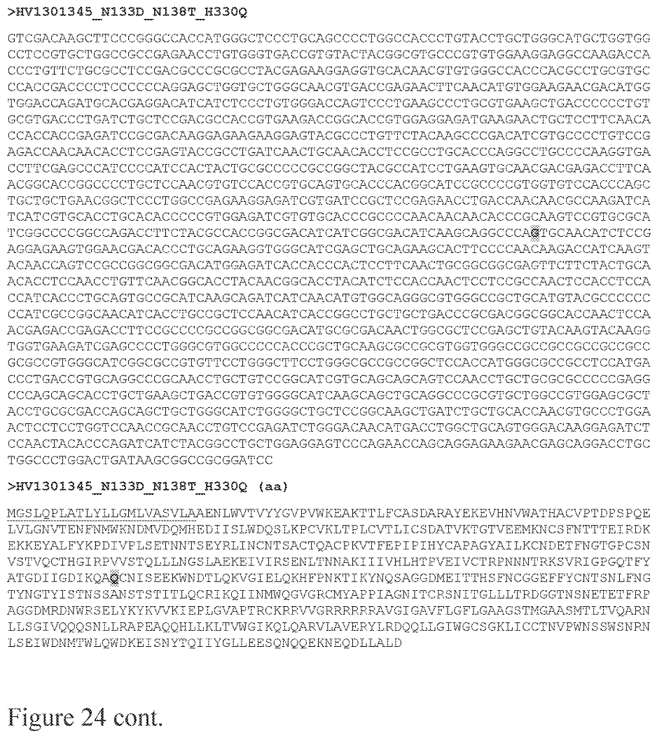

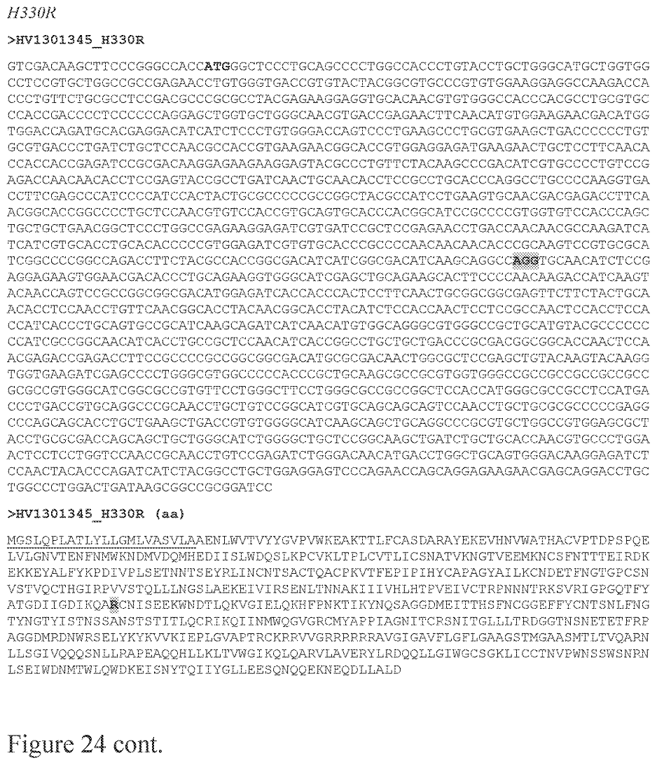

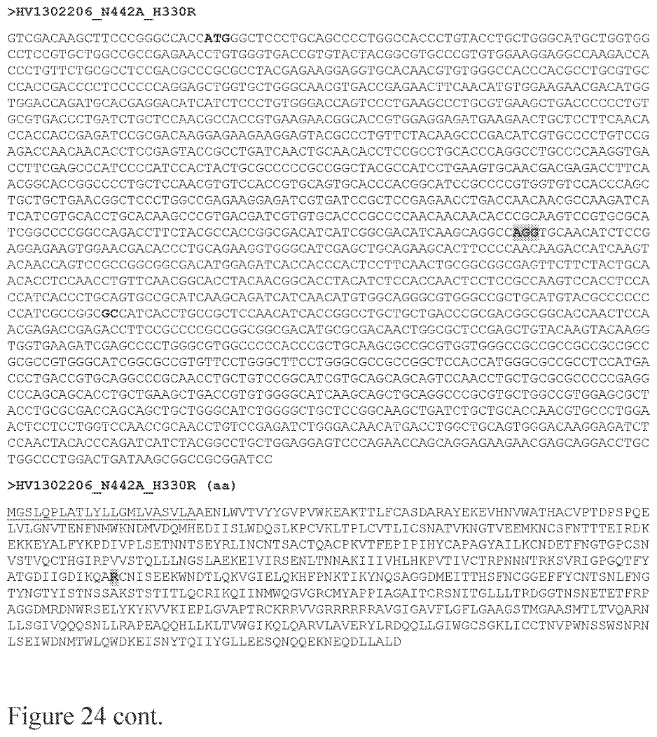

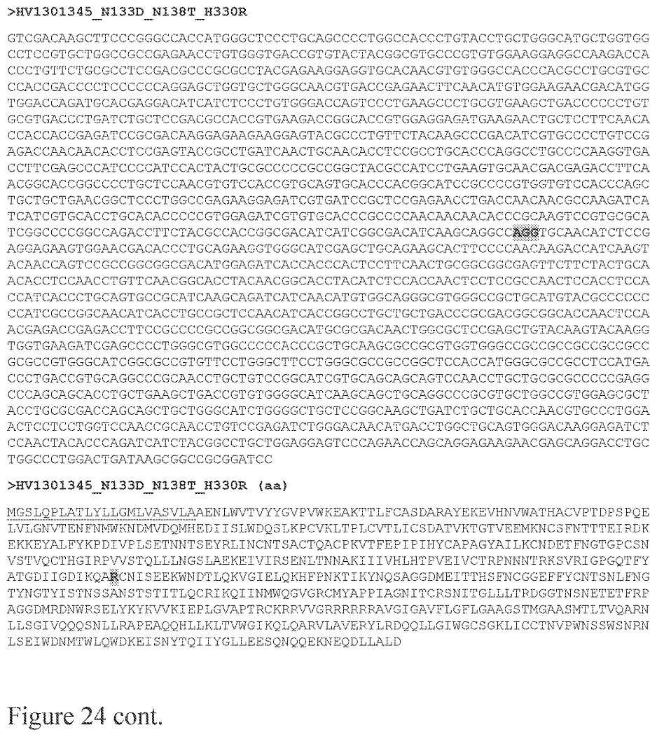

Non-limiting embodiments of envelope designs for use in sortase A reaction are shown in FIG. 24 B-D of US2020/0002383, incorporated by reference in its entirety.

Additional sortase linkers could be used so long as their position allows multimerization of the envelopes. In a non-limiting embodiment, a C-terminal tag is LPXTG, where X signifies any amino acid but most commonly Ala, Ser, Glu (SEQ ID NO: 10), or a N-terminal pentaglycine repeat tag is added to the envelope trimer gene. In a non-limiting embodiment, a C-terminal tag is LPXTGG, where X signifies any amino acid but most commonly Ala, Ser, Glu (SEQ ID NO: 11).

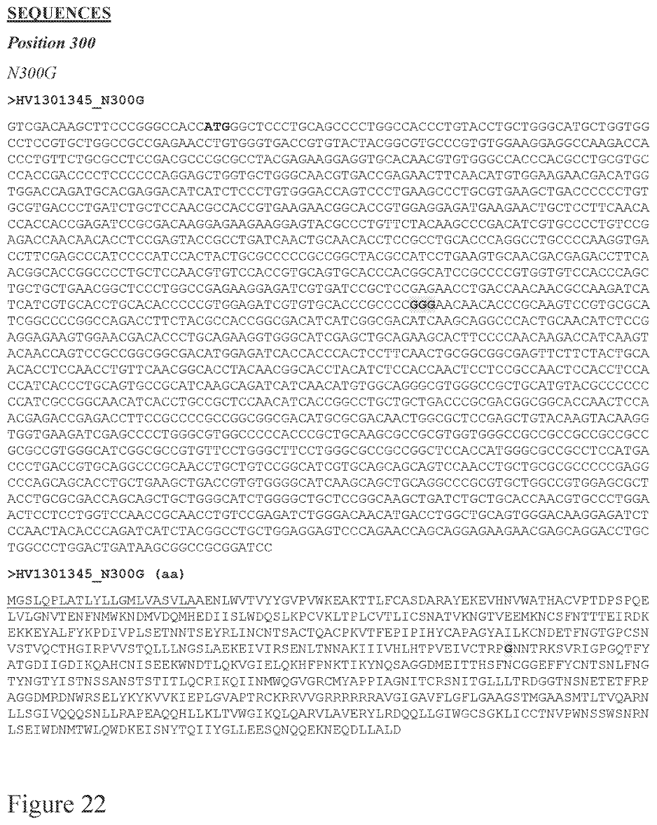

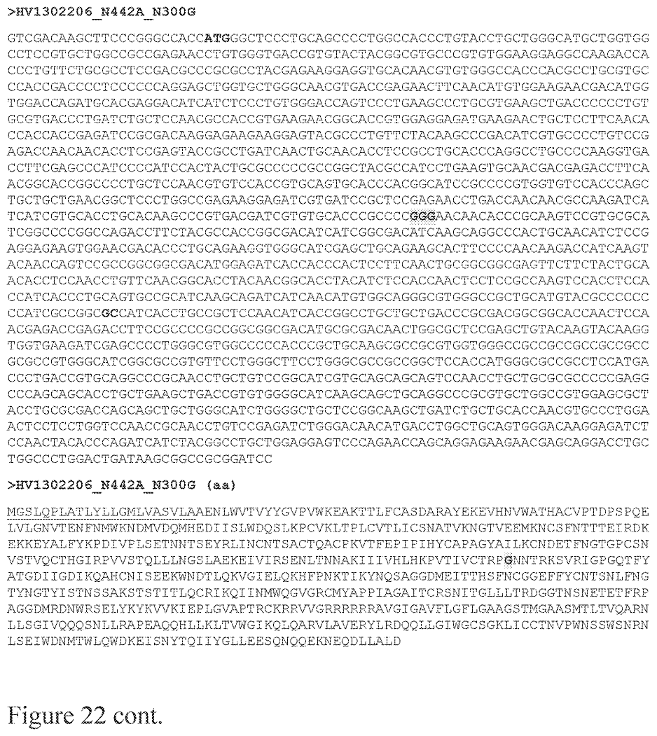

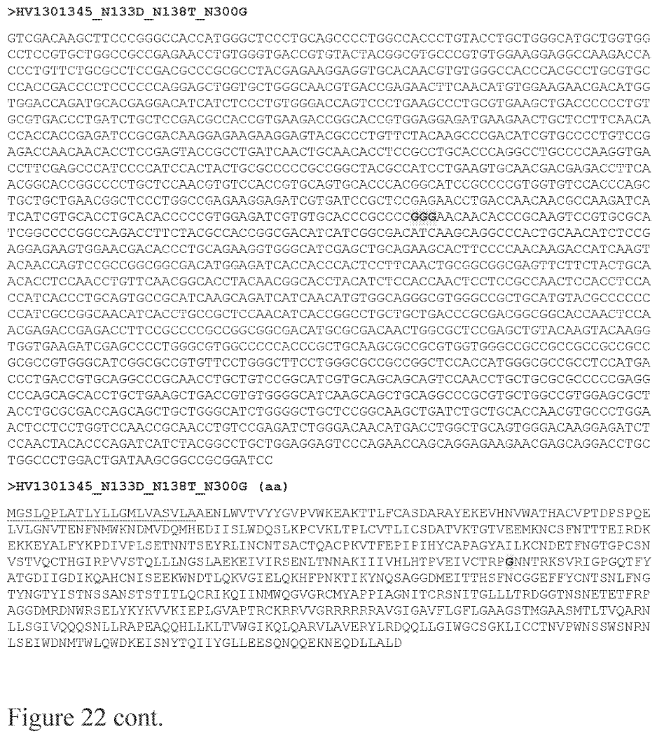

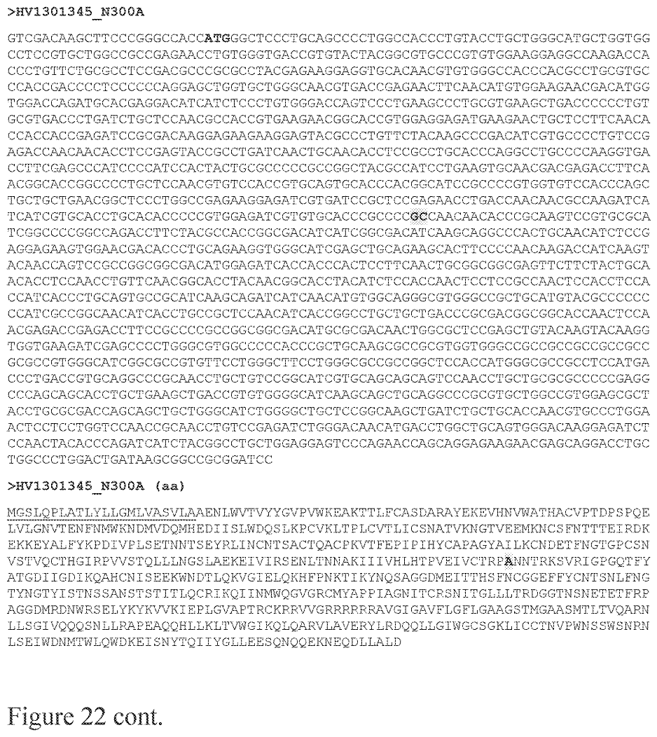

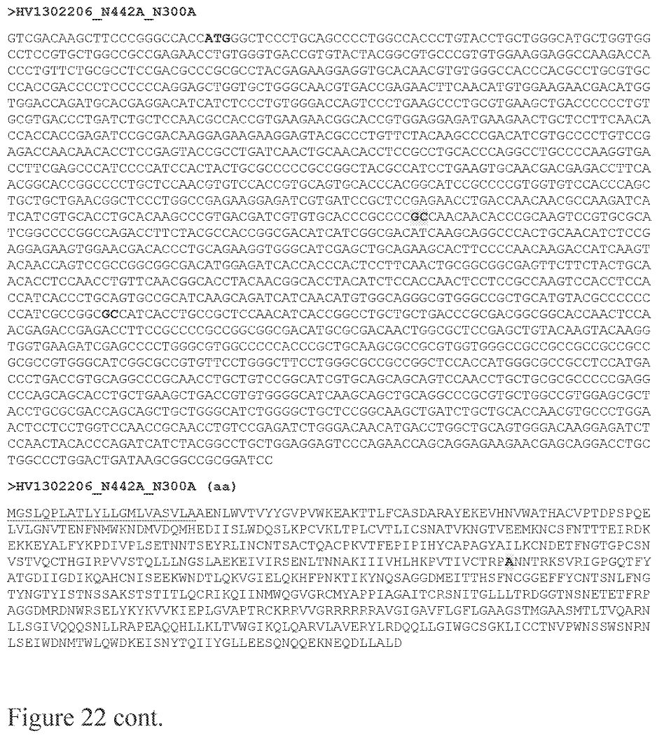

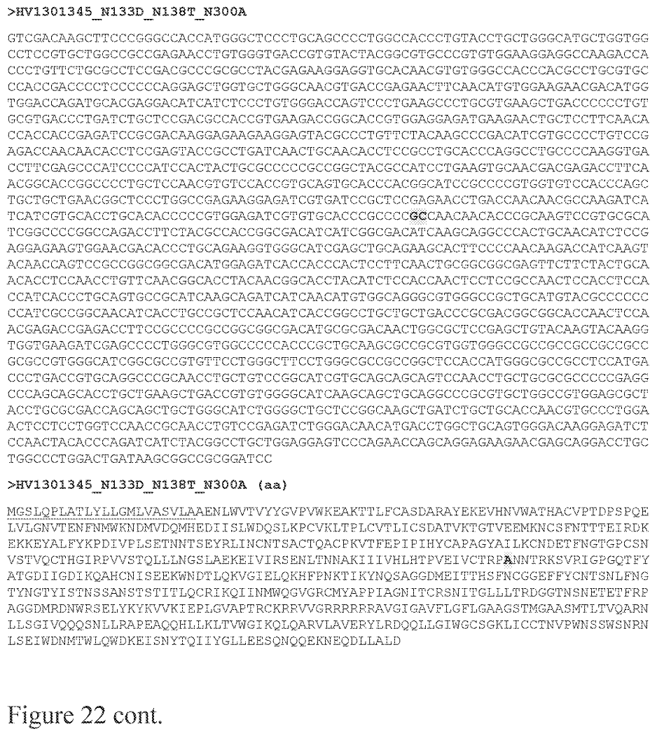

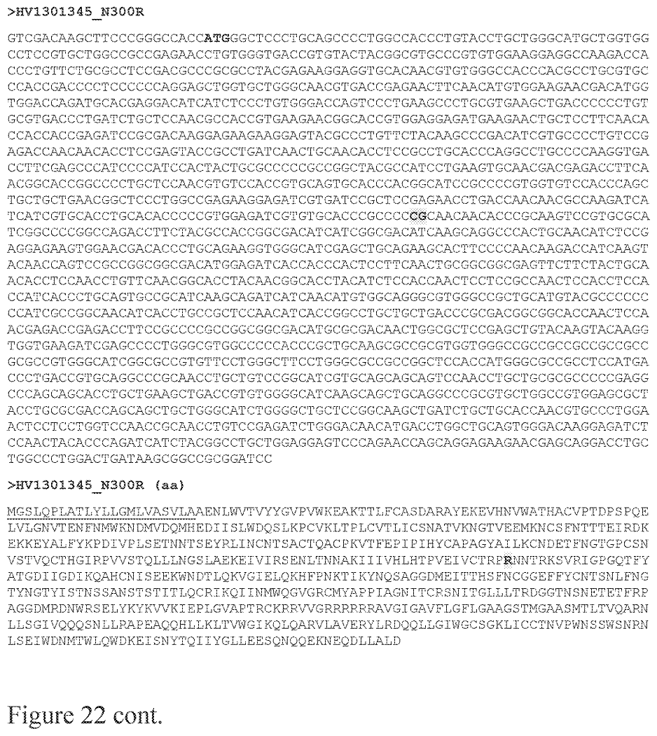

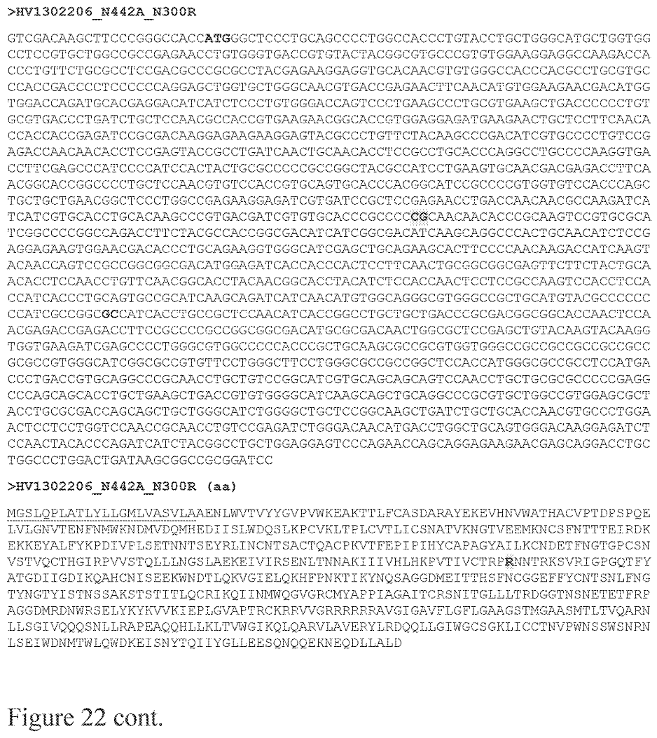

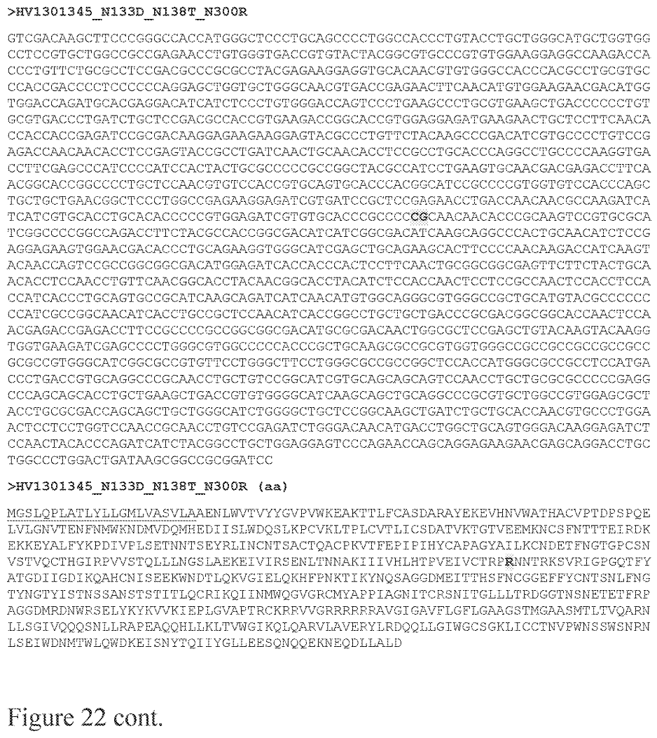

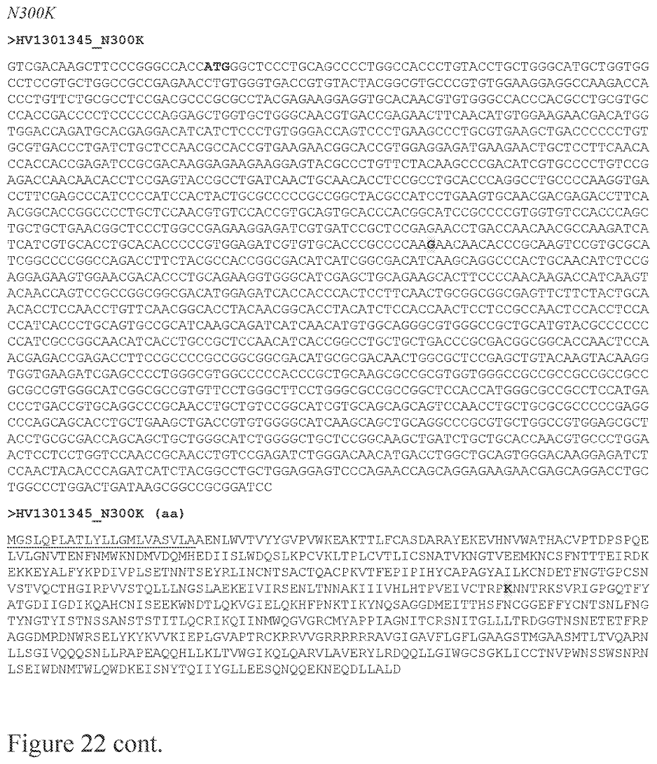

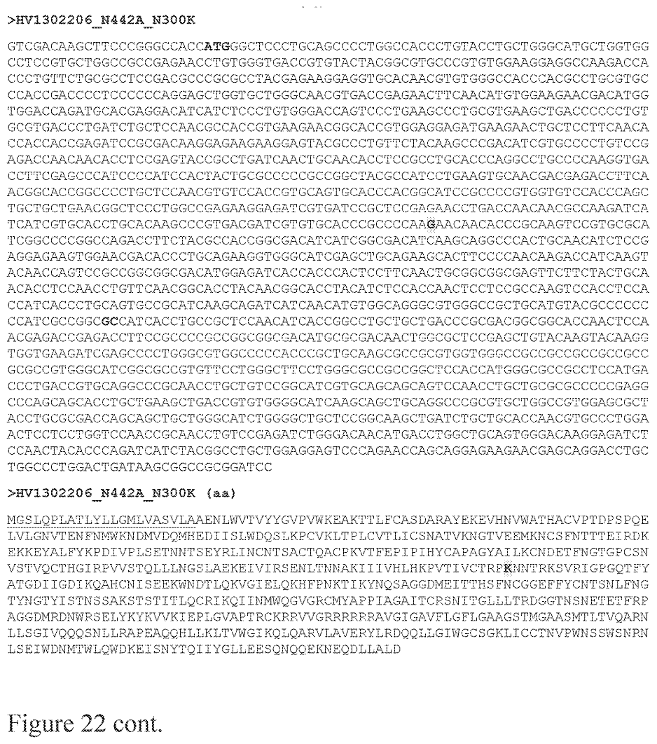

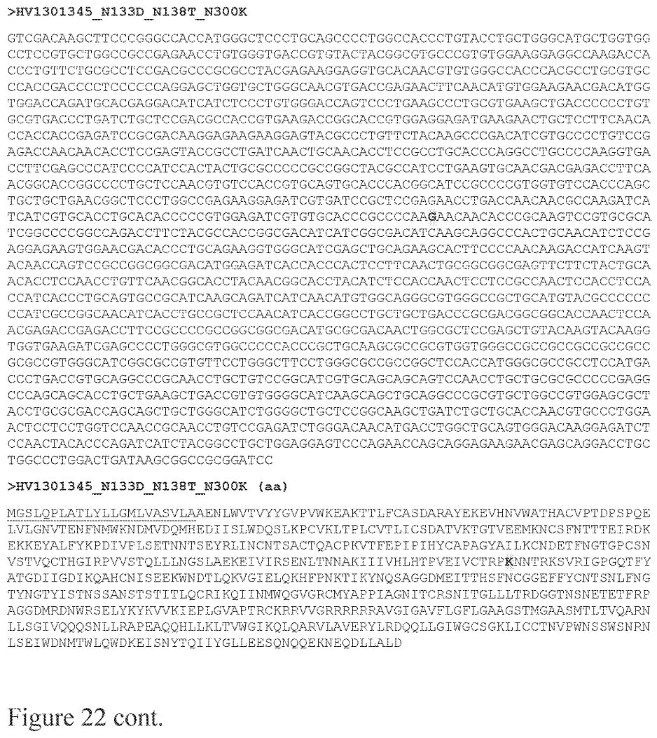

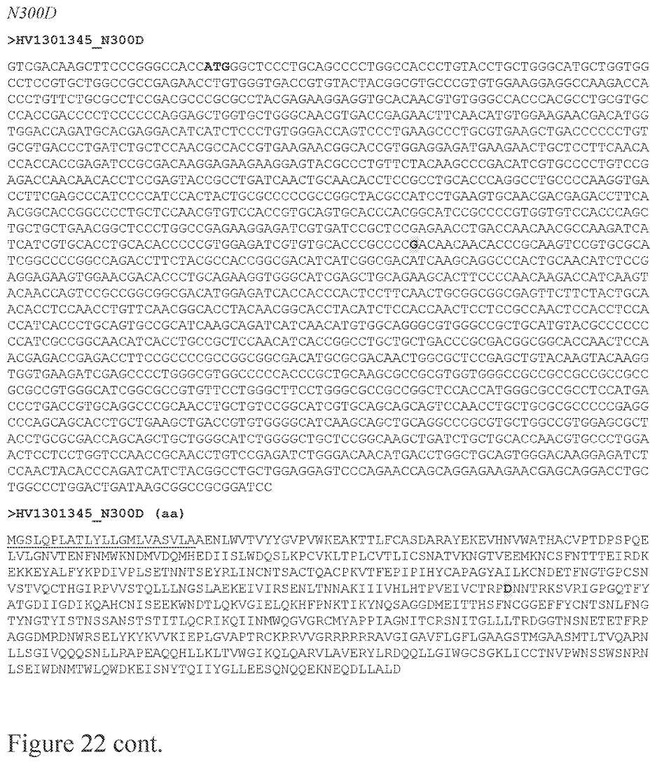

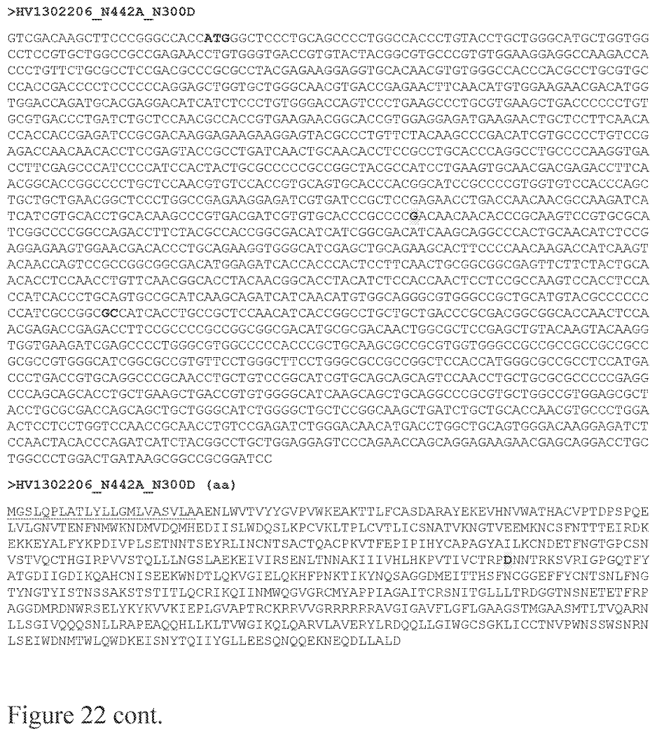

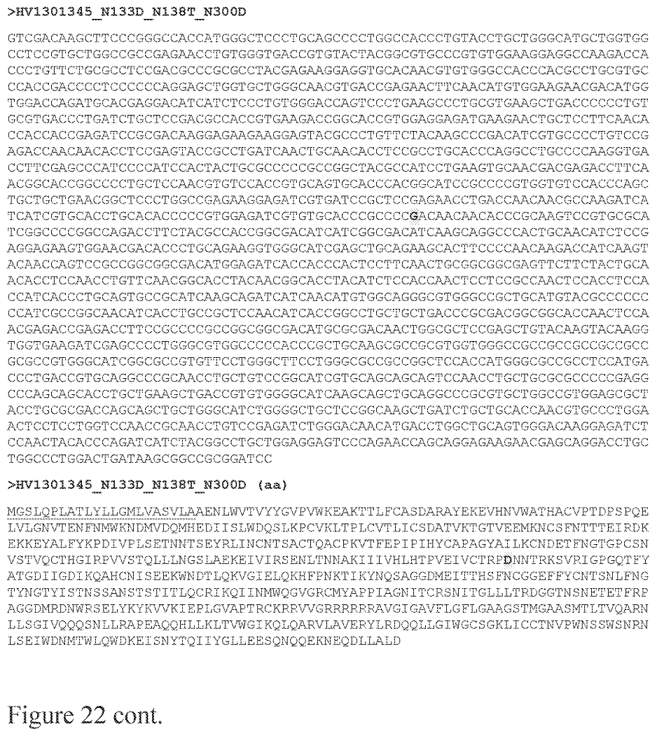

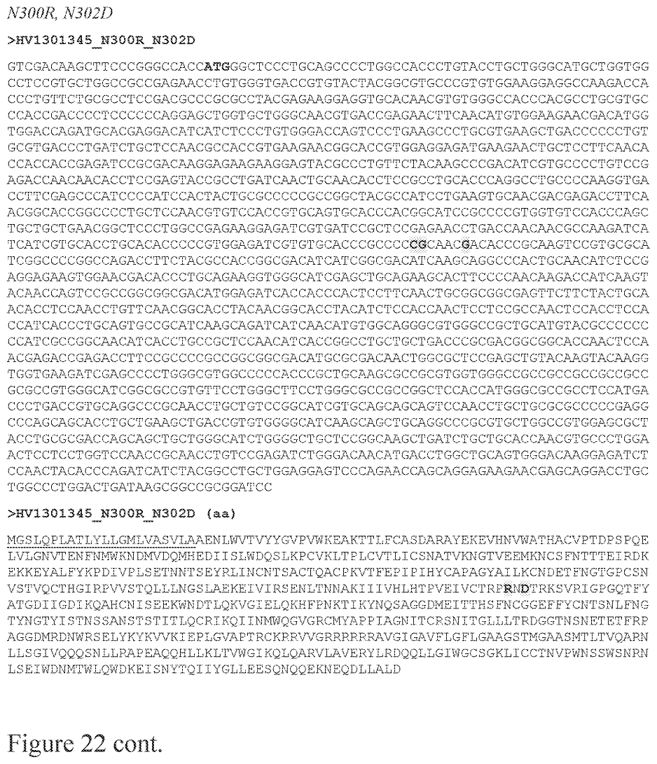

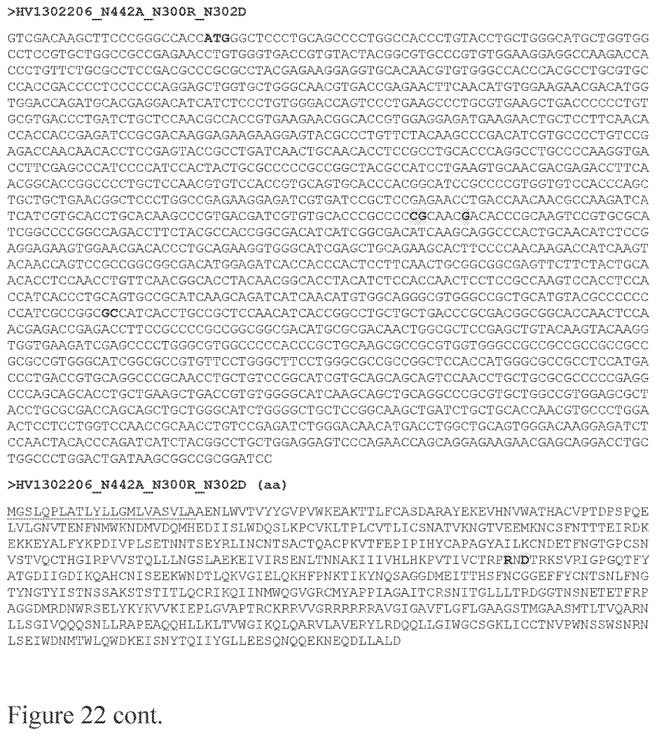

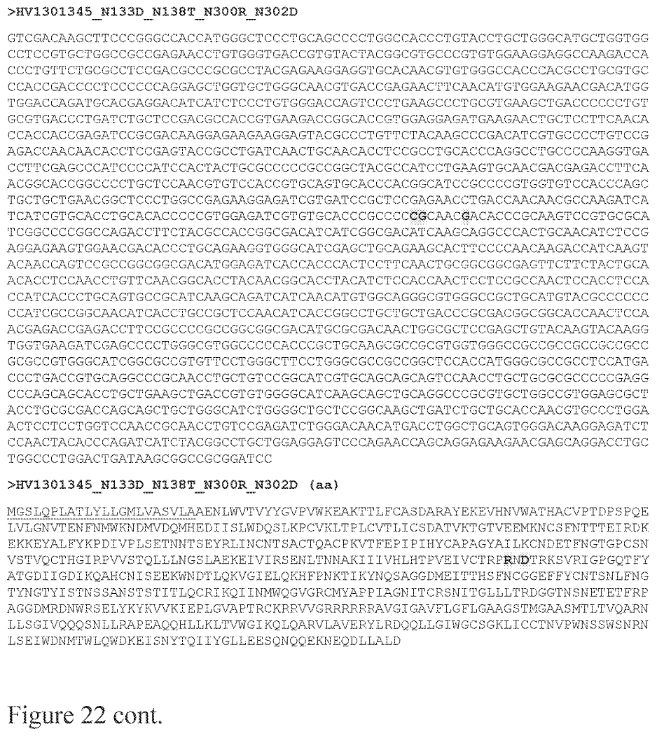

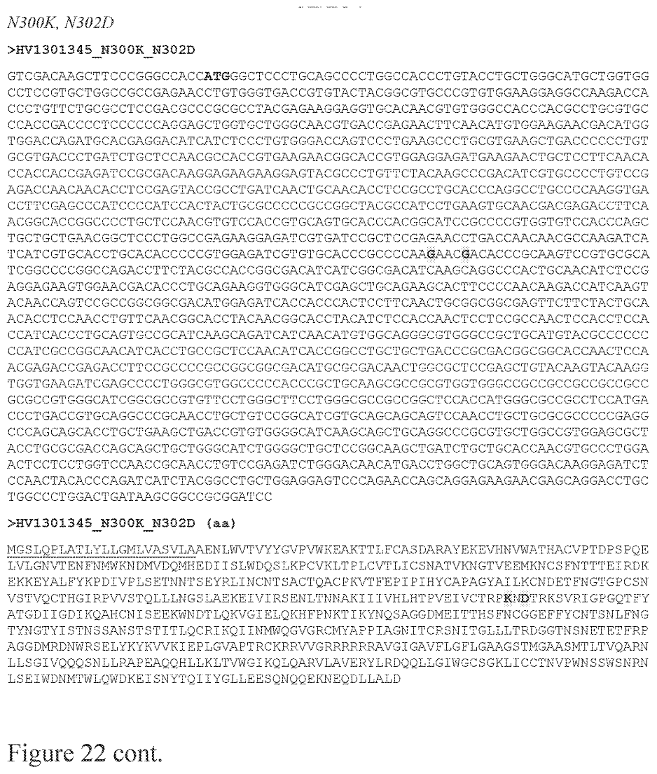

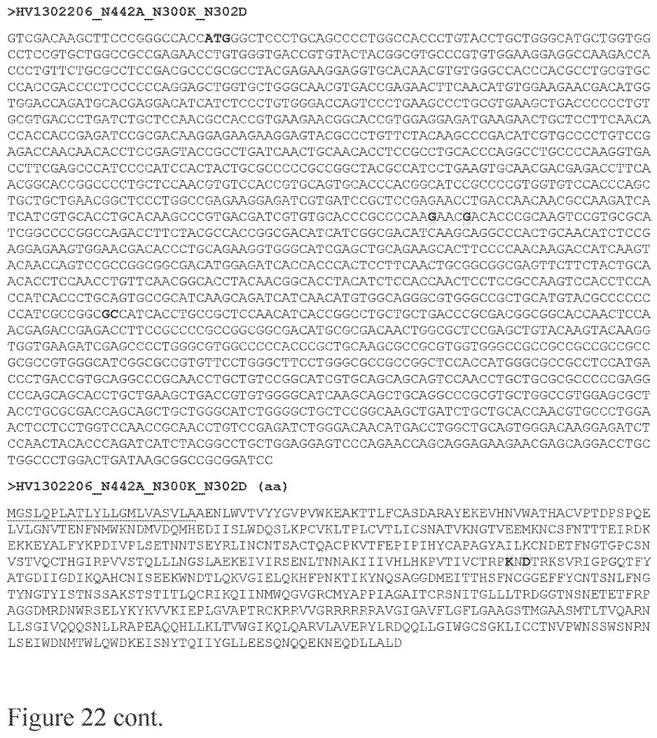

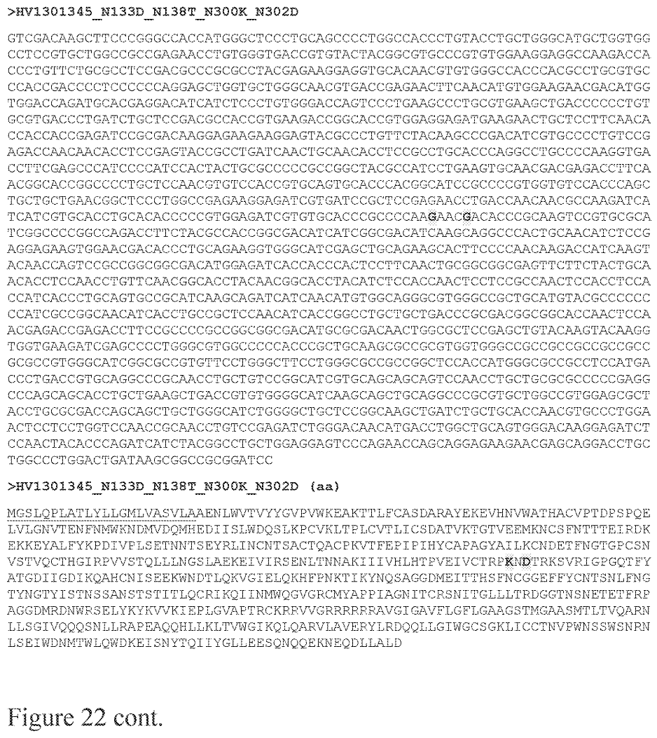

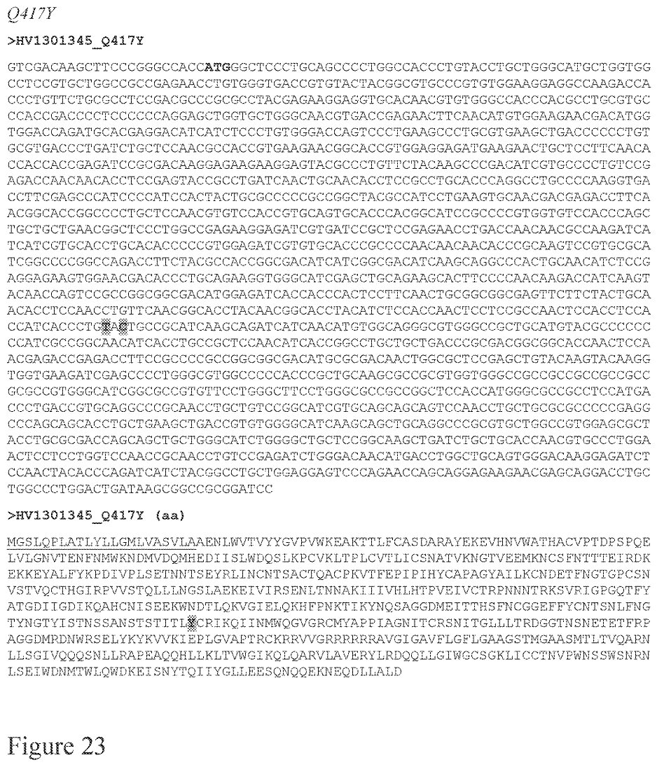

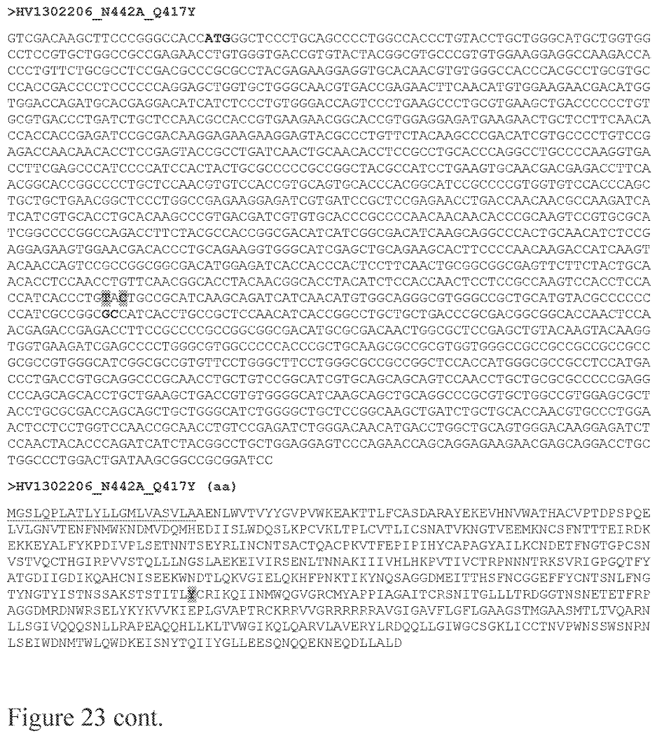

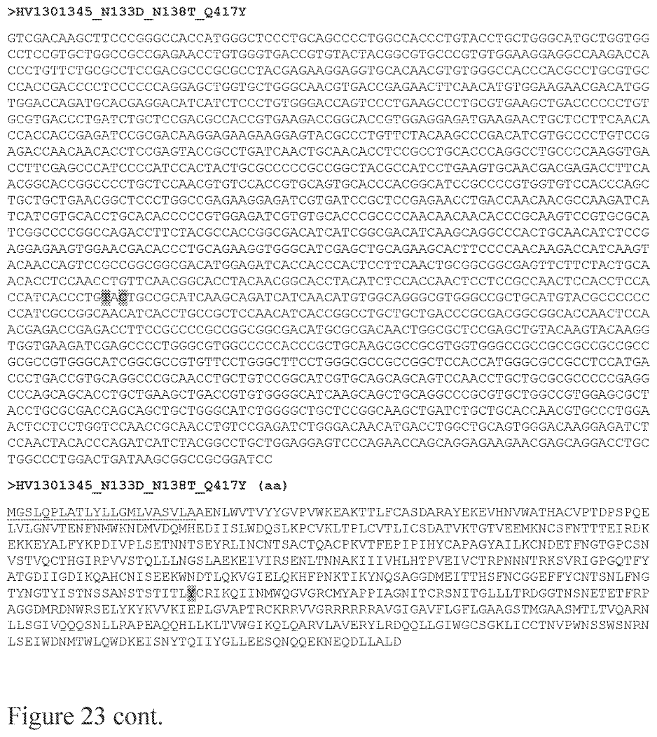

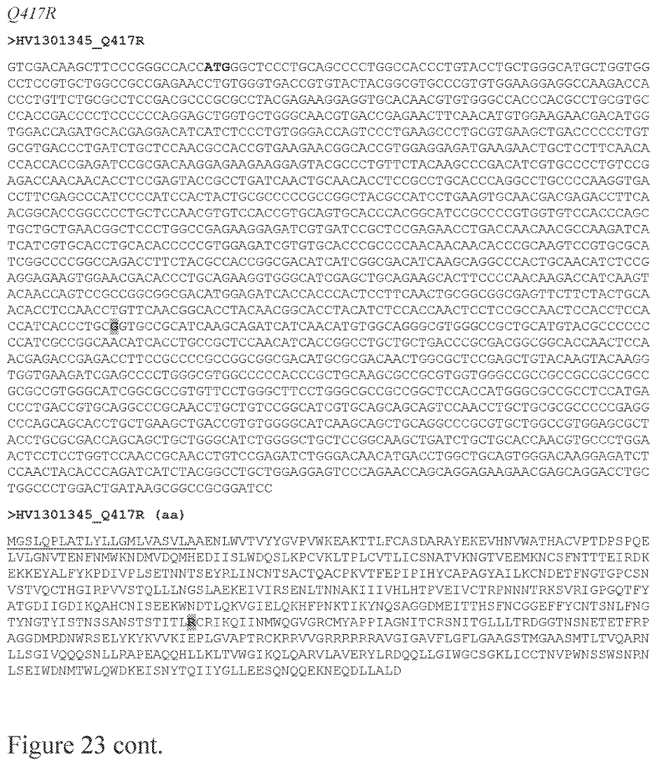

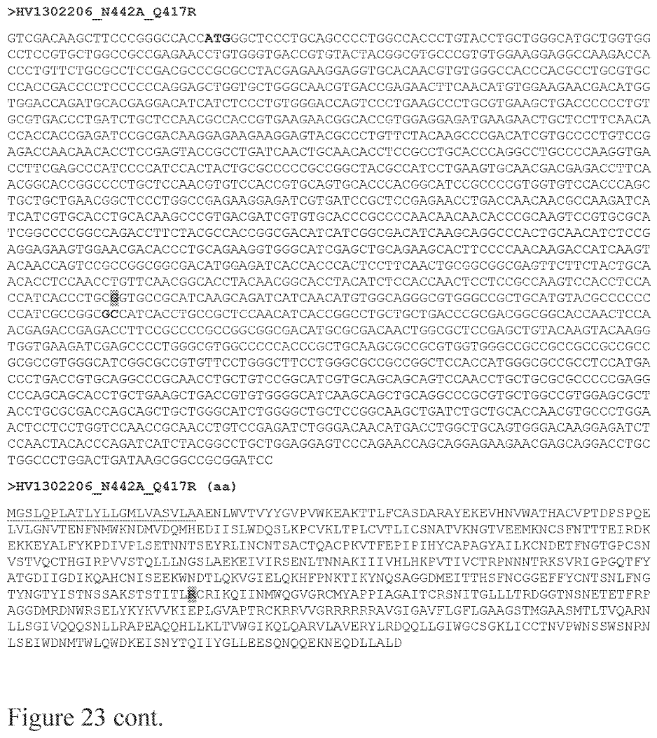

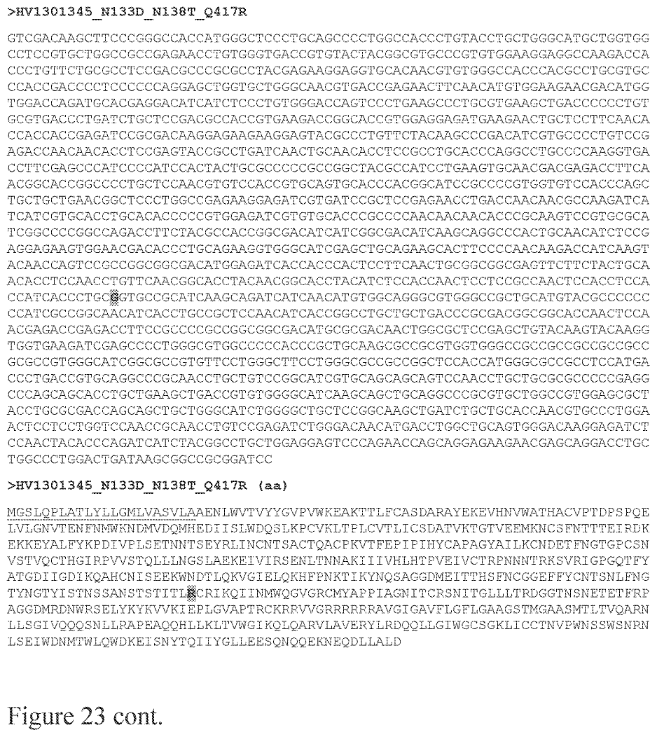

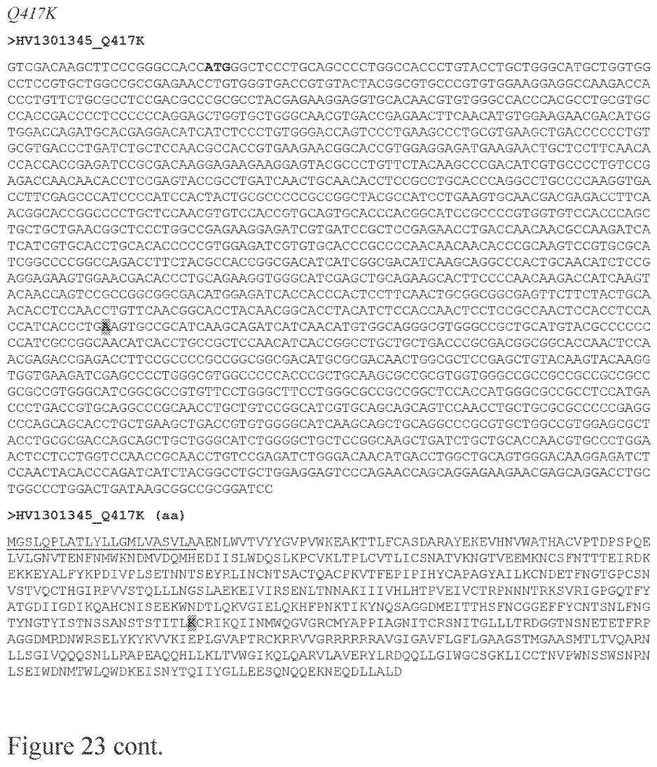

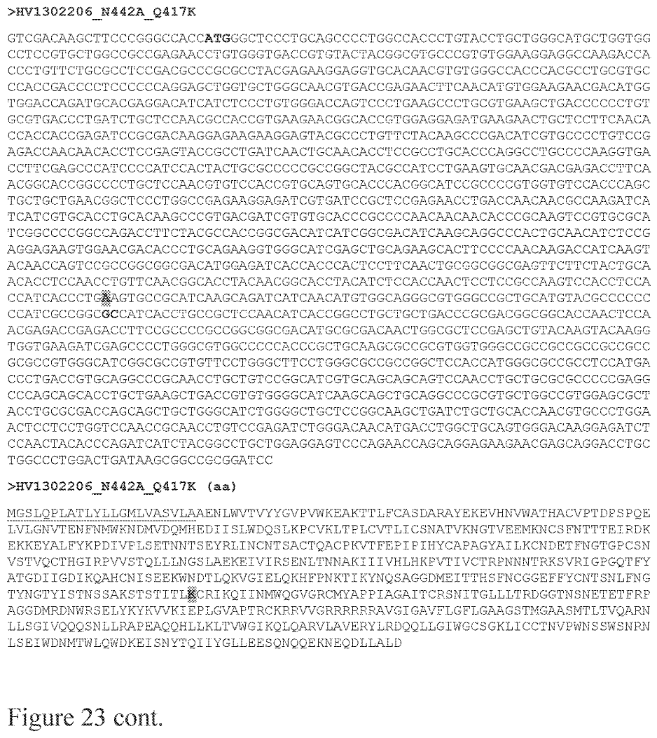

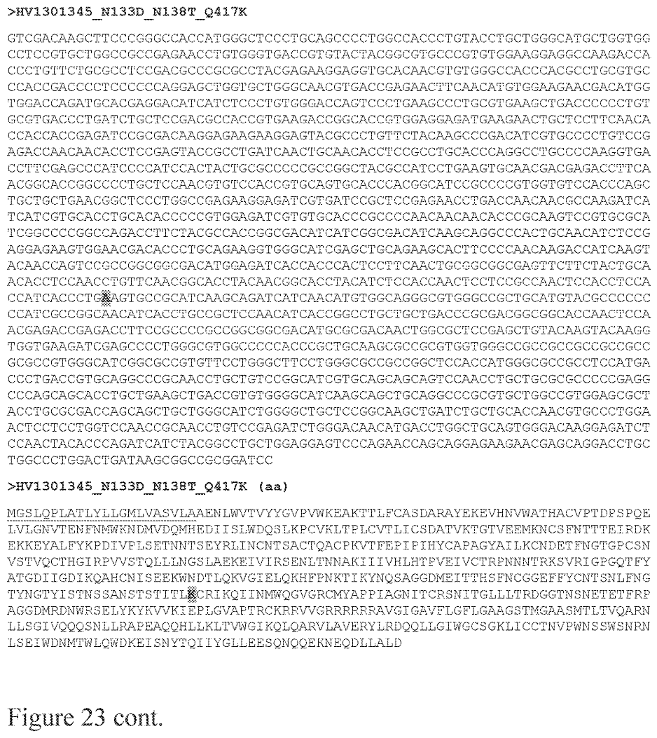

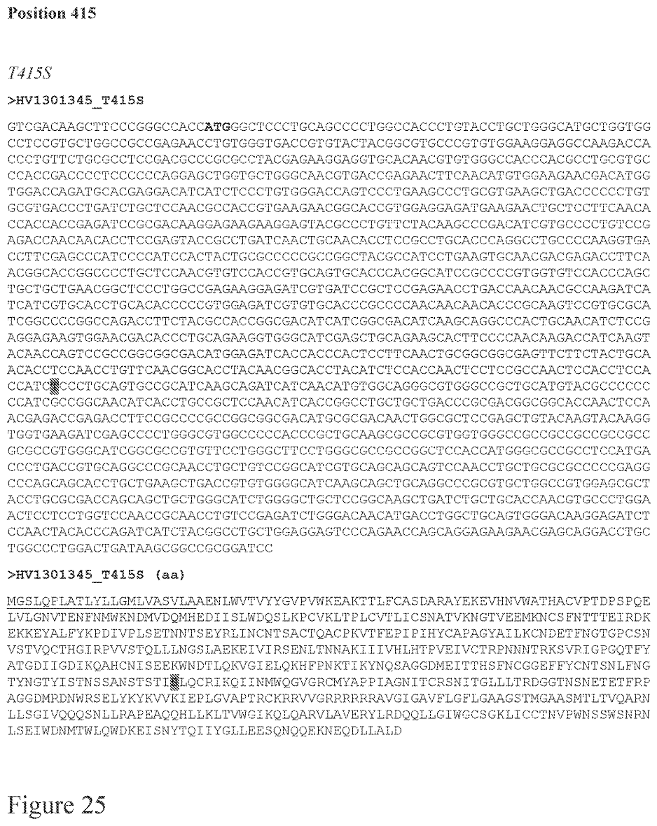

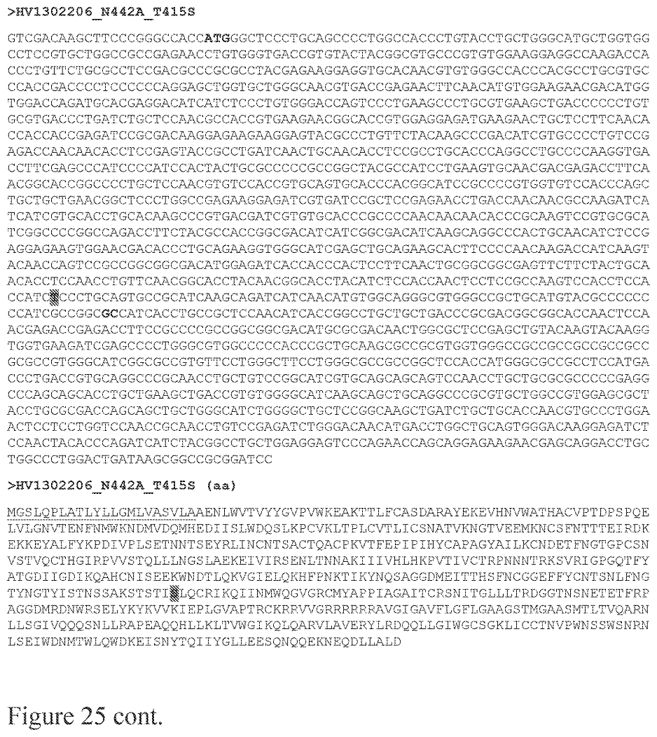

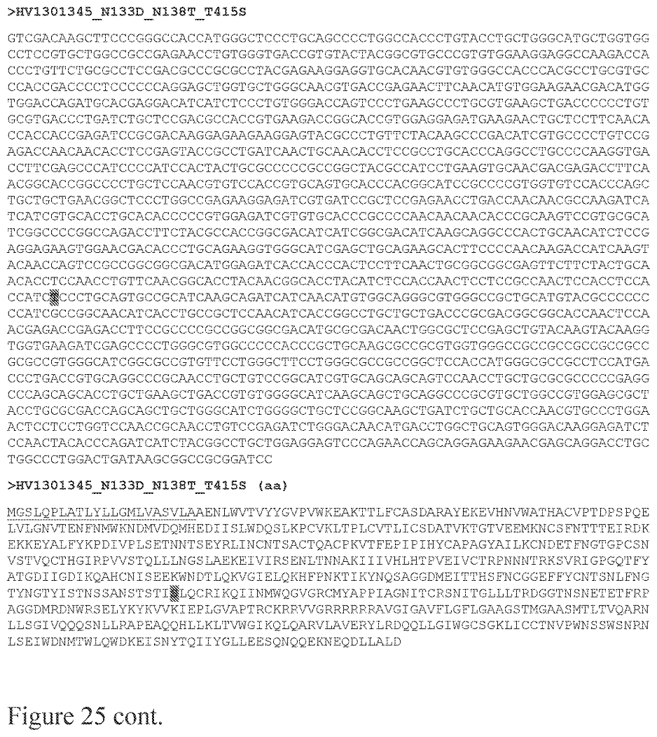

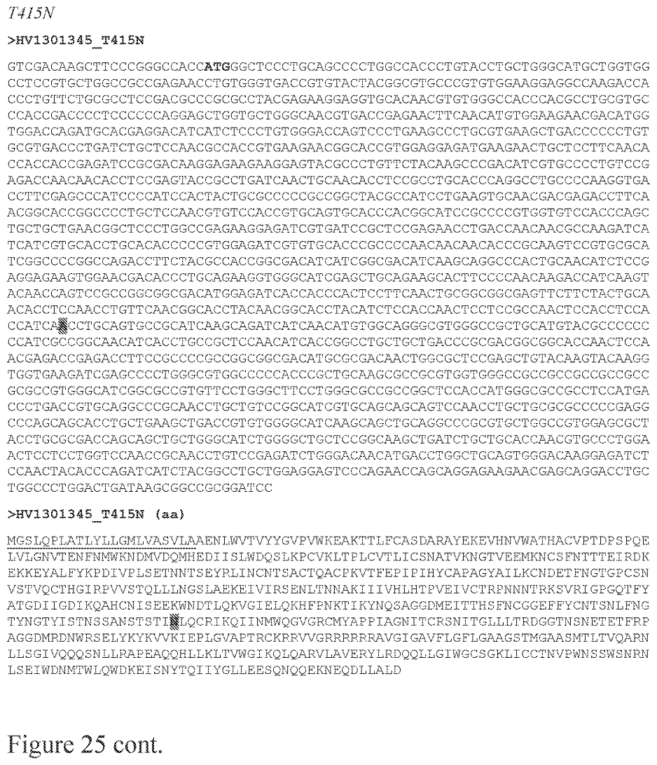

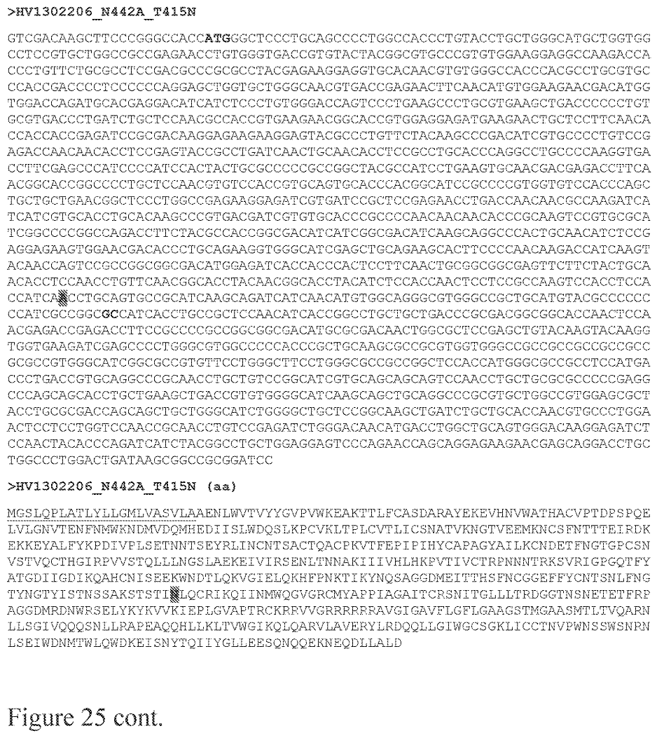

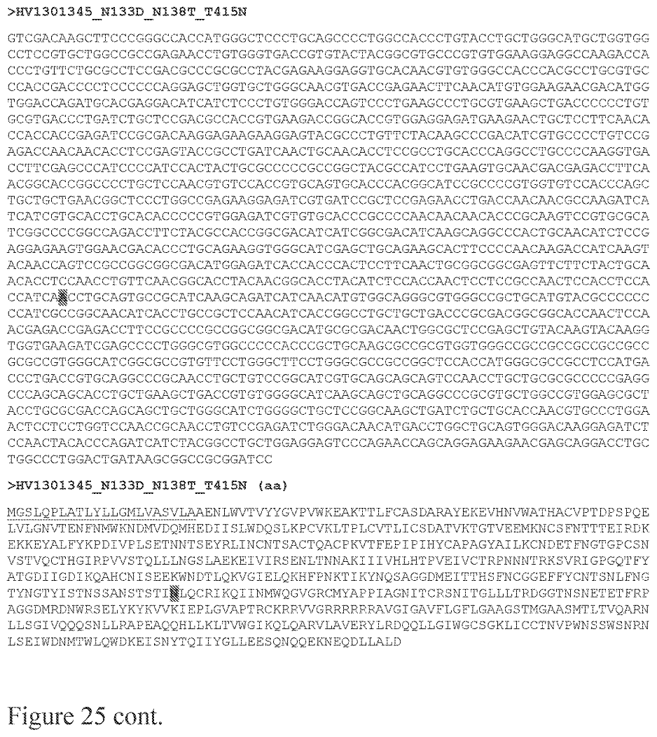

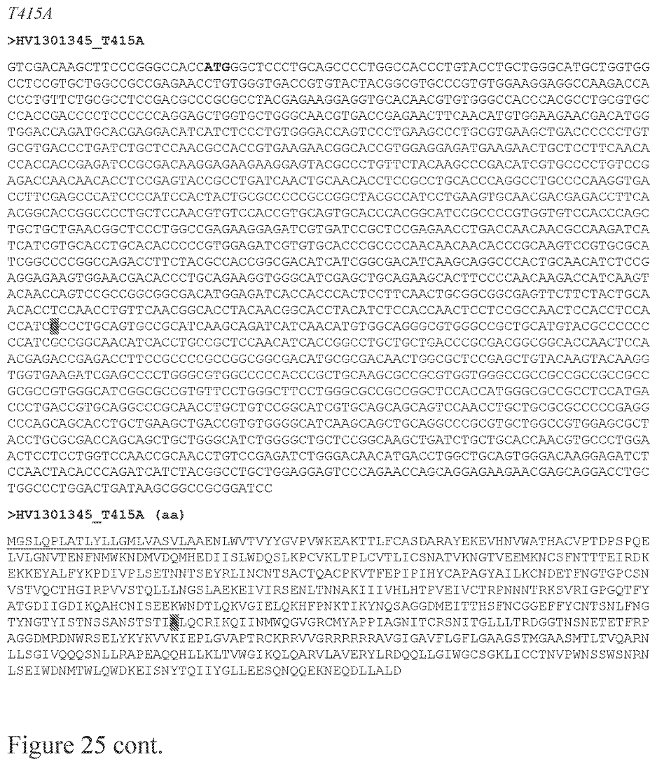

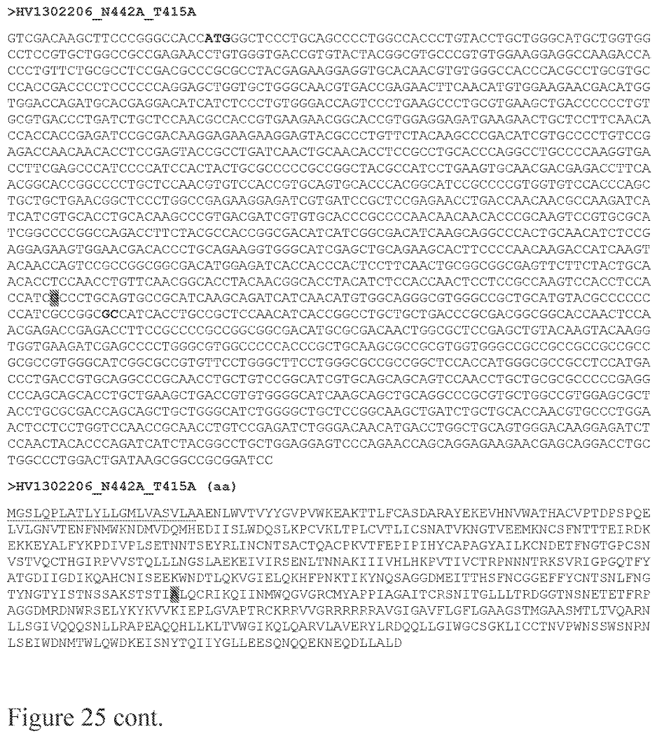

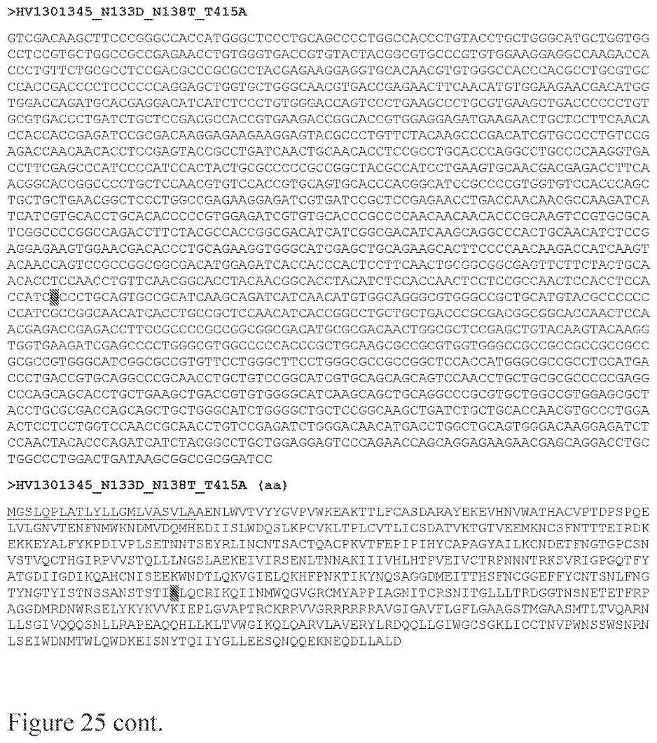

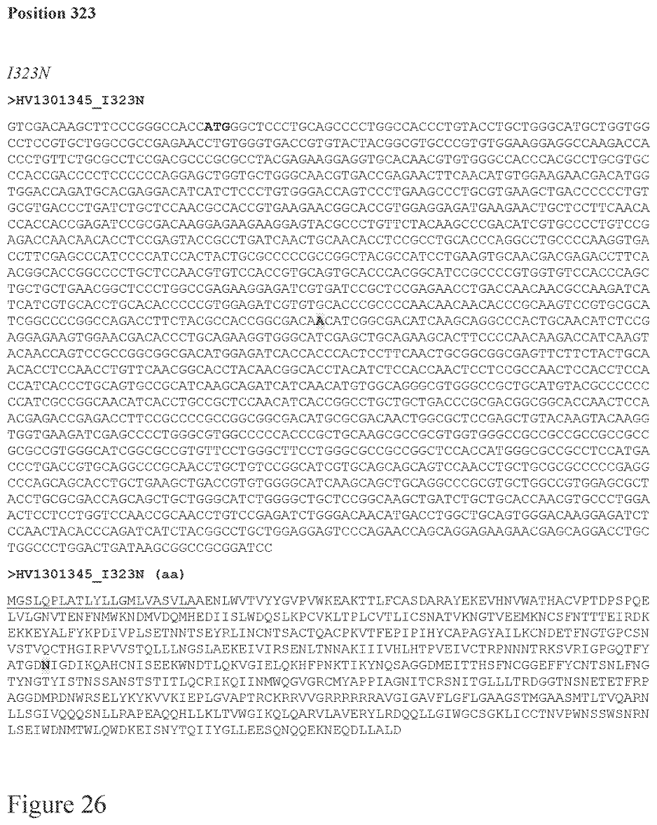

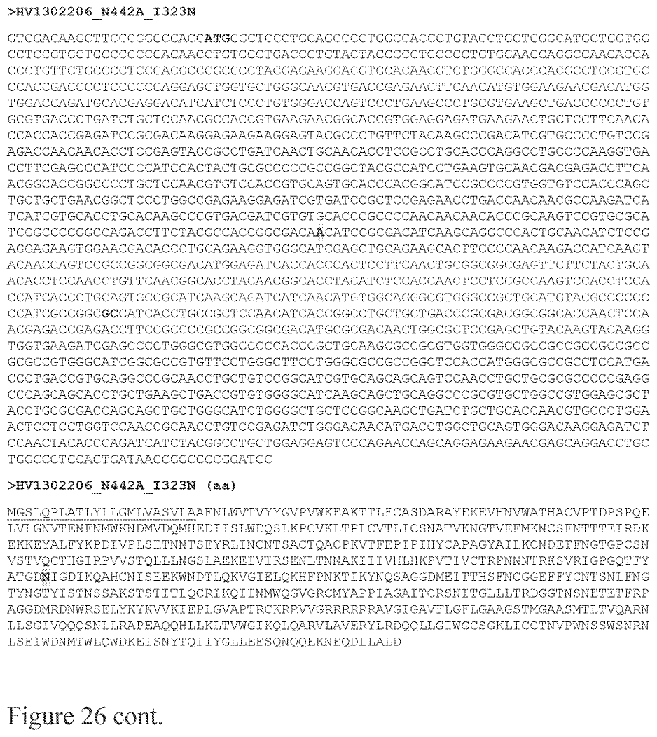

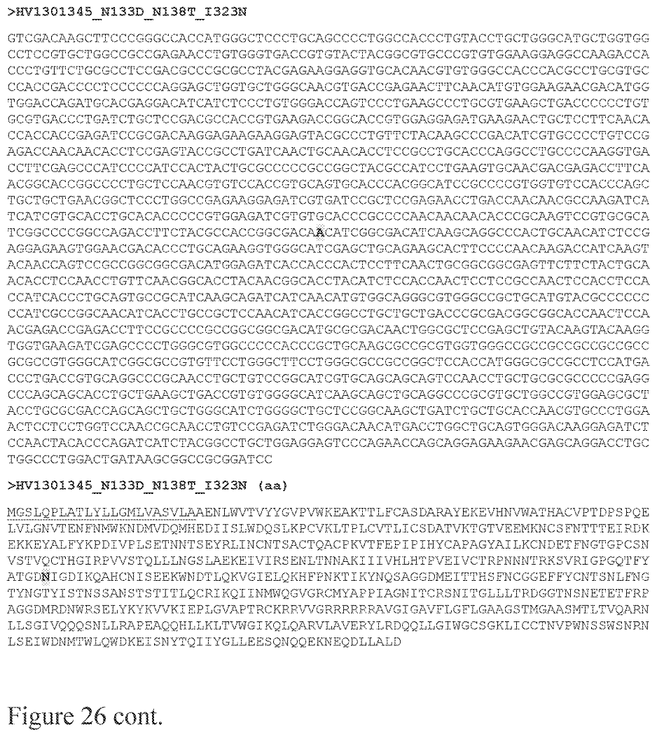

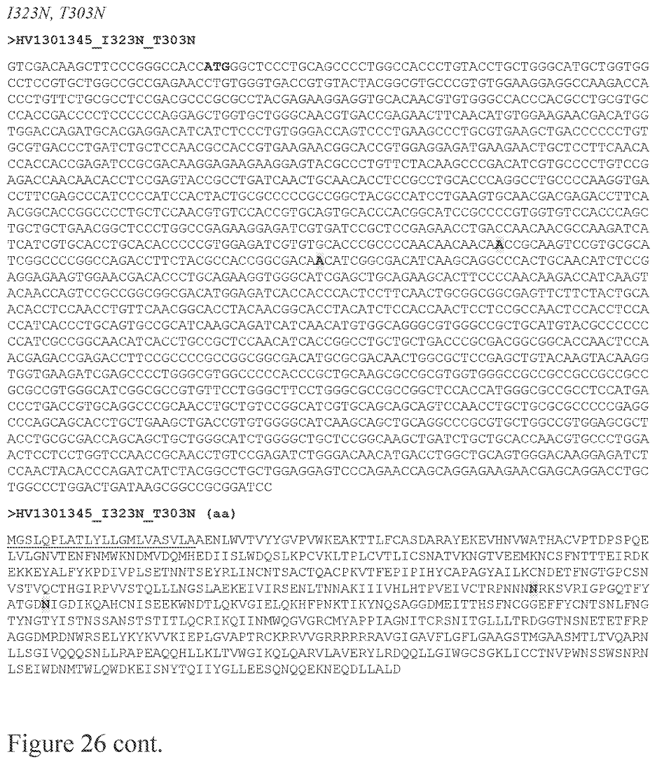

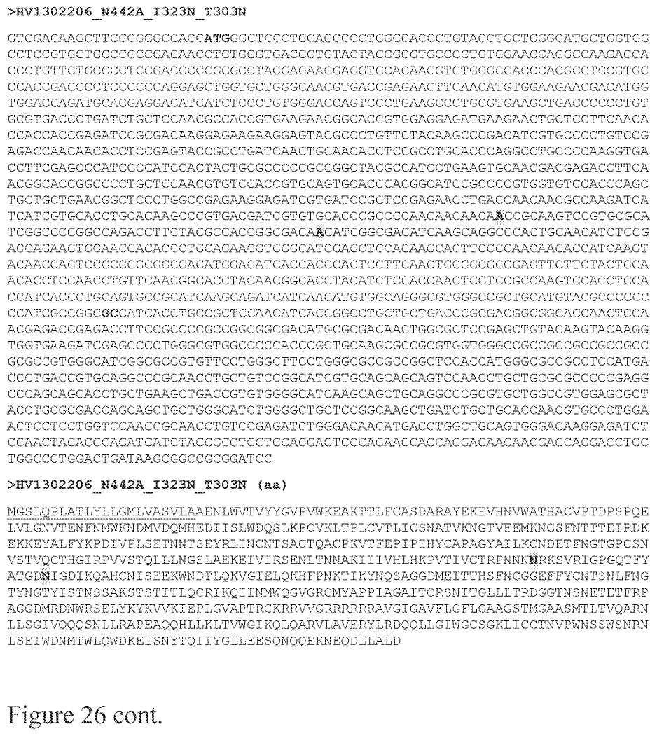

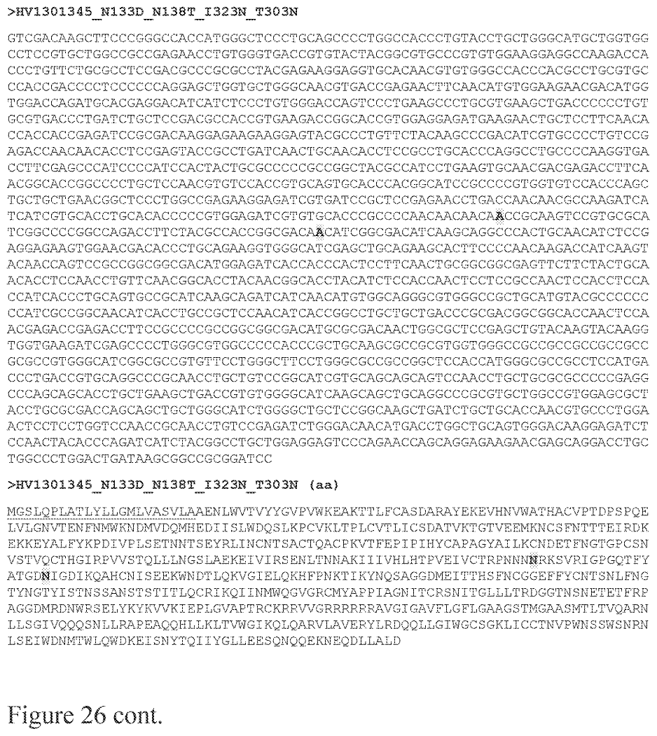

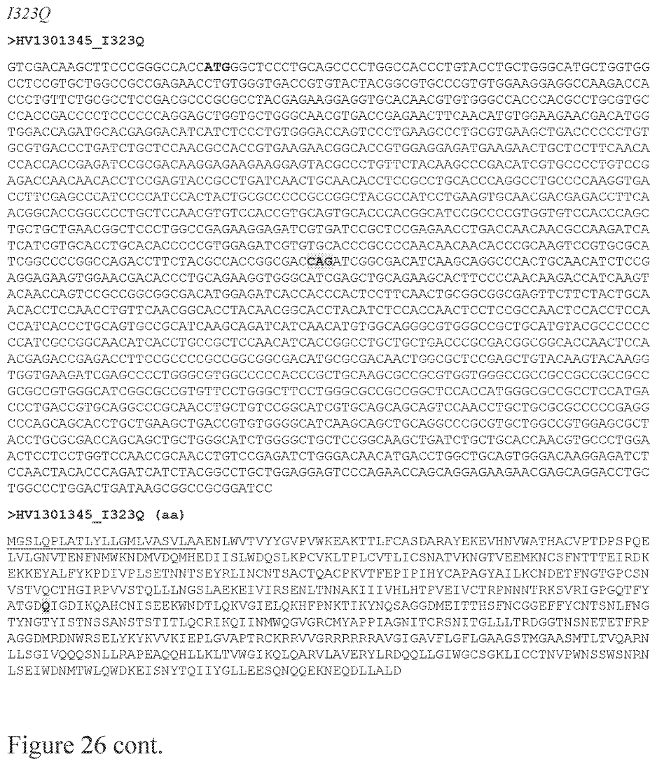

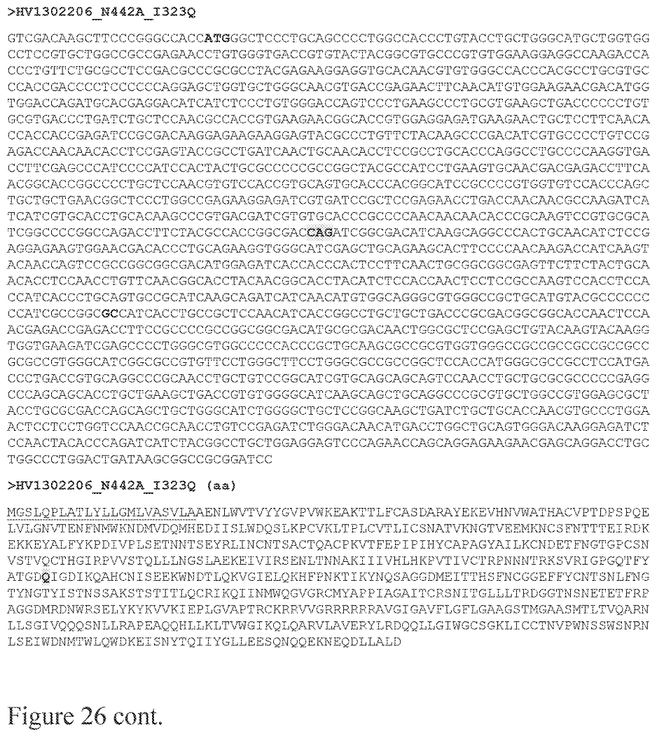

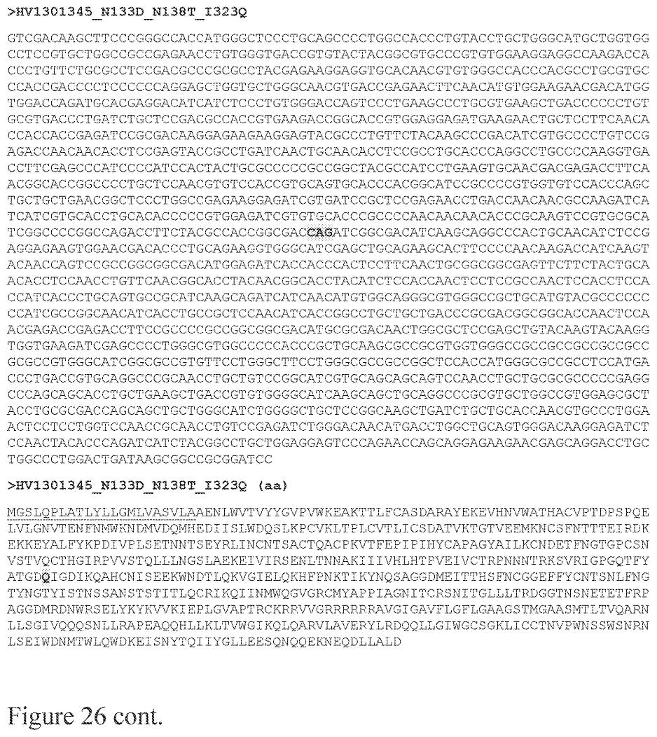

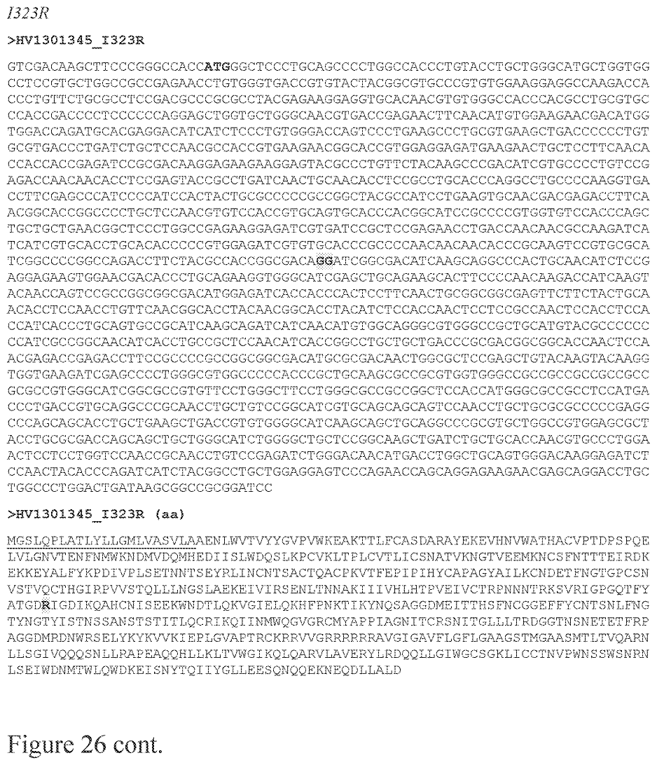

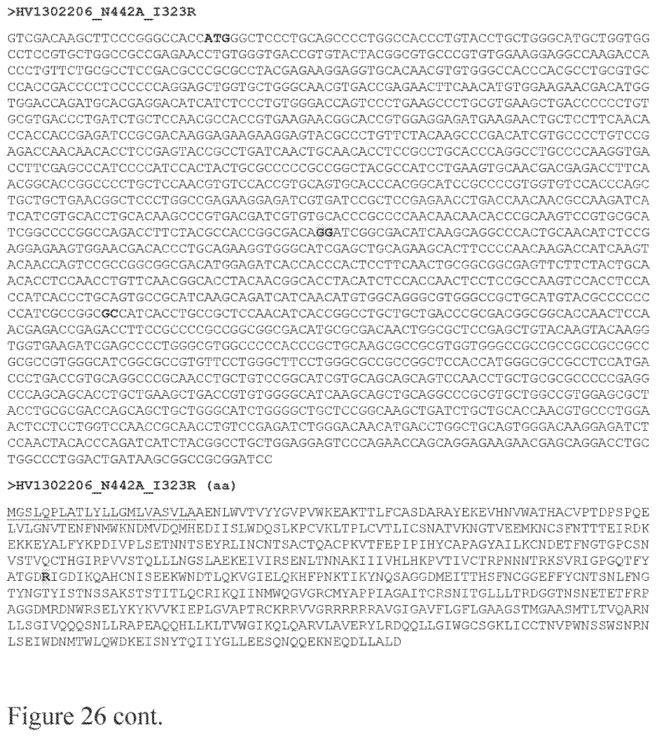

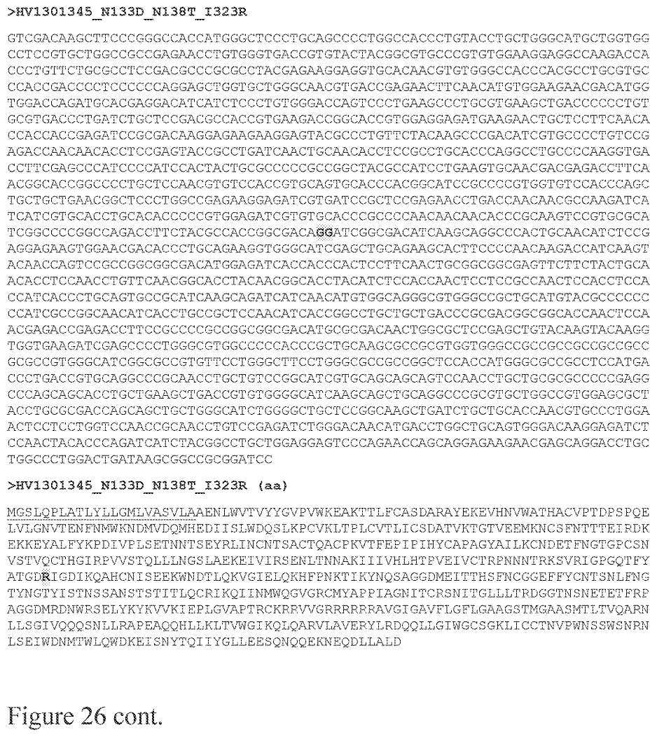

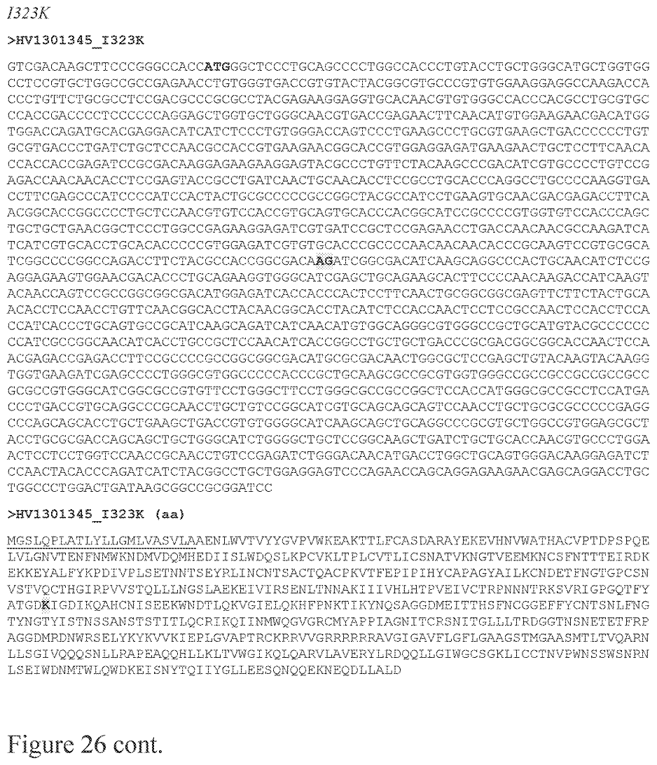

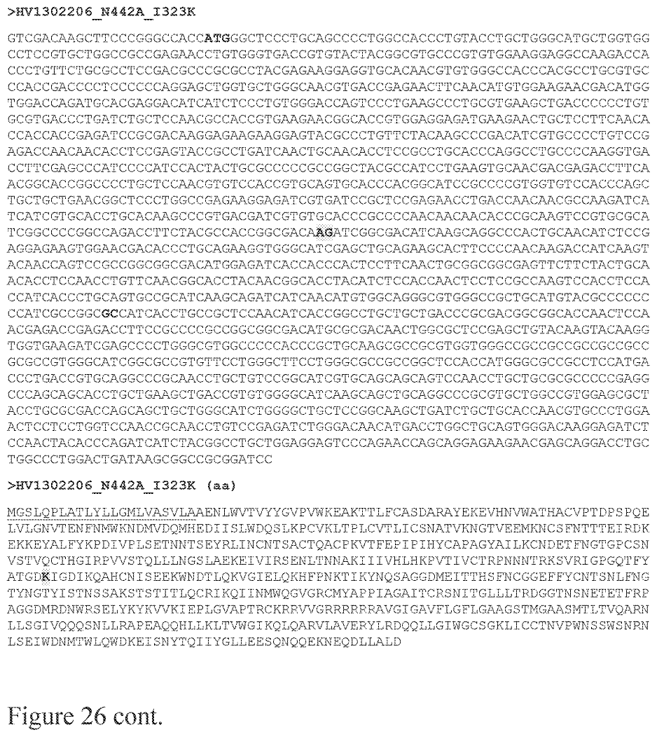

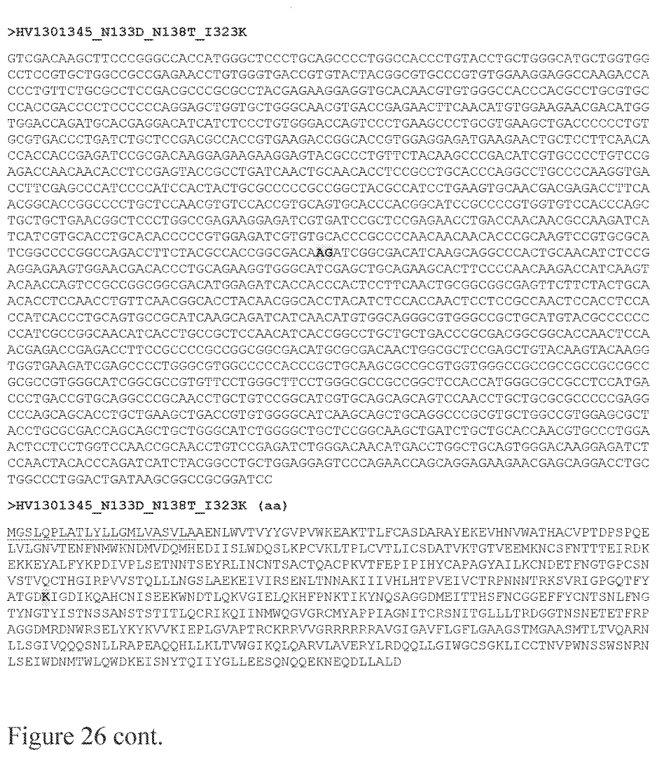

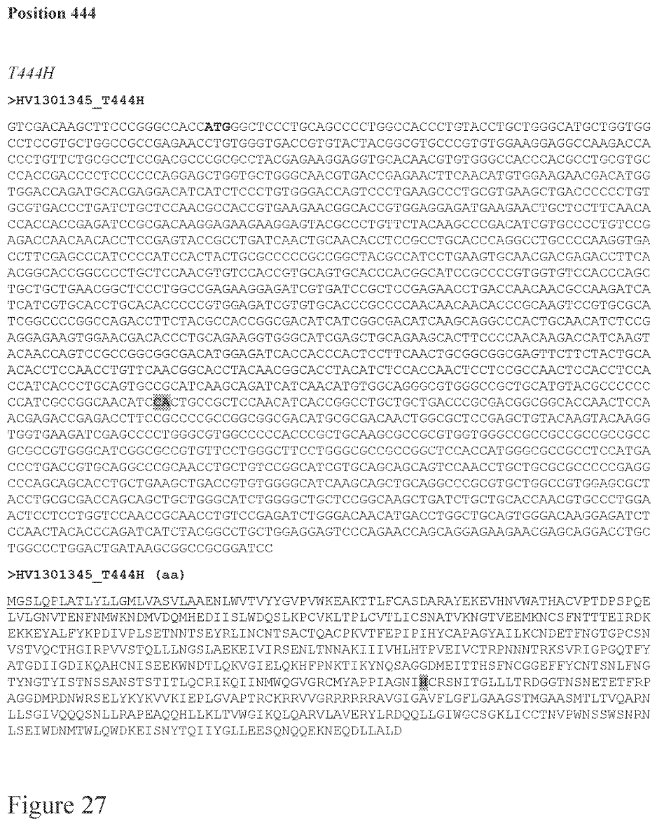

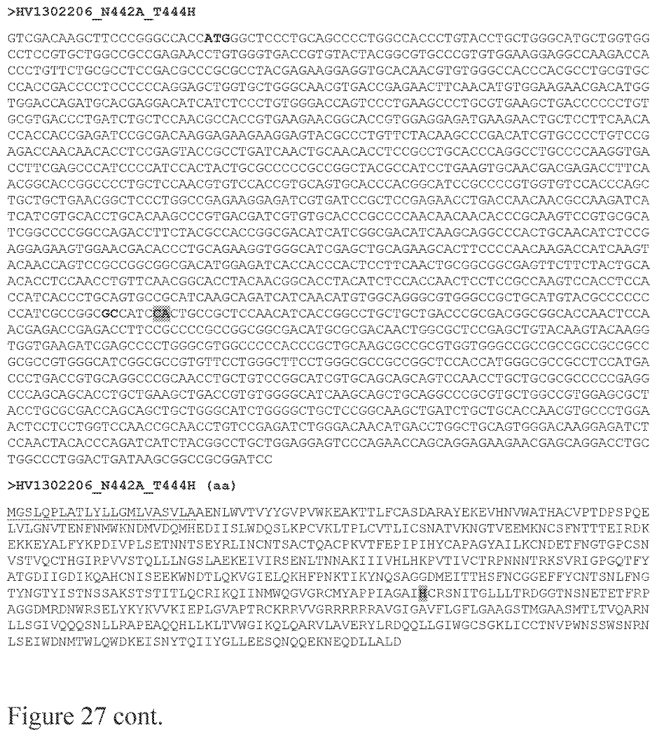

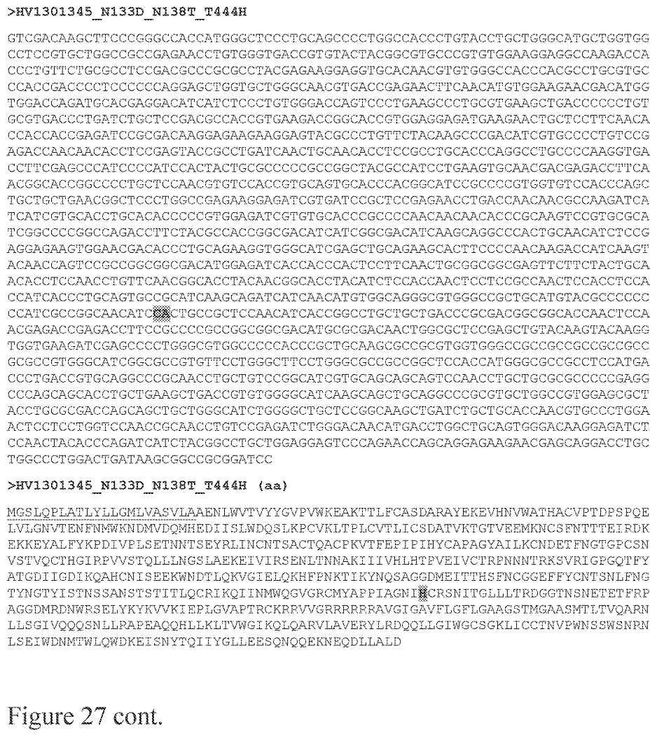

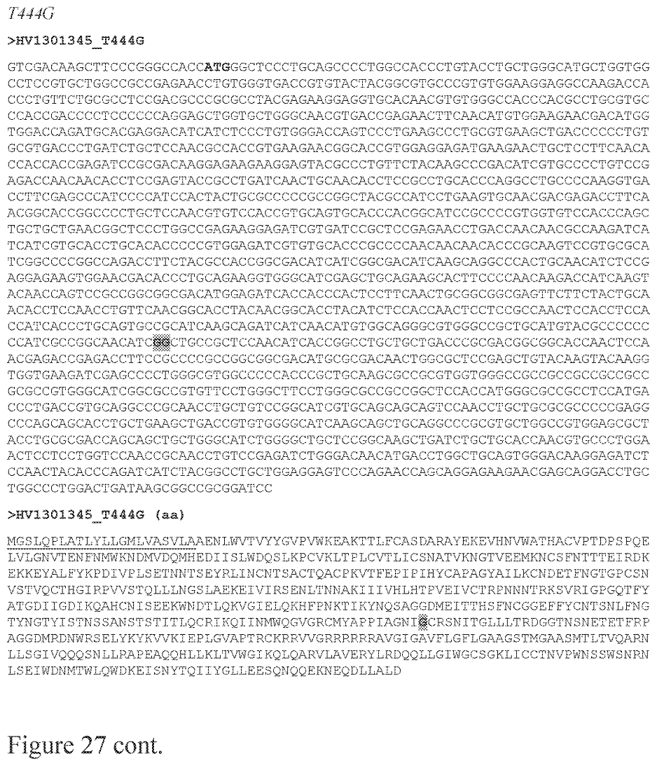

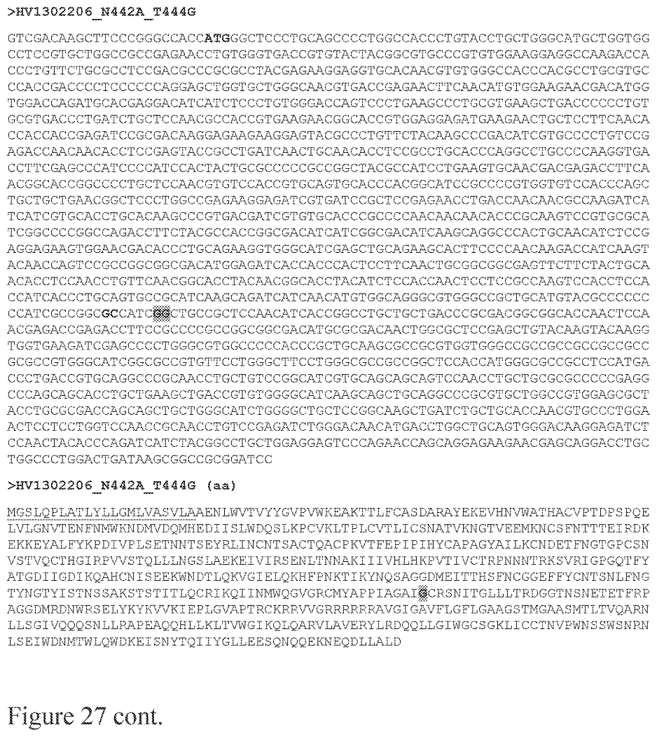

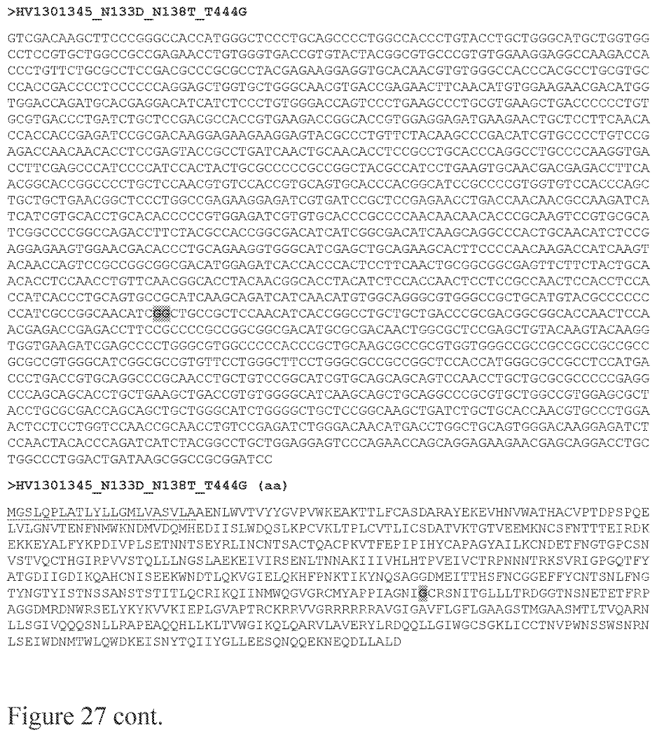

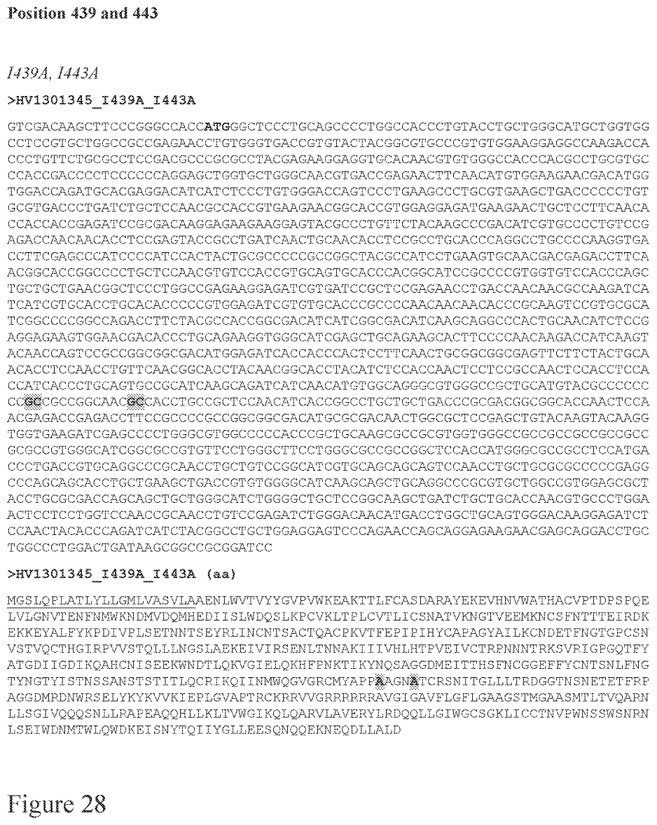

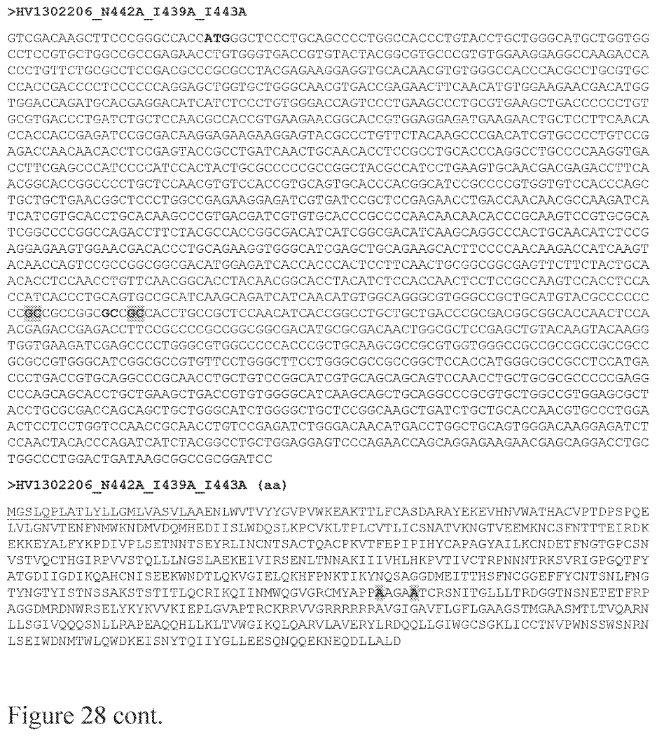

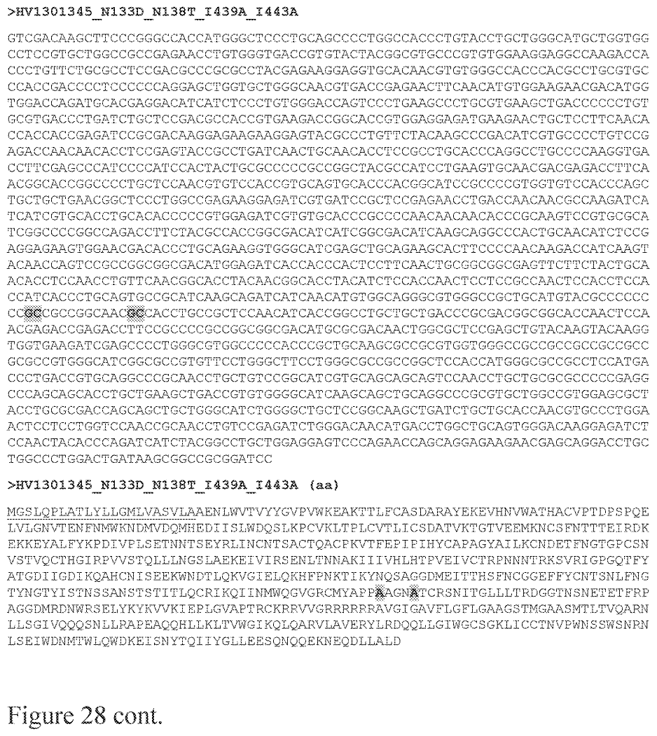

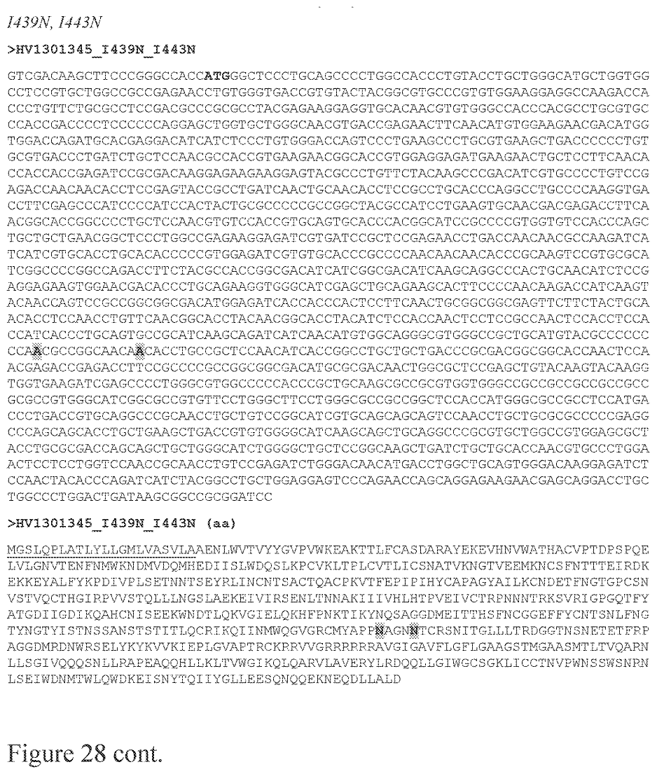

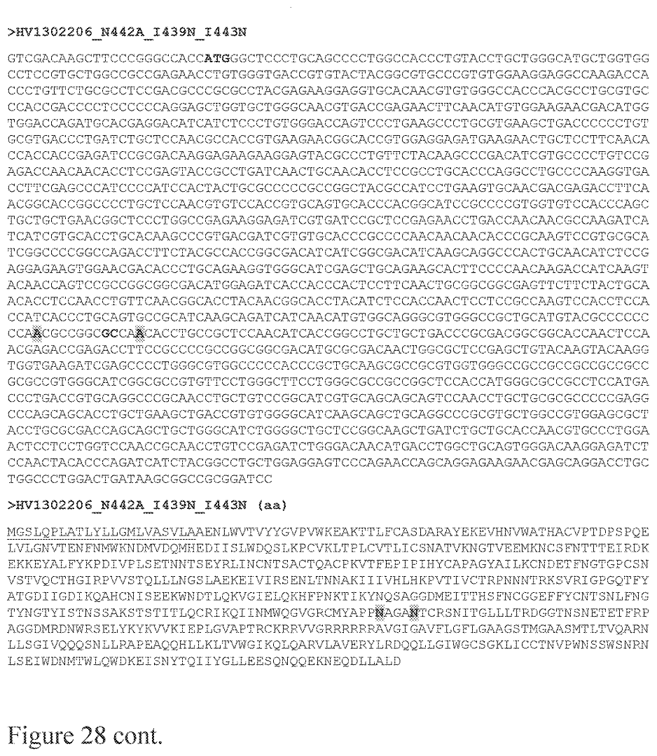

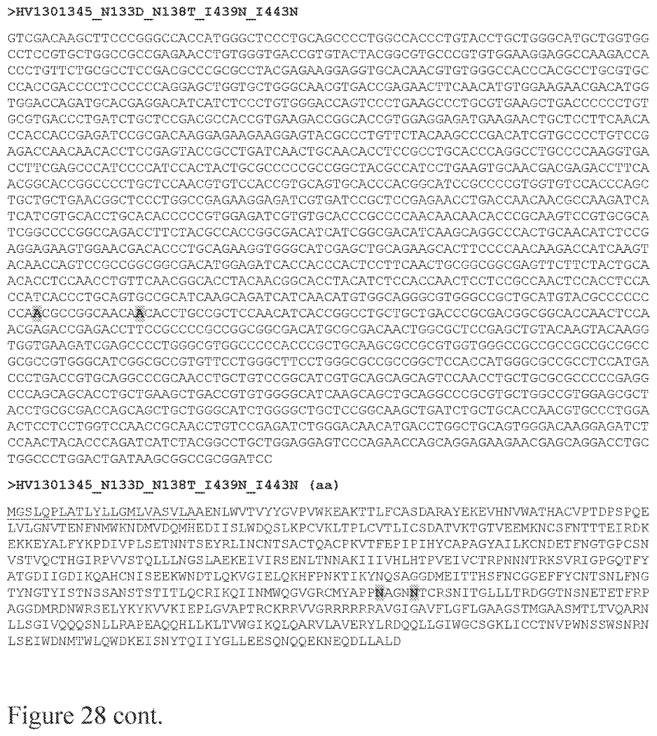

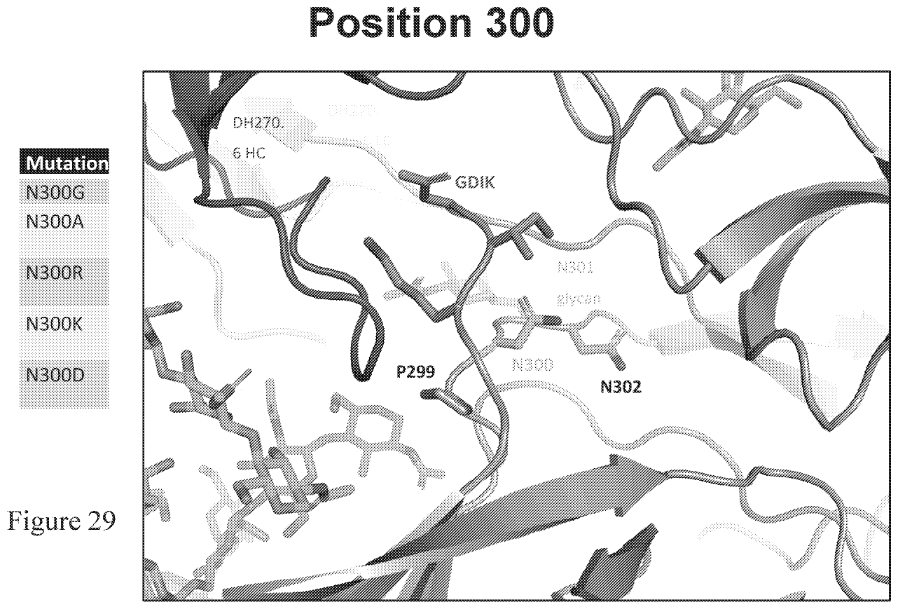

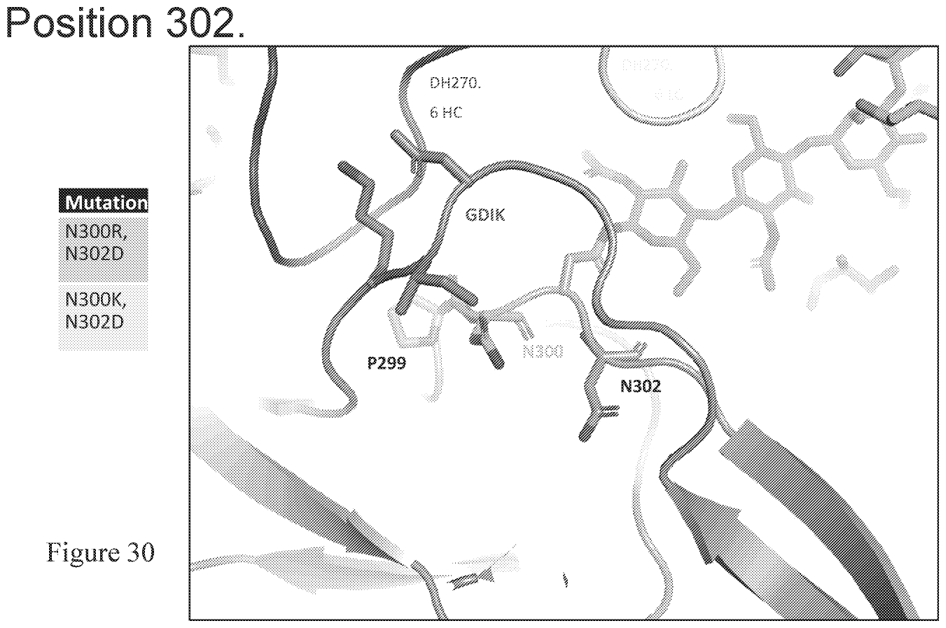

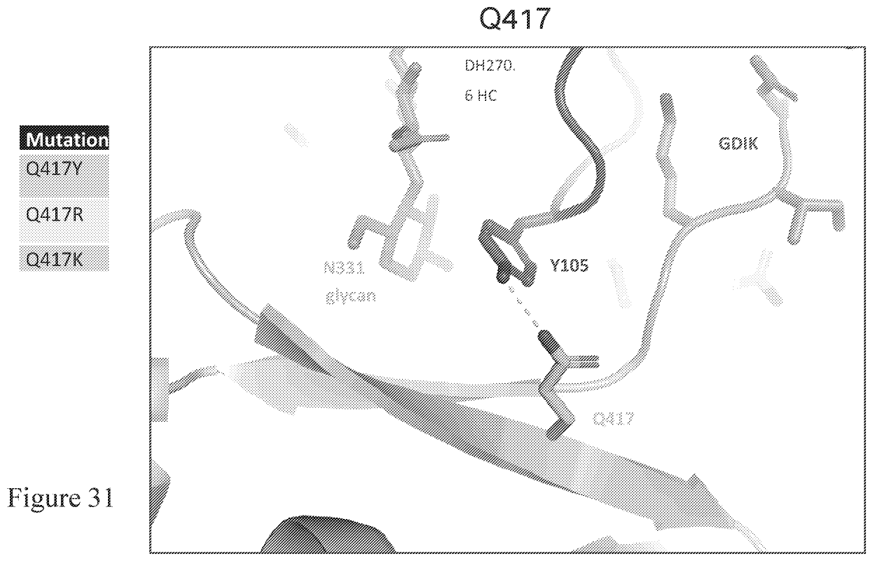

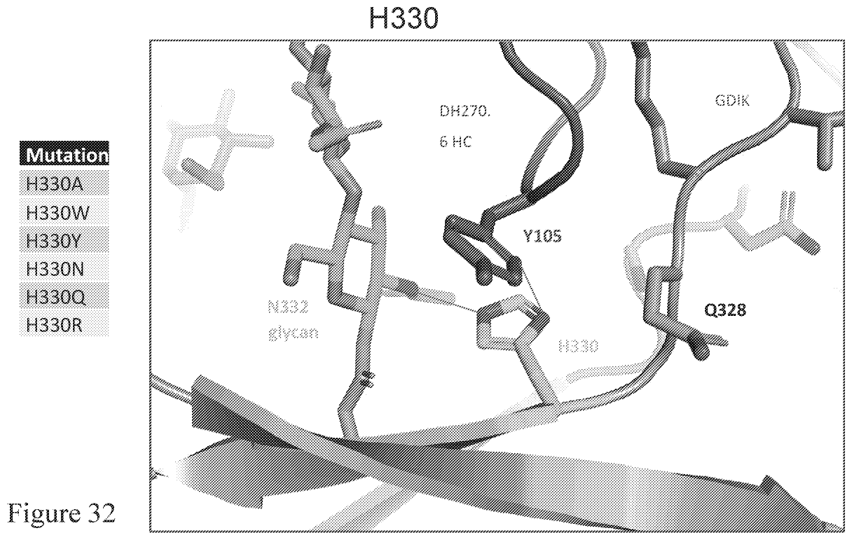

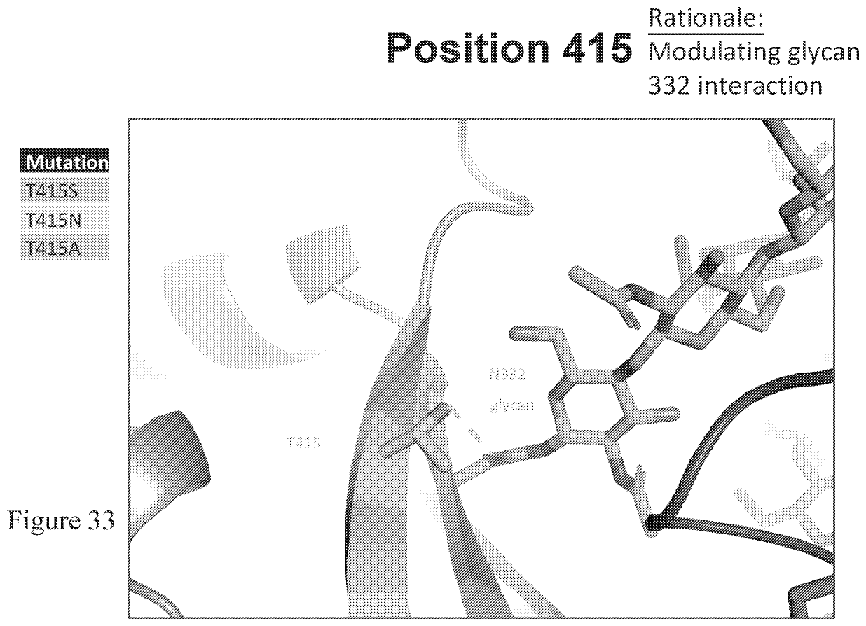

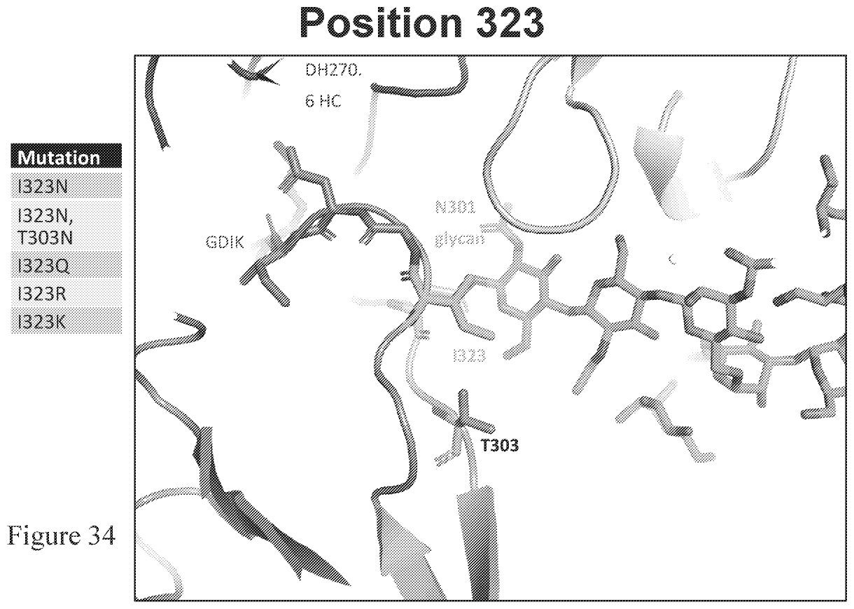

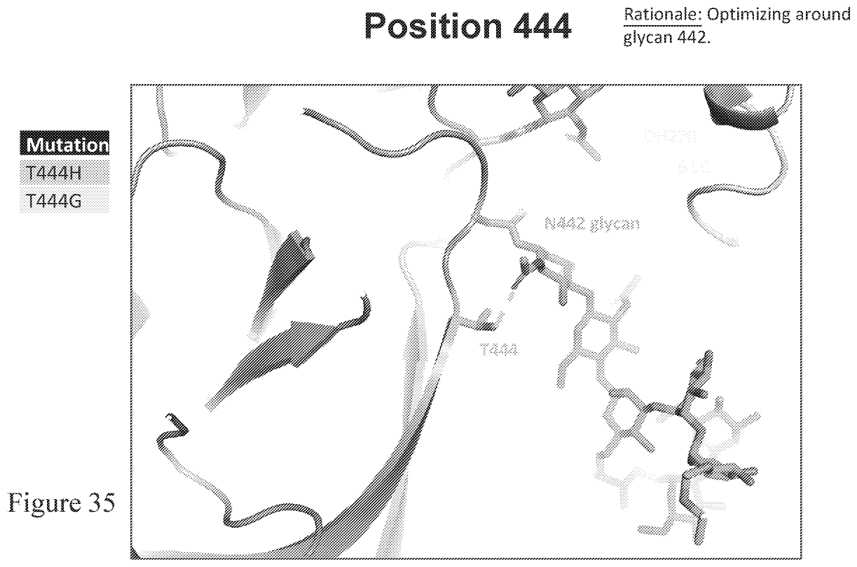

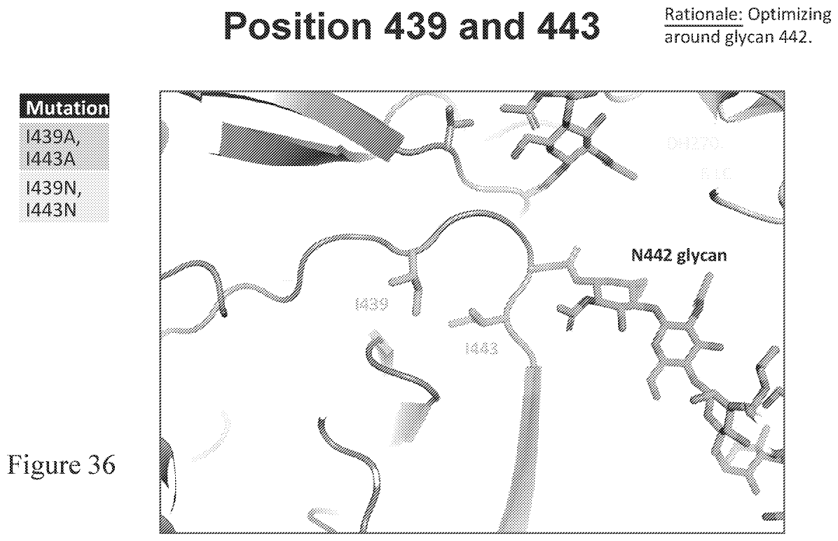

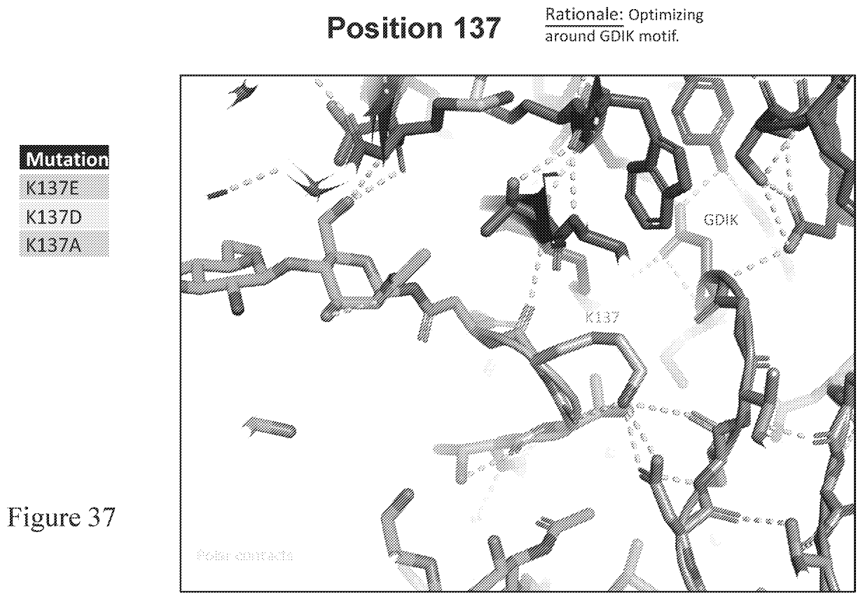

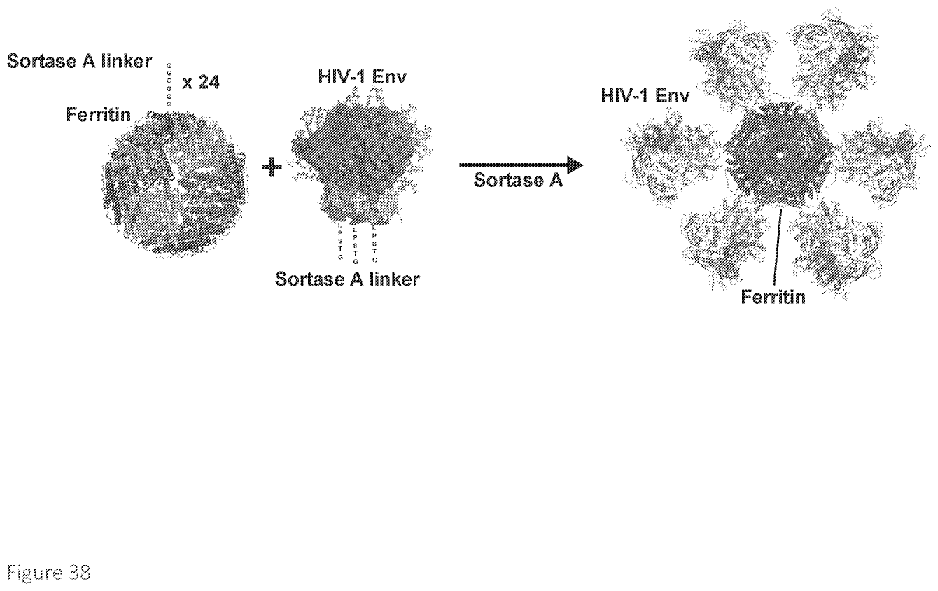

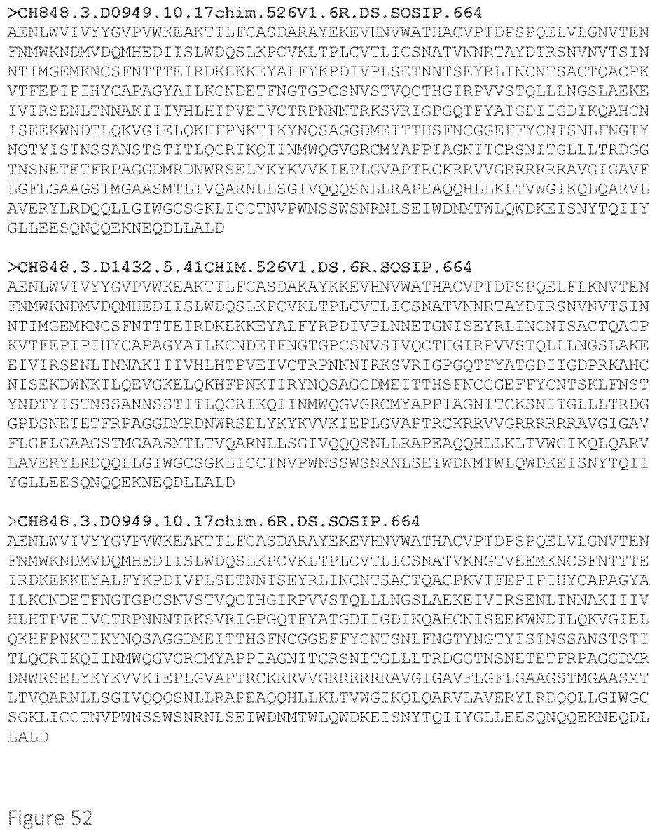

Table 1 shows a summary of sequences described herein.

| | | | | SEQ | SEQ | |
|---|---|---|---|---|---|---|
| | | | Name | ID | ID | |
| Mutation | Envelope | | in | NO: | NO: | |
| position | name | Design | Figure | (DNA) | (aa) | Figure |
| N300 | CH848.3.D0949.10.17chim.6R.DS.SOSIP.664_{—} | SOSIP | >HV1301345_{—} | 47 | 48 | FIG. 22 |
| | N300G | | N300G | | | |
| N300 | CH848.3.D0949.10.17chim.6R.DS.SOSIP.664_{—} | SOSIP | >HV1302206_{—} | 49 | 50 | FIG. 22 |
| | T238K_E241T_N353K_N442A_N300G | | N442A_N300G | | | |
| N300 | CH848.3.D0949.10.17chim.6R.DS.SOSIP.664_{—} | SOSIP | >HV1301345_{—} | 51 | 52 | FIG. 22 |
| | N133D_N138T_N300G | | N133D_N138T_{—} | | | |
| | | | N300G | | | |
| N300 | CH848.3.D0949.10.17chim.6R.DS.SOSIP.664_{—} | SOSIP | >HV1301345_{—} | 53 | 54 | FIG. 22 |
| | N300A | | N300A | | | |
| N300 | CH848.3.D0949.10.17chim.6R.DS.SOSIP.664_{—} | SOSIP | >HV1302206_{—} | 55 | 56 | FIG. 22 |
| | T238K_E241T_N353K_N442A_N300A | | N442A_N300A | | | |
| N300 | CH848.3.D0949.10.17chim.6R.DS.SOSIP.664_{—} | SOSIP | >HV1301345_{—} | 57 | 58 | FIG. 22 |
| | N133D_N138T_N300A | | N133D_N138T_{—} | | | |
| | | | N300A | | | |
| N300 | CH848.3.D0949.10.17chim.6R.DS.SOSIP.664_{—} | SOSIP | >HV1301345_{—} | 59 | 60 | FIG. 22 |
| | N300R | | N300R | | | |
| N300 | CH848.3.D0949.10.17chim.6R.DS.SOSIP.664_{—} | SOSIP | >HV1302206_{—} | 61 | 62 | FIG. 22 |
| | T238K_B241T_N353K_N442A_N300R | | N442A_N300R | | | |
| N300 | CH848.3.D0949.10.17chim.6R.DS.SOSIP.664_{—} | SOSIP | >HV1301345_{—} | 63 | 64 | FIG. 22 |
| | N133D_N138T_N300R | | N133D_N138T_{—} | | | |
| | | | N300R | | | |
| N300 | CH848.3.D0949.10.17chim.6R.DS.SOSIP.664_{—} | SOSIP | >HV1301345_{—} | 65 | 66 | FIG. 22 |
| | N300K | | N300K | | | |
| N300 | CH848.3.D0949.10.17chim.6R.DS.SOSIP.664_{—} | SOSIP | >HV1302206_{—} | 67 | 68 | FIG. 22 |
| | T238K_B241T_N353K_N442A_N300K | | N442A_N300K | | | |
| N300 | CH848.3.D0949.10.17chim.6R.DS.SOSIP.664_{—} | SOSIP | >HV1301345_{—} | 69 | 70 | FIG. 22 |
| | N133D_N138T_N300K | | N133D_N138T_{—} | | | |
| | | | N300K | | | |
| N300 | CH848.3.D0949.10.17chim.6R.DS.SOSIP.664_{—} | SOSIP | >HV1301345_{—} | 71 | 72 | FIG. 22 |
| | N300D | | N300D | | | |
| N300 | CH848.3.D0949.10.17chim.6R.DS.SOSIP.664_{—} | SOSIP | >HV1302206_{—} | 73 | 74 | FIG. 22 |
| | T238K_E241T_N353K_N442A_N300D | | N442A_N300D | | | |
| N300 | CH848.3.D0949.10.17chim.6R.DS.SOSIP.664_{—} | SOSIP | >HV1301345_{—} | 75 | 76 | FIG. 22 |
| | N133D_N138T_N300D | | N133D_N138T_{—} | | | |
| | | | N300D | | | |
| N300, | CH848.3.D0949.10.17chim.6R.DS.SOSIP.664_{—} | SOSIP | >HV1301345_{—} | 77 | 78 | FIG. 22 |
| N302 | N300R_N302D | | N300R_N302D | | | |
| N300, | CH848.3.D0949.10.17chim.6R.DS.SOSIP.664_{—} | SOSIP | >HV1302206_{—} | 79 | 80 | FIG. 22 |
| N302 | T238K_E241T_N353K_N442A_N300R_N302D | | N442A_N300R_{—} | | | |
| | | | N302D | | | |
| N300, | CH848.3.D0949.10.17chim.6R.DS.SOSIP.664_{—} | SOSIP | >HV1301345_{—} | 81 | 82 | FIG. 22 |
| N302 | N133D_N138T_N300R_N302D | | N133D_N138T_{—} | | | |
| | | | N300R_N302D | | | |
| N300, | CH848.3.D0949.10.17chim.6R.DS.SOSIP.664_{—} | SOSIP | >HV1301345_{—} | 83 | 84 | FIG. 22 |
| N302 | N300K_N302D | | N300K_N302D | | | |
| N300, | CH848.3.D0949.10.17chim.6R.DS.SOSIP.664_{—} | SOSIP | >HV1302206_{—} | 85 | 86 | FIG. 22 |
| N302 | T238K_E241T_N353K_N442A_N300K_N302D | | N442A_N300K_{—} | | | |
| | | | N302D | | | |
| N300, | CH848.3.D0949.10.17chim.6R.DS.SOSIP.664_{—} | SOSIP | >HV1301345_{—} | 87 | 88 | FIG. 22 |
| N302 | N133D_N138T_N300K_N302D | | N133D_N138T_{—} | | | |
| | | | N300K_N302D | | | |
| Q417 | CH848.3.D0949.10.17chim.6R.DS.SOSIP.664_{—} | SOSIP | >HV1301345_{—} | 89 | 90 | FIG. 23 |
| | Q417Y | | Q417Y | | | |
| Q417 | CH848.3.D0949.10.17chim.6R.DS.SOSIP.664_{—} | SOSIP | >HV1302206_{—} | 91 | 92 | FIG. 23 |
| | T238K_E241T_N353K_N442A_Q417Y | | N442A_Q417Y | | | |
| Q417 | CH848.3.D0949.10.17chim.6R.DS.SOSIP.664_{—} | SOSIP | >HV1301345_{—} | 93 | 94 | FIG. 23 |
| | N133D_N138T_Q417Y | | N133D_N138T_{—} | | | |
| | | | Q417Y | | | |
| Q417 | CH848.3.D0949.10.17chim.6R.DS.SOSIP.664_{—} | SOSIP | >HV1301345_{—} | 95 | 96 | FIG. 23 |
| | Q417R | | Q417R | | | |
| Q417 | CH848.3.D0949.10.17chim.6R.DS.SOSIP.664_{—} | SOSIP | >HV1302206_{—} | 97 | 98 | FIG. 23 |
| | T238K_E241T_N353K_N442A_Q417R | | N442A_Q417R | | | |
| Q417 | CH848.3.D0949.10.17chim.6R.DS.SOSIP.664_{—} | SOSIP | >HV1301345_{—} | 99 | 100 | FIG. 23 |
| | N133D_N138T_Q417R | | N133D_N138T_{—} | | | |
| | | | Q417R | | | |
| Q417 | CH848.3.D0949.10.17chim.6R.DS.SOSIP.664_{—} | SOSIP | >HV1301345_{—} | 101 | 102 | FIG. 23 |
| | Q417K | | Q417K | | | |
| Q417 | CH848.3.D0949.10.17chim.6R.DS.SOSIP.664_{—} | SOSIP | >HV1302206_{—} | 103 | 104 | FIG. 23 |
| | T238K_E241T_N353K_N442A_Q417K | | N442A_Q417K | | | |
| Q417 | CH848.3.D0949.10.17chim.6R.DS.SOSIP.664_{—} | SOSIP | >HV1301345_{—} | 105 | 106 | FIG. 23 |
| | N133D_N138T_Q417K | | N133D_N138T_{—} | | | |
| | | | Q417K | | | |
| H330 | CH848.3.D0949.10.17chim.6R.DS.SOSIP.664_{—} | SOSIP | >HV1301345_{—} | 107 | 108 | FIG. 24 |
| | H330A | | H330A | | | |
| H330 | CH848.3.D0949.10.17chim.6R.DS.SOSIP.664_{—} | SOSIP | >HV1302206_{—} | 109 | 110 | FIG. 24 |
| | T238K_E241T_N353K_N442A_H330A | | N442A_H330A | | | |
| H330 | CH848.3.D0949.10.17chim.6R.DS.SOSIP.664_{—} | SOSIP | >HV1301345_{—} | 111 | 112 | FIG. 24 |
| | N133D_N138T_H330A | | N133D_N138T_{—} | | | |
| | | | H330A | | | |
| H330 | CH848.3.D0949.10.17chim.6R.DS.SOSIP.664_{—} | SOSIP | >HV1301345_{—} | 113 | 114 | FIG. 24 |
| | H330W | | H330W | | | |
| H330 | CH848.3.D0949.10.17chim.6R.DS.SOSIP.664_{—} | SOSIP | >HV1302206_{—} | 115 | 116 | FIG. 24 |
| | T238K_E241T_N353K_N442A_H330W | | N442A_H330W | | | |
| H330 | CH848.3.D0949.10.17chim.6R.DS.SOSIP.664_{—} | SOSIP | >HV1301345_{—} | 117 | 118 | FIG. 24 |
| | N133D_N138T_H330W | | N133D_N138T_{—} | | | |
| | | | H330W | | | |
| H330 | CH848.3.D0949.10.17chim.6R.DS.SOSIP.664_{—} | SOSIP | >HV1301345_{—} | 119 | 120 | FIG. 24 |
| | H330Y | | H330Y | | | |
| H330 | CH848.3.D0949.10.17chim.6R.DS.SOSIP.664_{—} | SOSIP | >HV1302206_{—} | 121 | 122 | FIG. 24 |
| | T238K_B241T_N353K_N442A_H330Y | | N442A_H330Y | | | |
| H330 | CH848.3.D0949.10.17chim.6R.DS.SOSIP.664_{—} | SOSIP | >HV1301345_{—} | 123 | 124 | FIG. 24 |
| | N133D_N138T_H330Y | | N133D_N138T_{—} | | | |
| | | | H330Y | | | |
| H330 | CH848.3.D0949.10.17chim.6R.DS.SOSIP.664_{—} | SOSIP | >HV1301345_{—} | 125 | 126 | FIG. 24 |
| | H330N | | H330N | | | |
| H330 | CH848.3.D0949.10.17chim.6R.DS.SOSIP.664_{—} | SOSIP | >HV1302206_{—} | 127 | 128 | FIG. 24 |
| | T238K_E241T_N353K_N442A_H330N | | N442A_H330N | | | |
| H330 | CH848.3.D0949.10.17chim.6R.DS.SOSIP.664_{—} | SOSIP | >HV1301345_{—} | 129 | 130 | FIG. 24 |
| | N133D_N138T_H330N | | N133D_N138T_{—} | | | |
| | | | H330N | | | |
| H330 | CH848.3.D0949.10.17chim.6R.DS.SOSIP.664_{—} | SOSIP | >HV1301345_{—} | 131 | 132 | FIG. 24 |
| | H330Q | | H330Q | | | |
| H330 | CH848.3.D0949.10.17chim.6R.DS.SOSIP.664_{—} | SOSIP | >HV1302206_{—} | 133 | 134 | FIG. 24 |
| | T238K_E241T_N353K_N442A_H330Q | | N442A_H330Q | | | |
| H330 | CH848.3.D0949.10.17chim.6R.DS.SOSIP.664_{—} | SOSIP | >HV1301345_{—} | 135 | 136 | FIG. 24 |
| | N133D_N138T_H330Q | | N133D_N138T_{—} | | | |
| | | | H330Q | | | |
| H330 | CH848.3.D0949.10.17chim.6R.DS.SOSIP.664_{—} | SOSIP | >HV1301345_{—} | 137 | 138 | FIG. 24 |
| | H330R | | H330R | | | |
| H330 | CH848.3.D0949.10.17chim.6R.DS.SOSIP.664_{—} | SOSIP | >HV1302206_{—} | 139 | 140 | FIG. 24 |
| | T238K_E241T_N353K_N442A_H330R | | N442A_H330R | | | |
| H330 | CH848.3.D0949.10.17chim.6R.DS.SOSIP.664_{—} | SOSIP | >HV1301345_{—} | 141 | 142 | FIG. 24 |
| | N133D_N138T_H330R | | N133D_N138T_{—} | | | |
| | | | H330R | | | |
| T415 | CH848.3.D0949.10.17chim.6R.DS.SOSIP.664_{—} | SOSIP | >HV1301345_{—} | 143 | 144 | FIG. 25 |
| | T415S | | T415S | | | |
| T415 | CH848.3.D0949.10.17chim.6R.DS.SOSIP.664_{—} | SOSIP | >HV1302206_{—} | 145 | 146 | FIG. 25 |
| | T238K_E241T_N353K_N442A_T415S | | N442A_T415S | | | |
| T415 | CH848.3.D0949.10.17chim.6R.DS.SOSIP.664_{—} | SOSIP | >HV1301345_{—} | 147 | 148 | FIG. 25 |
| | N133D_N138T_T415S | | N133D_N138T_{—} | | | |
| | | | T415S | | | |
| T415 | CH848.3.D0949.10.17chim.6R.DS.SOSIP.664_{—} | SOSIP | >HV1301345_{—} | 149 | 150 | FIG. 25 |
| | T415N | | T415N | | | |
| T415 | CH848.3.D0949.10.17chim.6R.DS.SOSIP.664_{—} | SOSIP | >HV1302206_{—} | 151 | 152 | FIG. 25 |
| | T238K_E241T_N353K_N442A_T415N | | N442A_T415N | | | |
| T415 | CH848.3.D0949.10.17chim.6R.DS.SOSIP.664_{—} | SOSIP | >HV1301345_{—} | 153 | 154 | FIG. 25 |
| | N133D_N138T_T415N | | N133D_N138T_{—} | | | |
| | | | T415N | | | |
| T415 | CH848.3.D0949.10.17chim.6R.DS.SOSIP.664_{—} | SOSIP | >HV1301345_{—} | 155 | 156 | FIG. 25 |
| | T415A | | T415A | | | |
| T415 | CH848.3.D0949.10.17chim.6R.DS.SOSIP.664_{—} | SOSIP | >HV1302206_{—} | 157 | 158 | FIG. 25 |
| | T238K_E241T_N353K_N442A_T415A | | N442A_T415A | | | |
| T415 | CH848.3.D0949.10.17chim.6R.DS.SOSIP.664_{—} | SOSIP | >HV1301345_{—} | 159 | 160 | FIG. 25 |
| | N133D_N138T_T415A | | N133D_N138T_{—} | | | |
| | | | T415A | | | |
| I323 | CH848.3.D0949.10.17chim.6R.DS.SOSIP.664_{—} | SOSIP | >HV1301345_{—} | 161 | 162 | FIG. 26 |
| | I323N | | I323N | | | |
| I323 | CH848.3.D0949.10.17chim.6R.DS.SOSIP.664_{—} | SOSIP | >HV1302206_{—} | 163 | 164 | FIG. 26 |
| | T238K_E241T_N353K_N442A_I323N | | N442A_I3237 | | | |
| I323 | CH848.3.D0949.10.17chim.6R.DS.SOSIP.664_{—} | SOSIP | >HV1301345_{—} | 165 | 166 | FIG. 26 |
| | N133D_N138T_I323N | | N133D_N138T_{—} | | | |
| | | | I323N | | | |
| I323, | CH848.3.D0949.10.17chim.6R.DS.SOSIP.664_{—} | SOSIP | >HV1301345_{—} | 167 | 168 | FIG. 26 |
| T303 | I323N_T303N | | I323N_T303N | | | |
| I323, | CH848.3.D0949.10.17chim.6R.DS.SOSIP.664_{—} | SOSIP | >HV1302206_{—} | 169 | 170 | FIG. 26 |
| T303 | T238K_E241T_N353K_N442A_I323N_T303N | | N442A_I323N_{—} | | | |
| | | | T303N | | | |
| I323, | CH848.3.D0949.10.17chim.6R.DS.SOSIP.664_{—} | SOSIP | >HV1301345_{—} | 171 | 172 | FIG. 26 |
| T303 | N133D_N138T_I323N_T303N | | N133D_N138T_{—} | | | |
| | | | I323N_T303N | | | |
| I323 | CH848.3.D0949.10.17chim.6R.DS.SOSIP.664_{—} | SOSIP | >HV1301345_{—} | 173 | 174 | FIG. 26 |
| | I323Q | | I323Q | | | |
| I323 | CH848.3.D0949.10.17chim.6R.DS.SOSIP.664_{—} | SOSIP | >HV1302206_{—} | 175 | 176 | FIG. 26 |
| | T238K_E241T_N353K_N442A_I323Q | | N442A_I323Q | | | |
| I323 | CH848.3.D0949.10.17chim.6R.DS.SOSIP.664_{—} | SOSIP | >HV1301345_{—} | 177 | 178 | FIG. 26 |
| | N133D_N138T_I323Q | | N133D_N138T_{—} | | | |
| | | | I323Q | | | |
| I323 | CH848.3.D0949.10.17chim.6R.DS.SOSIP.664_{—} | SOSIP | >HV1301345_{—} | 179 | 180 | FIG. 26 |
| | I323R | | I323R | | | |
| I323 | CH848.3.D0949.10.17chim.6R.DS.SOSIP.664_{—} | SOSIP | >HV1302206_{—} | 181 | 182 | FIG. 26 |
| | T238K_E241T_N353K_N442A_I323R | | N442A_I323R | | | |
| I323 | CH848.3.D0949.10.17chim.6R.DS.SOSIP.664_{—} | SOSIP | >HV1301345_{—} | 183 | 184 | FIG. 26 |
| | N133D_N138T_I323R | | N133D_N138T_{—} | | | |
| | | | I323R | | | |
| I323 | CH848.3.D0949.10.17chim.6R.DS.SOSIP.664_{—} | SOSIP | >HV1301345_{—} | 185 | 186 | FIG. 26 |
| | I323K | | I323K | | | |
| I323 | CH848.3.D0949.10.17chim.6R.DS.SOSIP.664_{—} | SOSIP | >HV1302206_{—} | 187 | 188 | FIG. 26 |
| | T238K_E241T_N353K_N442A_I323K | | N442A_I323K | | | |
| I323 | CH848.3.D0949.10.17chim.6R.DS.SOSIP.664_{—} | SOSIP | >HV1301345_{—} | 189 | 190 | FIG. 26 |
| | N133D_N138T_I323K | | N133D_N138T_{—} | | | |
| | | | I323K | | | |
| T444 | CH848.3.D0949.10.17chim.6R.DS.SOSIP.664_{—} | SOSIP | >HV1301345_{—} | 191 | 192 | FIG. 27 |
| | T444H | | T444H | | | |
| T444 | CH848.3.D0949.10.17chim.6R.DS.SOSIP.664_{—} | SOSIP | >HV1302206_{—} | 193 | 194 | FIG. 27 |
| | T238K_E241T_N353K_N442A_T444H | | N442A_T444H | | | |
| T444 | CH848.3.D0949.10.17chim.6R.DS.SOSIP.664_{—} | SOSIP | >HV1301345_{—} | 195 | 196 | FIG. 27 |
| | N133D_N138T_T444H | | N133D_N138T_{—} | | | |
| | | | T444H | | | |
| T444 | CH848.3.D0949.10.17chim.6R.DS.SOSIP.664_{—} | SOSIP | >HV1301345_{—} | 197 | 198 | FIG. 27 |
| | T444G | | T444G | | | |
| T444 | CH848.3.D0949.10.17chim.6R.DS.SOSIP.664_{—} | SOSIP | >HV1302206_{—} | 199 | 200 | FIG. 27 |
| | T238K_E241T_N353K_N442A_T444G | | N442A_T444G | | | |
| T444 | CH848.3.D0949.10.17chim.6R.DS.SOSIP.664_{—} | SOSIP | >HV1301345_{—} | 201 | 202 | FIG. 27 |
| | N133D_N138T_T444G | | N133D_N138T_{—} | | | |
| | | | T444G | | | |
| I439, | CH848.3.D0949.10.17chim.6R.DS.SOSIP.664_{—} | SOSIP | >HV1301345_{—} | 203 | 204 | FIG. 27 |
| I443 | I439A_I443A | | I439A_I443A | | | |
| I439, | CH848.3.D0949.10.17chim.6R.DS.SOSIP.664_{—} | SOSIP | >HV1302206_{—} | 205 | 206 | FIG. 28 |
| I443 | T238K_E241T_N353K_N442A_I439A_I443A | | N442A_I439A_{—} | | | |
| | | | I443A | | | |
| I439, | CH848.3.D0949.10.17chim.6R.DS.SOSIP.664_{—} | SOSIP | >HV1301345_{—} | 207 | 208 | FIG. 28 |
| I443 | N133D_N138T_I439A_I443A | | N133D_N138T_{—} | | | |
| | | | I439A_I443A | | | |
| I439, | CH848.3.D0949.10.17chim.6R.DS.SOSIP.664_{—} | SOSIP | >HV1301345_{—} | 209 | 210 | FIG. 28 |
| I443 | I439N_I443N | | I439N_I443N | | | |
| I439, | CH848.3.D0949.10.17chim.6R.DS.SOSIP.664_{—} | SOSIP | >HV1302206_{—} | 211 | 212 | FIG. 28 |
| I443 | T238K_E241T_N353K_N442A_I439N_I443N | | N442A_I439N_{—} | | | |
| | | | I443N | | | |
| I439, | CH848.3.D0949.10.17chim.6R.DS.SOSIP.664_{—} | SOSIP | >HV1301345_{—} | 213 | 214 | FIG. 28 |
| I443 | N133D_N138T_I439N_I443N | | N133D_N138T_{—} | | | |
| | | | I439N_I443N | | | |
| K327R | CH848.3.D0949.10.17chim.6R.DS.SOSIP.664 | SOSIP | >CH848.3.D0949.10.17chim.6R.DS.SOSIP.664 | | 403 | FIG. 52 |
| | | | Plus_K327R_mutation | | | |

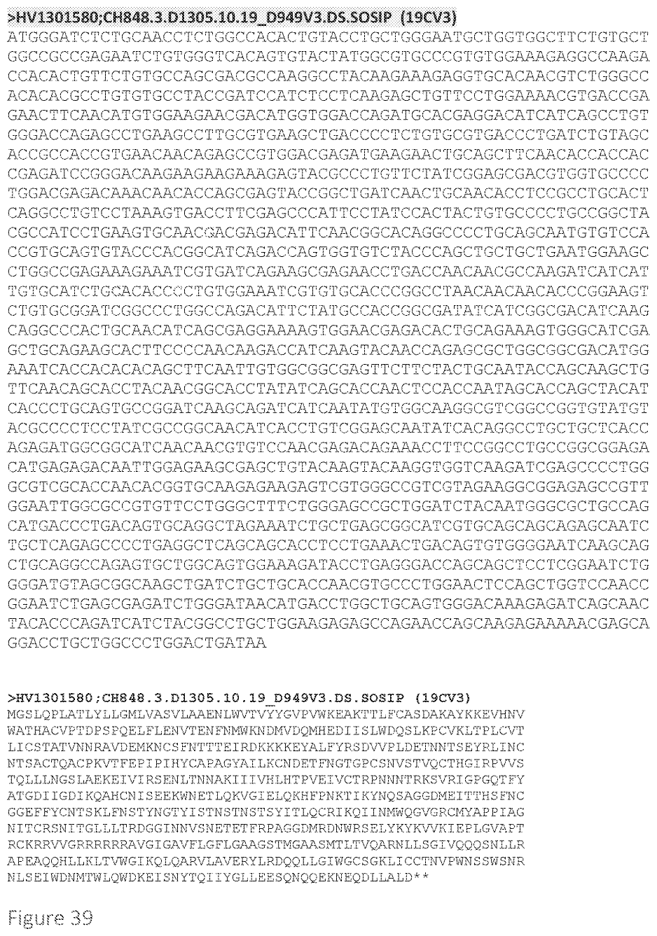

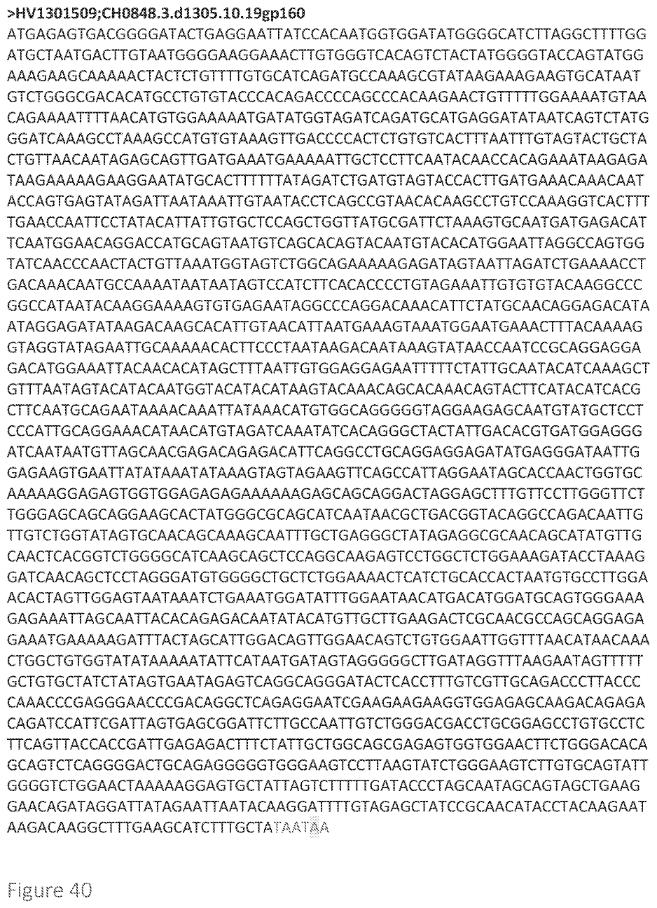

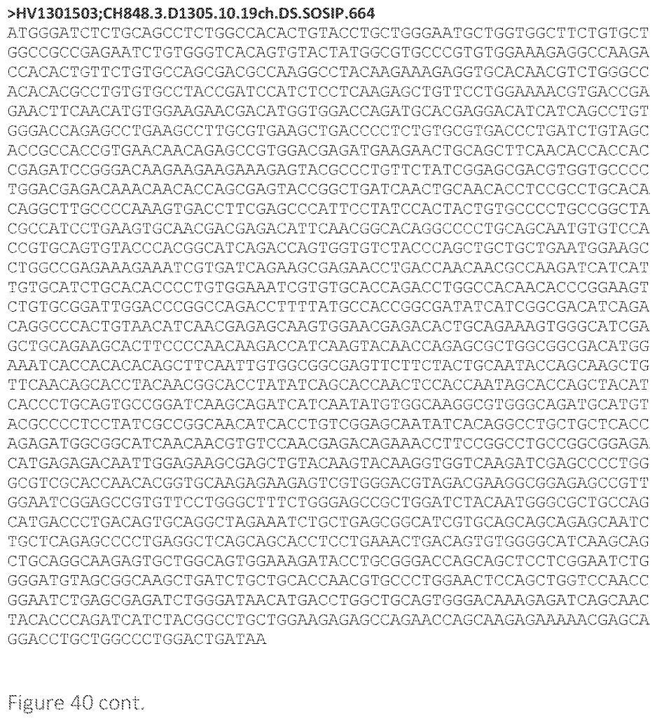

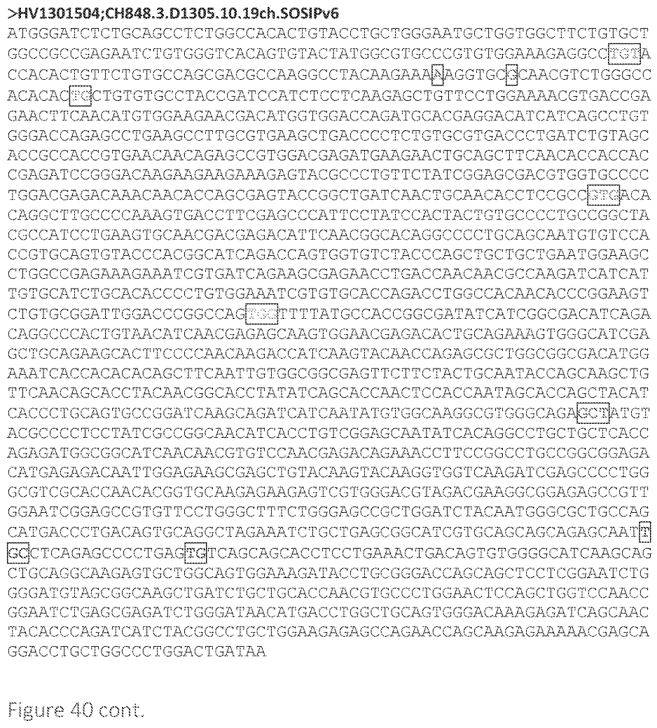

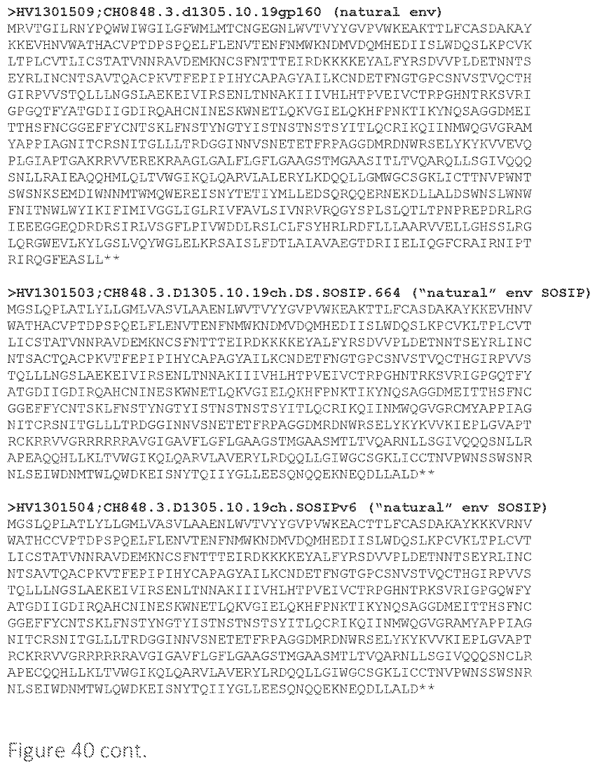

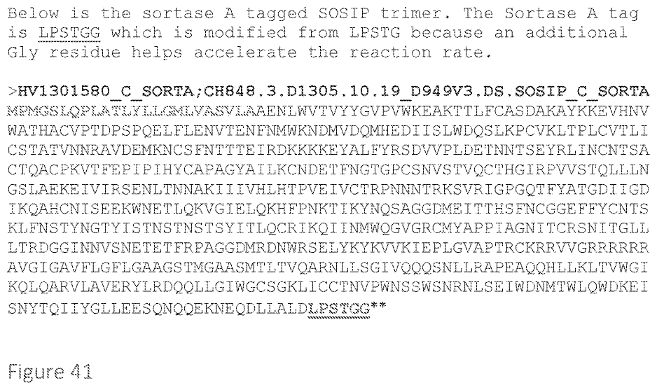

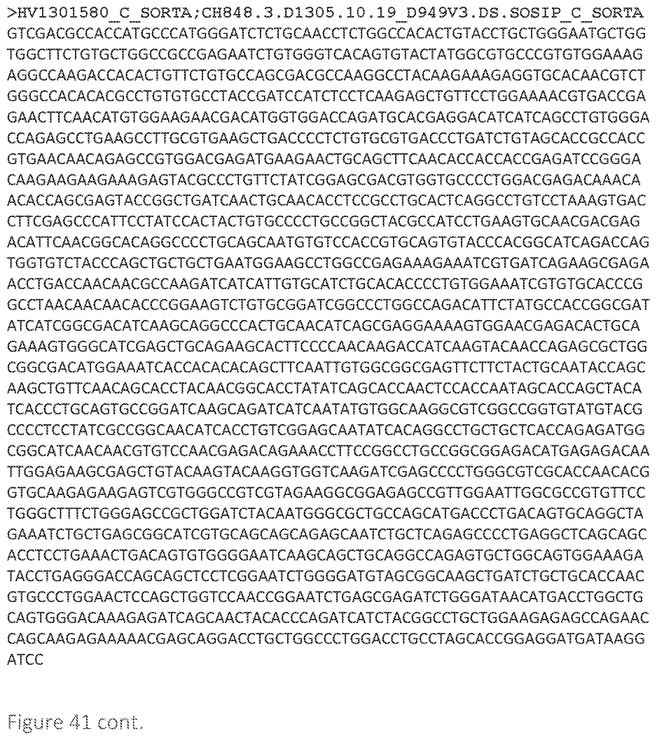

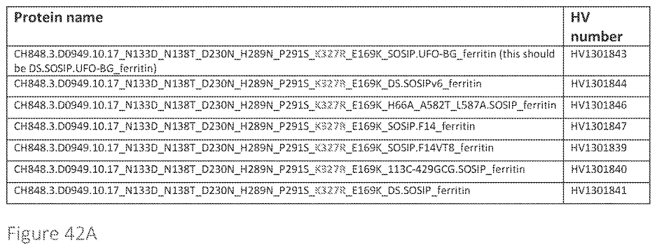

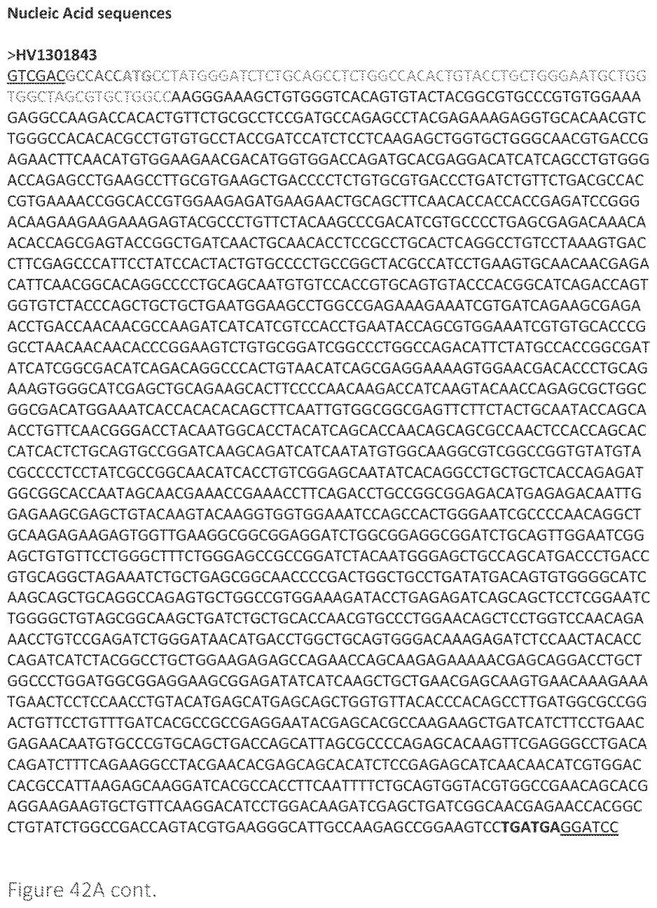

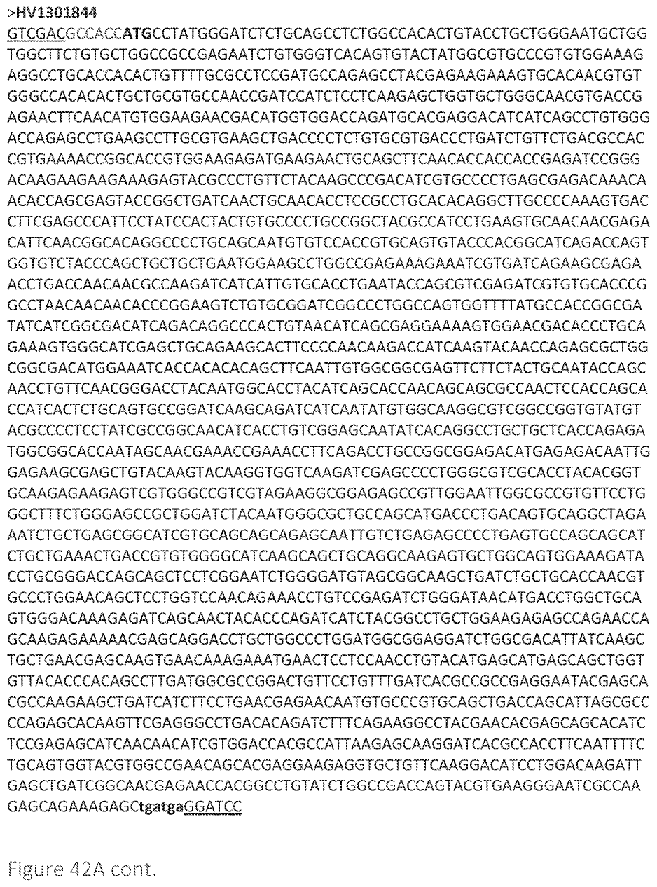

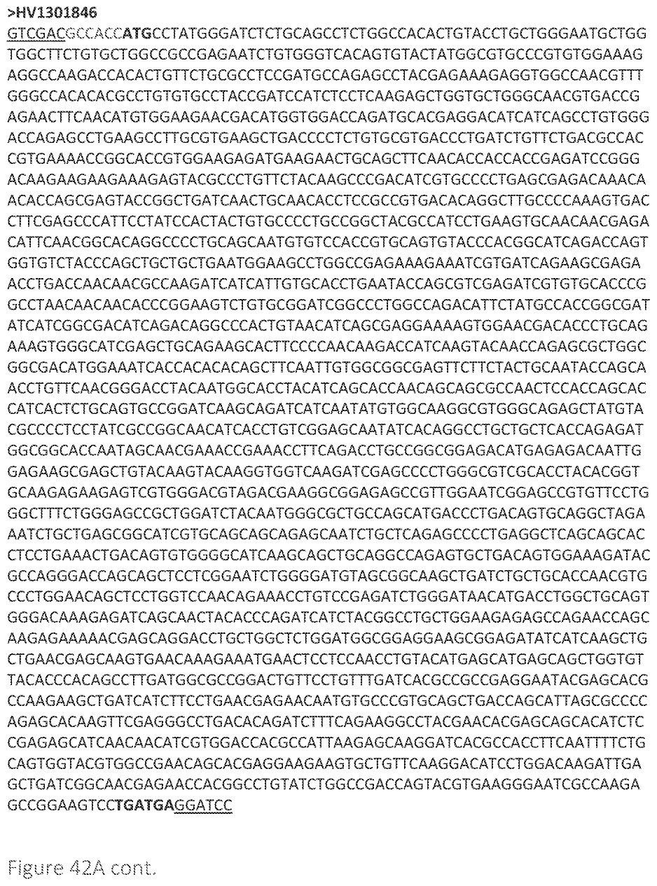

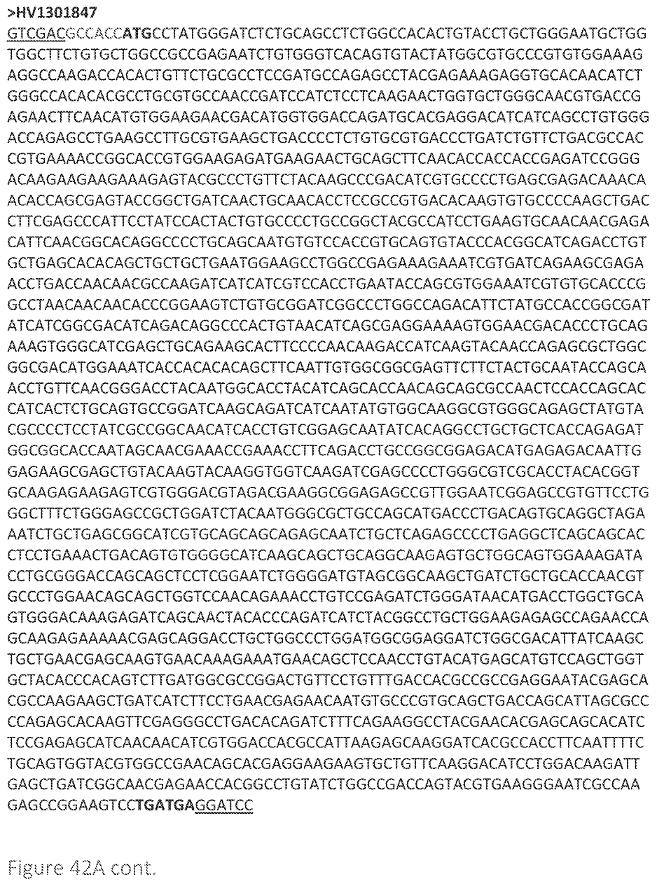

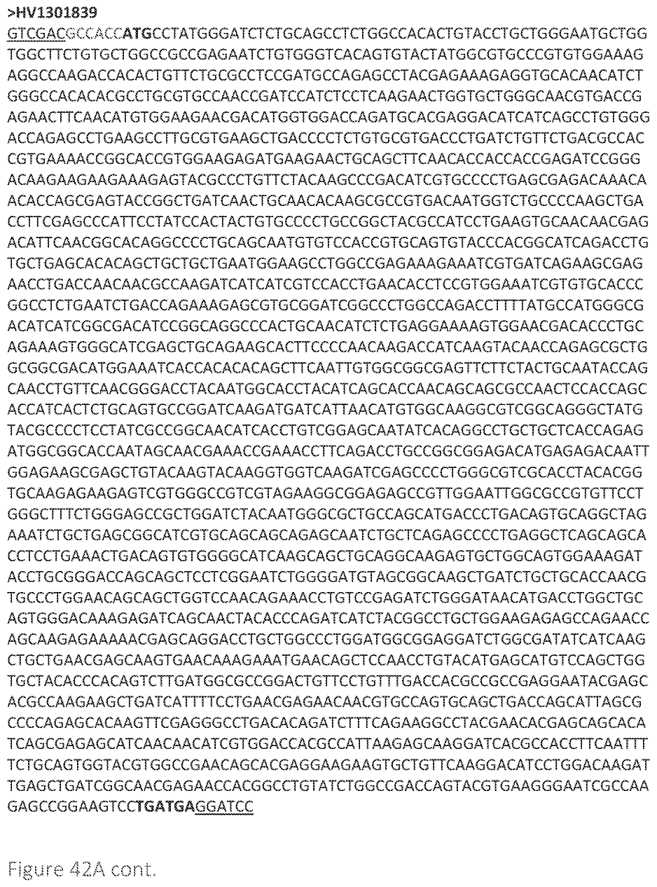

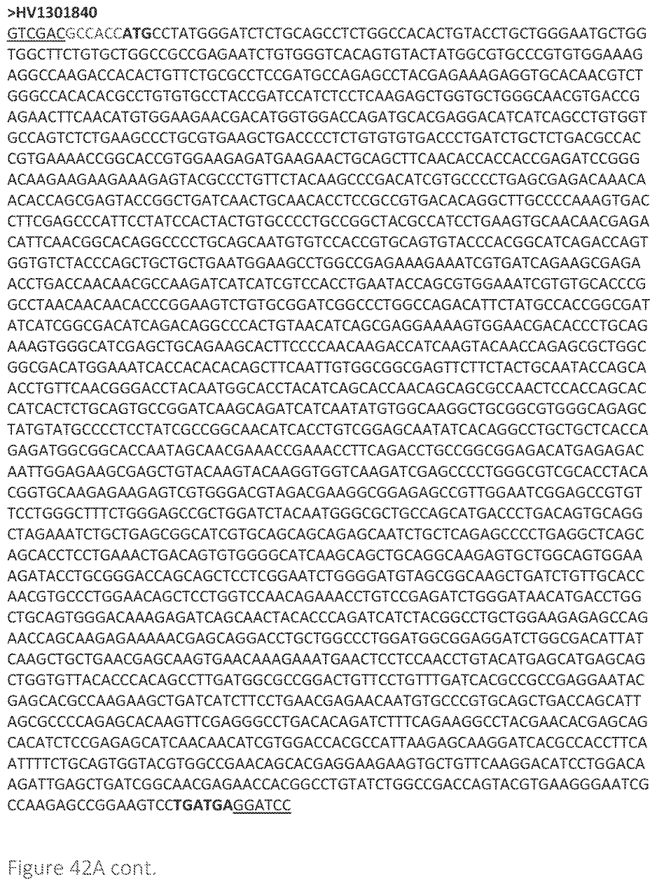

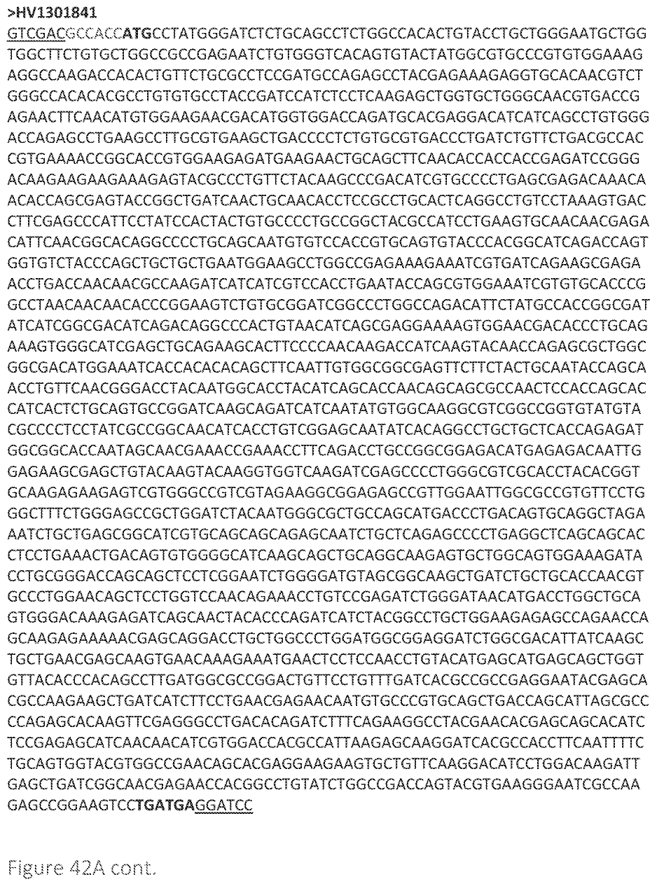

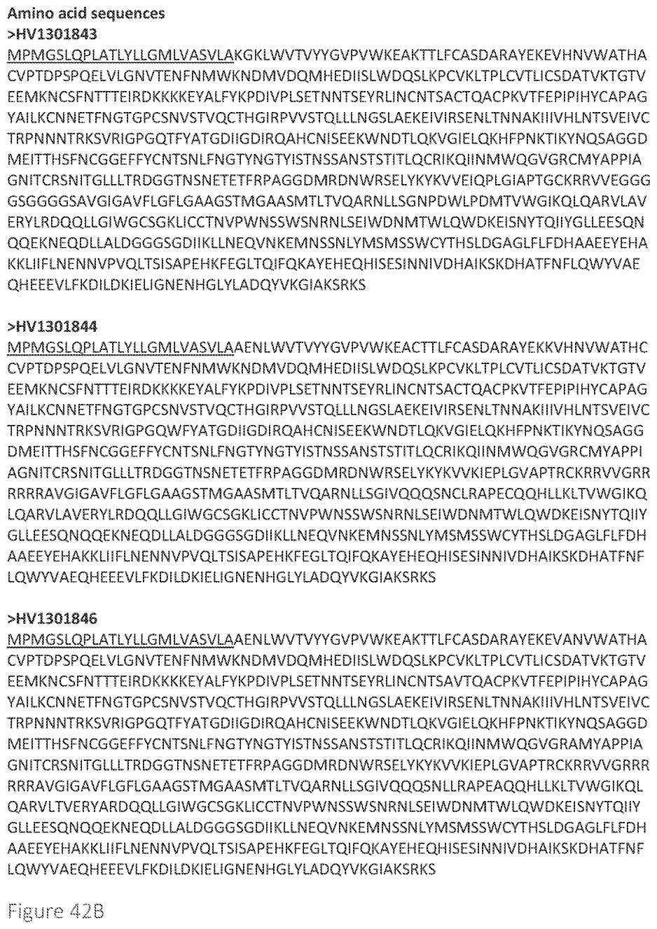

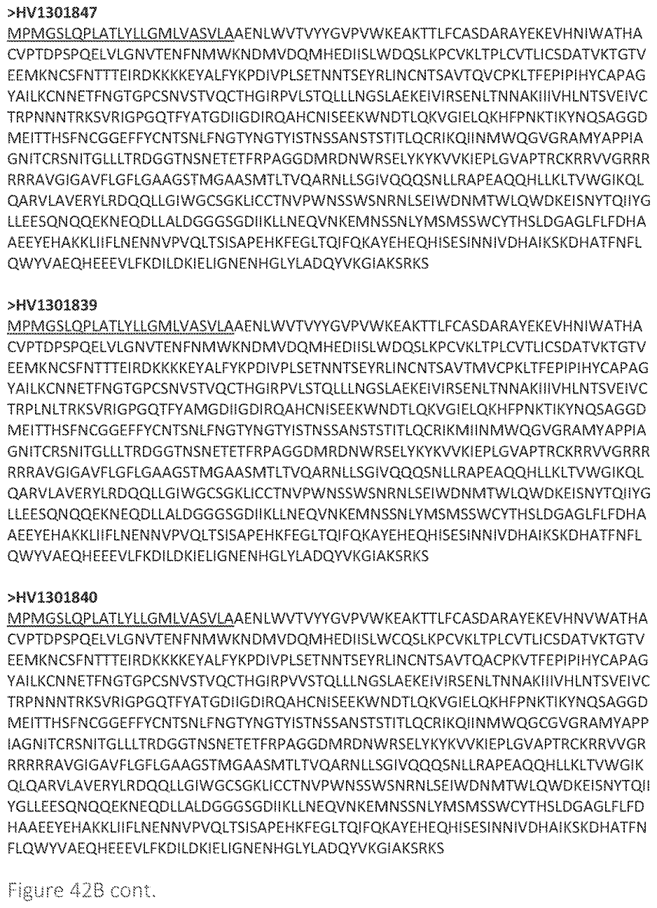

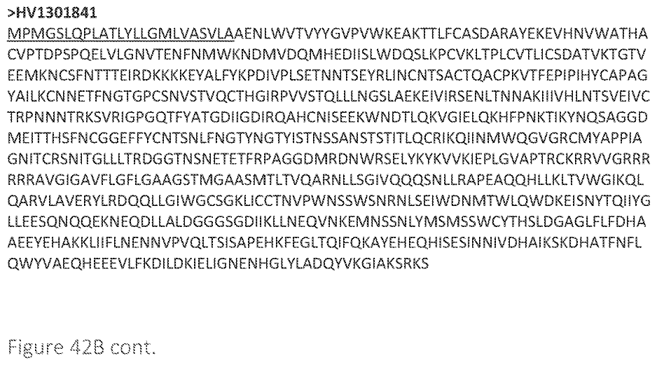

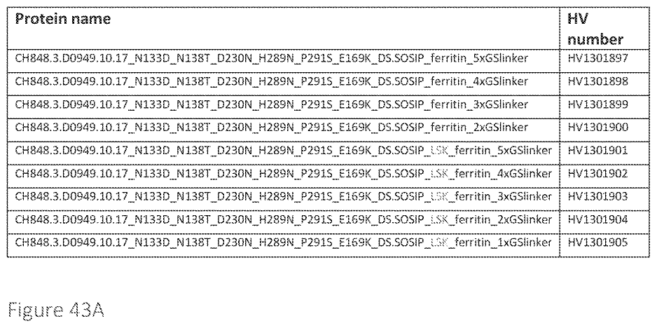

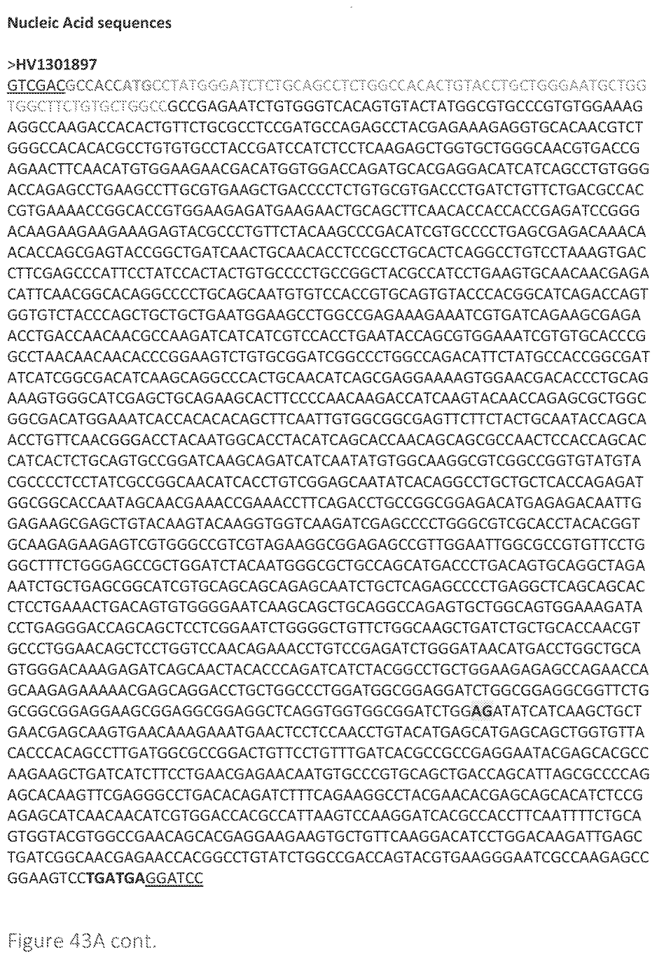

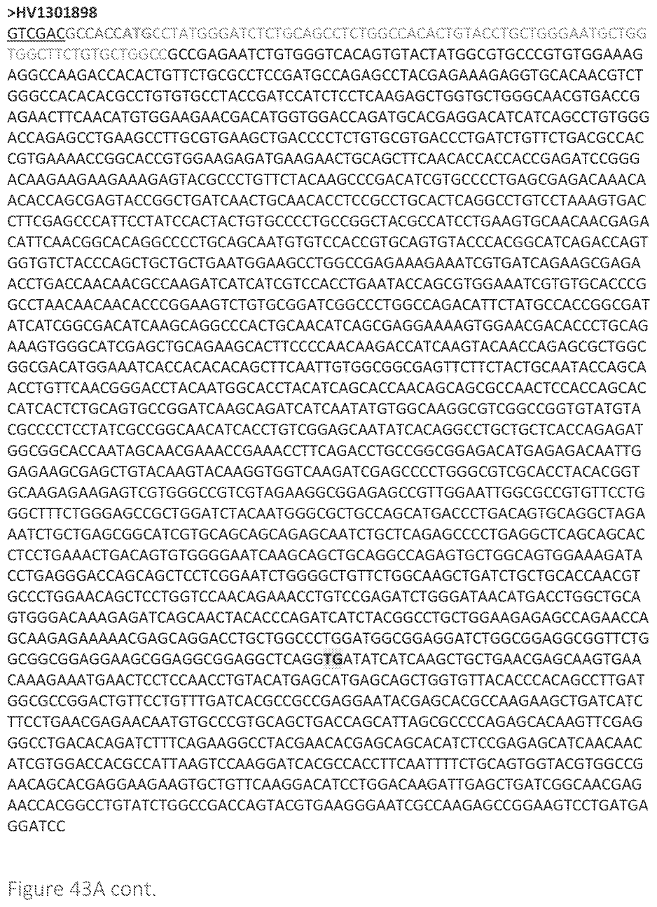

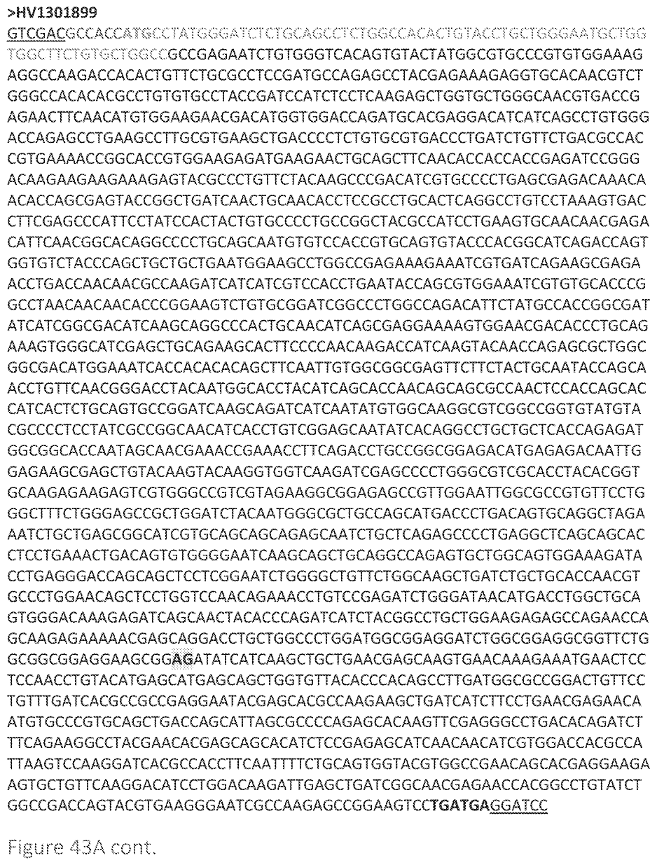

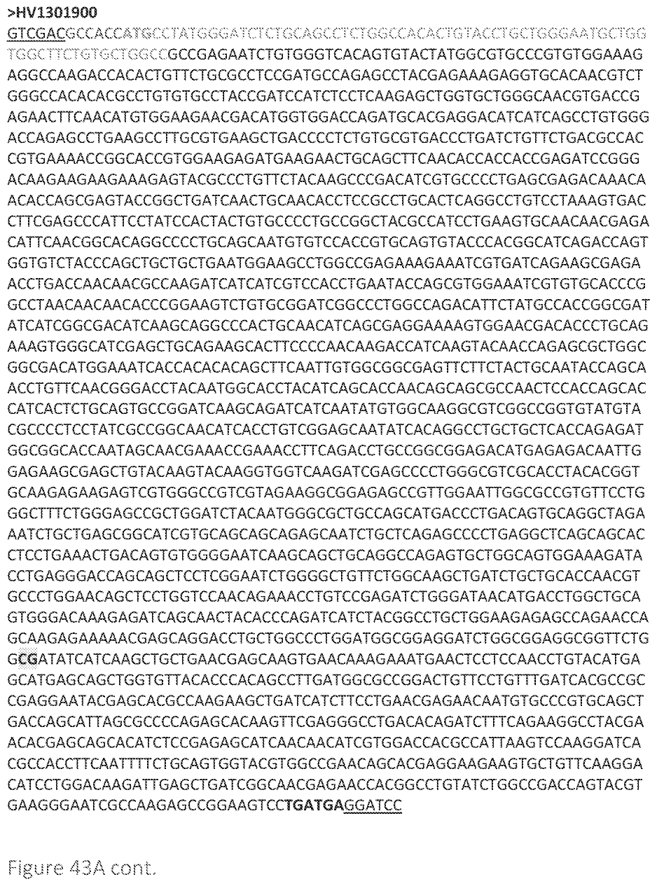

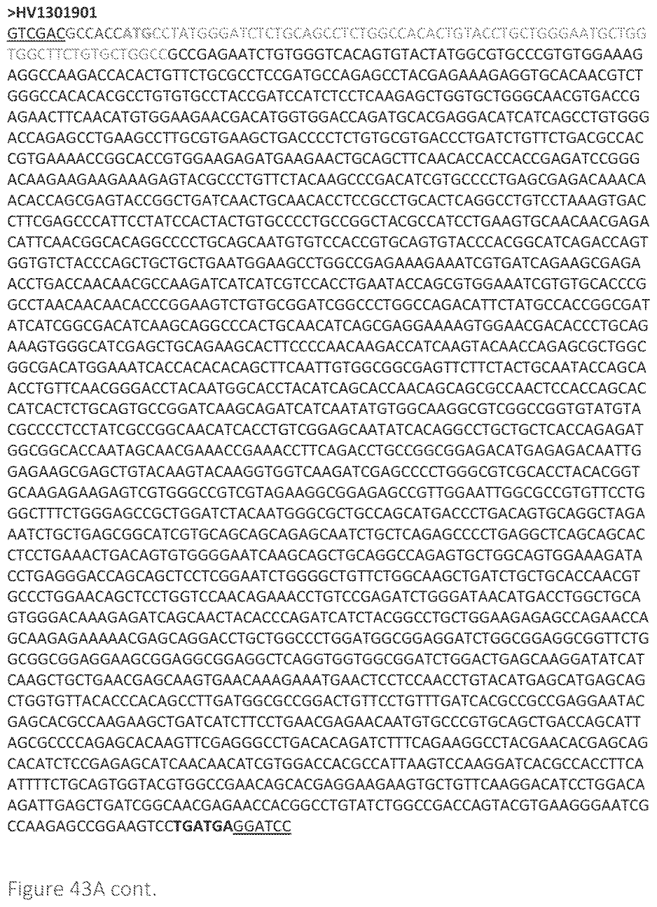

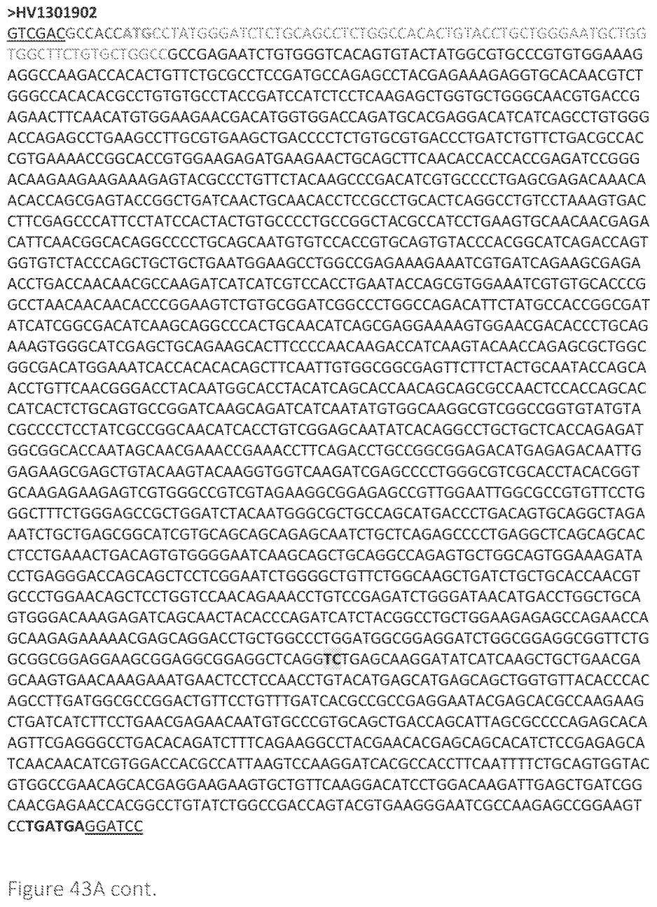

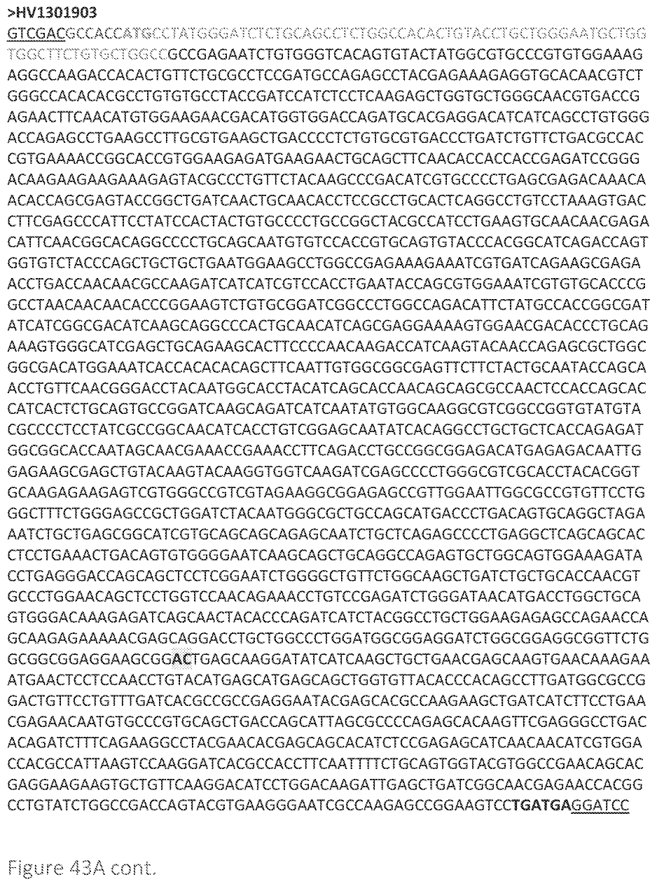

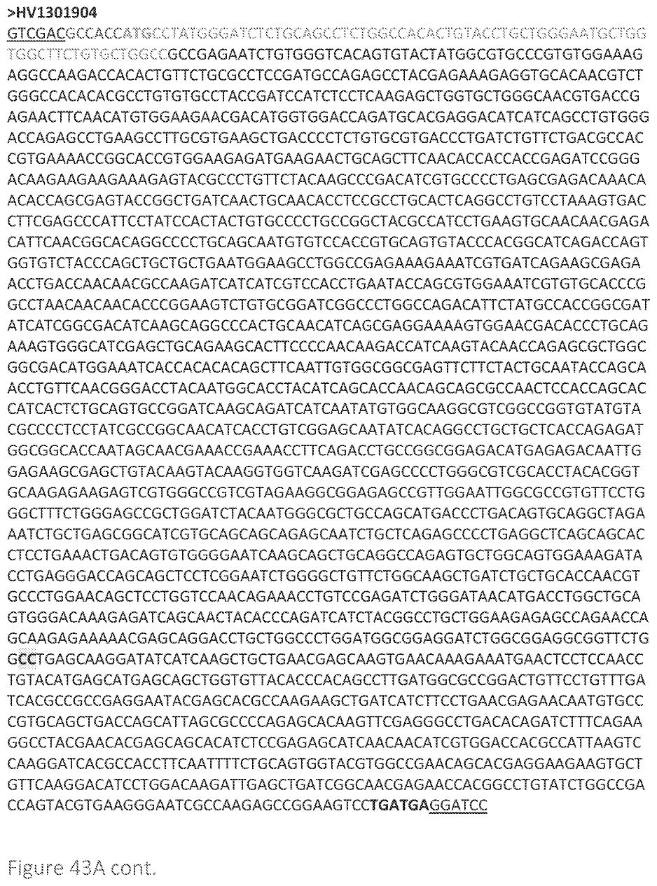

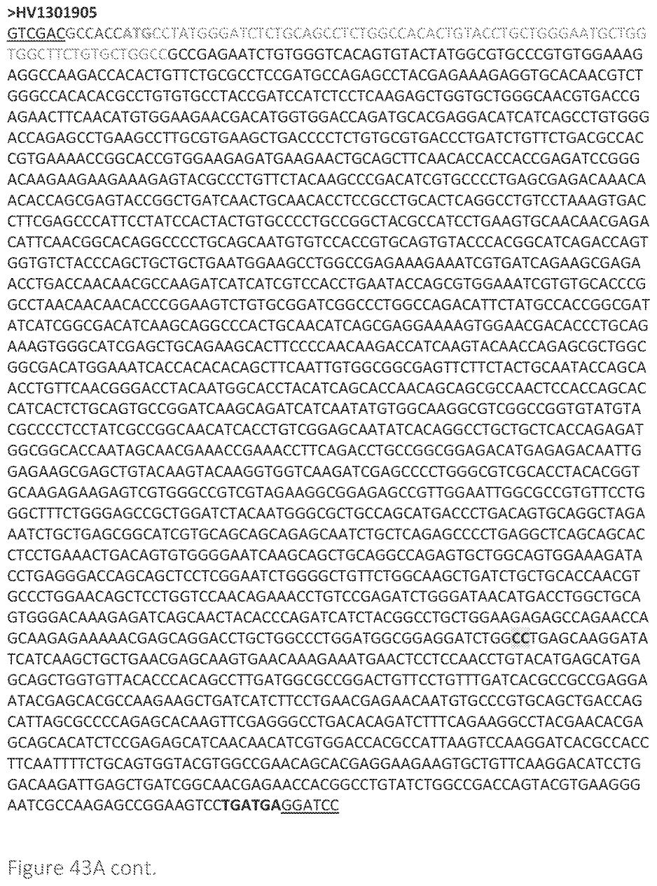

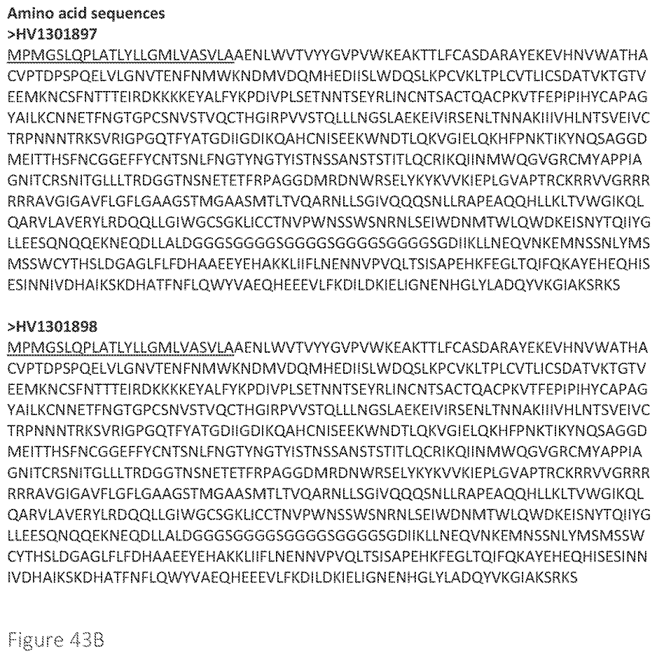

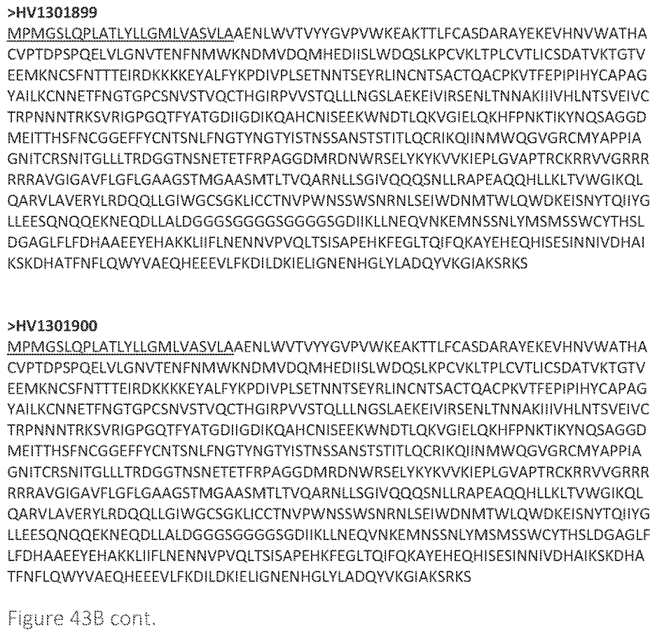

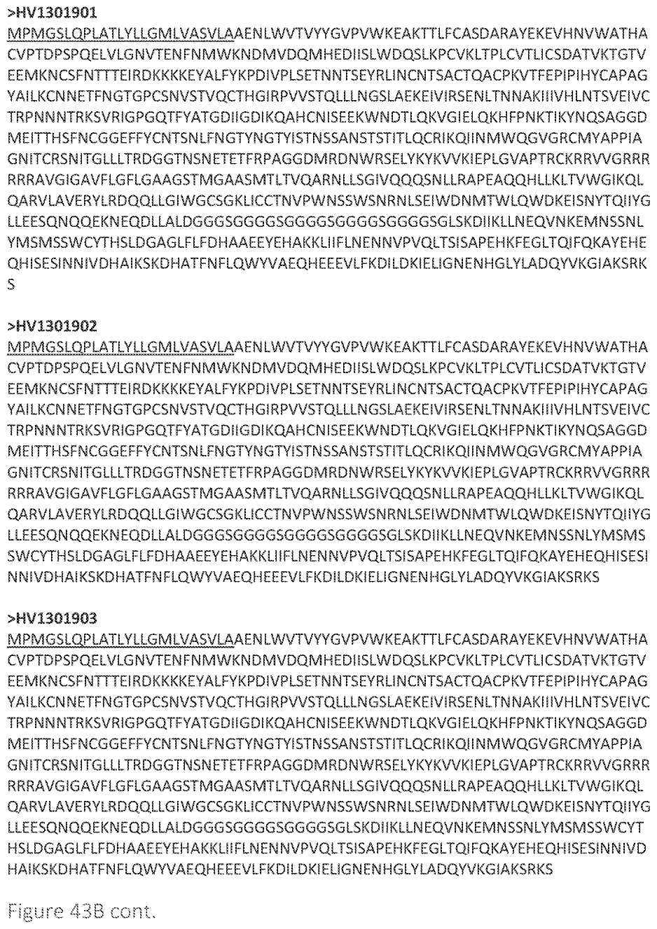

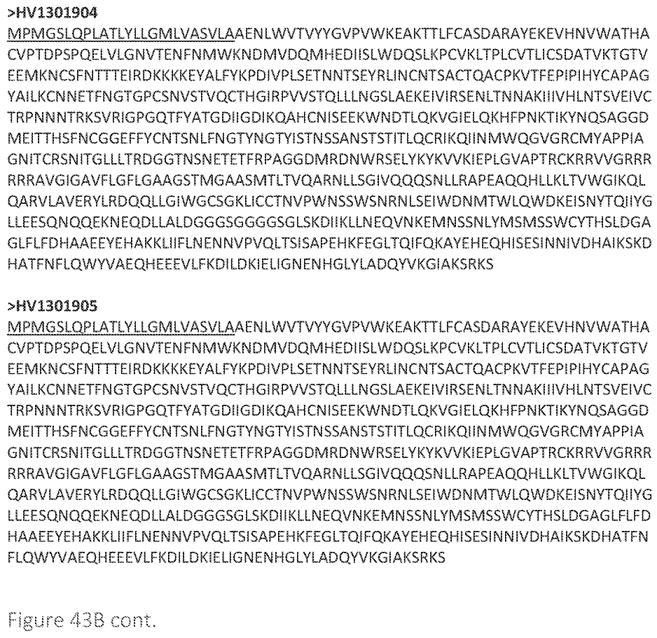

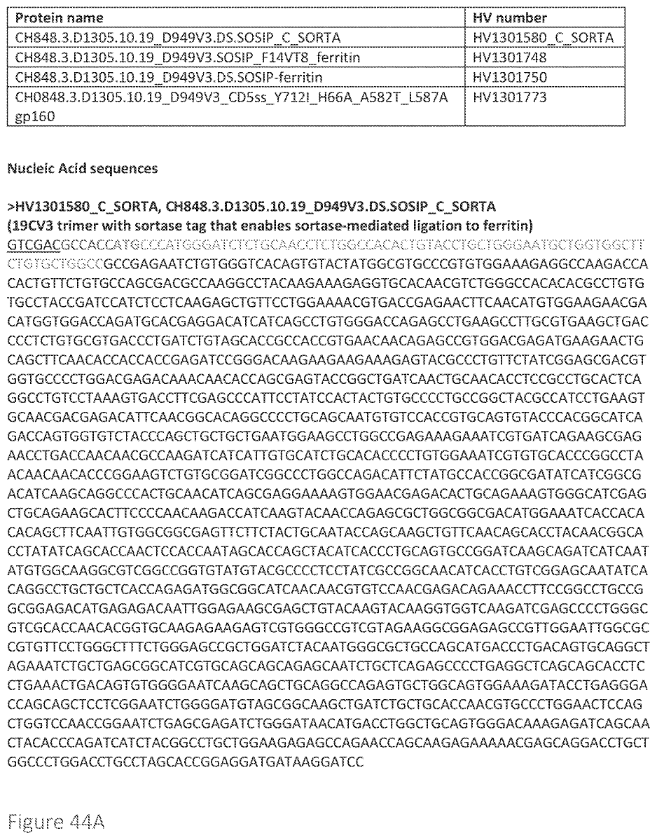

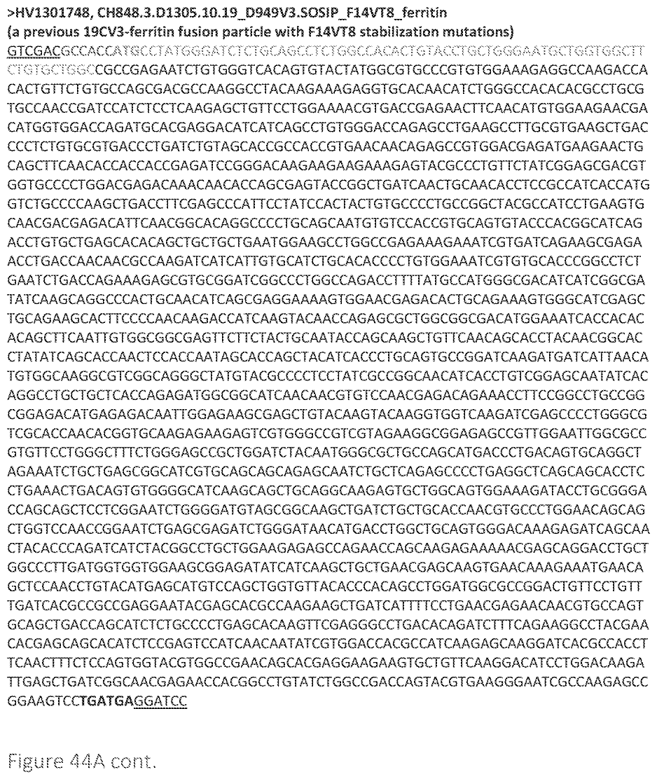

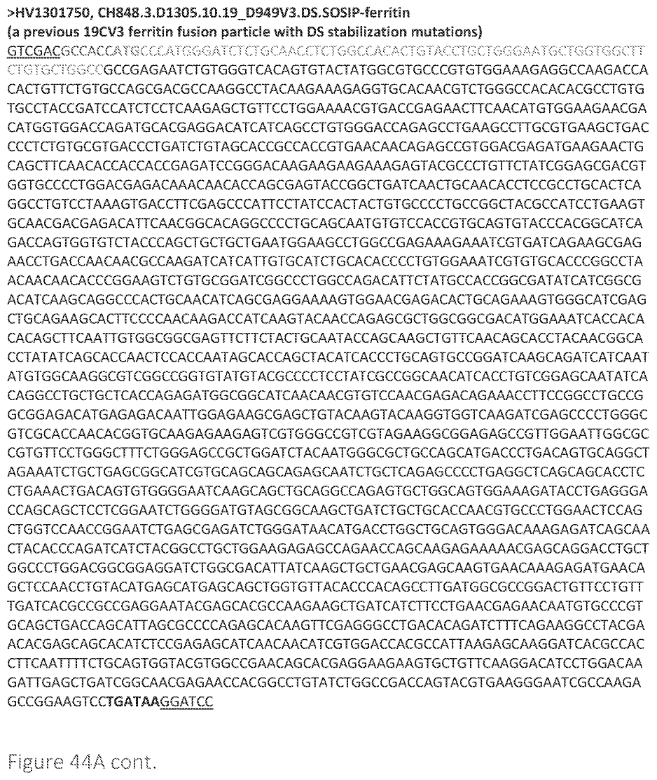

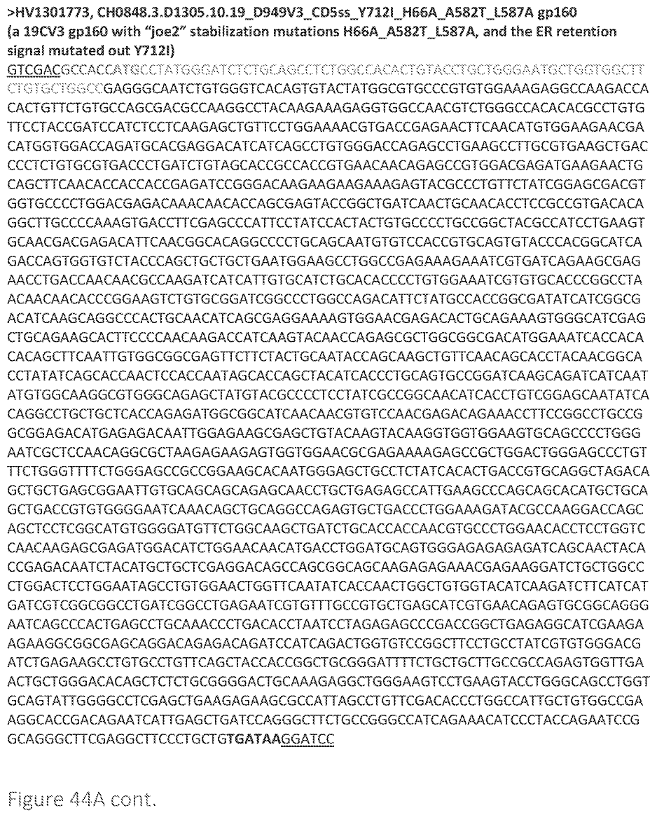

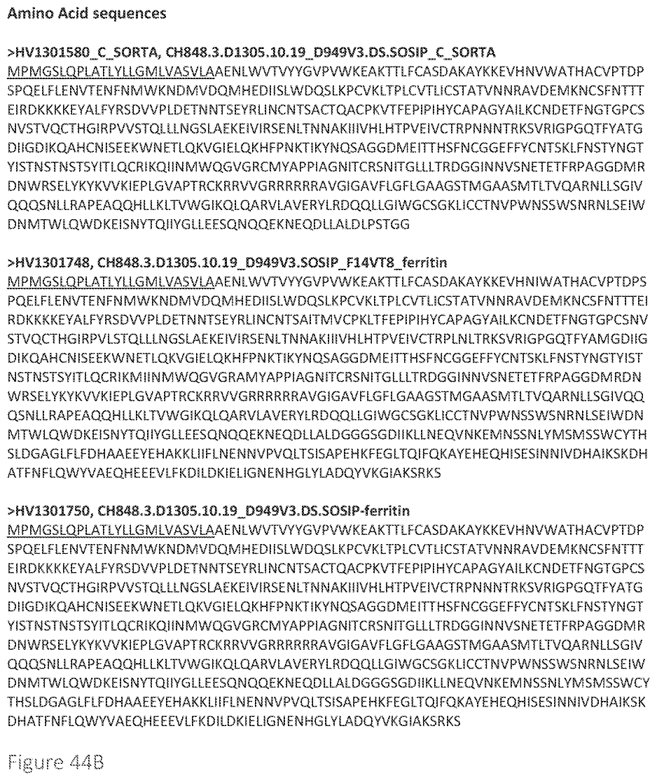

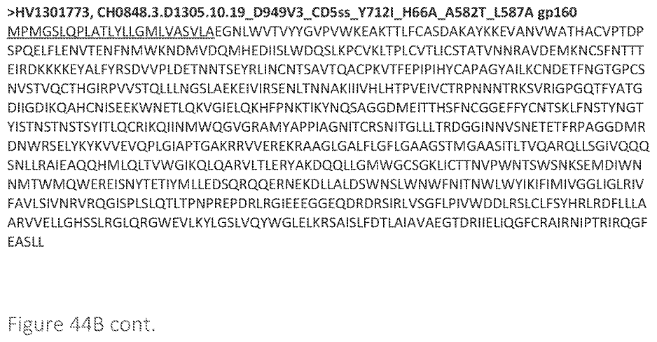

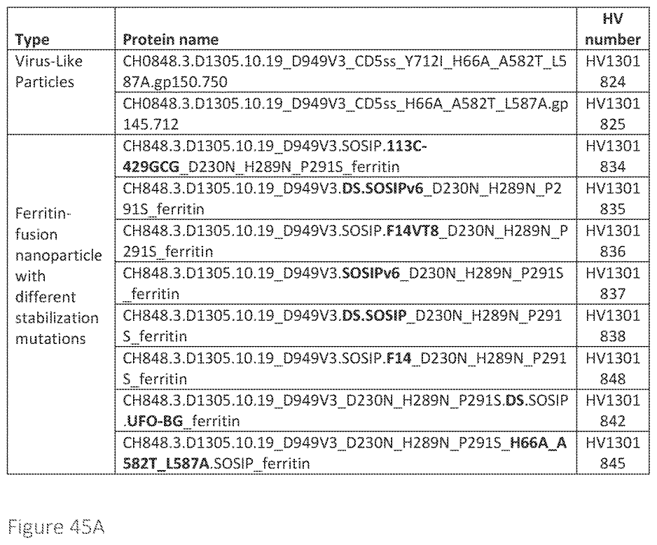

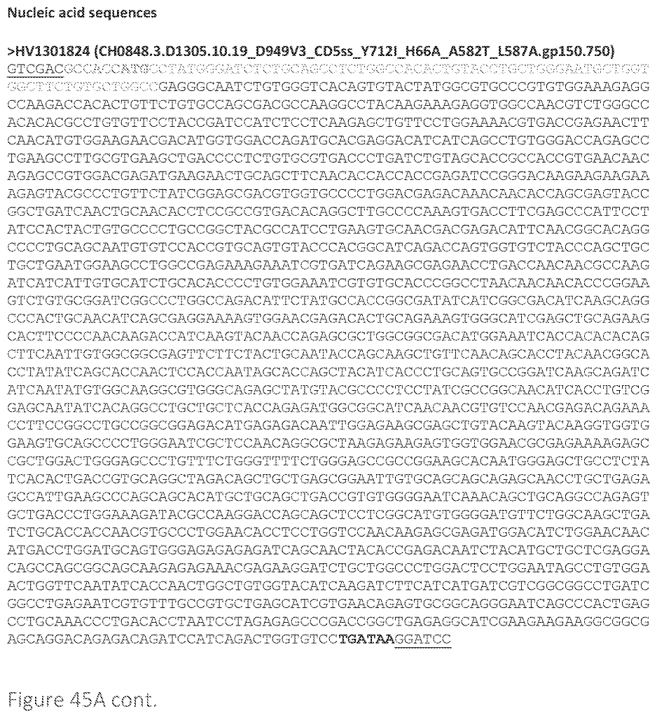

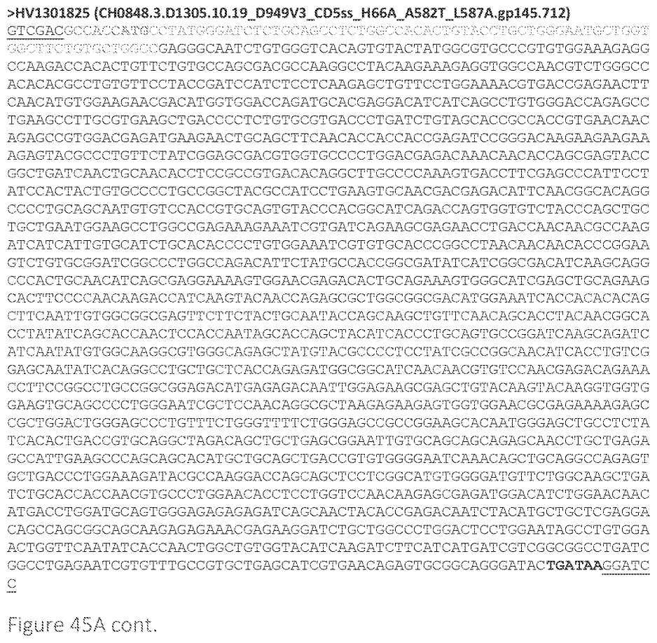

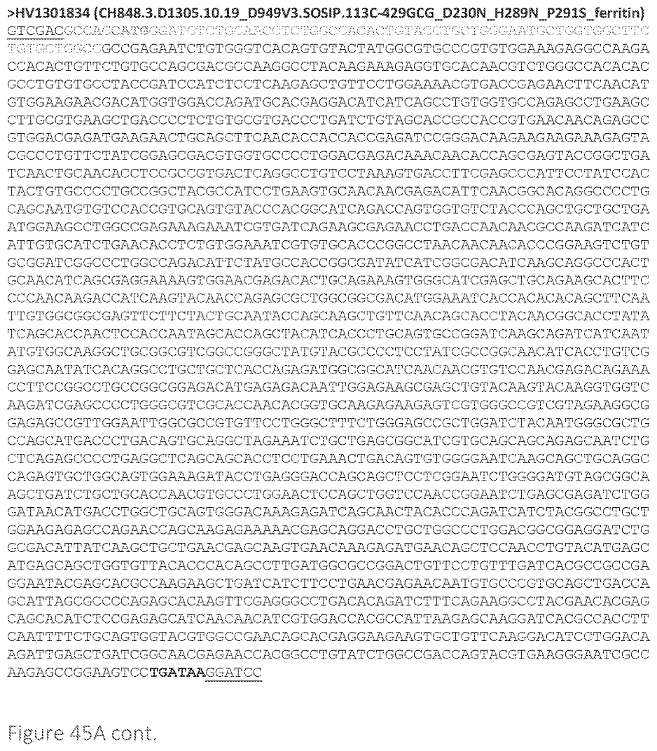

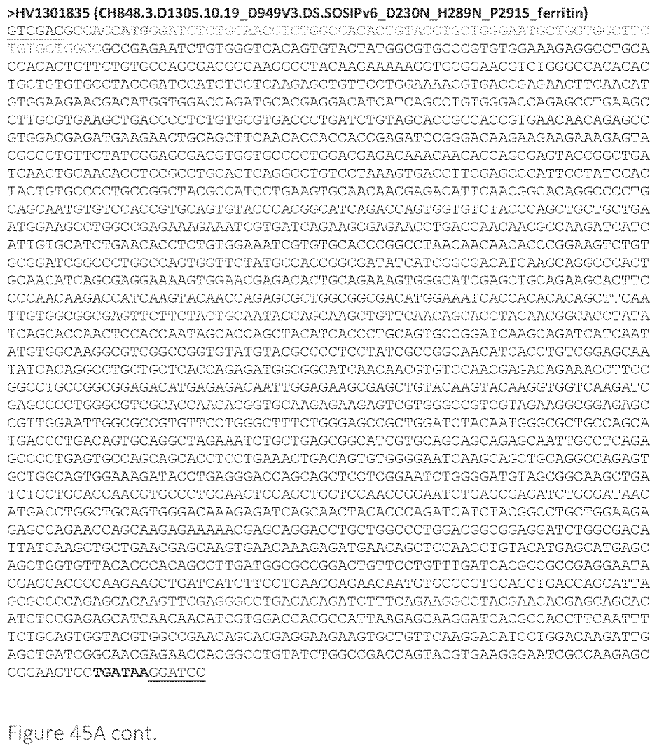

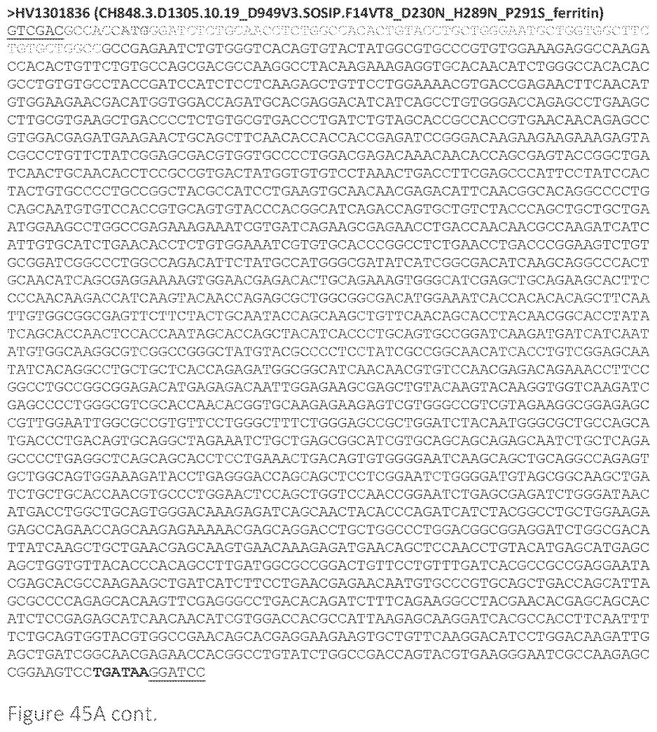

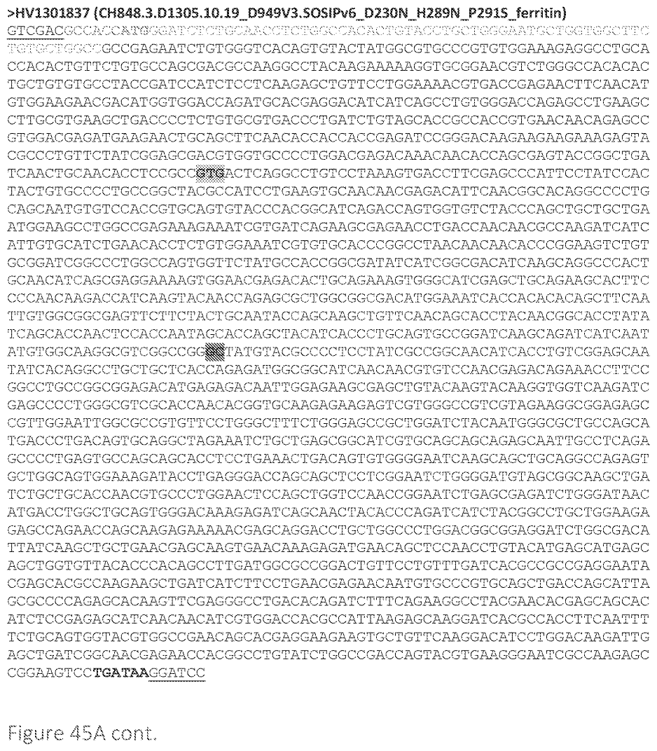

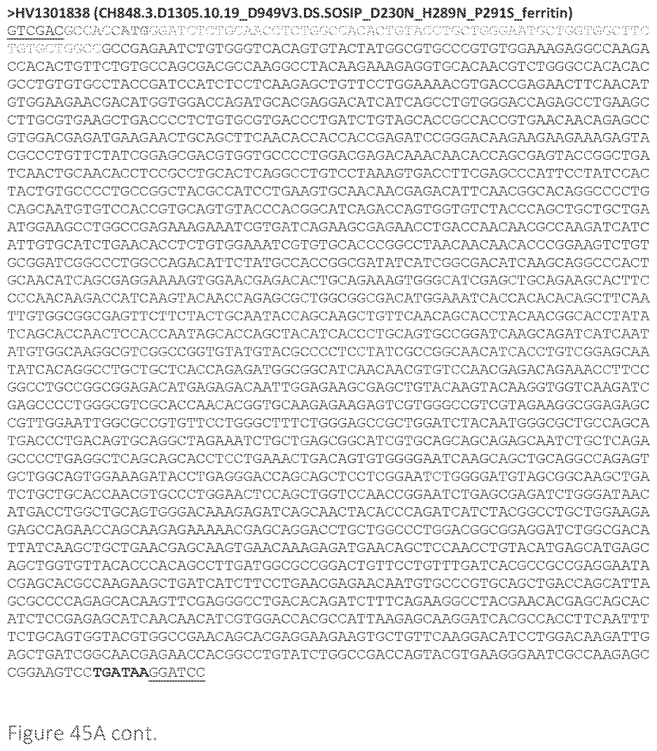

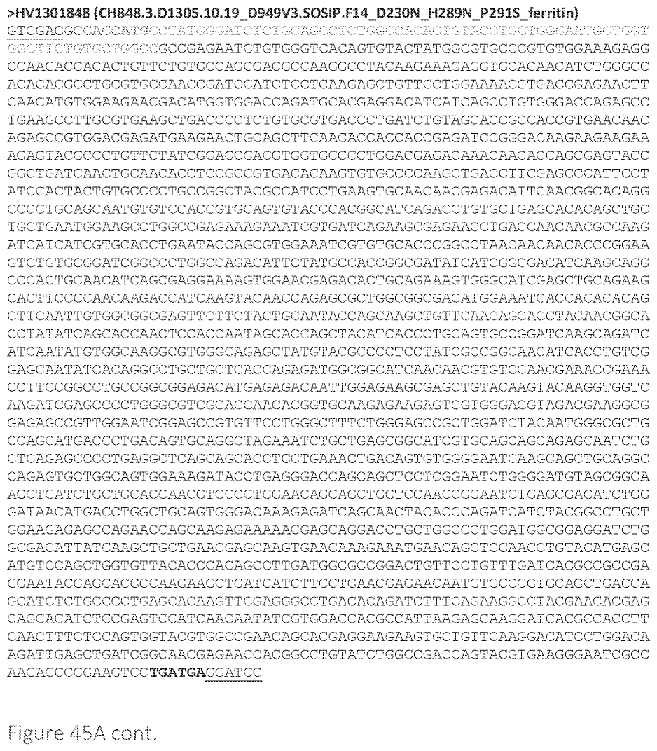

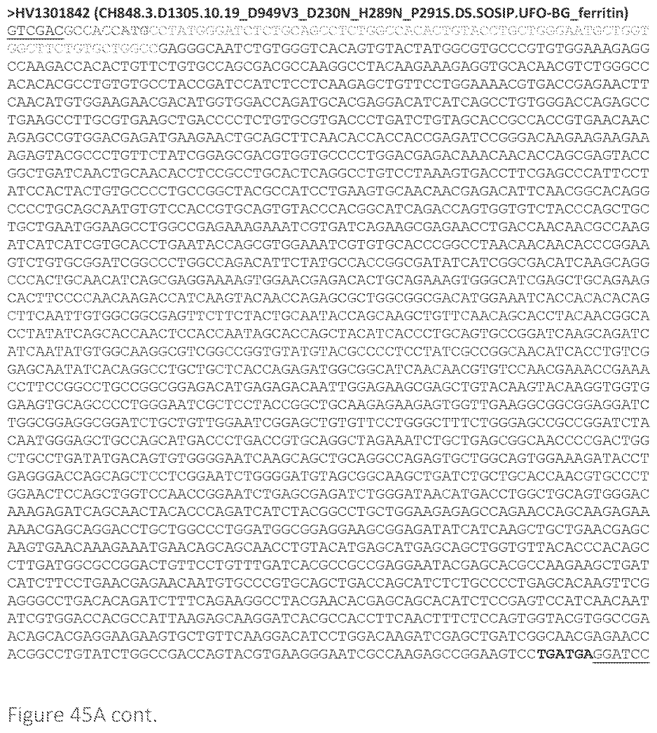

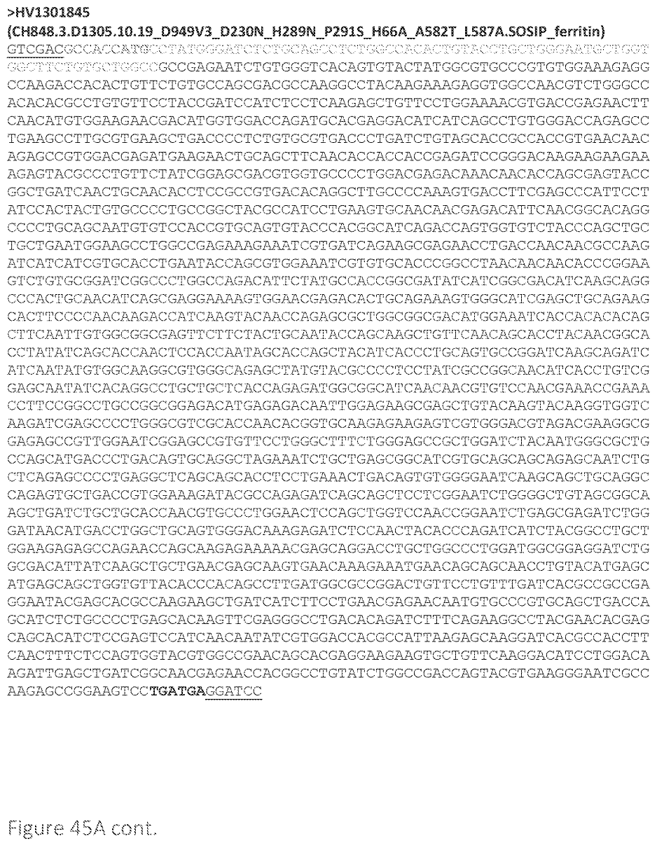

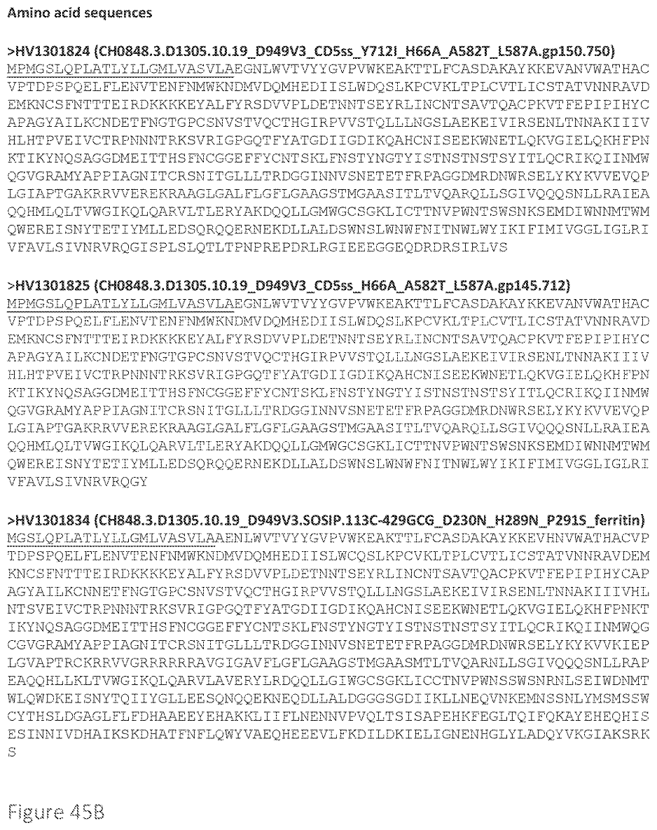

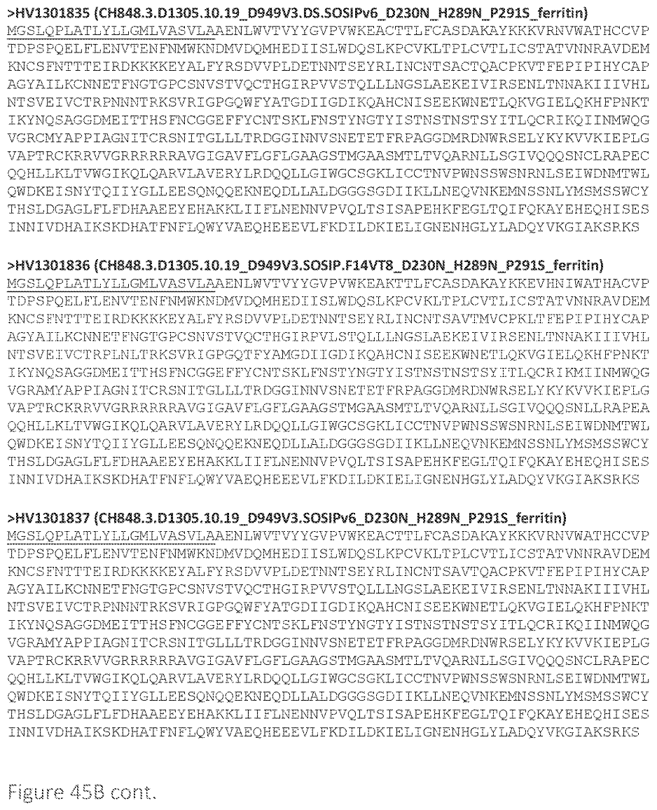

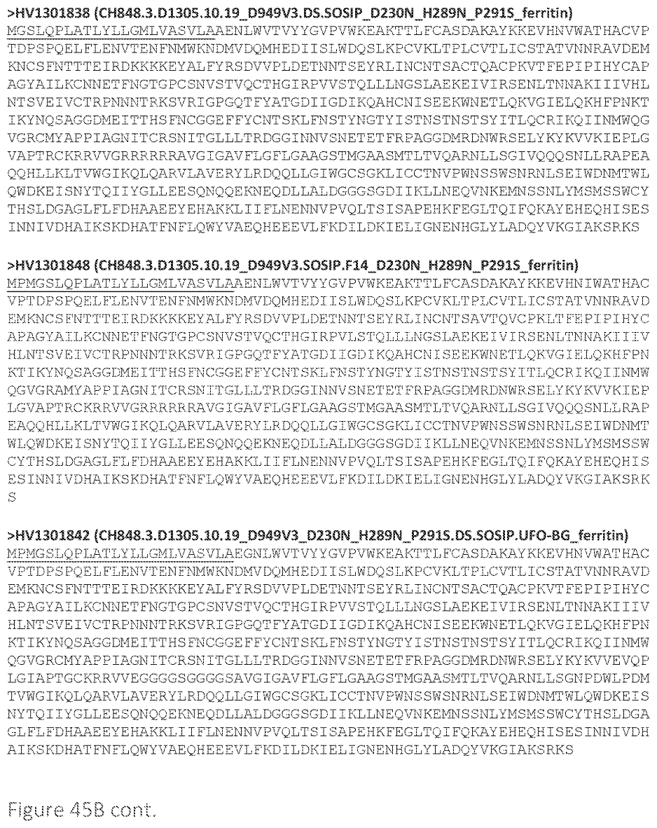

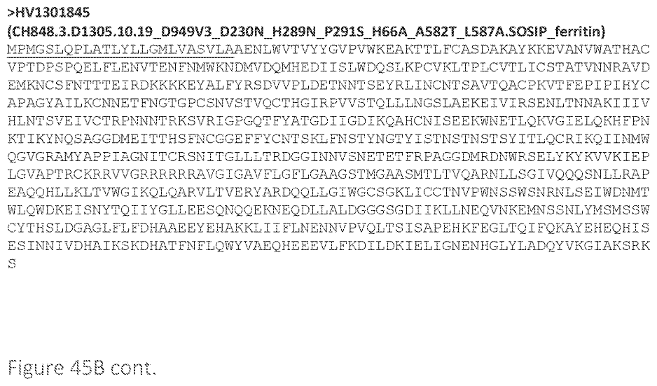

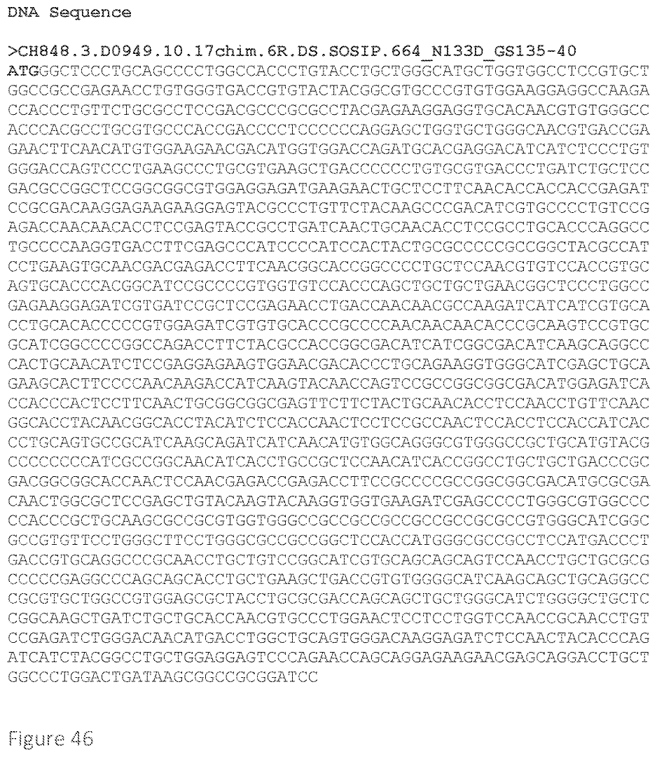

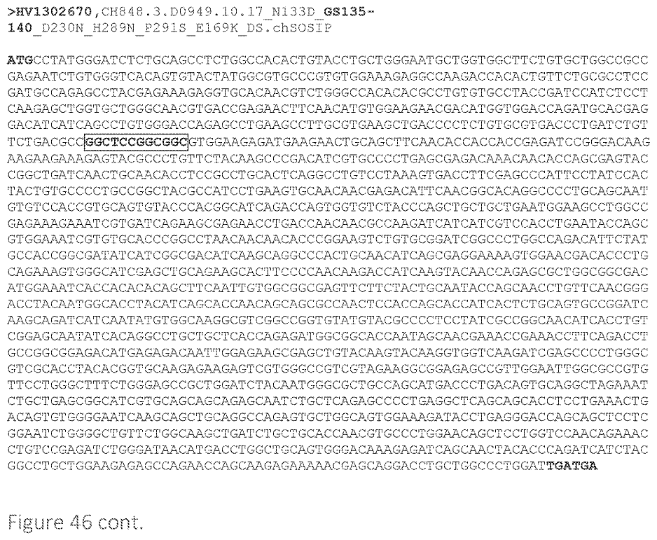

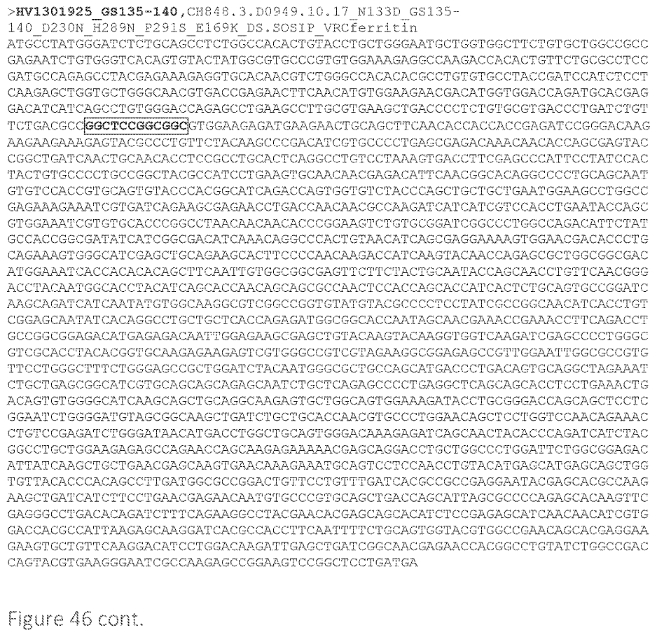

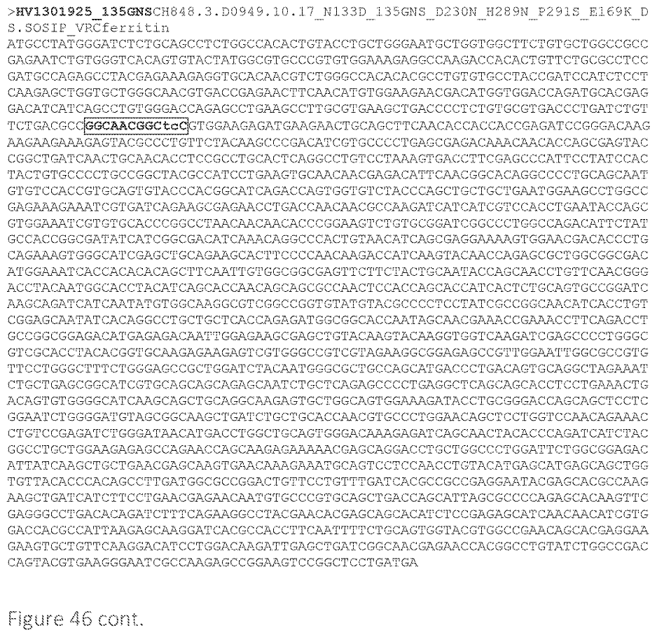

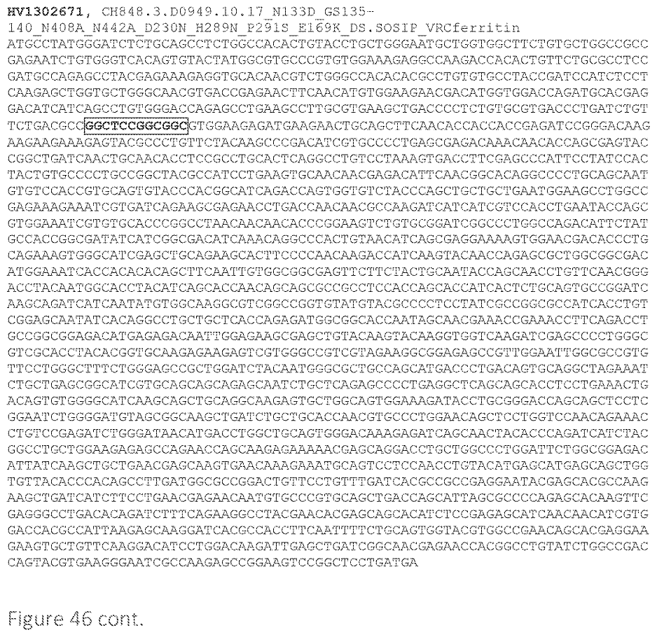

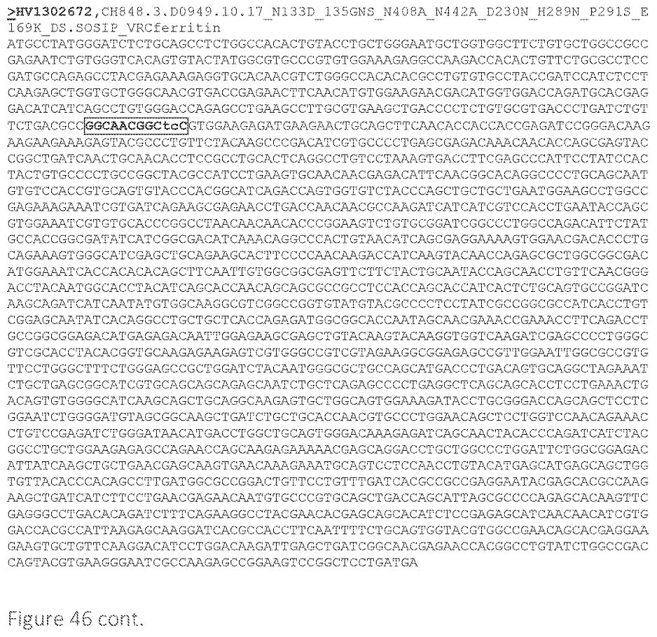

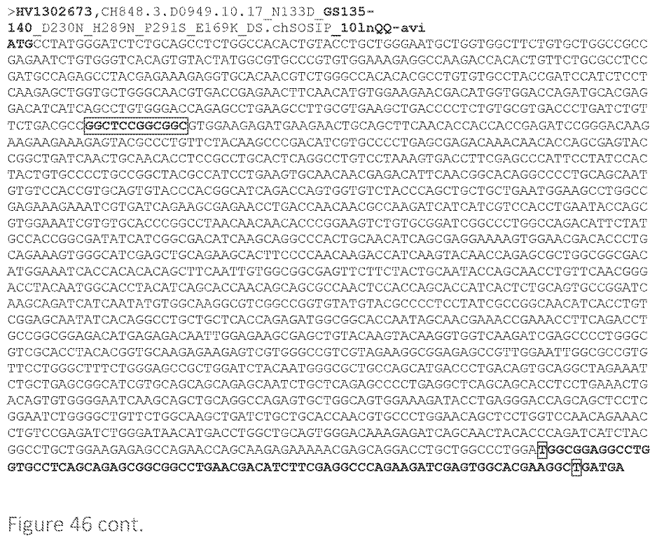

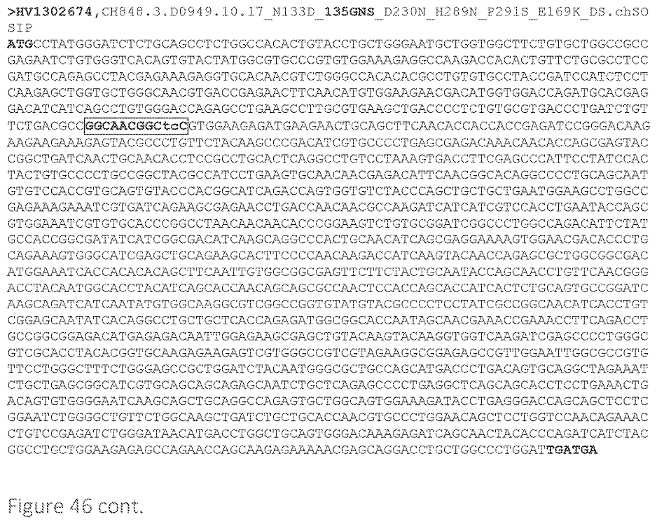

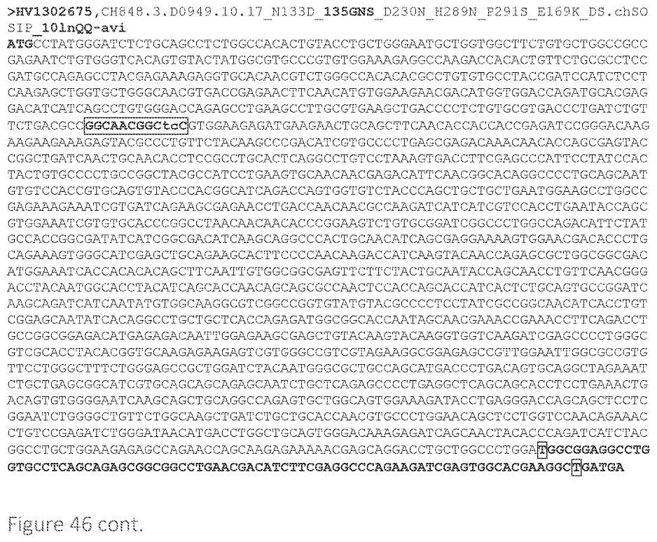

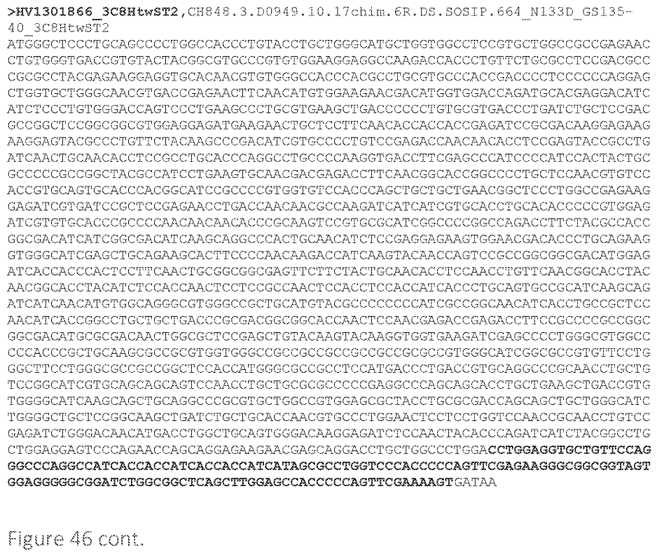

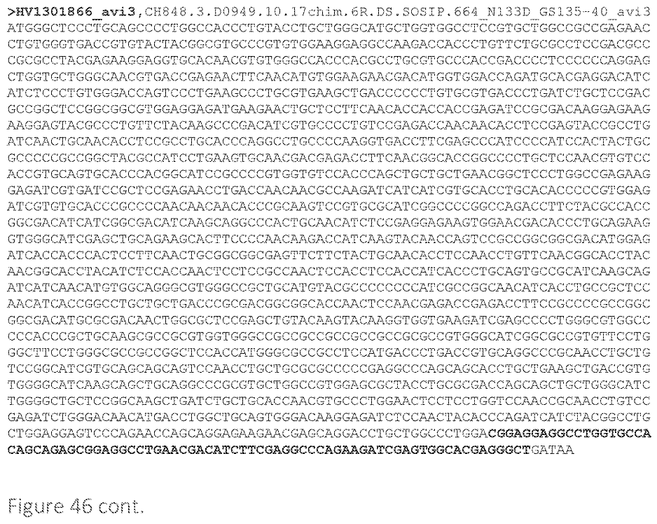

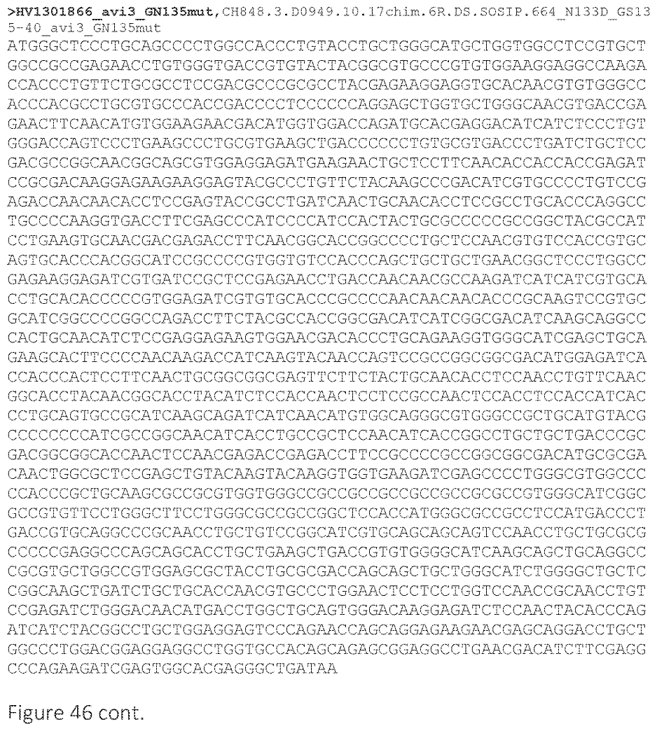

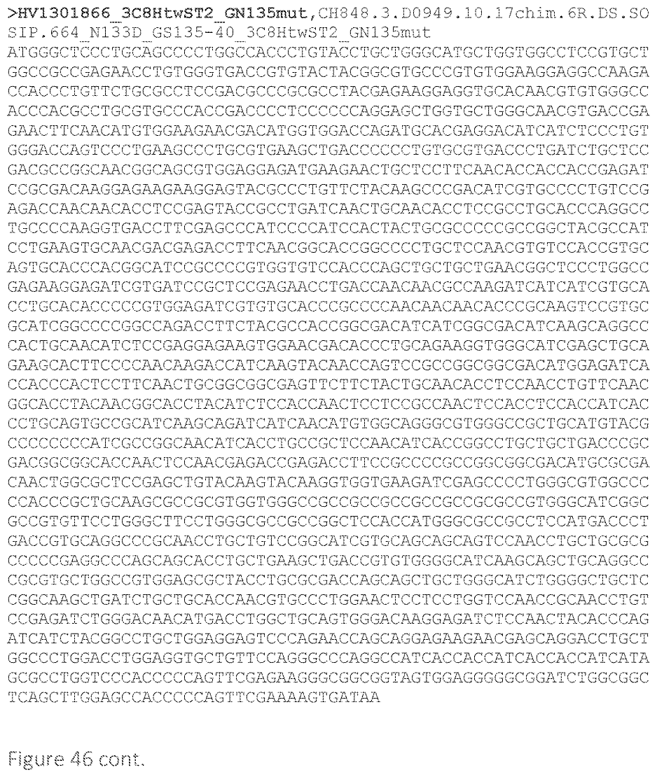

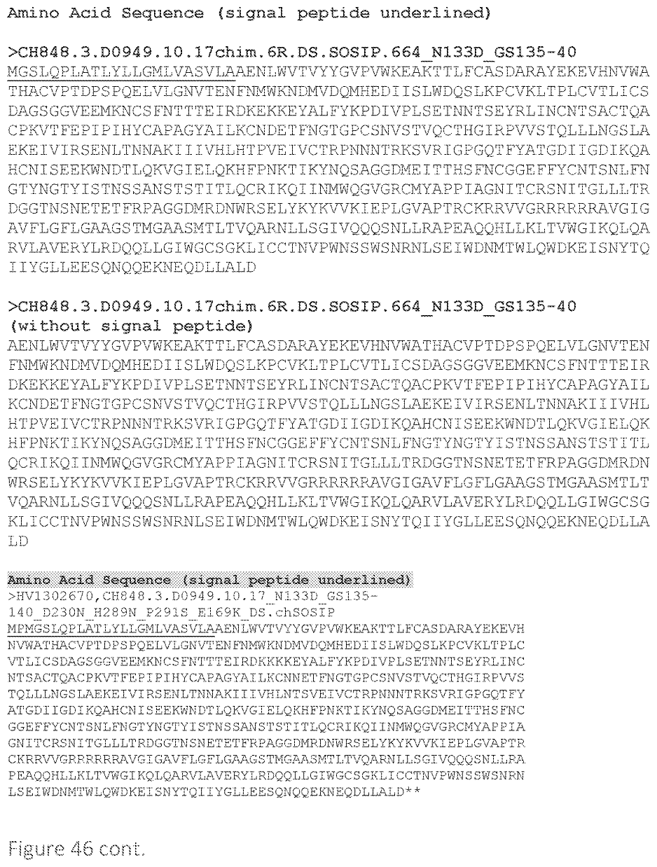

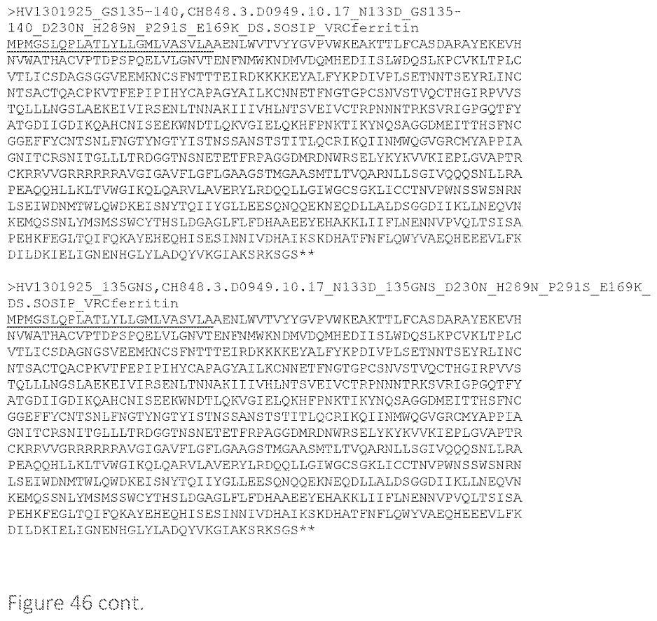

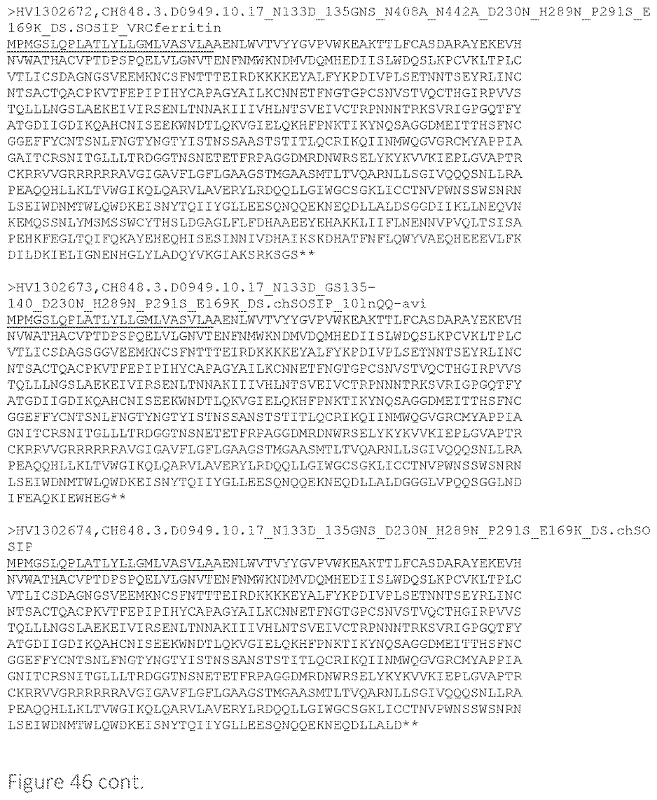

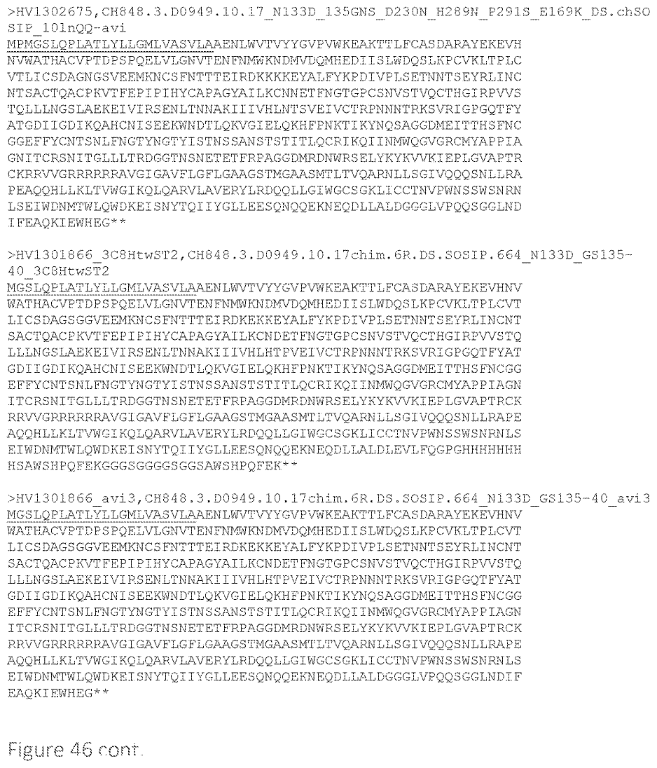

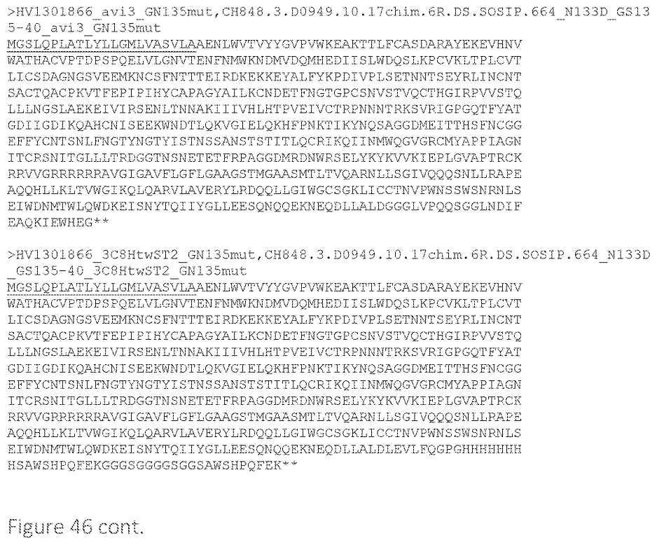

Table 2A shows a summary of modifications to envelopes described herein

| | Figure/SEQ | V1 | V3 glycosylation | UCA and other |
|---|---|---|---|---|
| Envelope | ID No | region | sites | Ab binding |
| 10.17 | See | 17aa | N301 and N332 | |
| | US2020/0113997 | | | |
| | incorporated | | | |
| | herein by | | | |
| | reference in | | | |
| | its entirety | | | |
| | including FIGS. | | | |
| | 39A-B and SEQ | | | |
| | ID NOs | | | |
| | disclosed therein | | | |
| 10.17 DT | See | 17aa N133D | N301 and N332 | DH270UCA |
| | US2020/0113997 | N138T | | |
| | incorporated | effectively | | |
| | herein by | lacks | | |
| | reference in | glycosylation | | |
| | its entirety | sites | | |
| | including FIG. | | | |
| | 49A, 50B, 51, | | | |
| | 52A, 53D, 54A- | | | |
| | C, 77D-G, 77I | | | |
| | and SEQ ID NOs | | | |
| | disclosed | | | |
| | therein- | | | |
| 10.19 | FIG. 39 | 17aa V1 region | No glycosylation | CH01 UCA |
| | | lacks N133 and | sites at N295, | |
| | | N138 | N301, N332 | |
| | | glycosylation | | |
| | | sites | | |
| 10.19 plus | FIGS. 40-41 | 17aa V1 region | Add V3 regions | CH01 UCA |
| V3 loop of | | lacks N133 and | from 10.17 has | DH270UCA |
| 10.17 | | N138 | five aa | VRC26 UCA |
| (19CV3) | | glycosylation | difference from | |
| | | sites | 10.19 | |
| 10.19 env | See e.g., | | At least changes | |
| based with | FIGS. 42A-B | | #2, 4, 5, and/or | |
| fewer than | | | “GDIR” | |
| five aa | | | sequence (SEQ | |
| changes | | | ID NO: 2) | |
| compared | | | | |
| to 19CV3; | | | | |
| “GDIR/K” | | | | |
| Ferritin | See e.g., | | | |
| Linker | FIGS. 43A-B | | | |
| E169K | See e.g., | | | |
| | FIGS. 42A-B | | | |
| Glycan | See e.g., | | | |
| hole filled | FIGS. 41A-B, | | | |
| | 42A-B, 45A-B | | | |
| GS design | See e.g, | | | |
| | FIG. 46 | | | |

Table 2B shows a summary of additional modifications to envelopes described herein

| | | | |
|---|---|---|---|
| CH848.3.D0949.10.17 | See FIG. 52 | V1 loop | DH270.6 |
| | >CH848.3.D0949.10.17chim.526V1.6R.DS.SOSIP.664 | replaced by | bnAb |
| | | d0526.25.02 V1 | path |
| CH848.3.D1432.5.41 | See FIG. 52 | V1 loop | DH270.6 |
| | >CH848.3.D1432.5.41CHIM.526V1.DS.6R.SOSIP.664 | replaced by | bnAb |
| | | d0526.25.02 V1 | path |

DH270 light chain binds to N301 glycan. In some embodiments, a N301 gly site is used (e.g. change #2 in row 5 of Table 2A., supra).

DH270 heavy chain binds to N332 glycan. In some embodiments, a N332 gly site is used (e.g. changes #4 and #5 in row 5 of Table 2A, supra).

V3 glycan Abs bind GDIR (SEQ ID NO: 2). In some embodiments, a change #3 to “GDIR” (SEQ ID NO: 2) is needed (e.g. “GDIR” sequence (SEQ ID NO: 2) in row 5 of Table 2A, supra).

GDIR/K motif: V3-glycan broadly neutralizing antibodies typically contact the c-terminal end of the third variable region on HIV-1 envelope. There are four amino acids, Gly324, Asp325, Ile326, and Arg327 (SEQ ID NO: 2), bound by V3-glycan neutralizing antibodies. While Arg327 is highly conserved among HIV-1 isolates, Lys327 also occurs at this site. The CH848.3.1D0949.10.17 isolate naturally encodes the less common Lys327. In contrast to CH848.3.D0949.10.17 with the Lys327, the precursor antibody of the DH270 V3-glycan broadly neutralizing antibody lineage barely binds to CH848.3.D0949.10.17 encoding Arg327. Thus, Arg327 is critical for the precursor to bind and the lineage of neutralizing antibodies to begin maturation. However, somatically mutating antibodies on the path to developing neutralization breadth bind better to Env encoding Arg327. Thus, Env must encode Lys327 to initiate DH270 lineage development. However, to best interact with affinity maturing DH270 lineage members the Env should encode Arg327. Thus, a plausible vaccine regimen to initiate and select for developing bnAbs would include a priming immunogen encoding, Lys327 and a boosting immunogen encoding Arg327. The Arg327 boosting immunogen would optimally target the affinity maturing DH270 lineage members, while not optimally binding the DH270 antibodies that lack affinity maturation. Non-limiting embodiments of vaccination regimens could include: priming with CH848.3.D0949.10.17 based envelope design also with Lys327, followed by administering of CH848.3.D0949.10.17 based envelope design with Arg327. Non-limiting embodiments of vaccination regimens could include: priming with 19CV3 based envelope design also with Lys327, followed by administering of CH848.3.D0949.10.17 based envelope design with Arg327.

E169K modification: One approach to designing a protective HIV-1 vaccine is to elicit broadly neutralizing antibodies (bnAbs). However, bnAbs against two or more epitopes will likely need to be elicited to prevent HIV-1 escape. Thus, optimal HIV-1 immunogens should be antigenic for multiple bnAbs in order to elicit bnAbs to more than one epitope. The CH848.D949.10.17 HIV-1 isolate was antigenic for V3-glycan antibodies but lacked binding to VIV2-glycan antibodies. Not all viruses from the CH848 individual lacked binding to VIV2-glycan antibodies. For example, the CH848.D1305.10.19 isolate bound well to V1V2-glycan antibody PGT145. The sequence of CH848.D949.10.17 and CH848.D1305.10.19 in the region that is contacted by V1 V2-glycan antibodies in crystal structures were compared (McLellan J S, Pancera M, Carrico C, Gorman J, Julien J P, Khayat R, et al. Structure of HIV-1 gp120 V1N2 domain with broadly neutralizing antibody PG9. Nature. 2011; 480(7377):336-43). Interestingly, the CH848.D949.10.17 and CH848.D1305.10.19 differed in sequence at a known contact site for VIV2-glycan antibodies-position 169 (Doria-Rose N A, Georgiev I, O'Dell S, Chuang G Y, Staupe R P, McLellan J S, et al. A short segment of the HIV-1 gp120 V1/V2 region is a major determinant of resistance to V/N2 neutralizing antibodies. J Virol. 2012; 86(15):8319-23). It has been previously shown that mutation of lysine at position 169 eliminates binding to VIV2-glycan antibody PG9 (Doria-Rose N A, Georgiev I, O'Dell S, Chuang G Y, Staupe R P, McLellan J S, et al. A short segment of the HIV-1 gp2 V1/V2 region is a major determinant of resistance to V1/V2 neutralizing antibodies. J Virol. 2012; 86(15):8319-23). CH848.D1305.10.19 sequence encoded a lysine at position 169 whereas CH848.D949.10.17 sequence encoded a glutamate. Thus, the glutamate (E) was changed to lysine (K) at position 169 of CH848.D949.10.17. This single change in CH848.D949.10.17 enabled V1V2-glycan antibody binding to the envelope. Thus, the E169K adds the V1V2-glycan epitope to the other bnAb epitopes present on CH848.D949.10.17-based envelopes. Overall, the result of the E169K is a CH848.D949.10.17 envelope capable of eliciting more different types of bnAbs.

The invention contemplates any other design, e.g. stabilized trimer, of the sequences described here in. For non-limiting embodiments of additional stabilized trimers see US2015/0366961 (DU4061), US2020/0002383 (DU4716), US2021/0187091 (DU4918) and US2020/0113997 (DU4918), F14 and/or VT8 designs (US2021/0379177) all of which are incorporated by reference in their entirety.

In certain embodiments the invention provides an envelope comprising 17aa V1 region without N133 and N138 glycosylation, and N301 and N332 glycosylation sites, and further comprising “GDIR” motif (SEQ ID NO: 2), wherein the envelope binds to UCAs of V1V2 Abs and V3 Abs.

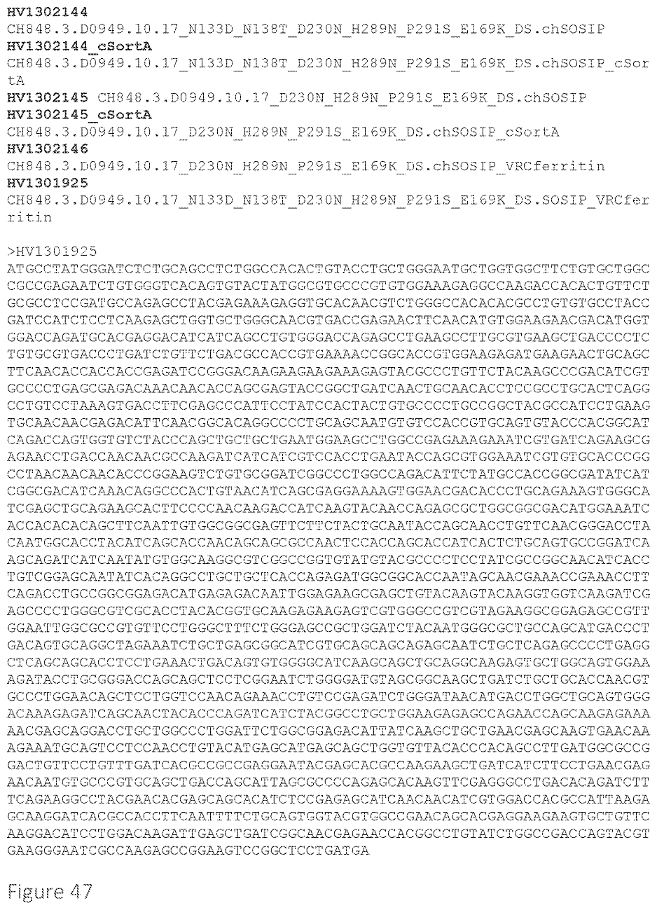

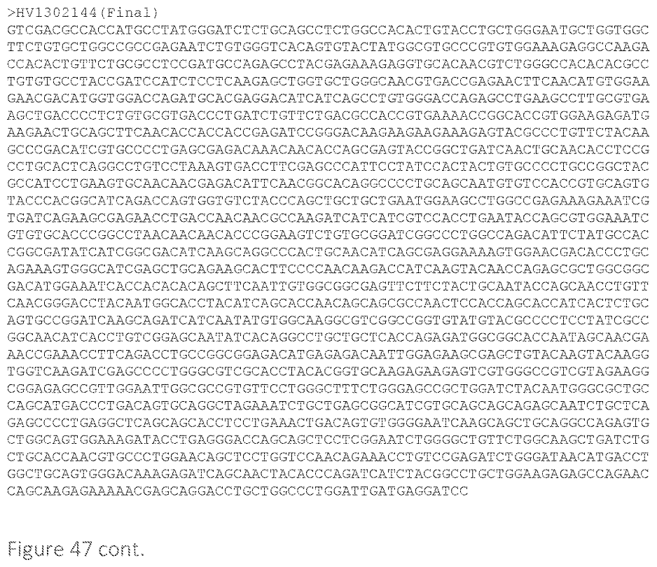

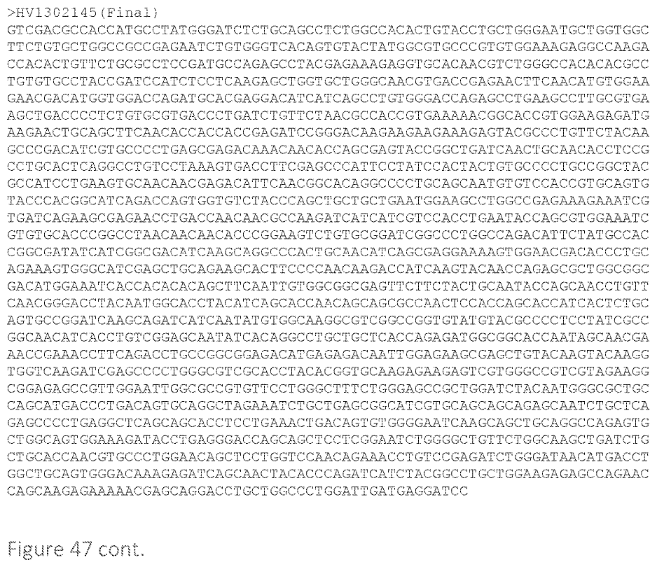

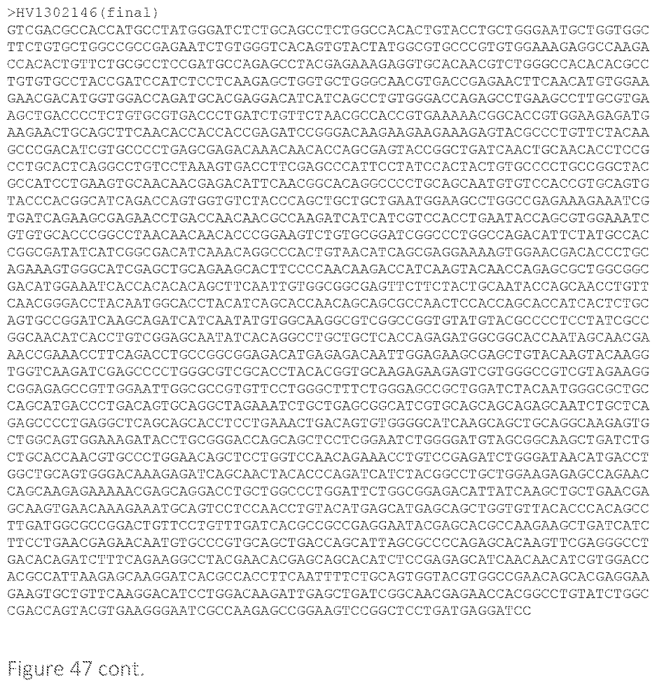

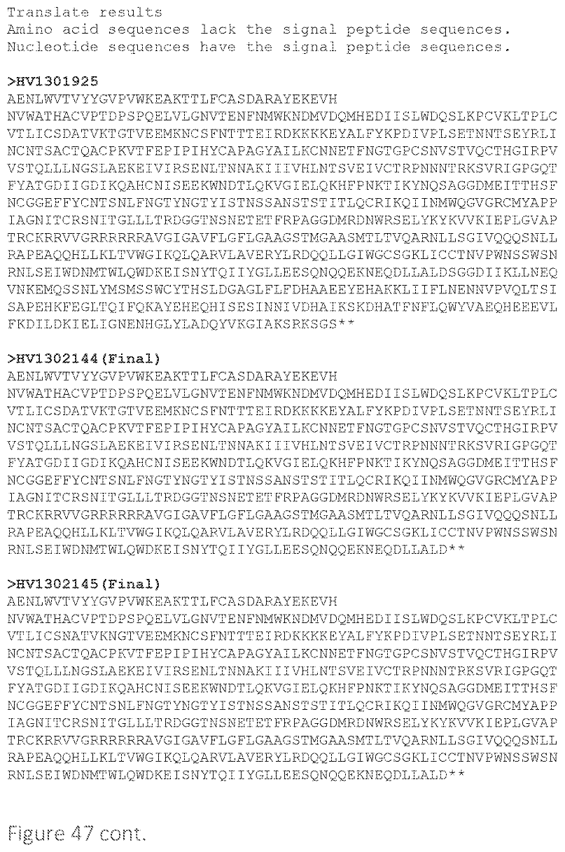

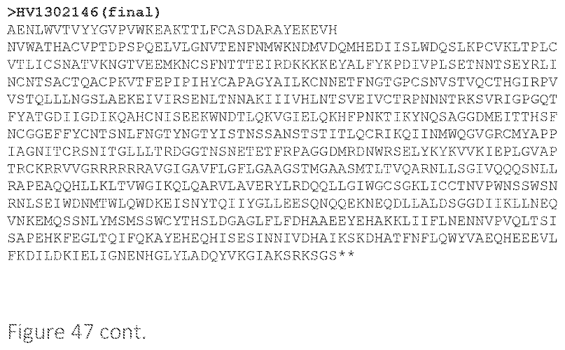

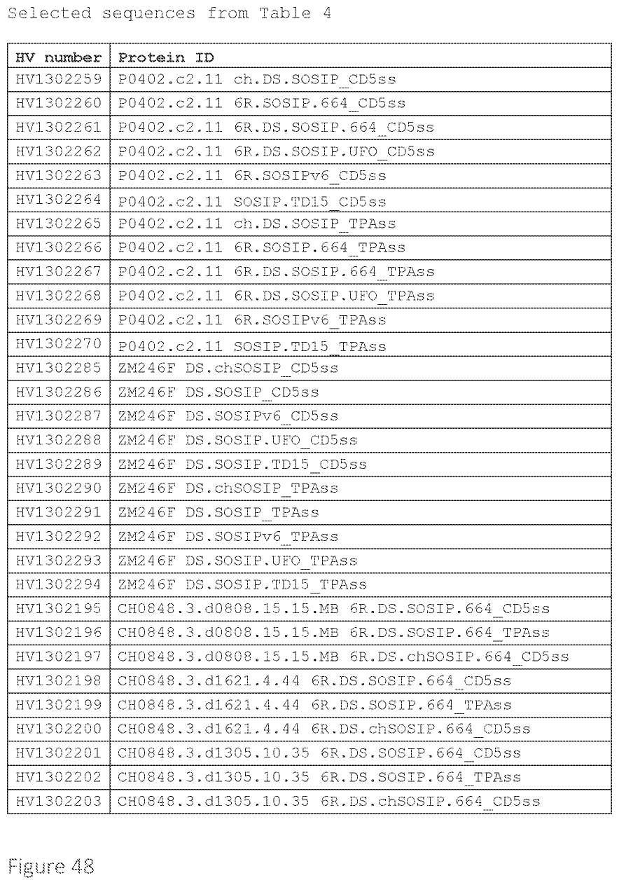

TABLE 3
Summary of envelope designs for use in prime and boost regimens

| HV | Envelope to be used as | Linker | Ferritin for | |
|---|---|---|---|---|
| name | protein and/or mRNA | sequence | multimerization | Figure |
| HV1302144 | 10.17 DT protein trimer | no | No | FIG. 47 |
| | CH848.3.D0949.10.17_N133D_N138T_D230N_{—} | | | |
| | H289N_P291S_E169K_DS.chSOSIP | | | |
| HV1302144_cSortA | 10.17 DT protein sortaNP | For Sortase | Any Ferritin | |
| | CH848.3.D0949.10.17_N133D_N138T_D230N_{—} | conjugation | is added via | |
| | H289N_P291S_E169K_DS.chSOSIP_cSortA | | Sortase | |
| | | | conjugation | |
| | | | at C- | |
| | | | terminus | |
| HV1301925 | 10.17 DT protein NP | SGG | could be any | FIG. 47 |
| | CH848.3.D0949.10.17_N133D_N138T_D230N_{—} | | suitable | |
| | H289N_P291S_E169K_DS.chSOSIP_VRCferritin | | ferritin | |
| HV1302145 | 10.17 protein trimer | no | | FIG. 47 |
| | CH848.3.D0949.10.17_D230N_H289N_P291S_{—} | | | |
| | E169K_DS.chSOSIP | | | |
| HV1302145_cSortA | CH848.3.D0949.10.17_D230N_H289N_P291S_{—} | For Sortase | Any Ferritin | |
| | E169K_DS.chSOSIP_cSortA | conjugation | is added via | |
| | | | Sortase | |
| | | | conjugation | |
| | | | at C- | |
| | | | terminus | |
| HV1302146 | CH848.3.D0949.10.17_D230N_H289N_P291S_{—} | SGG | could be any | FIG. 47 |
| | E169K_DS.chSOSIP_VRCferritin | | suitable | |
| | | | ferritin | |

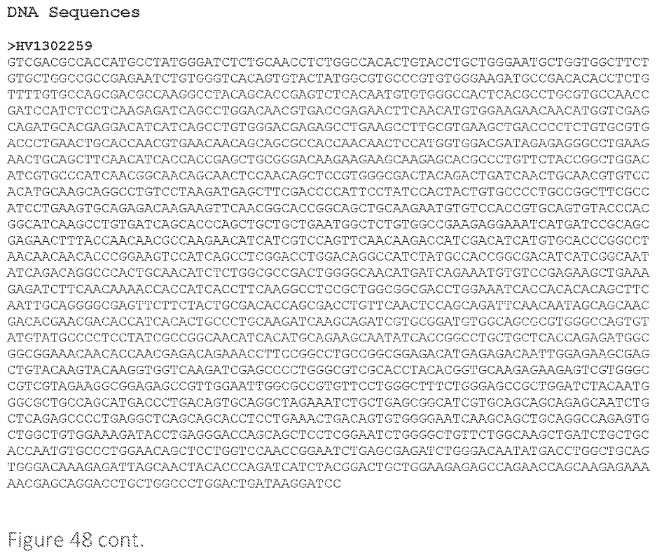

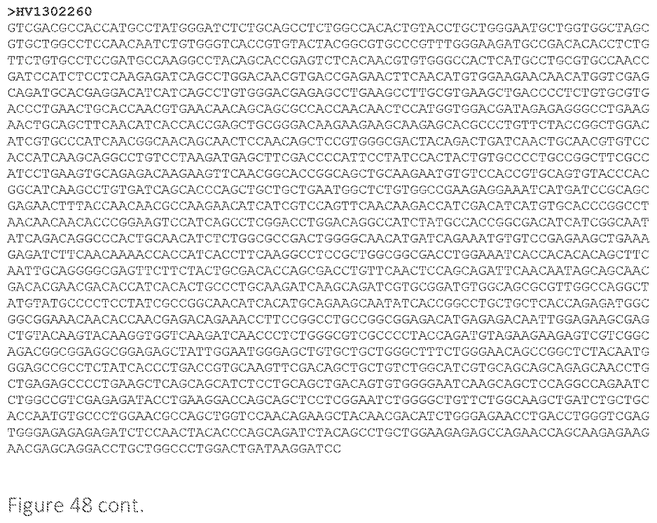

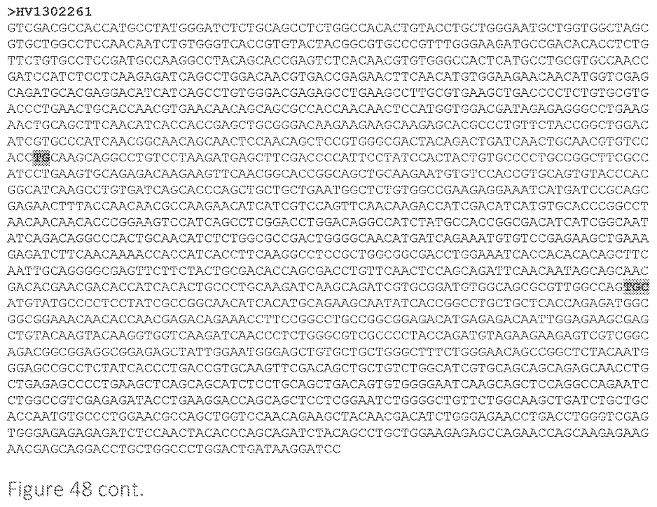

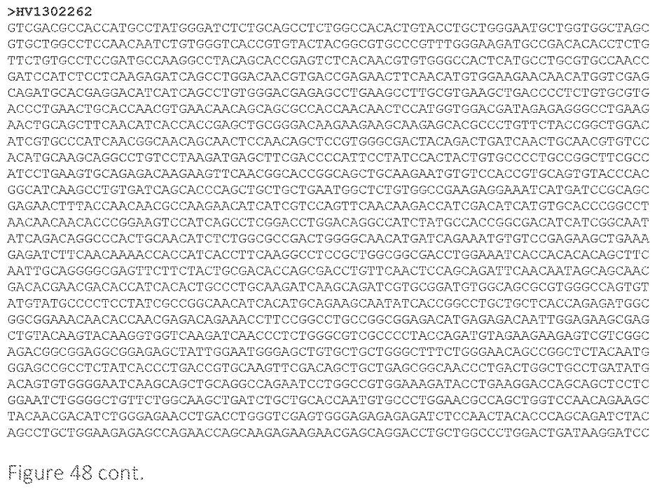

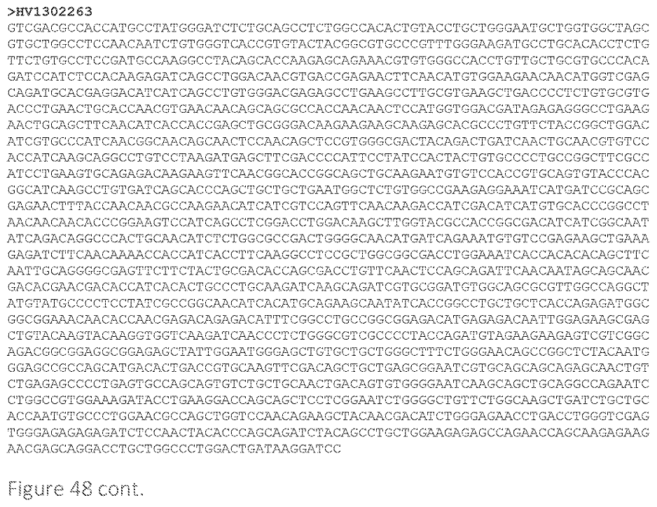

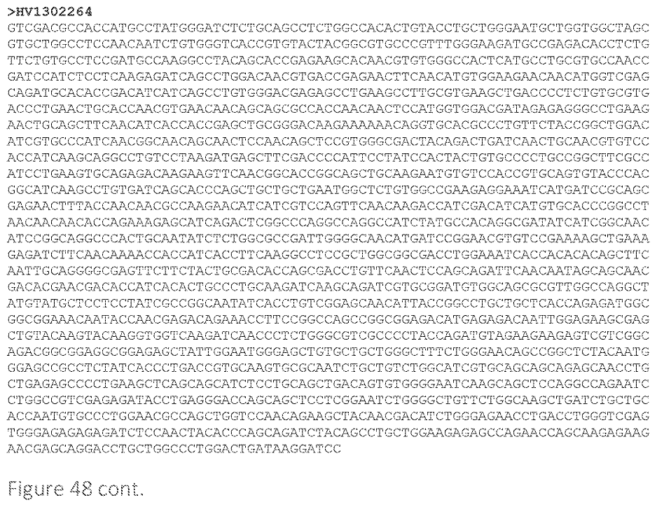

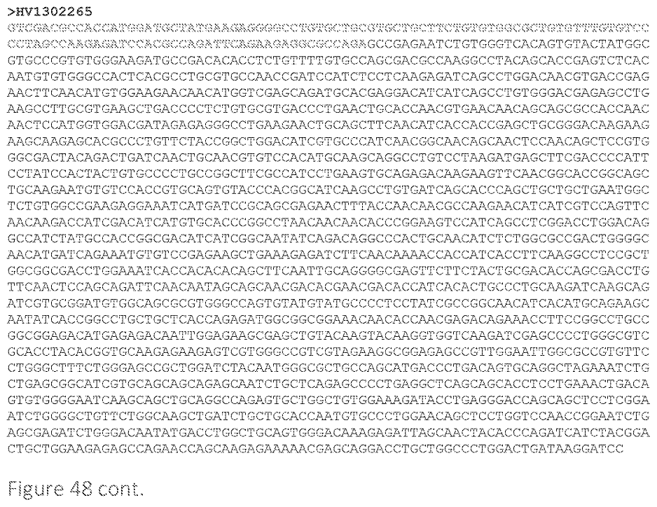

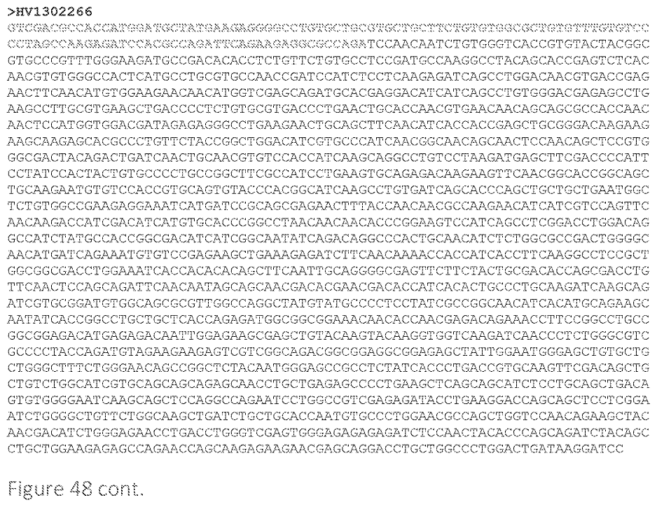

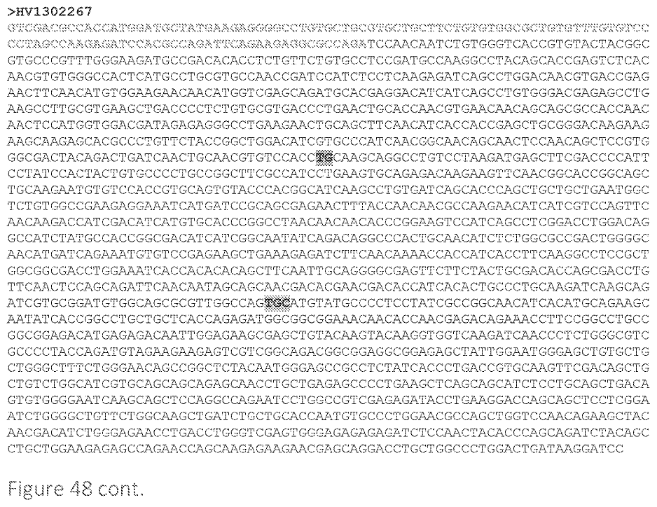

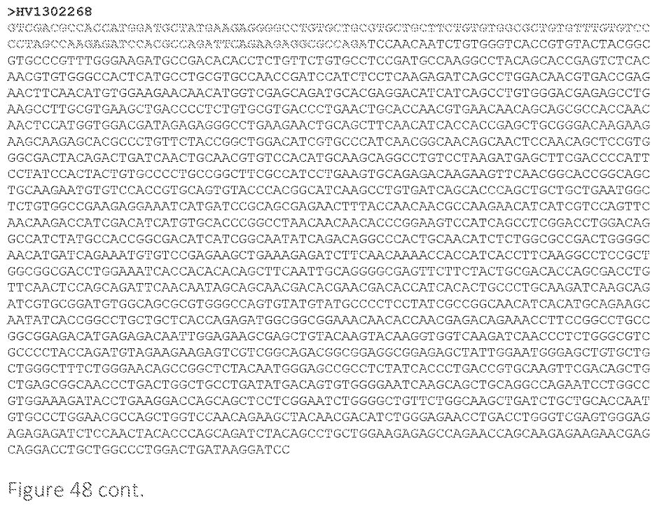

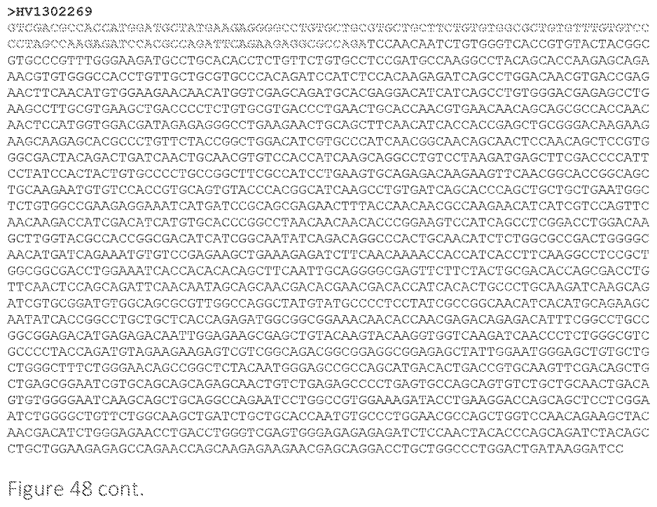

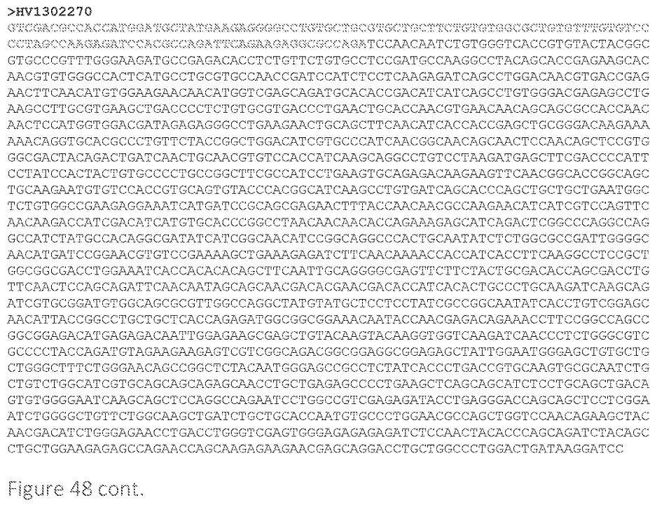

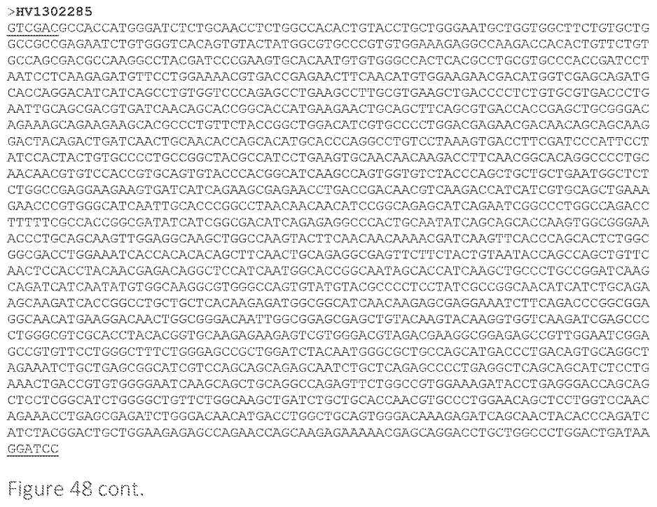

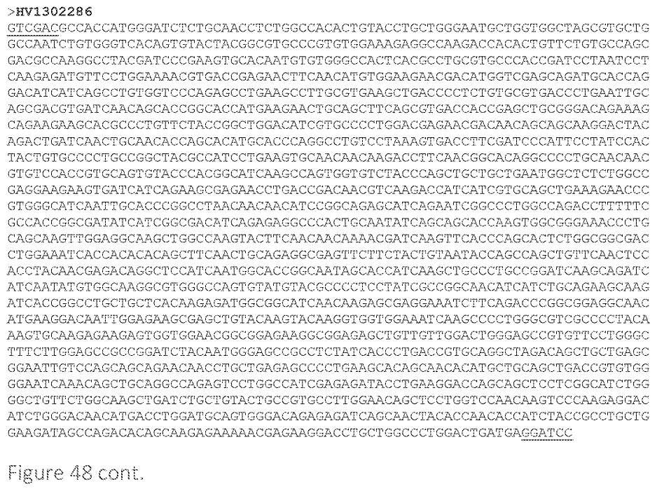

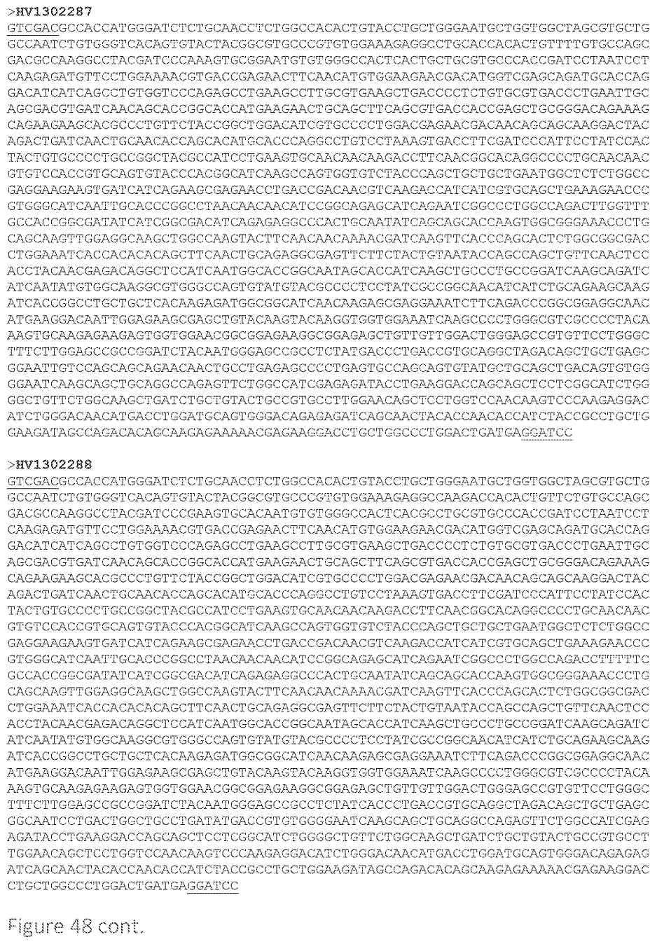

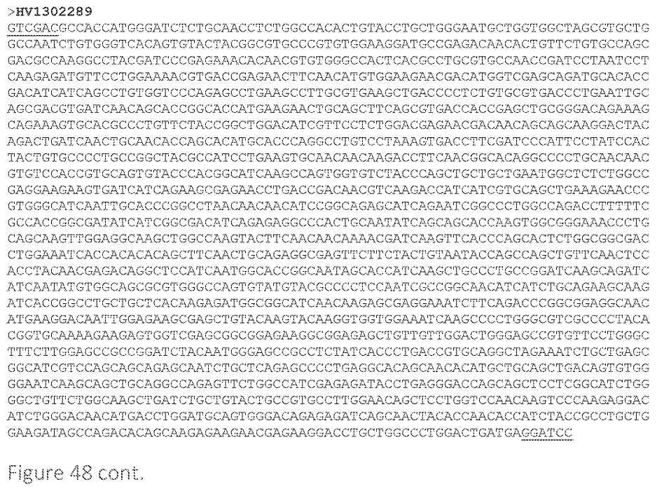

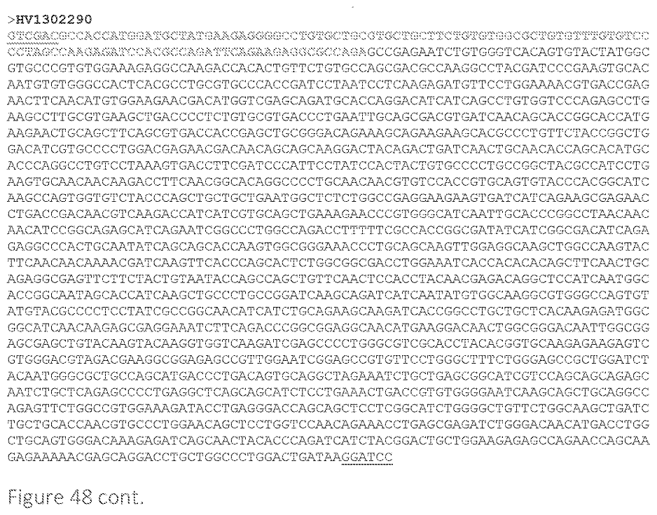

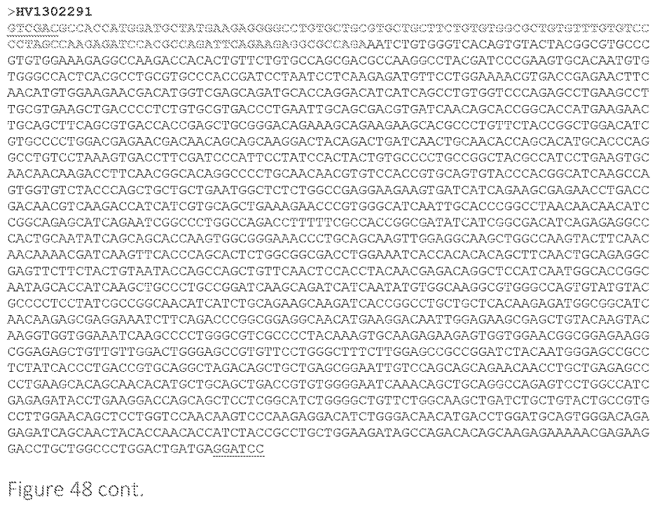

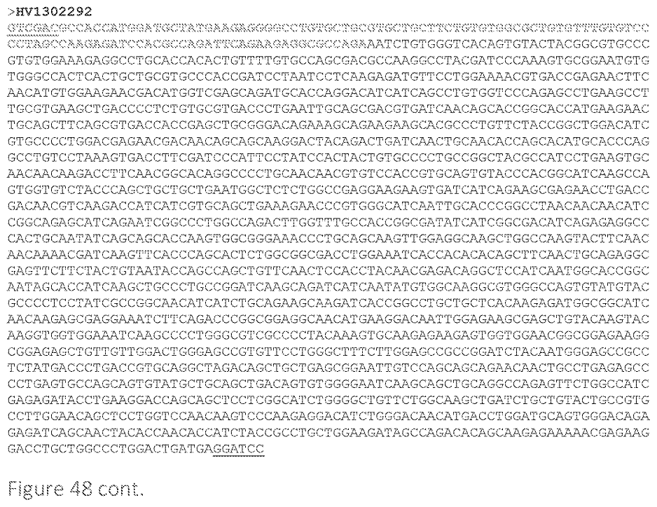

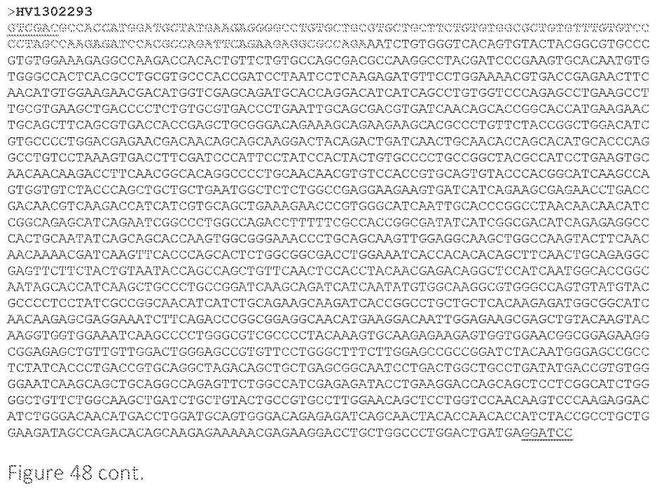

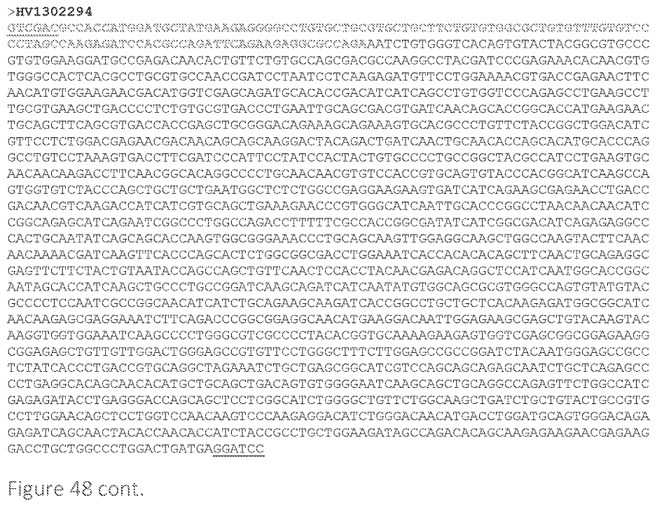

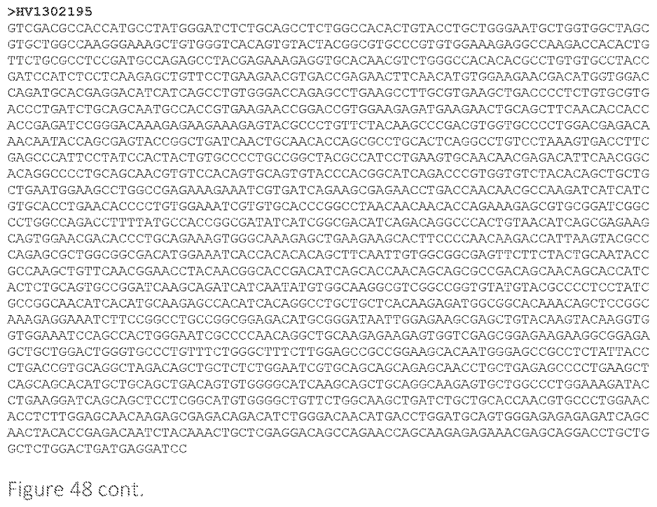

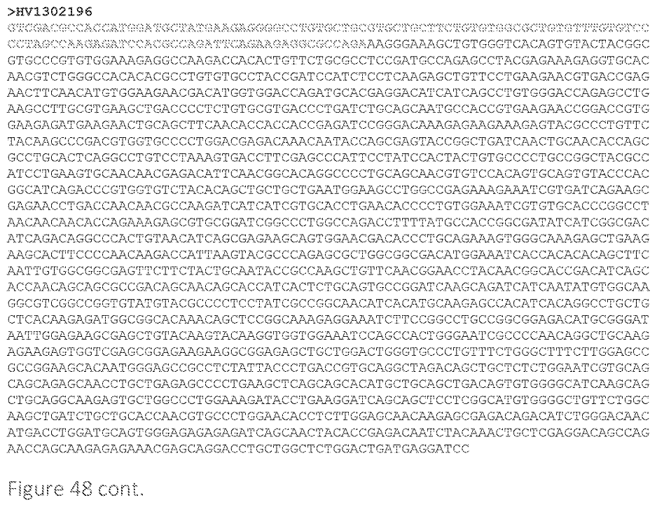

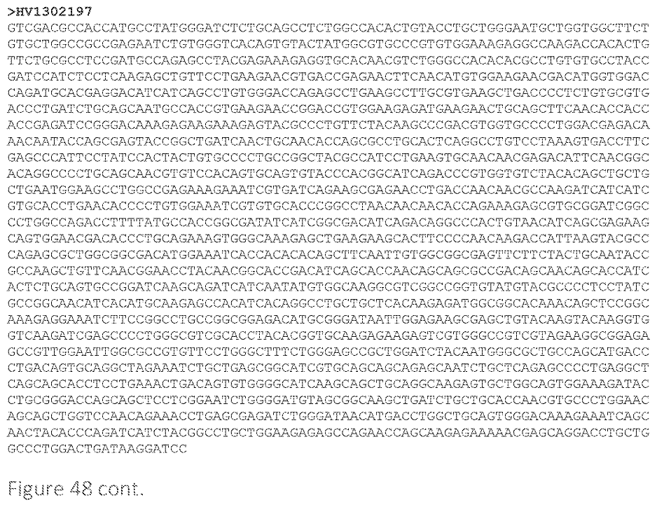

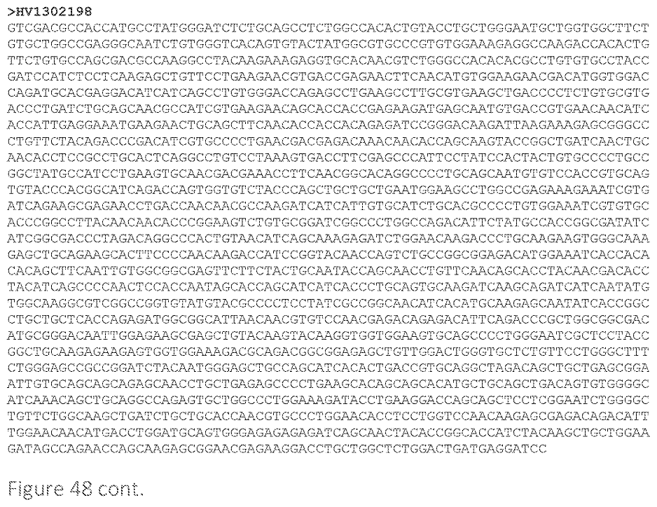

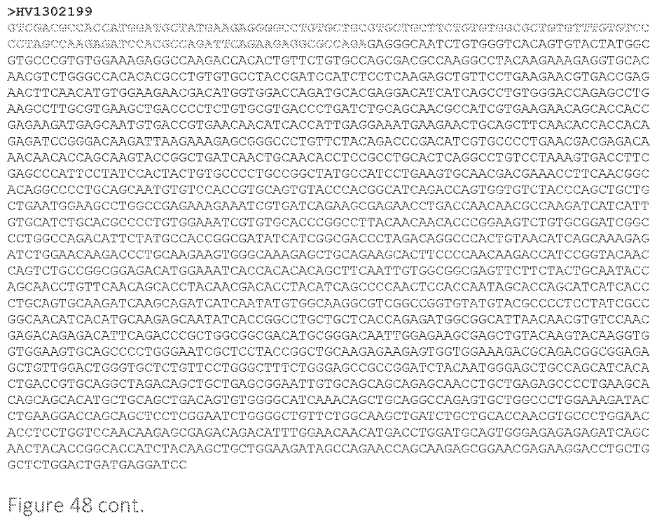

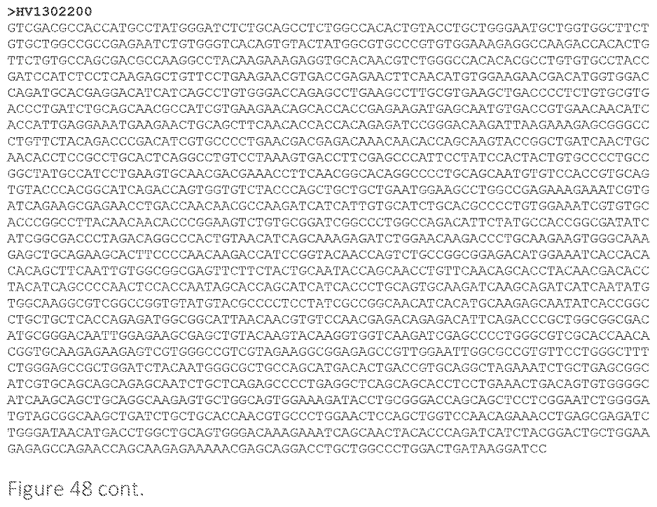

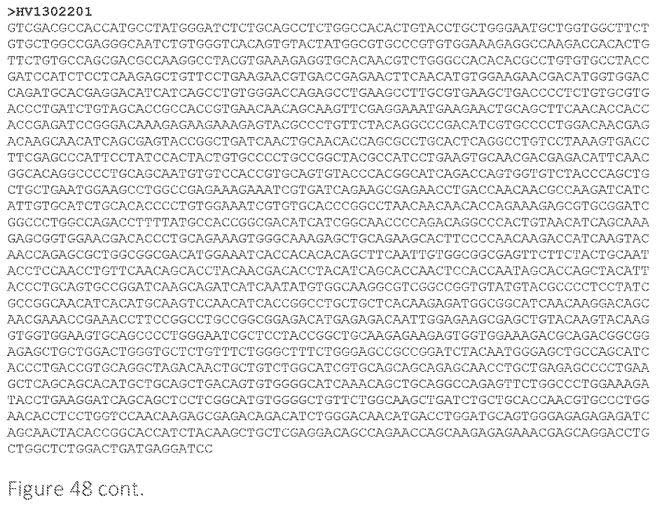

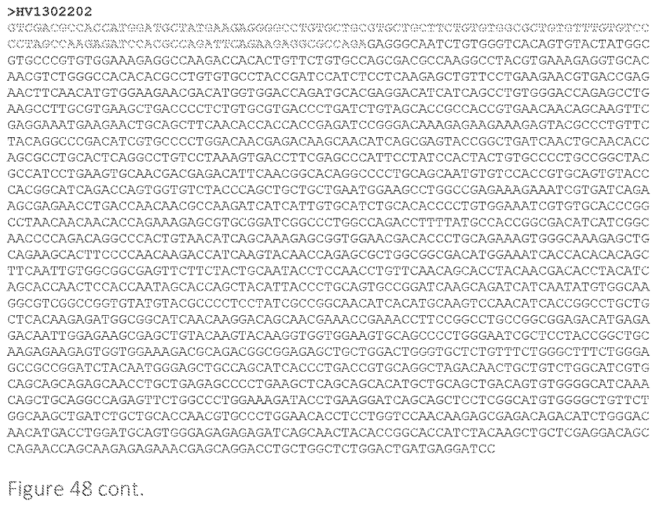

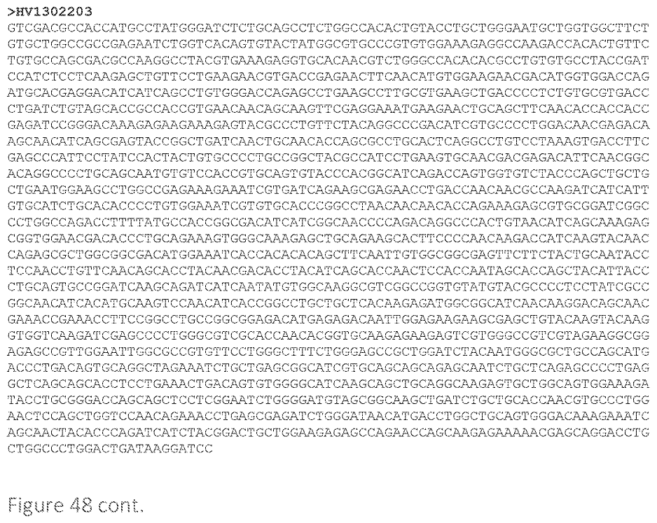

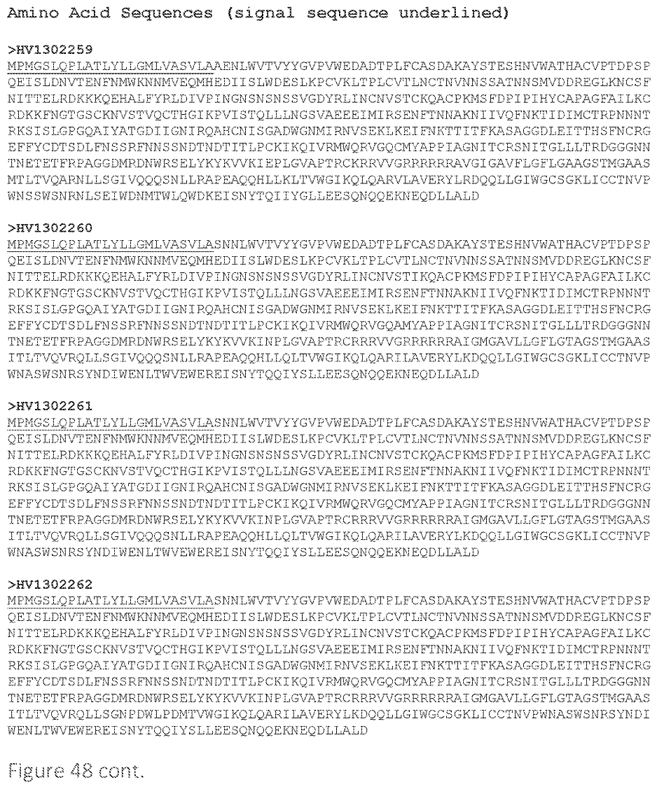

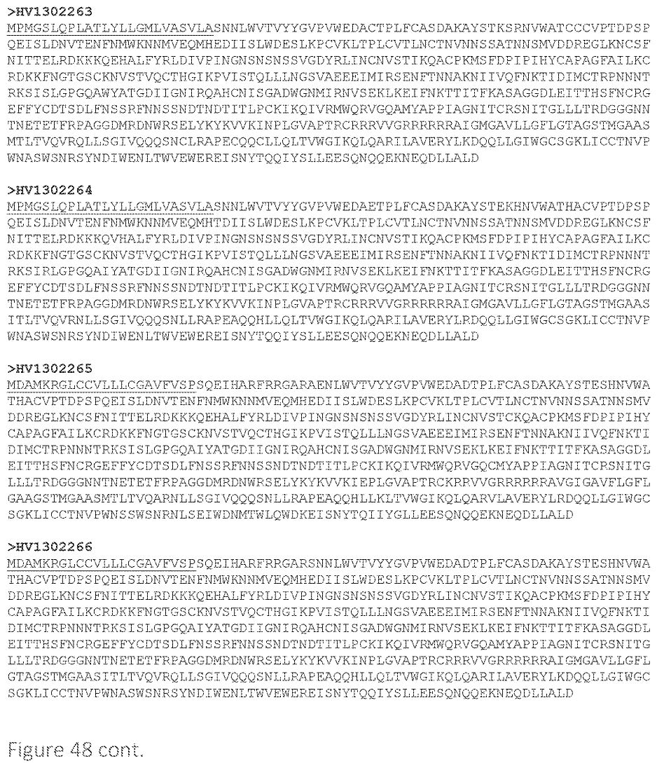

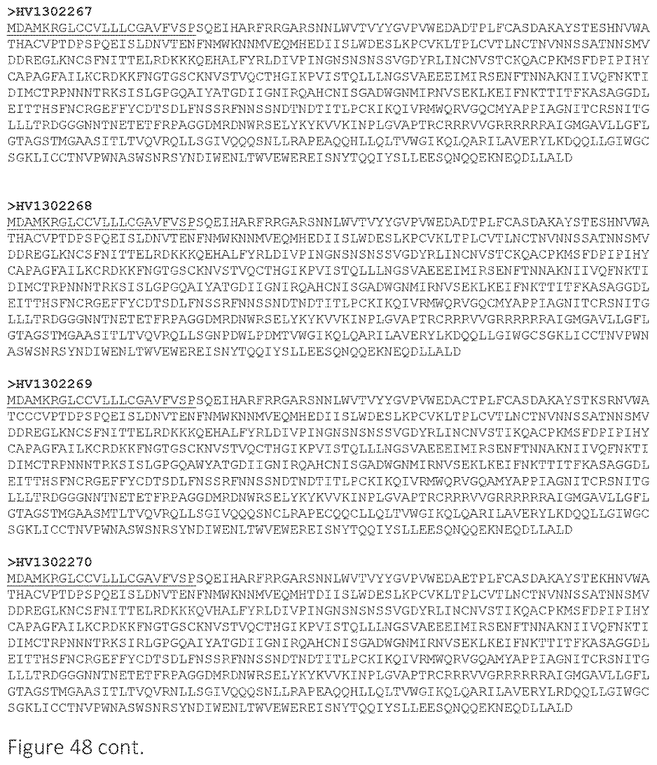

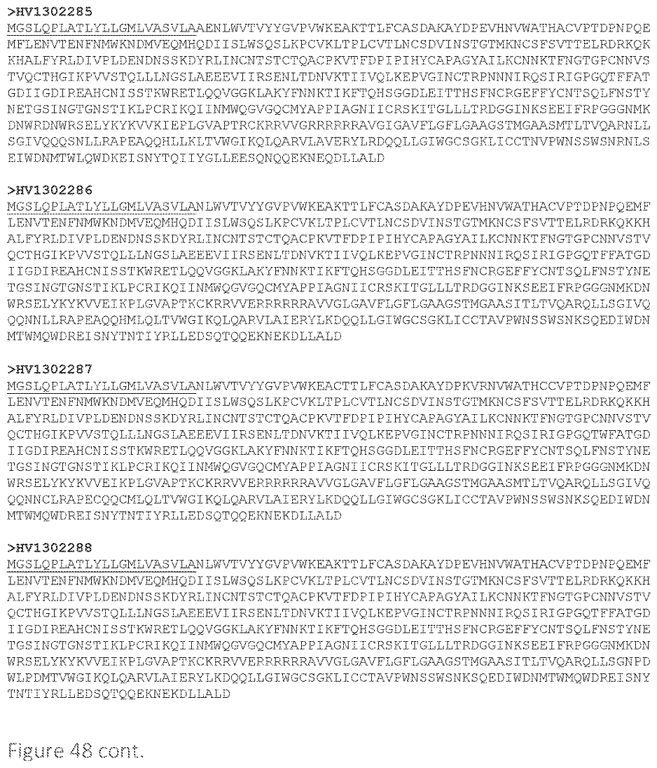

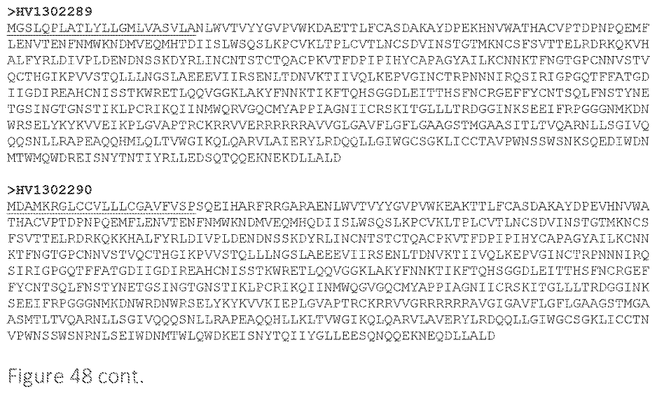

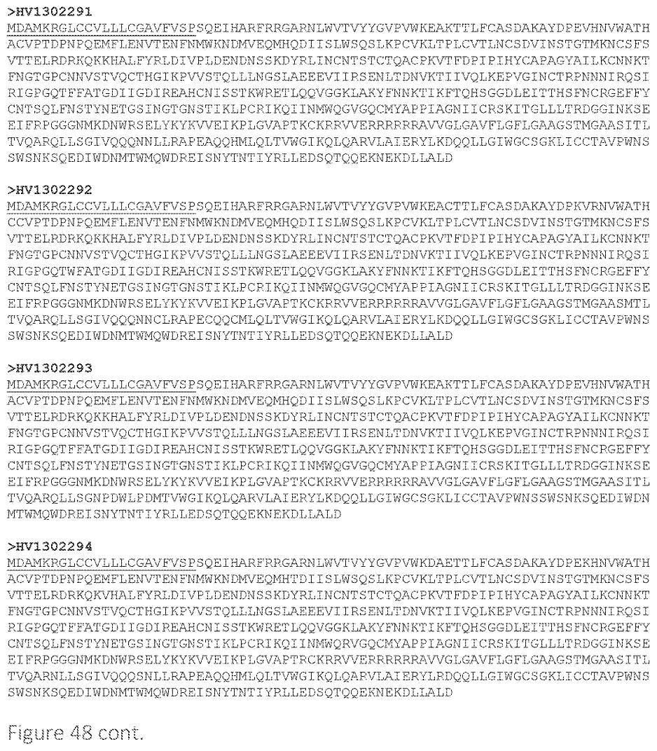

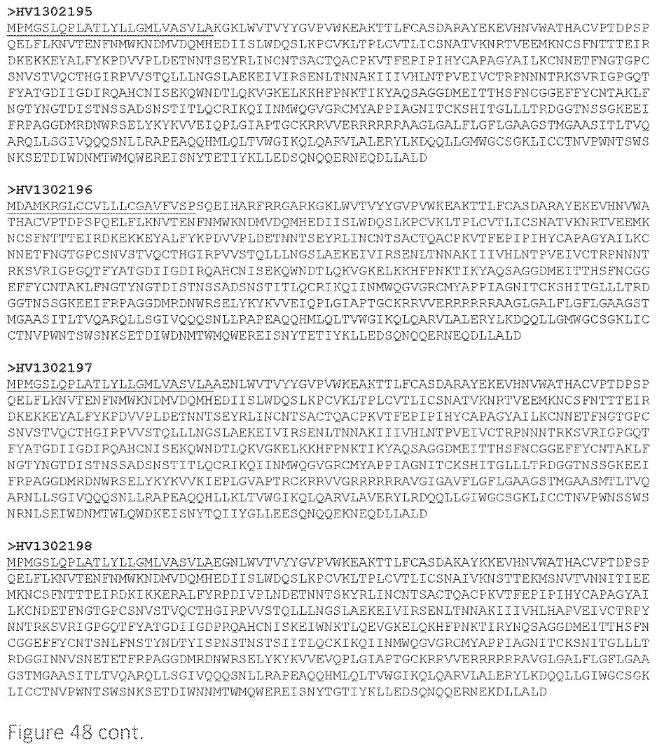

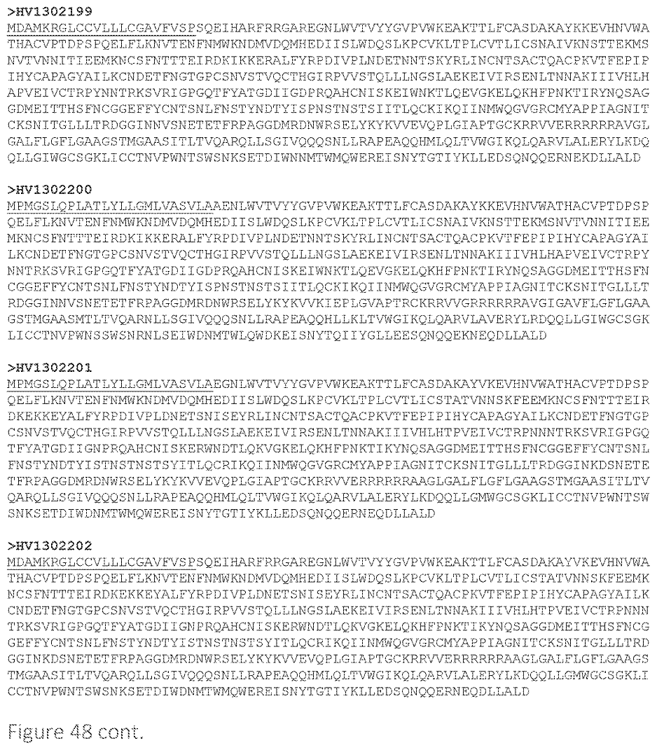

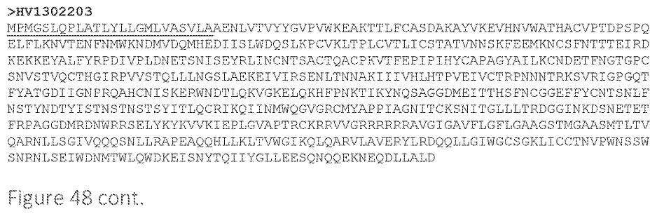

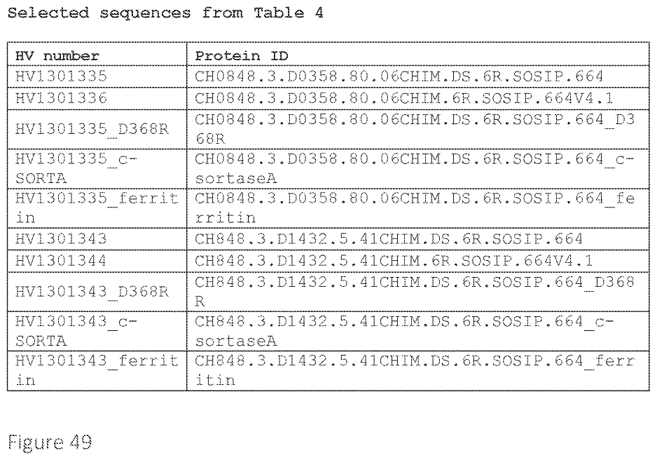

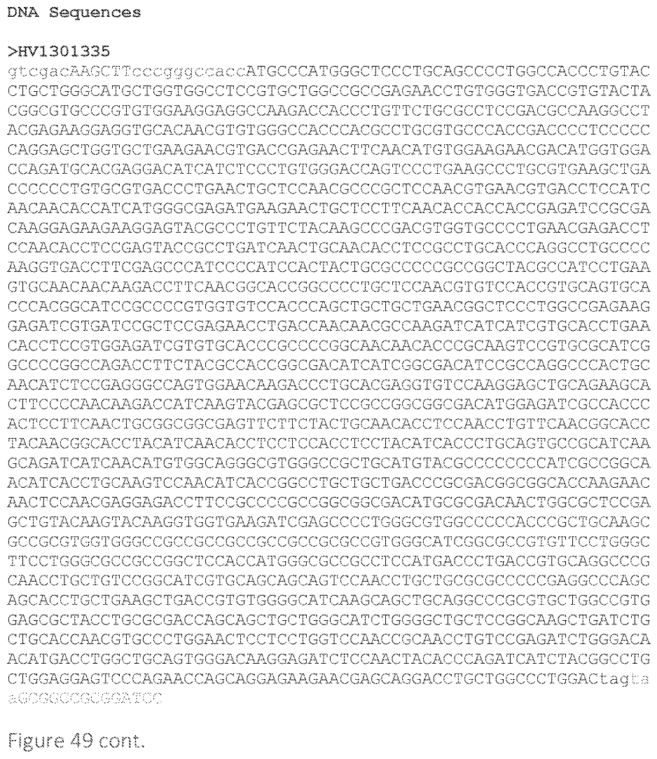

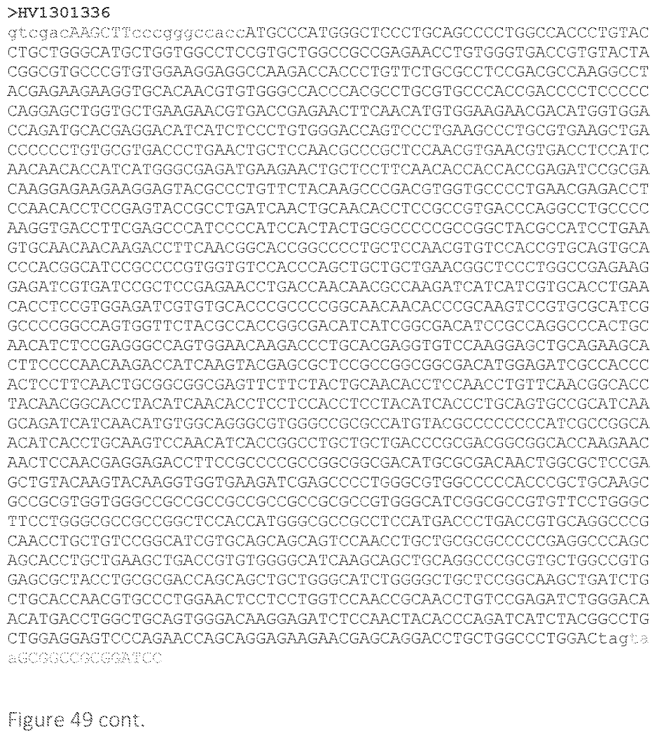

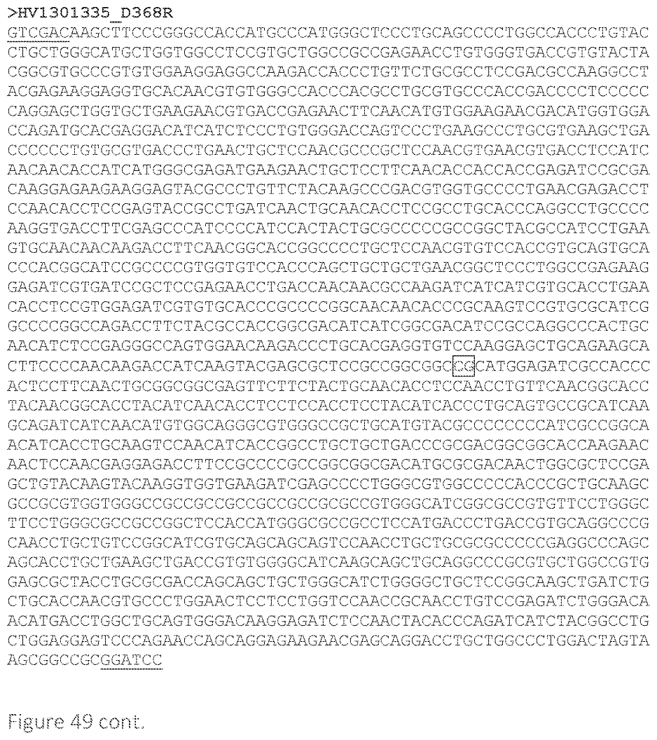

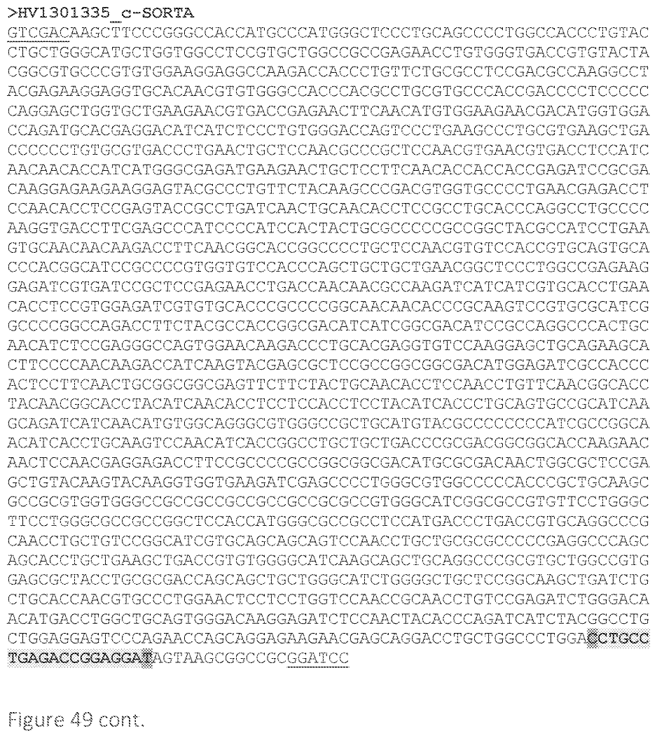

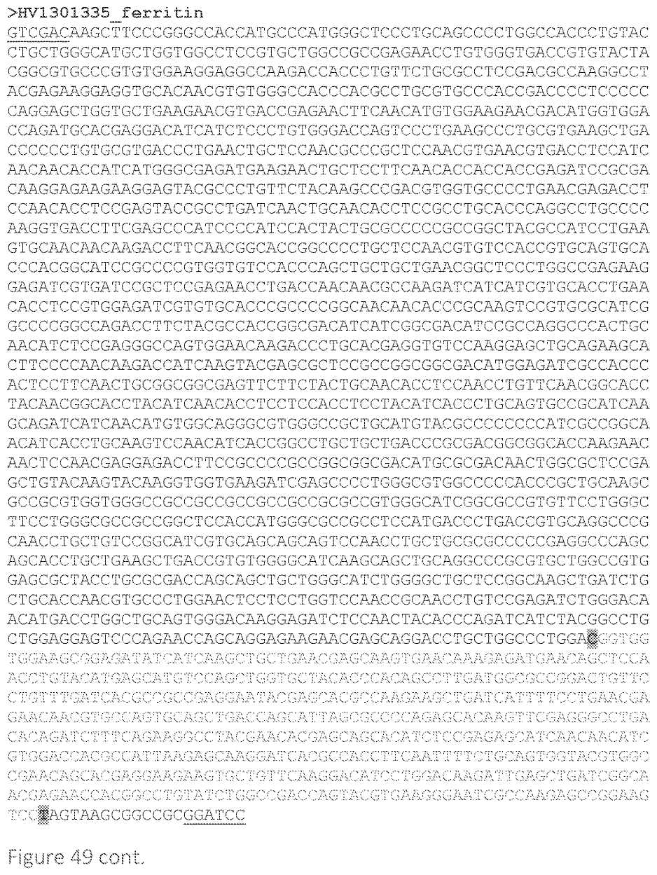

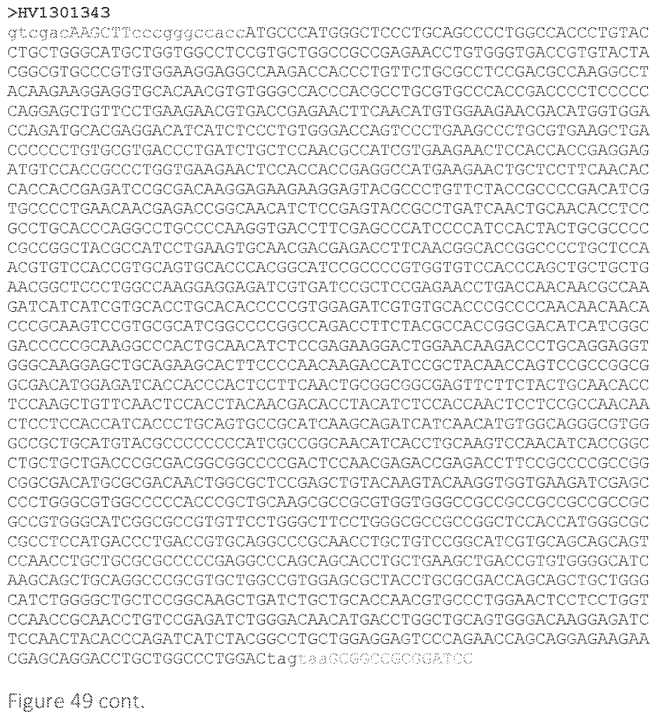

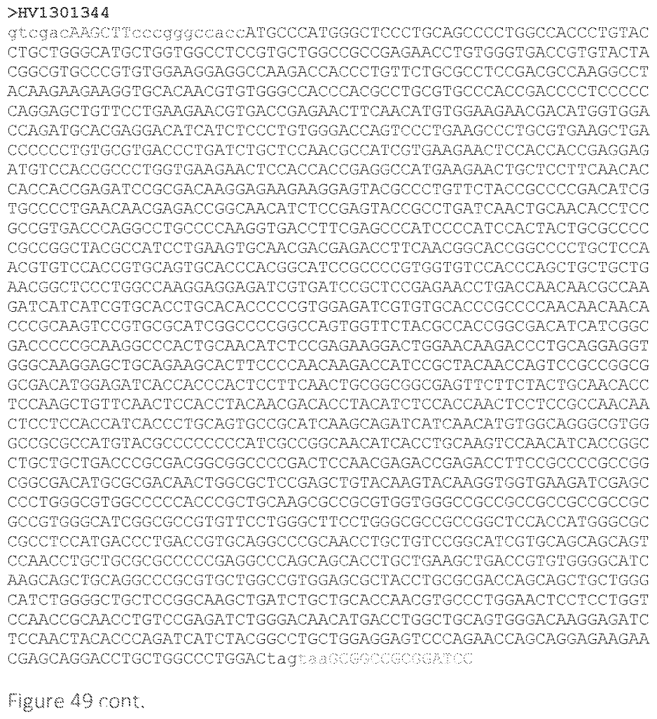

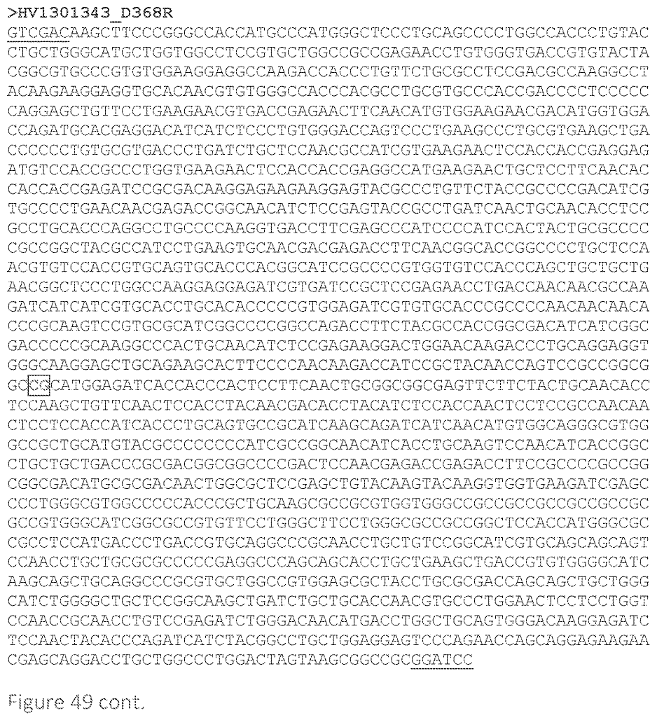

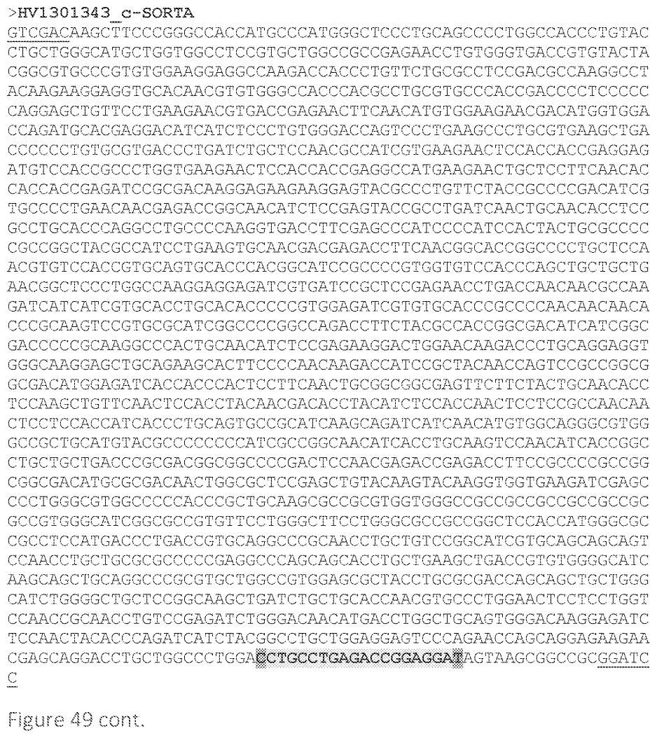

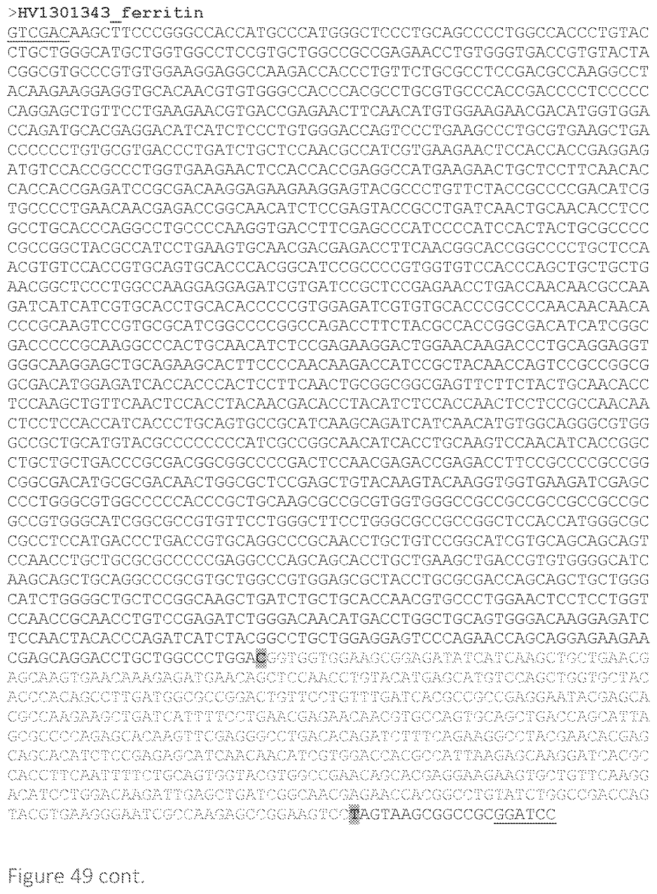

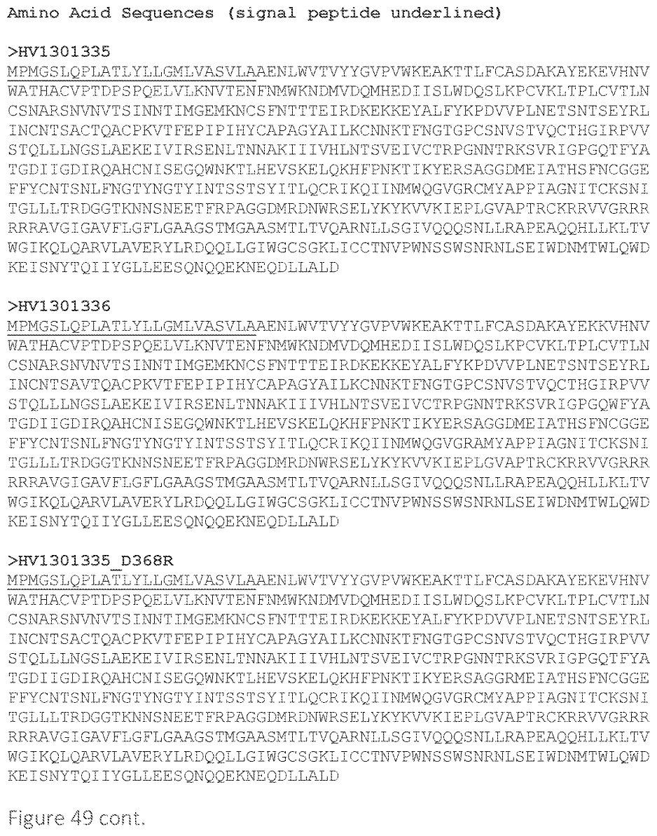

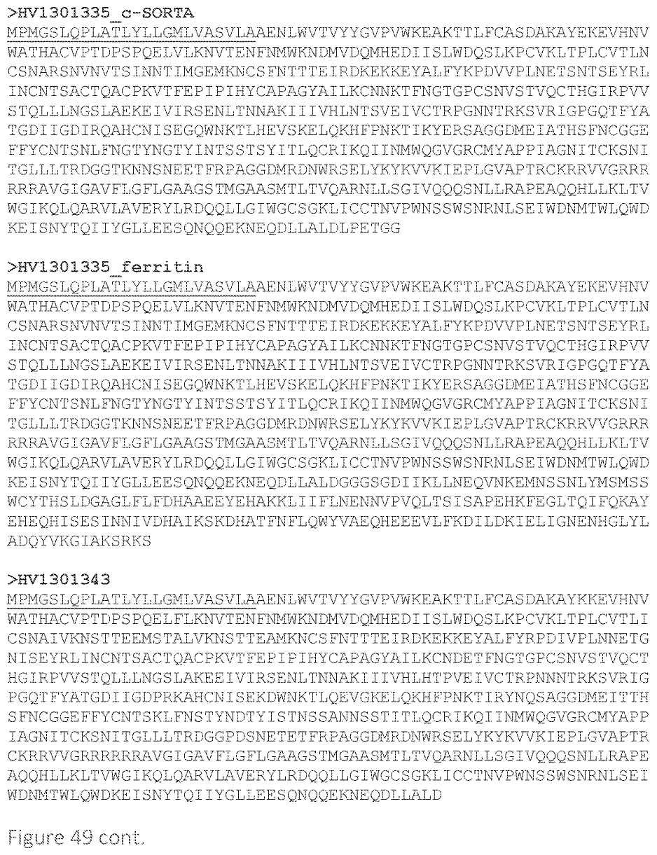

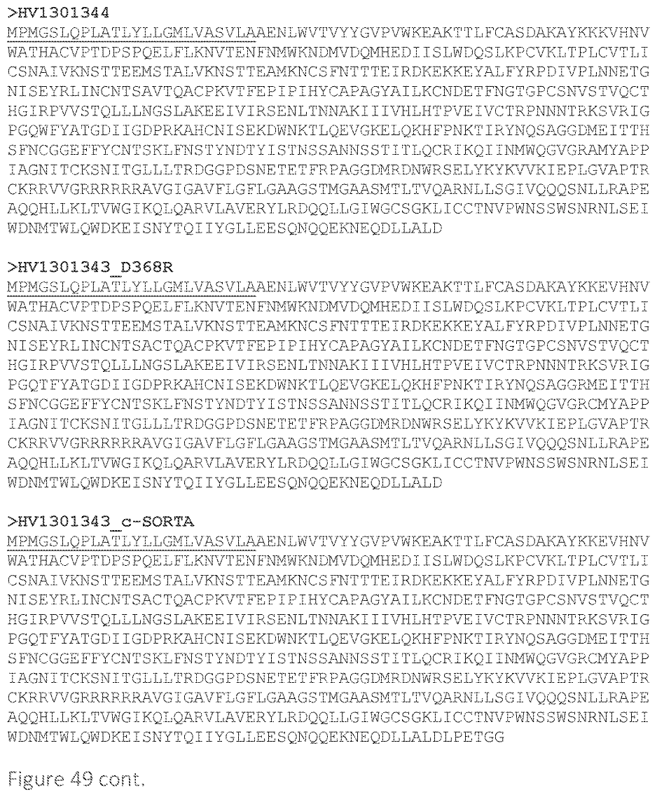

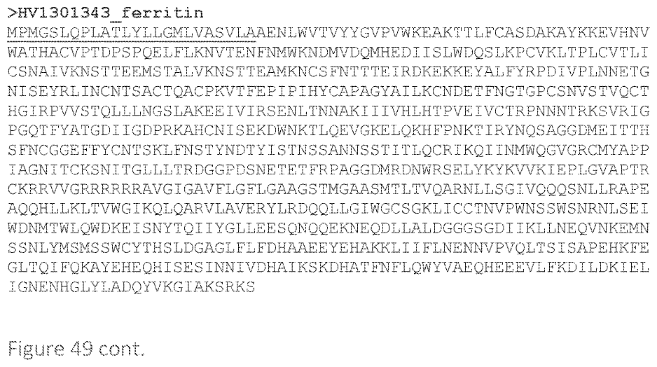

TABLE 4
Summary of selection of immunogens for induction of neutralizing antibodies

| | | NEW | One | aa | Envelope |
|---|---|---|---|---|---|
| | | selection | embodiment | Figures | protomer |
| prime | | CH848.0949.10.17DT | See Table 3, FIG. 47 | | Various protomer |
| | | | | | designs, including |
| | | | | | without limitation various |
| | | | | | stabilized designs. |
| boost | x | CH848.0949.10.17 | See Table 3, FIG. 47 | | Various protomer |
| | | | | | designs, including |
| | | | | | without limitation various |
| | | | | | stabilized designs. |
| boost | | CH848.0808.15.15 | CH0848.3.d0808.15.15.MB | FIG. 48 | CH0848.3.d0808.15.15.MB |
| | | | 6R.DS.SOSIP.664_CD5ss | | 6R.DS.SOSIP.664 |
| | | | | | With various signal |
| | | | | | peptides |
| | | | | | Various protomer |
| | | | | | designs, including |
| | | | | | without limitation various |
| | | | | | stabilized designs. |
| boost | x | CH848.0358.80.06 | | FIG. 49 | |
| boost | x | CH848.1432.5.41 | | FIG. 49 | |
| boost | | CH848.1621.4.44 | CH0848.3.d1621.4.44 | FIG. 48 | CH0848.3.d1621.4.44 |
| | | | 6R.DS.SOSIP.664_CD5ss | | 6R.DS.SOSIP.664 |
| | | | | | With various signal |
| | | | | | peptides |
| | | | | | Various protomer |
| | | | | | designs, including |
| | | | | | without limitation various |
| | | | | | stabilized designs |
| boost | | CH848.1305.10.35 | CH0848.3.d1305.10.35 | FIG. 48 | CH0848.3.d1305.10.35 |
| | | | 6R.DS.SOSIP.664_CD5ss | | 6R.DS.SOSIP.664 |
| | | | | | With various signal |
| | | | | | peptides |
| | | | | | Various protomer |
| | | | | | designs, including |
| | | | | | without limitation various |
| | | | | | stabilized designs |
| boost | | P0402.c2.11 (G) | | FIG. 48 | Various protomer |
| | | | | | designs, including |
| | | | | | without limitation various |
| | | | | | stabilized designs |
| boost | | ZM246F (C) | | FIG. 48 | Various protomer |
| | | | | | designs, including |
| | | | | | without limitation various |
| | | | | | stabilized designs |

(x) indicates non-limiting embodiments of boost envelopes described in Table 3.

Throughout the specification, the name CH848.d0949.10.17 DT is interchangeably used as CH848.d0949.10.17.N133D.N138T. Throughout the specification, the name CH848.d0949.10.17 is interchangeably used as CH848.d0949.10.17WT. In certain embodiments, CH848.d0949.10.17DT envelope comprises additional modifications D230N.H289N.P291S.E169K and is referred to as CH848.d0949.10.17 DTe. In certain embodiments, CH848.d0949.10.17 envelope comprises additional modifications D230N.H289N.P291 S.E169K and is referred to as CH848.d0949.10.17WTe.

Any suitable signal peptide could be used. In designs comprising ferritin for multimerization, any suitable linker could be used between the envelope sequence and a ferritin sequence.

Non-limiting embodiments of DNA sequences of encoding proteins listed in Table 1 and non-limiting embodiments of nucleic acid sequences and amino acid sequences of proteins listed in Table 1 are provided in FIGS. 22-28.

### EXAMPLES

### Example 1—Structural Basis for Development of Breadth in a HIV-1 Neutralizing Antibody

Antibody affinity maturation allows adaptive immune responses to a broad range of pathogens. In some individuals this process leads to broadly neutralizing antibodies that can recognize pathogens with extensive sequence diversity. Vaccine design for pathogens such as influenza, HIV-1, and coronaviruses have therefore focused on recapitulating affinity maturation of these types of antibodies. Here, we determined structures for all antibodies of a broadly neutralizing HIV-1 antibody lineage to examine affinity maturation as neutralization breadth developed from the germline encoded sequence. The results indicate key mutations at differing stages of antibody development resulted in either optimal or suboptimal solutions to antigen engagement. Together, these structures resolve affinity maturation at high spatial resolution and identify specific routes to vaccine development.

Antibody affinity maturation from germline encoded heavy and light chain immunoglobulin genes involves iterative rounds of somatic hypermutation and selection followed by B cell expansion and differentiation, ultimately leading to enhanced pathogen neutralization (Vajda, Porter & Kozakov (2021) Current Opinion in Structural Biology 67, 226-231). The advent of high-throughput next generation sequencing and methods for tracing antibody development has allowed close monitoring of the affinity maturation process (Mishra & Mariuzza (2018) Front Immunol 9, 117; Wardemann & Basse (2017) Trends Immunol 38, 471-482). Extensive structure-based studies of antibodies alone or in contact with cognate antigen have revealed that affinity gains involve acquisition of mutations that improve antibody-antigen contacts, shape complementarity, paratope rigidity, and antibody conformation (Mishra & Mariuzza (2018) Front Immunol 9, 117; McCarthy et al., (2019) Proceedings of the National Academy of Sciences 116, 26745-26751; Schmidt et al., (2013) Proceedings of the National Academy of Sciences 110, 264; Henderson et al., (2019) Nature Communications 10, 654; Bonsignori et al., (2016) Cell 165, 449-463; Zhou et al., (2020) Front Immunol 11, 1529). Maturation also involves affinity-independent diversification, which, together, leads to a broad range of potential solutions to the affinity optimization problem (McCarthy et al., (2019) Proceedings of the National Academy of Sciences 116, 26745-26751; Eisen (2014) Cancer Immunol Res 2, 381-392). Antibodies with neutralization breadth can effectively bind to target antigen despite sequence variability and are a major vaccine design target for pathogens such as influenza, HIV-1, and now, SARS-CoV-2 and related coronaviruses (Stephenson et al., (2020) Annual Review of Immunology 38, 673-703; Laursen & Wilson (2013) Antiviral Research 98, 476-483; Saunders et al., (2021) Nature 594, 553-559). While influenza and SARS-infections are usually cleared within a relatively short time after infection, HIV-1 is a chronic infection and antibody maturation resulting in potent neutralizing antibodies with great breadth tends to occur over years. Defining antibody maturation pathways to strategically inform antigen design and enable acceleration of this process through vaccination is a primary goal of current HIV-1 vaccine efforts (Mu, Haynes & Cain (2021) Current Opinion in Virology 51, 172-178). Such broadly neutralizing antibodies (bnAbs) have been isolated from infected individuals and provide the basis for many strategies aiming to induce bnAbs. Development of HIV-1 directed bnAbs typically require a defined set of specifically positioned mutations in specific heavy/light chain pairs to occur in one of many branches of the antibody lineage, presenting a formidable challenge in immunogen design aimed at recapitulating bnAb development. A detailed understanding of the affinity maturation steps leading to broad neutralization, including discriminating between acquired mutations that lead to the desired bnAb response versus those that culminate in less productive off-target responses, is therefore essential to identify the determinants of mutant selection.

Studying the coevolution of virus and antibody lineages during HIV infection informs vaccine design by defining the HIV-1 Env variants that evolve during bnAb development, thus providing a blueprint for iterative immunogen design (Haynes et al., (2012) Nat Biotech 30, 423-433; Liao et al., (2013) Nature 496, 469-476; Bonsignori et al., (2016) Cell 165, 449-463; Bonsignori et al., (2017) Sci Transl Med 9). The target for HIV-1 bnAbs is the envelope (Env) spike fusion protein (Saunders et al., (2019) Science 366, eaay7199; Ward & Wilson (2015) Trends in biochemical sciences 40, 101-107; Steichen et al., (2019) Cell reports 20, 1805-1817; Torrents de la Peña (2017) Cell reports 20, 1805-1817). The native Env is a trimer of gp120-gp41 heterodimers that are heavily shielded from the host immune systems by N-linked glycosylation and further protected from neutralization by conformational masking and considerable sequence variability. A glycosylated region near the third variable loop (V3) of HIV-1 Env forms a supersite of vulnerability that is targeted by antibodies originating from diverse germline genes in multiple HIV-1 infected individuals (Moyo & Moore (2020) Expert Opinion on Therapeutic Targets 24, 499-509). The development of a broadly neutralizing V3-glycan antibody was studied in an HIV-infected African male from Malawi (CH848), who was followed from the time of infection up to 5 years after transmission (Bonsignori et al., (2017) Sci Transl Med 9). In this individual, the early appearance of two B cell lineages (DH272 and DH475) led to the selection of viral escape variants, which in turn stimulated the DH270 lineage that further developed to acquire potent neutralization breadth (Bonsignori et al., (2017) Sci Transl Med 9). DH270 antibodies were detected in the CH848 individual at week 186 and coincided with the appearance of HIV-1 with shortened variable loop 1 (V1) (Bonsignori et al., (2017) Sci Transl Med 9). The DH270 unmutated common ancestor (DH270.UCA) does not neutralize heterologous HIV-1, although a single amino acid change at position 57 of the heavy chain that substituted a glycine for an arginine (G57R) resulted in heterologous HIV-1 neutralization, albeit with limited breadth. As the DH270 lineage affinity matured and antibodies accumulated mutations in their heavy and light chain variable regions (V_{H} and V_{L}), heterologous breadth and potency improved. The early DH270 lineage members were able to neutralize heterologous viruses with short V1 loops. As the lineage evolved, it gained capacity to neutralize viruses with longer V1 loops, although with reduced potency (Bonsignori et al., (2017) Sc Transl Med 9). Similar inverse correlation between potency and V1 length was observed for other V3-glycan bnAbs 10-1074, PGT121, and PGT128 (Griffith & McCoy (2021) Frontiers in Immunology 12; Anthony et al., (2017) J Virol 91). The rich dataset of co-evolving antibodies and Envs available for the DH270 lineage (Bonsignori et al., (2017) Sci Transl Med 9) presented an opportunity to study structural aspects of affinity maturation and virus coevolution at an unprecedented level of detail and to define how this information will inform vaccine design aimed at eliciting bnAbs targeting the V3-glycan supersite.

Three key discoveries have informed our current understanding of the DH270 lineage. First, though somatic hypermutation is a stochastic process, hot spots and cold spots in the antibody sequence exist leading to differences in mutation site and mutation residue probabilities. HIV-1 bnAbs, including those in the DH270 lineage, are enriched for key, low probability mutations (Wiehe et al., (2018) Cell host & microbe 23, 759-765.e756). Second, of the forty-two mutations in the most broad and potent member, only twelve are needed to reach ninety percent of its breadth allowing us to pinpoint the most critical regions of the antibody that need to be optimized (Swanson et al., (2021) Cell Reports 36, 109561). Finally, key for considering affinity maturation in the context of vaccine development, we have shown that complimentary immunogens can induce development of the lineage from germline (Saunders et al., (2019) Science 366, eaay719). From this context, to better understand antibody evolution at the structural level, we used cryo-EM to visualize the interactions of the complete DH270 lineage tree with the co-evolving virus Env. We define the defenses mounted by the virus to shield its V3-glycan epitope during infection, and how the DH270 lineage develops to first engage Env and then matures to effectively circumvent these barriers to achieve broad neutralization breadth. Our results show that lineage development involved sequential solutions to affinity gain at specific antibody-antigen contact sites. At each stage of development, the acquisition of a solution was followed by branching leading to differential gains in affinity gain and neutralization breadth. The gains permitted increasing breadth allowing the DH270 antibodies to overcome the considerable hurdles presented by differing V1 loop lengths and epitope presentation. Our results shed light on antibody affinity maturation and inform vaccine design that targets the development of broad pathogen neutralization.

### Structural Determination of the Env Bound DH270 Lineage Antibody Fabs

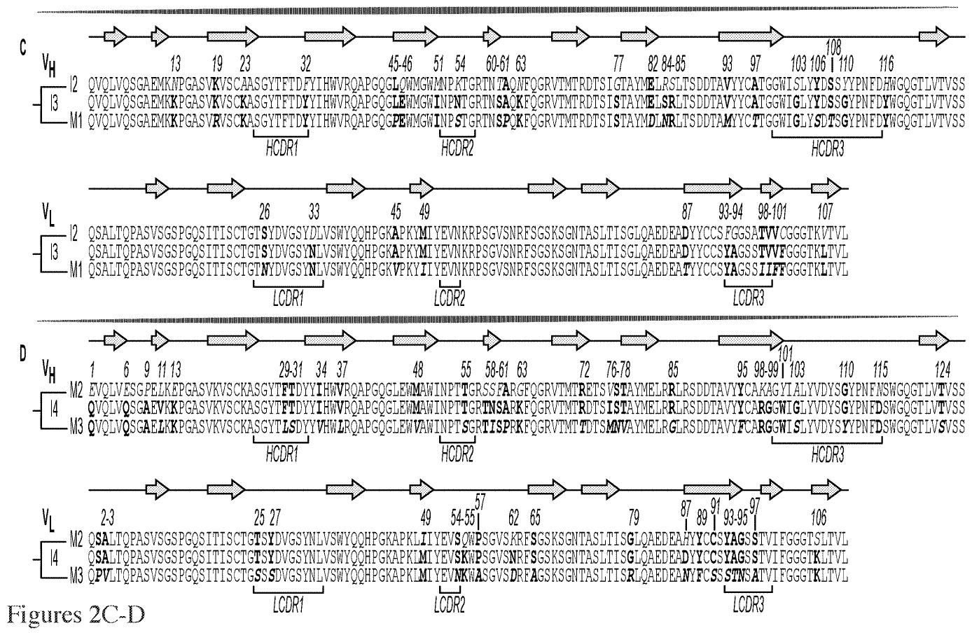

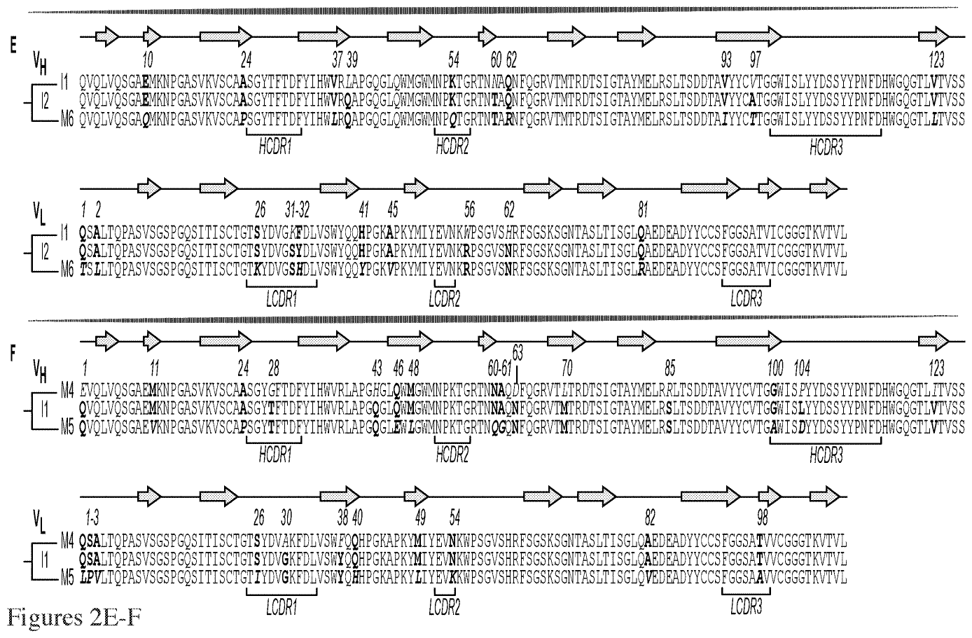

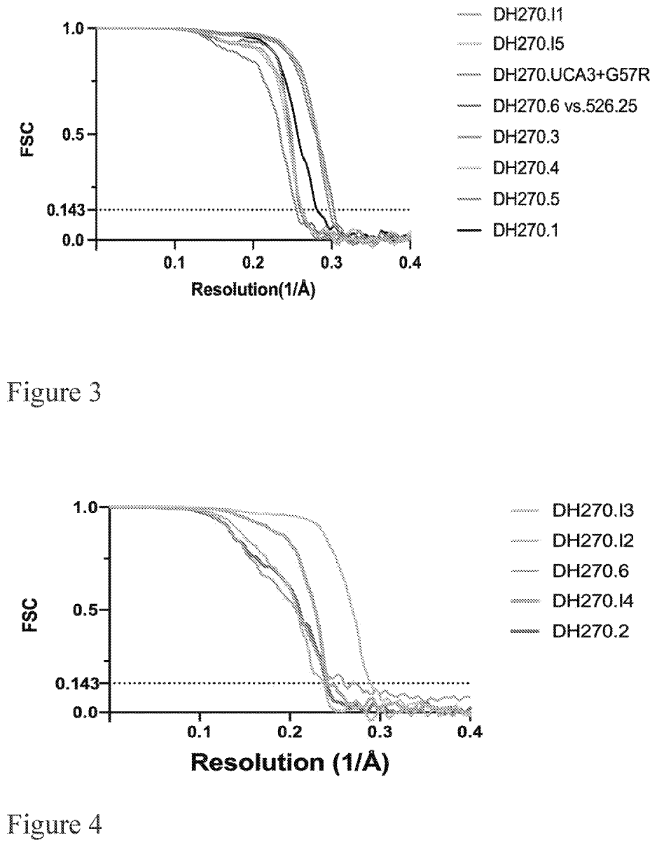

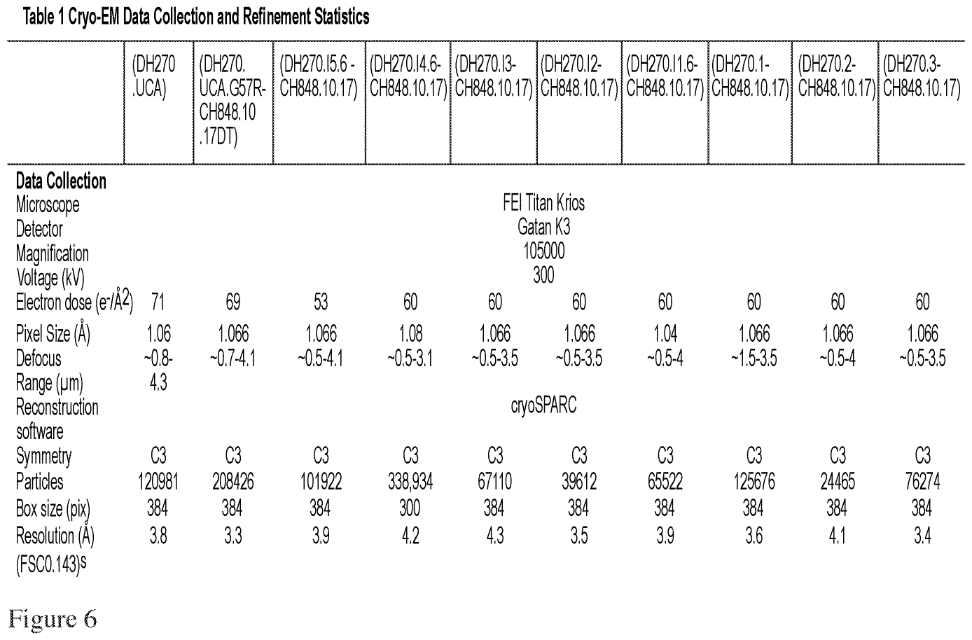

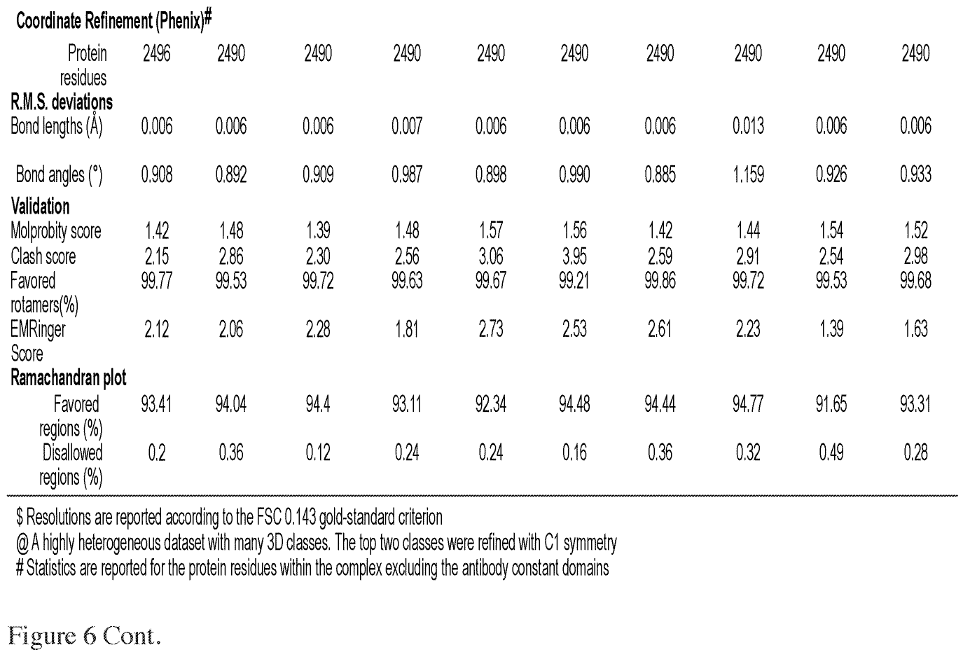

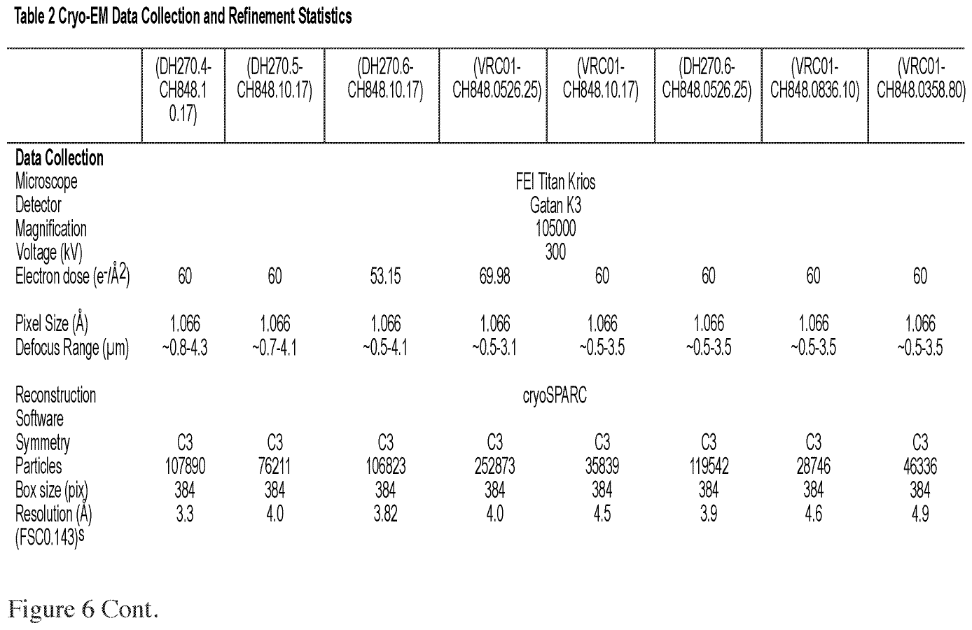

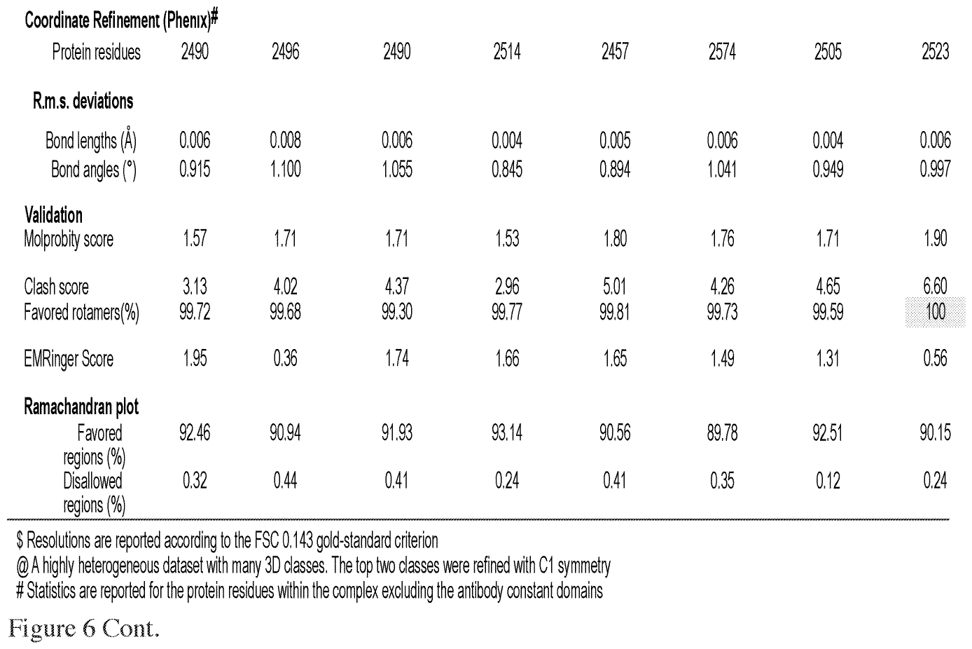

The structures of several DH270 antigen binding fragments (Fabs) have been previously determined, including inferred DH270 UCAs, DH270.3, DH270.5, and DH270.6. Additionally, structures have been determined of a DH270.1 scFv, a Mannose associated DH270.3, a V3-glycan peptide associated DH270.6 scFv, and soluble Env ectodomain bound DH270 UCA and DH270.6 Fabs (Bonsignori et al., (2017) Sci Transl Med 9; Saunders et al., (2019) Science 366, eaay7199; Fera et al., (2018) Nature Communications 9, 1111). While the V3-glycan associated DH270.6 structure clarifies the role of individual residues critical for neutralization breadth, it does not reveal the structural pathway underlying the evolution of this breadth, nor how off-track mutations limit breadth and potency in different DH270 sub-lineages. We therefore sought to determine the impact of mutations at each stage of maturation by determining cryo-EM structures of HIV-1 Env associated DH270 lineage Fabs from each intermediate, and all mature forms (FIGS. 1 and 2). Unless otherwise noted, a soluble SOSIP trimer derived from a virus isolated from the CH848 individual at day 949 post infection was used for preparing complexes with DH270 lineage Fabs for structural studies. Map resolutions ranged from 3.2-5.9 Å with local resolutions at the Fab-Env interface in the range of 3-5 Å (FIGS. 3-6).

Mapping the DH270 lineage mutations on the cryo-EM structures revealed a host of clustered sites that are sequentially mutated along the lineage tree (FIG. 1A). These clusters mapped to seven distinct sites of Ab/Env contact: (1) interaction of the N332-glycan distal D1 arm with the cleft formed between the Fab V_{H}/V_{L} involving the LCRD2 and HCDR3 regions, (2) interaction of the N332-glycan GlcNac base with the antibody HCDR3 region, (3) contact of the V1 loop, and of the conserved GDIR/K motif in the V3 loop with HCDR3 and HCDR2, (4) interaction of the N156-glycan D arms with the antibody framework region, (5) interaction of the N301-glycan base GlcNac-1 and residues around the V3 GDIR/K motif with LCDR3, (6) contact of the N301-glycan branch point and D arms with the LCDR1/V_{L}N-terminus site, and (7) contact of the N442-glycan with LCDR1 (FIG. 1A). These together comprise the entirety of the interactive surface available to the DH270 lineage antibodies.

The cryo-EM reconstructions were well-resolved at the Ab/Env interface. Clear patterns of mutations emerged along the DH270 lineage tree at the Env interactive sites described above that were suggestive of the DH270 lineage evolving to resolve specific, sequential structural bottlenecks on the path of affinity maturation (FIG. 1B). The first intermediate, I5, acquired mutations that strengthened interactions at clusters (3) and (6) by improving protein-protein contacts as well as the interactions of the antibody with the N301 glycan, resulting in ~8-fold improved Env binding affinity relative to the UCA. After intermediate I5, the lineage splits into two distinct paths along an upper branch to I3 and a lower branch to I4. This consequential split leads to the acquisition of greater neutralization breadth and potency in the upper branch, accompanying a steady gradient of affinity gain measured for the upper branch that leads to the DH270.6 bnAb (FIG. 1B) (Bonsignori et al., (2017) Sci Transl Med 9). Both branches acquired mutations around the N332-glycan interaction site but at differing positions. The upper branch 13 intermediate improves interaction with the distal N332-glycan D1 arm (cluster (1)) while the lower branch 14 intermediate improves interaction with the N332-glycan GlcNac base (cluster (2)). Each intermediate then split into two branches, revealing a conserved Env region that is targeted by three of the four mature/intermediate antibodies. Lower branch mature antibody DH270.3 (M3), and upper branch mature antibody DH270.1 (M1) and intermediate antibody 12 show several mutations clustered in or adjacent to the LCDR3 region, presumably improving interactions with the N301-glycan base (cluster (5)). The lower branch mature antibody DH270.2 (M2) acquired mutations focused on cluster (1), improving interactions at the N332-glycan D1 arm in a manner similar to the 13 intermediate. The 12 intermediate acquired additional mutations at clusters (3), (4), and (7). A minimally mutated DH270 antibody was designed (Swanson et al., (2021) Cell Reports 36, 109561) where only 12 of the 42 mutations in DH270.6 were needed to reach ~90% of the breadth and potency of DH270.6. Importantly, the mutations included only those that appeared in I5, I3, and I2. Consistent with this observation, mutations after I2 occurred at more distal sites and provided less clear advantages to those that appeared in the previous lineage members, as described below. The DH270.5 (M5) and DH270.6 (M6) antibodies acquired several mutations near the LCDR1/V_{L} N-terminus where interactions with the N301-glycan D arms may occur. The cryo-EM reconstructions in this region were poorly resolved, precluding a precise definition of these interactions. Together, these results indicate that DH270 lineage maturation involved sequential affinity gain at distinct sites, and that specific structural solutions were necessary for affinity gain and development of neutralization breadth. This is exemplified in the dichotomy between neutralization gains facilitated by mutations in the lower branch and mutations gained in the upper branch. The interaction of the antibody LCDR3 with the N301-glycan base was, for example, a clear target of maturation after the I3 and I4 intermediates.

### UCA to I5: Early Mutations in Lineage Development Set the Stage for Downstream Maturation

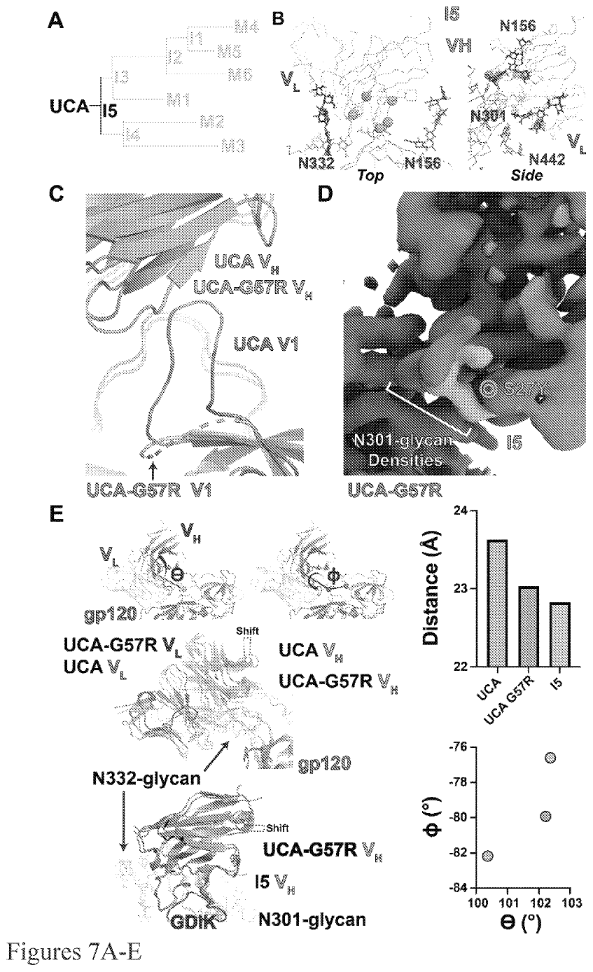

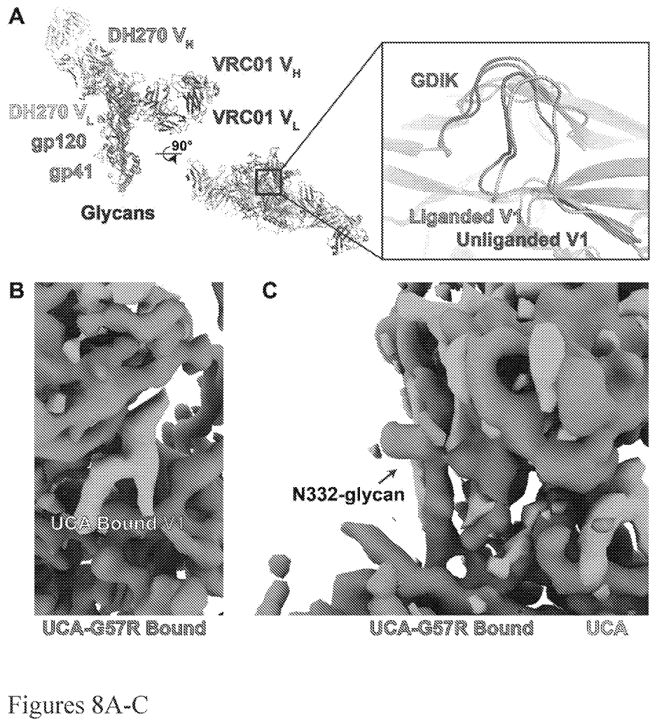

We next studied the specific structural arrangements underlying maturation at each stage, first examining mutations in the 15 intermediate (FIGS. 7A and 7B). We previously determined structures for the DH270 UCA and the mature DH270.6 Fabs bound to the CH848 day 949 timer with V1 loop glycans at positions 133 and 138 removed (Saunders et al., (2019) Science 366, eaay7199). In the UCA-bound structure the V1 loop interacts with both the antibody as well as the GDIK motif (SEQ ID NO: 1) and adjacent V3 residues, while in the DH270.6-bound Env the V1 loop was displaced from this position. This displacement was attributed to the V_{H} G57R mutation that occurs in the first intermediate (Saunders et al., (2019) Science 366, eaay7199. To visualize the V1 loop conformation when not bound to an antibody we determined the structure of the CH848 day 949 SOSIP Env in complex with the CD4 binding site Ab VRC01. We found that the conformation of the unbound V1 loop resembled that of the UCA-bound V1 loop (FIG. 8A). We further determined the structure of DH270.UCA incorporating the V_{H} G57R mutation in complex with the CH848 day 949 SOSIP Env (FIGS. 7C and 8B). The V1 loop in the UCA+G57R structure was displaced from its position over the GDIK motif (SEQ ID NO: 1) observed in its free and UCA-bound forms. The displacement of the V1 loop in the UCA+G57R structure is accompanied by a marked shift in the Ab orientation, rotating the Fab V_{H}/V_{L} toward the space previously occupied by the V1 loop, drawing the antibody closer to the Env (FIGS. 7C and 8C). Comparing the structure of the UCA+G57R-bound complex with the I5-bound complex revealed further shifts in the orientation of the antibody orchestrated by the other acquired mutations. The V_{L} S27Y substitution near the β-Mannose branchpoint of the N301-glycan is the likely culprit, with the bound N301 glycan adopting an altered orientation in the 15-bound complex relative to the UCA+G57R bound complex (FIG. 7D). We next quantified these shifts in antibody orientation using vector-based angles between the antibody Fᵥ and gp120 epitope. Centroids for the epitope, the Fᵥ, and an anchor point in the V_{H} were used to determine Fᵥ to epitope distance and angle (theta) with an additional anchor point in the epitope added to examine rotation (phi) of the antibody relative to the epitope (FIG. 7E). The results indicated that the distance between the antibody and the epitope is reduced by ~0.5 Å in the UCA+G57R bound structure relative to the UCA-bound structure, with further minor reductions observed in I5 associated with the additional I5 mutations (FIG. 7E). Comparison of the angle and dihedral dispositions of the UCA and UCA+G57R structures showed a rotation in each of ~2°. The I5 intermediate showed further rotation about the phi dihedral in the direction of the S27Y mutation without additional changes in the theta rotation angle. Together, these results demonstrate that early mutations in the DH270 lineage facilitate improved contacts, shift the position of the antibody relative to the bound Env, and alter Env conformation particularly of the V1 loop and N301 glycan, with the angular and conformational changes further strengthening antibody-Env interactions.

### The I5 Branch Point: Mutations in I3 Set the Path Toward Broad Neutralization.

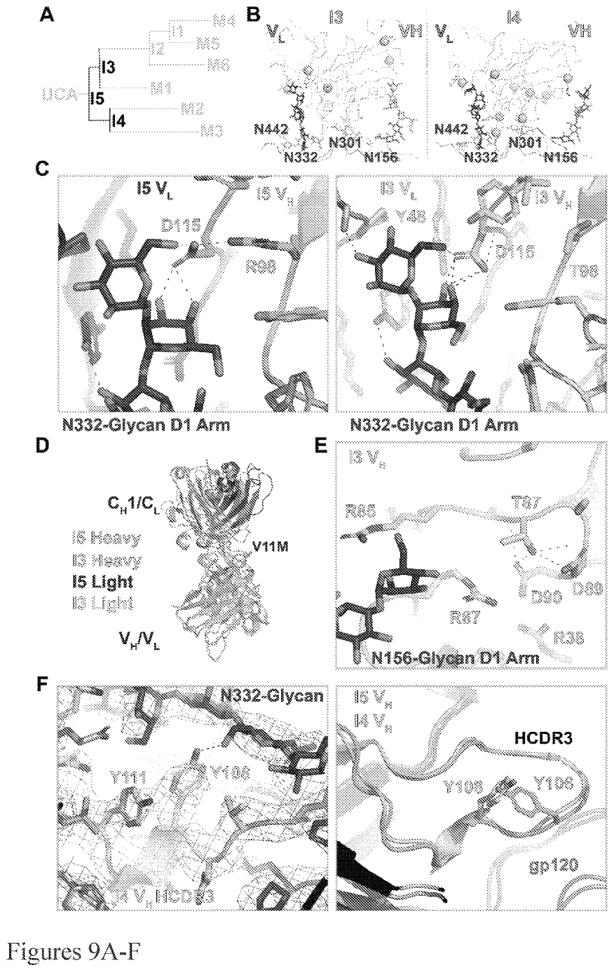

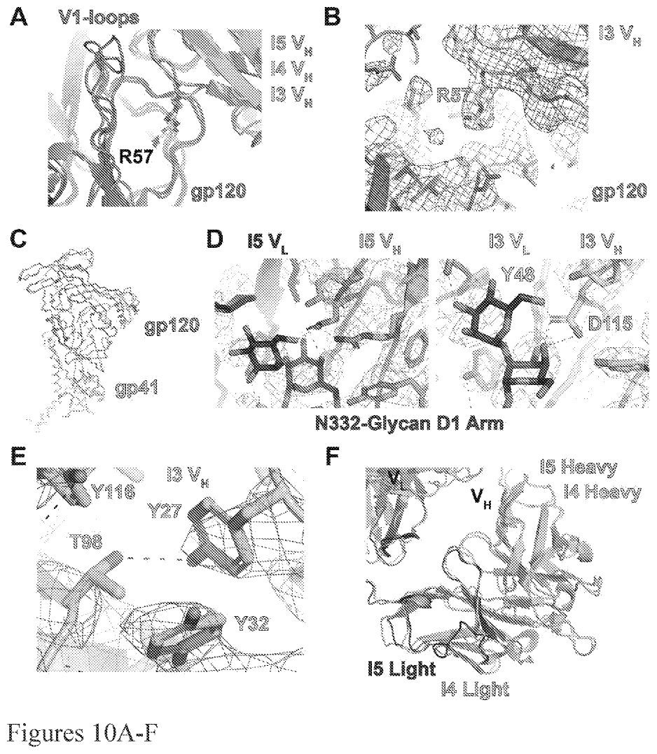

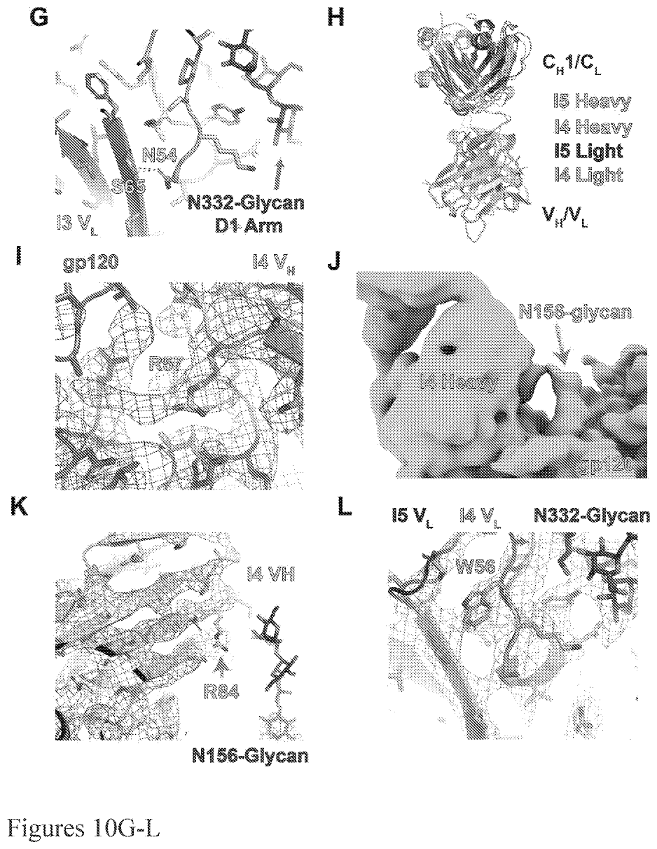

We next examined the I5 branch point (FIGS. 9A and 9B). The I3 intermediate occurs after I5 in the branch leading to the mature DH270.6. Heavy chain mutations include V11M, R87T, and the improbable R98T mutation, while light chain mutations include L48Y and S54N. The cryo-EM reconstruction of the I3 Fab bound Env was determined to a resolution of 4.5 Å. The V1 loop conformational change induced by the V_{H} residue R57 was retained as was the interaction of V_{L} residue Y27 with the N301-glycan (FIGS. 9B, 10A and 10B). No major differences were observed in the Env structure (RMSDs~0.9 Å; FIG. 10C). As previously described, the V_{L} L48Y and V_{H} R98T mutations introduce new hydrogen bonding contacts, with the Y48 hydroxyl interacting with the N332-glycan, and the release of D115 through the R98T mutation allowing closer interaction of D115 with the N332-glycan (FIGS. 9C 10D). Additionally, the I3 V_{H} R98T substitution introduced a hydrogen bond between the side chains of residues T98 and the V_{H} Y27 (FIG. 10E), thus bolstering internal stability of the antibody structure by compensating for the loss of the cation-p interaction of R98 with Y27. The V_{H} R87T and V_{L} S54N mutations are distant from the gp120-interactive region of the antibody. Close examination of the local regions of these mutations suggested potential roles of these substitutions in augmenting internal stability of the antibody. The V_{H} R87T substitution relieves electrostatic strain contributed by three spatially clustered arginines, R67, R85 and R87 (FIG. 9E). Notably, a second Arg in this cluster, R85, is mutated to Ser at the next step as I3 transitions to I2. Moreover, the T87 side chain makes a hydrogen bond with main chain nitrogen of residue V_{H} D89, thus augmenting stability of local structure. The effect of V_{L} S54N was more subtle with N54 appearing to stabilize interaction with an adjacent beta strand via hydrogen bonding of its side chain with the main chain of light chain residue 66 (FIG. 10G).

As predicted by previous molecular dynamics simulations (Henderson et al., (2019) Nature Communications 10, 654), the V1 IM mutation induced a marked shift in the antibody Fab elbow hinge (FIG. 9D). Unlike I3, the I4 intermediate did not acquire an elbow mutation and therefore did not show a distinct shift in the elbow region (FIG. 10H). As in I3, the R57 residue in I4 showed evidence of shifting the V1 position (FIG. 10I). However, additional density in this region is consistent with a closed V1 and suggestive of multistate behavior. The 14 intermediate acquired a larger number of V_{H} mutations including two in the HCDR3, Y106V and S108Y. Consequently, Y106 is positioned closer to the Env surface relative to the Y108 in I4, resulting in N332 GlcNac-2 to I4 Y108 hydroxyl hydrogen bonding. (FIG. 9F). The overall fold of the HCDR3 region was not affected by these mutations (FIG. 9F). The S84R mutation is in proximity to the distal sugar units of the N156-glycan. Densities for the sidechain and this portion of the N156-glycan were, however, not visible in the cryo-EM reconstruction (FIGS. 10J and 10K). The remaining mutations in the 14 heavy chain at positions G49A, N54T, Q62R, and Y116S play no clear role in the interaction with the antibody or in modifying the antibody structure. The light chain contains two non-paratope mutations, R56W and V1001. No changes were observed at the V1001 position relative to the I5 or 13. Residue R59W of the 14 light chain that is adjacent to the LCDR2 region displayed a modest rearrangement that shifts the position of distal N332-glycan interactive residues (FIG. 10L). This was not associated with apparent changes in antibody interactions with the glycan though it may confer stabilization of the LCDR2 loop and therefore the glycan interaction. These observations show that both branches modified interaction with the N332-glycan with possible impacts on downstream maturation.

### The I4 Branch Point: DH270.2 and DH270.3 Attempt Improvements in N332-Glycan Interactions or GDIK (SEQ ID NO: 1) and N301-Glycan Interactions.

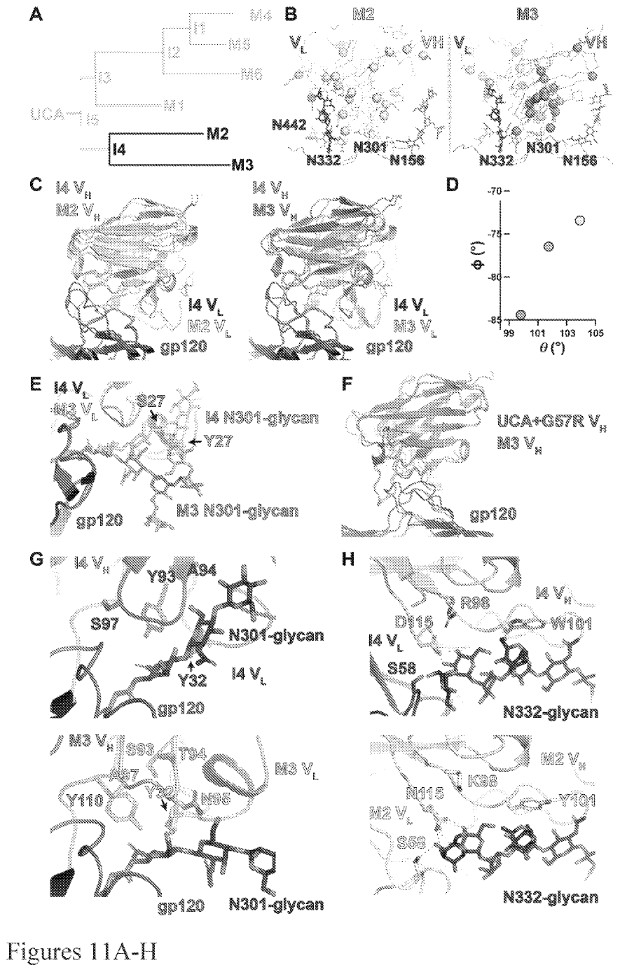

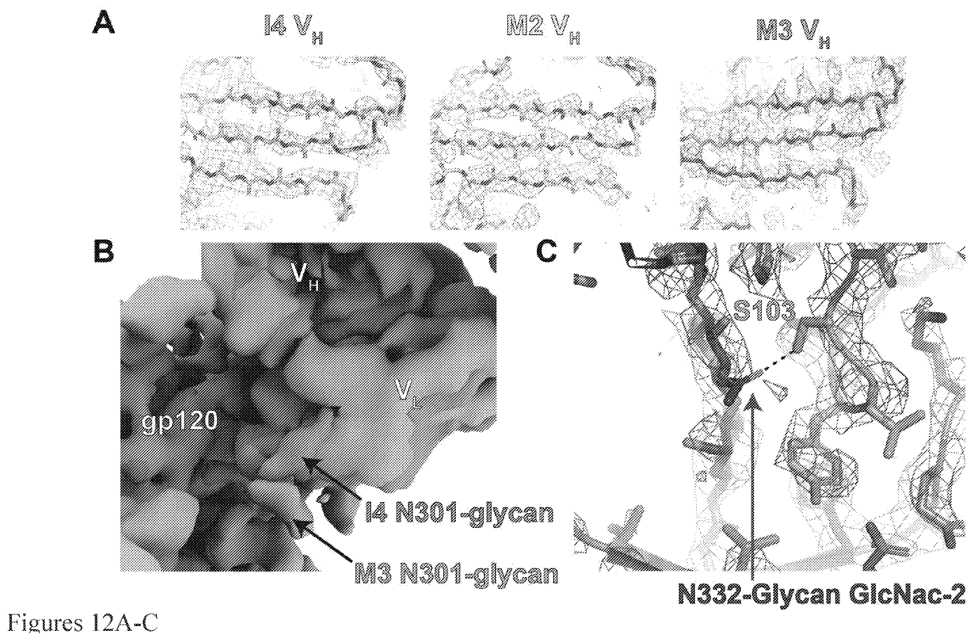

The lower branch of the lineage leads from I4 to mature antibodies DH270.2 and DH270.3 (FIGS. 11A and 11B), with DH270.2 showing greater breadth and potency than DH270.3 with each neutralizing ten and fifteen members from a twenty-four virus heterologous panel, respectively, although both displayed weaker neutralization breadth and potency compared to the upper branch mature antibodies that neutralize sixteen to seventeen members of the heterologous panel (Saunders et al., (2019) Science 366, eaay7199). Alignment of the Env-bound structures revealed distinct shifts in the orientation of each antibody compared to the I4 intermediate (FIGS. 11C and 12A). Inspection of the theta and phi angles indicated DH270.3 had predominantly shifted its rotation about the epitope (FIG. 11D). The most notable mutation in DH270.3 was the light chain Y27S reversion. A clear shift in the position of the N301-glycan was observed relative to the 14 intermediate (FIGS. 11E and 12B). Consistent with the observed rotation of the bound antibody in the 15/Env complex relative to the UCA+G57R/Env complex, the Y27S reversion resulted in DH270.3 occupying an orientation matching UCA+G57R (FIG. 11F). This reversion of antibody orientation caused by the reversion of the S27Y substitution that was acquired previously in the I5 intermediate suggests that the S27Y substitution is the driver of the antibody rotation and N301-glycan re-orientation that was observed on going from the UCA to 15.

A cluster of mutations, including an improbable V_{H} G110Y mutation, occur in and near the DH270.3 LCDR3, near the contact between a loop adjacent to the GDIK motif (SEQ ID NO: 1) and the N301-glycan GlcNac base. Additional mutations, F93S, A94T, G95N, and S97A, result in remodeling of the N301-glycan interaction, shifting the position of the LCDR1 Y32 sidechain for interaction with the first N301-GlcNac and interaction of N95 with the second N301-GlcNac (FIG. 11G). In addition to the angular shift and LCDR3 related mutations, a G103S mutation in HCDR3 adds a hydrogen bond with the N332-glycan GlcNac-2 N-acetyl moiety (FIG. 12C). While DH270.3 mutations optimized interactions with the N301-glycan, the DH270.2 mutations focused instead on the D1 arm N332-glycan interaction. Of the three heavy chain mutations R98K, W101Y, and D115N, the D115N showed the clearest shift in interaction. Loss of negative charge at D115N allowed the asparagine sidechain to shift away from proximity to K98 to form an extensive hydrogen bonding network with the N332-glycan D1 arm (FIG. 11H). This mirrored the R98T mutation, where D115 was freed from its interaction with the positively charged V_{H} residue R98 to engage the N332-glycan. Together, analysis of the Env-bound DH270.2 and DH270.3 structures suggests each followed differing paths of development with each targeting distinct sites. The greater breadth of the DH270.2 antibody compared to DH270.3 is likely a combined effect of improvement in the N332-glycan interaction in DH270.2 and the Y27S reversion in DH270.3.

### The I3 Branch Point: a Complex Series of Mutations in I2 Resolve Epitope Clashes

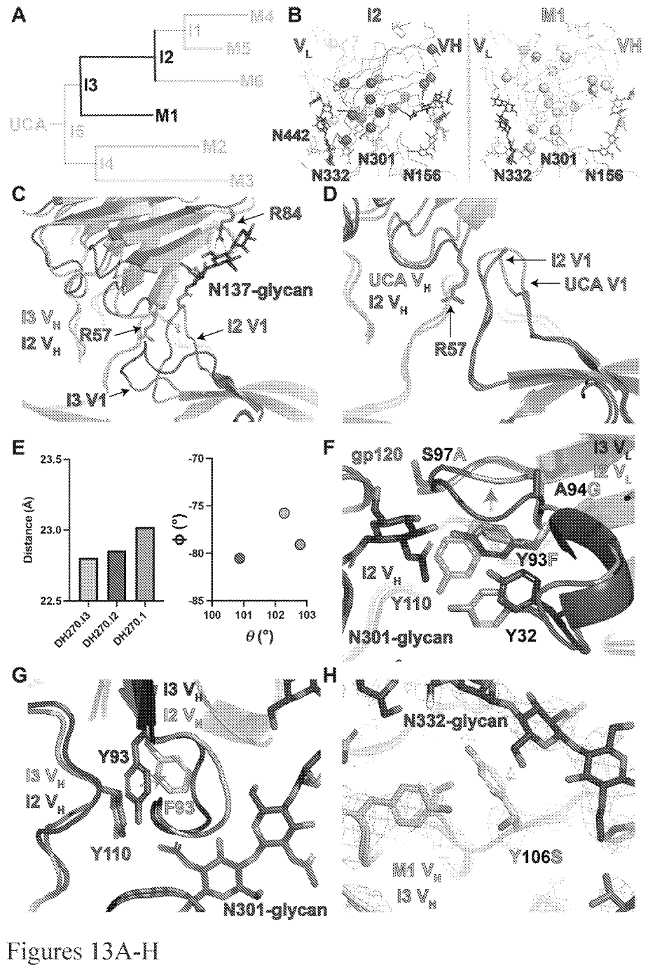

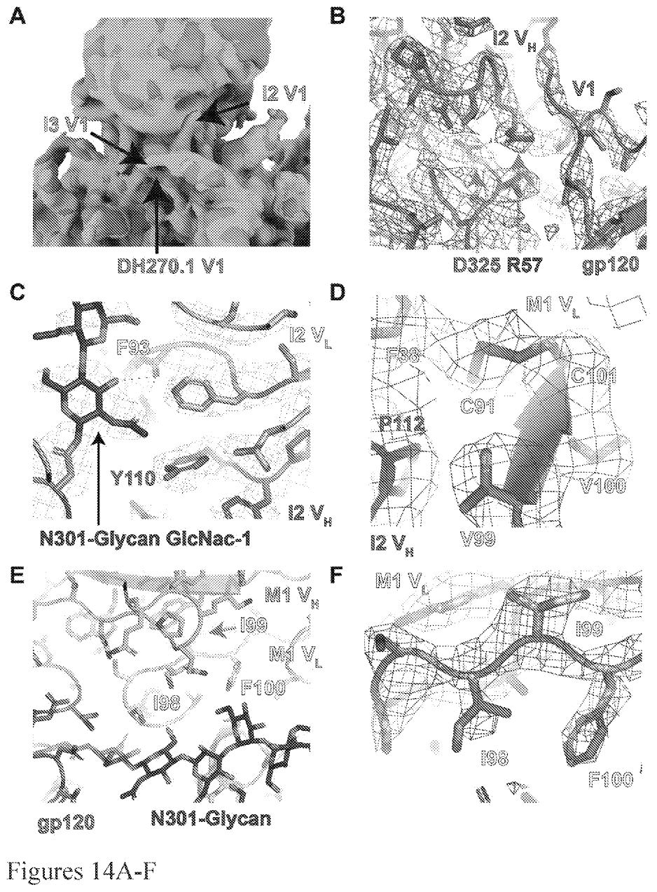

The next stage of development in the upper branch of the DH270 lineage led to the most substantial gains in Env binding affinity (FIG. 1B) and HIV-1 neutralization breadth (FIG. 1B) (Bonsignori et al., (2017) Sci Transl Med 9). The 13 intermediate branches into the I2 intermediate and DH270.1, both of which acquire mutations that are primarily focused on the LCDR3 region (FIGS. 13A and 13B). A notable feature in the cryo-EM reconstruction of the I2-Env complex is the well-resolved V1 loop with an altered configuration. Well-resolved density was also observed for the V1 loop N137-glycan, which contacted the I2 heavy chain S84R mutation site (FIGS. 13C and 14A). This mutation also occurred in the I4 intermediate which showed similar, albeit less clear, densities for a closed V1. Re-orientation of the V1 loop shifted R57 toward the GDIK motif (SEQ ID NO: 1), resulting in its interaction with D325 (FIG. 14B). The V1 loop closing over the V3 loop was similar to the UCA bound trimer structure with a kink in the loop introduced to accommodate R57 (FIG. 13D). Quantification of the antibody orientation showed that the I2 V_{H}/V_{L} have rotated away from the V1 loop to accommodate this loop rearrangement (FIG. 13E).

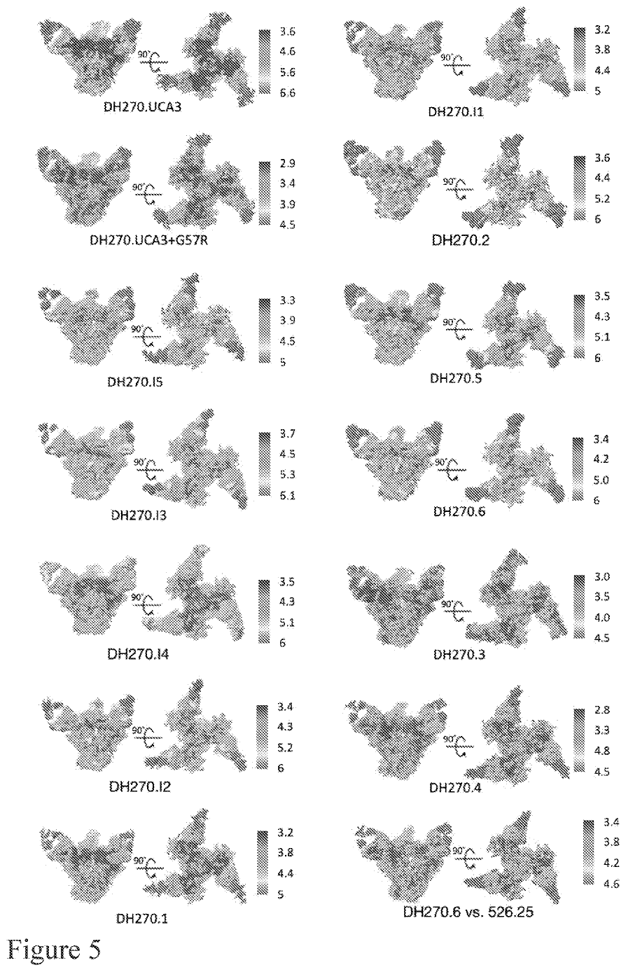

The I2 intermediate and DH270.1 both acquire several LCDR3 modifying mutations like those observed in DH270.3 (FIGS. 13F, 13G, 14C and 14D). In DH270.1 several mutations occurred at the LCDR3 base that were distal to the epitope, and together, they likely stabilized the LCDR3 region (FIGS. 14E and 14F). Similar mutations occur in the 12 intermediate, most notably a F101C mutation spatially adjacent to the LCDR3 that results in the formation of an additional disulfide bond in the antibody between residues 91 and 101 (FIG. 14D). This mutation is among the previously described minimal set of mutations needed to reach ~90% of DH270.6 breadth (Swanson et al., (2021) Cell Reports 36, 109561). A considerable number of mutations in and affecting LDCR3 were also a part of the minimal construct and have distinct effects on epitope interaction in the I2 intermediate bound structure. These included the V_{L} Y93F, A94G, and S97A that work in concert with VH G110Y to cause a rearrangement in the LCDR3 region, creating a pocket in which the N301 GlcNac-1 N-acetyl moiety rests (FIG. 5F). An important impact of the improbable HCDR3 G110Y mutation in creating this pocket is the shifting of the F93 rotamer via steric occlusion (FIG. 13G). Additionally, a HCDR3 Y106S mutation occurs in DH270.1 that resulted in two effects. First, the loss of a hydrogen bond between the side chain of Y106 with the main chain of W101 and, second, release of the interaction of the Y106 side chain with the N332 glycan; together these may act to modulate the interaction of glycan N332 with HCDR3 by allowing it to engage the W101 side chain more effectively (FIG. 13H). These results, together with the observed LCDR3 changes in DH270.3, suggest modifications of the interaction at this site was essential for affinity maturation.

### The I2 Branchpoint: Maturation Shifts to the Epitope Periphery

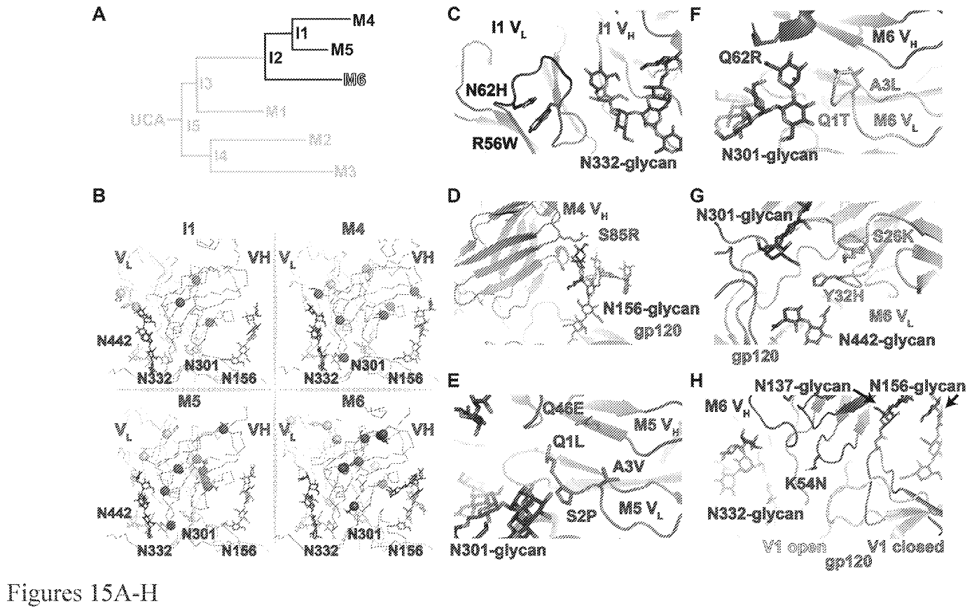

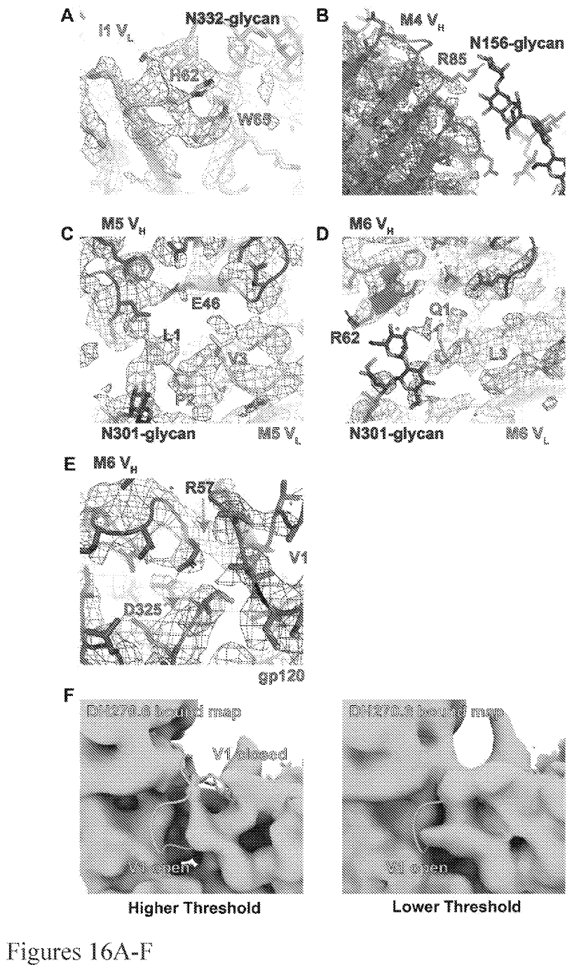

From the I2 intermediate the lineage splits into two branches, both culminating in similar neutralization breadth, albeit with DH270.6 (M6) having greatest potency (FIGS. 15A and 15B) (Bonsignori et al., (2017) Sci Transl Med 9). The upper branch from I2 leads to the I1 intermediate and then on to the mature DH270.4 and DH270.5 antibodies. Mutations in this upper branch were generally further from the antibody-antigen interactive surface with less clear structural impacts. In I1, two mutations with possible impacts on the LCDR2 loop, N62H and R65W, could affect N332-glycan D1 arm interactions (FIGS. 15C and 16A). This loop was, however, poorly resolved, limiting our ability to determine what, if any, impact these mutations have. In DH270.4, a V_{H} S85R mutation is positioned for interaction with the N156-glycan (FIGS. 15D and 16B). However, as was the case for the I1 intermediate mutations, this region is poorly resolved and the interaction cannot, therefore, be confirmed. The DH270.5 V_{H} and V_{L} acquired several mutations in proximity to the N301-glycan D arms (FIGS. 15E and 16C). This was also observed in the DH270.6 V_{H} and V_{L}(FIGS. 15F and 16D). In both cases the map densities left some question as to whether the N301-glycan D arm rests in this position. Additional mutations in DH270.6 occurred near the N442-glycan and the open state V1 loop (FIGS. 15G and 15H). Densities consistent with both the open and closed state V1 loop were apparent in the DH270.6 bound trimer (FIGS. 15H, 16E and 16F). Examination of these structures indicates mutations after I2 occurred primarily at regions more distal to the epitope, consistent with the ability of I2 to neutralize most of the viruses neutralized by DH270.6.

Together, structural determination of the entire lineage revealed an intricate process through which HIV-1 neutralization breadth was developed in the V3-glycan supersite directed DH270 lineage. The early G57R and S27Y mutations of the I5 intermediate had profound impacts on antibody orientation that are likely essential for how the antibodies developed downstream. Branching from I5 to the I3 and I4 intermediates resulted in both acquiring improvements in N332-glycan interaction further highlighting the importance of N332-glycan engagement for the DH270 lineage. Each, however, targeted distinct sites of the glycan, with I3 stabilizing the distal D1 arm and I4 stabilizing the GlcNac base. Downstream maturation from I4 to DH270.2 suggests this was not an ideal solution to the N332-glycan interaction problem, as DH270.2 acquires mutations like those in I3 for the N332-glycan D1 arm. If DH270.2 shows that I4 selection was not ideal, DH270.3 shows the trouble with moving on from a critical juncture too soon. The post-I3 branch, leading to I2 and DH270.1 along with DH270.3, all show a considerable number of mutations in the LDCR3 and adjacent residues. While DH270.3 acquires several residues critical for neutralization, including G110Y and S94A, the selection of the Y27S may have limited its breadth. Where DH270.1 acquired stabilizing LCDR3 mutations, I2, which developed considerable neutralization breadth, modified the LCDR3 conformation, stabilizing a N301-glycan base rest point. This was, critically, mediated by at least five residues, each of which alone may have provided limited affinity gain. In the case of DH270.3, had it first stabilized the N332-glycan D1 arm, the limited gains associated with optimizing LCDR3 site might have been tractable. Paratope modifications downstream from the I2 intermediate were distant from the primary epitope and likely acted to fine tune affinity, breadth, and potency. The minimal design for DH270 (ref) required only mutations from I5 to I2, consistent with this observation. These results show that selection during affinity maturation can be narrowed to a series of structural bottlenecks with optimal and suboptimal solutions.

### Envelope Structural Evolution Optimizes DH270 Lineage Affinity Maturation

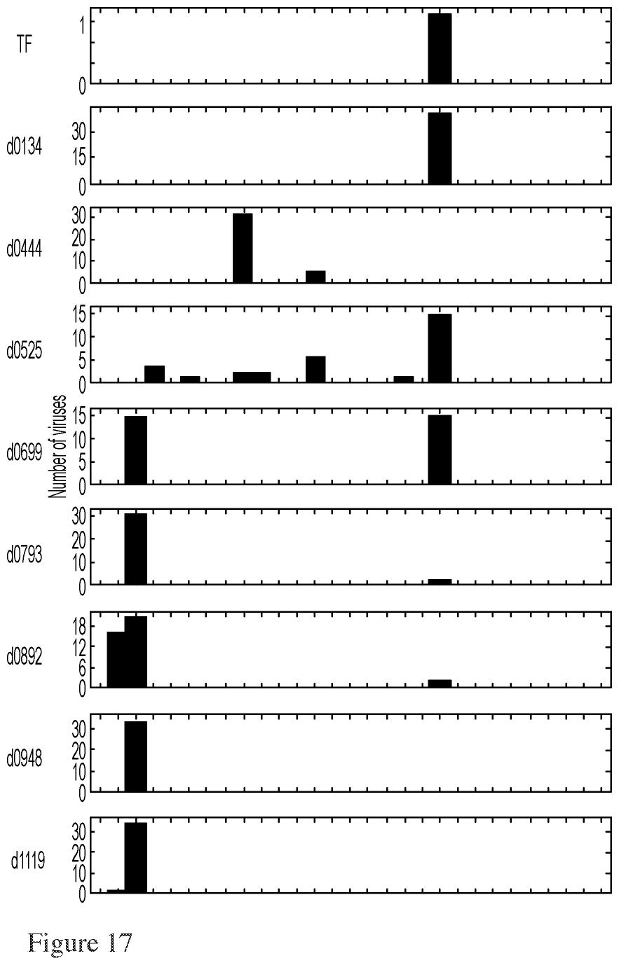

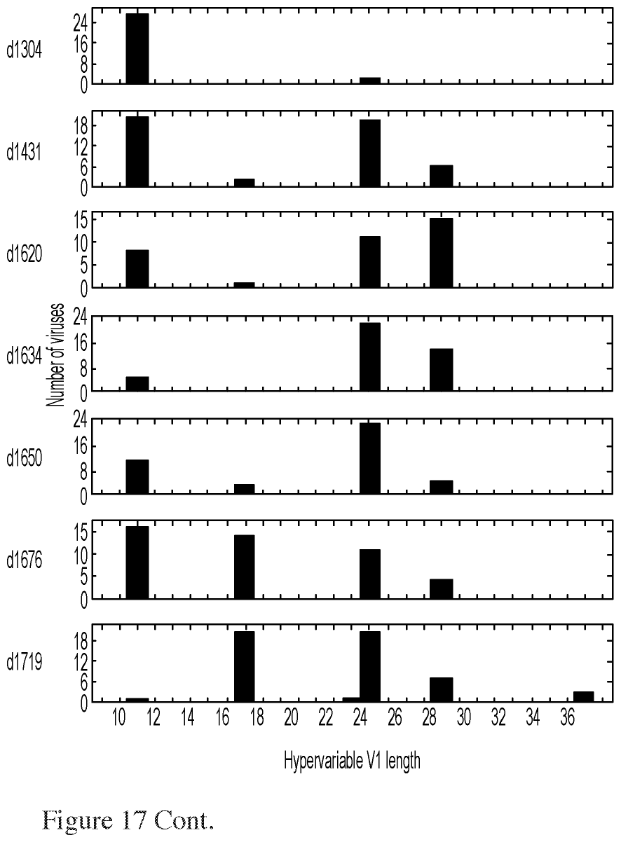

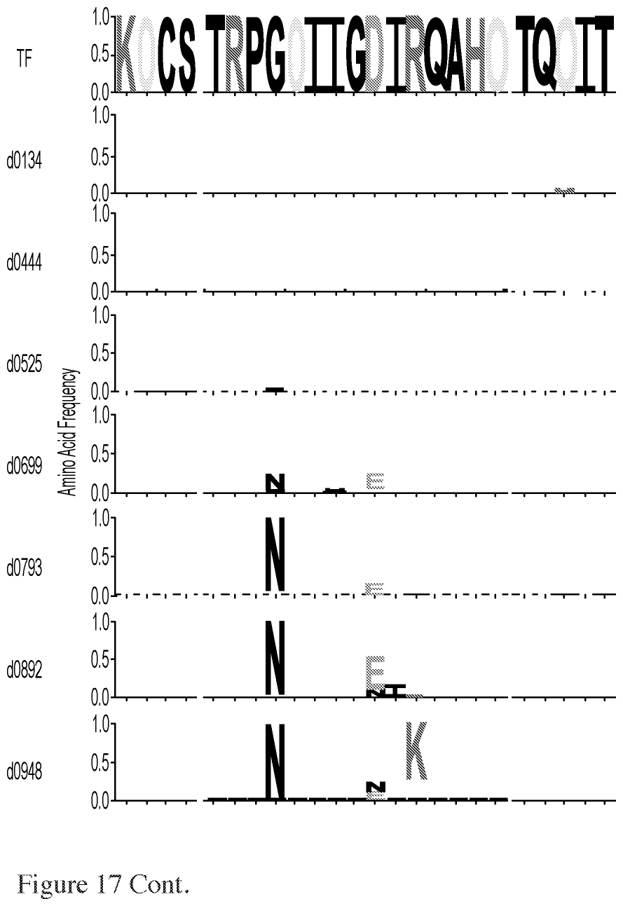

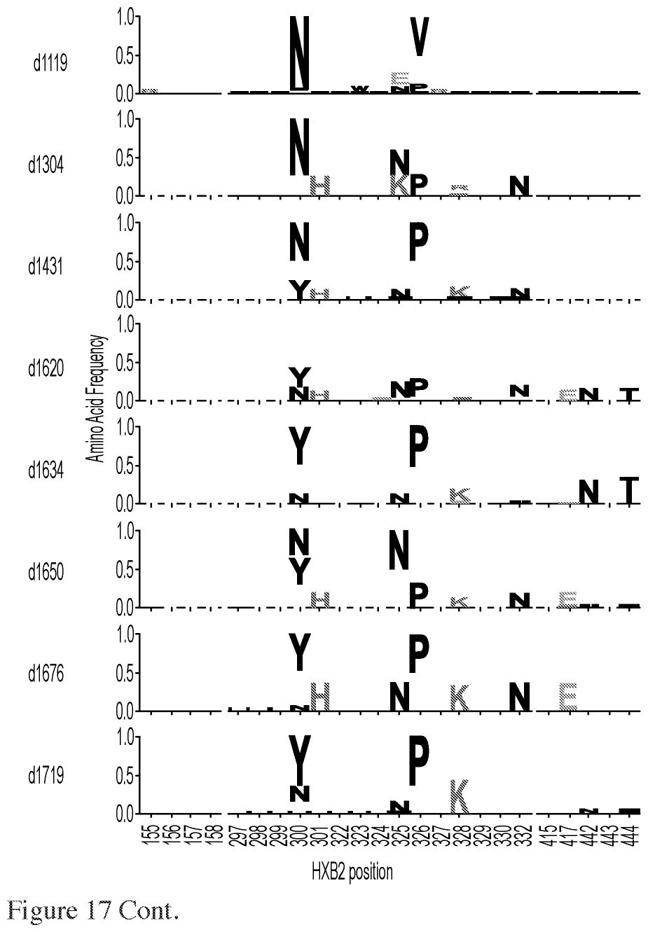

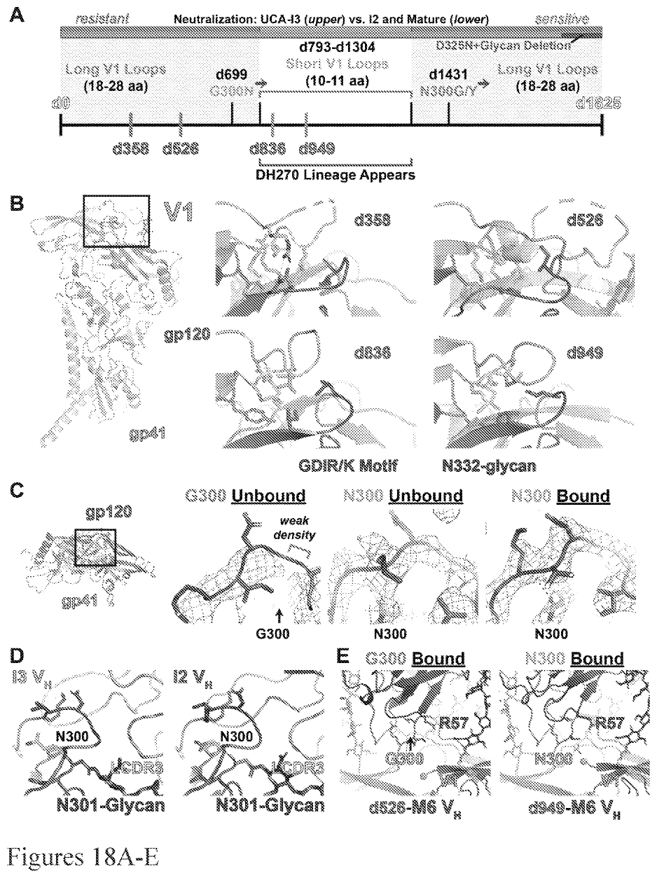

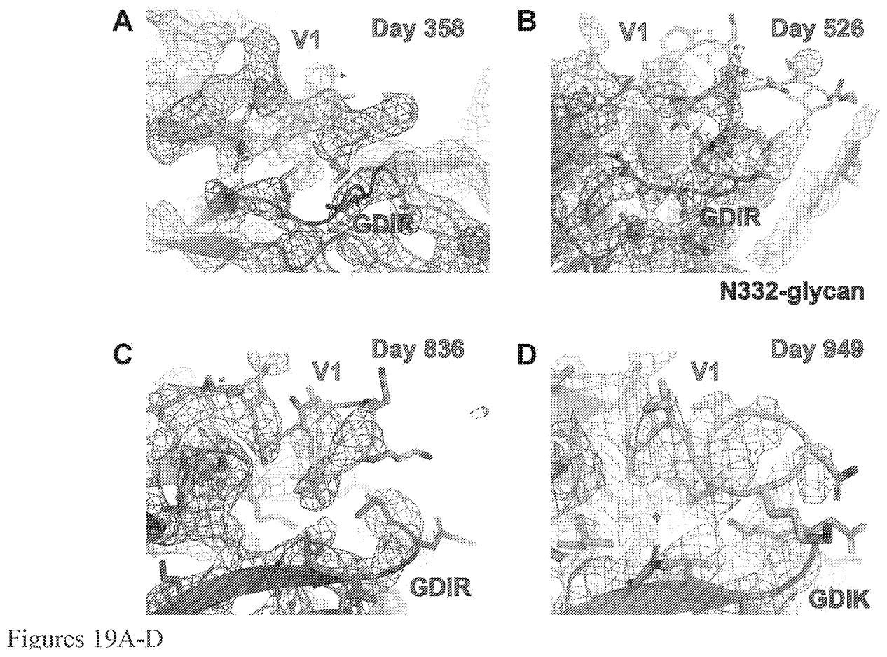

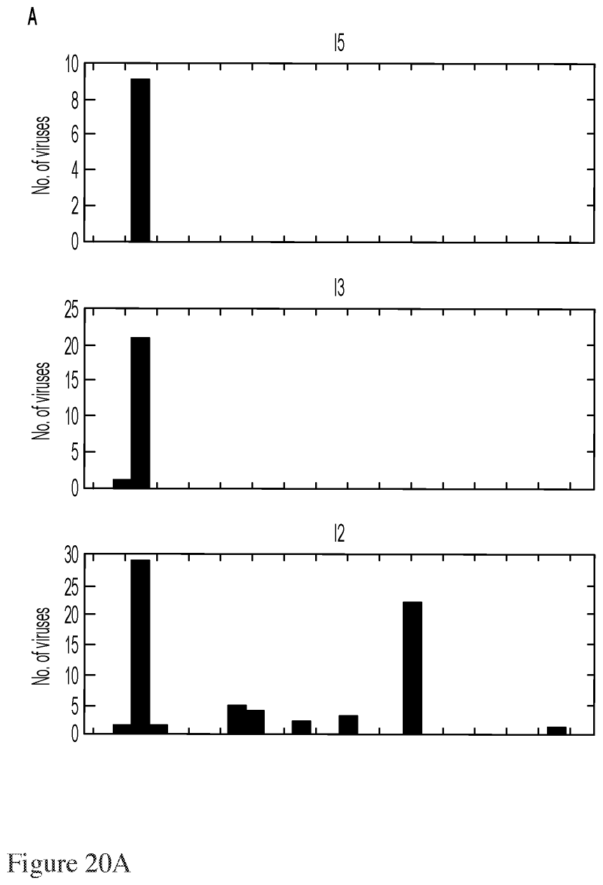

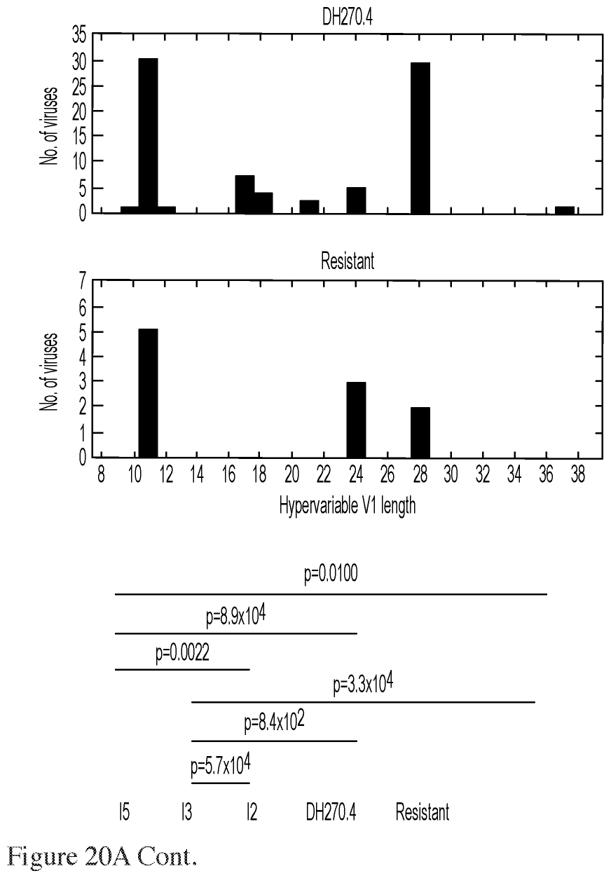

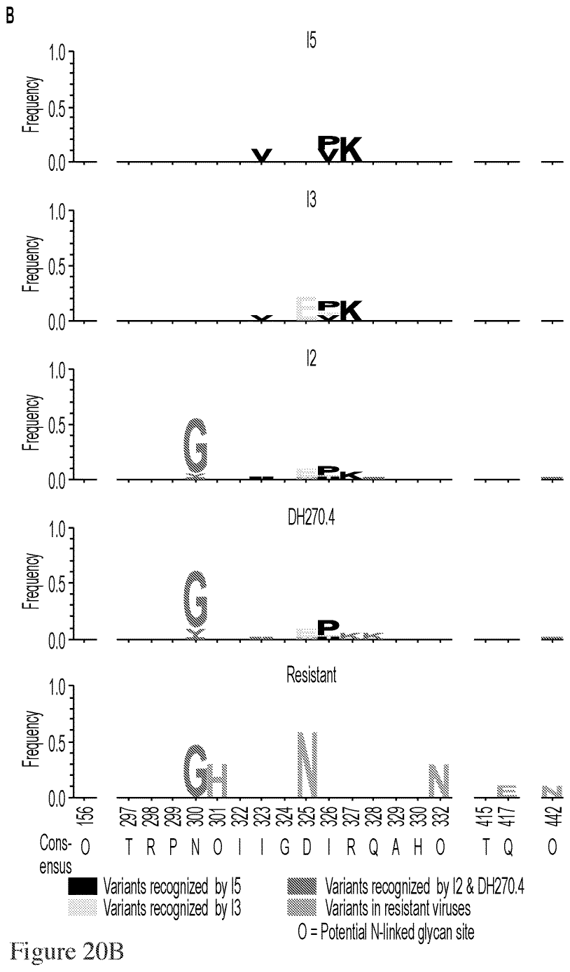

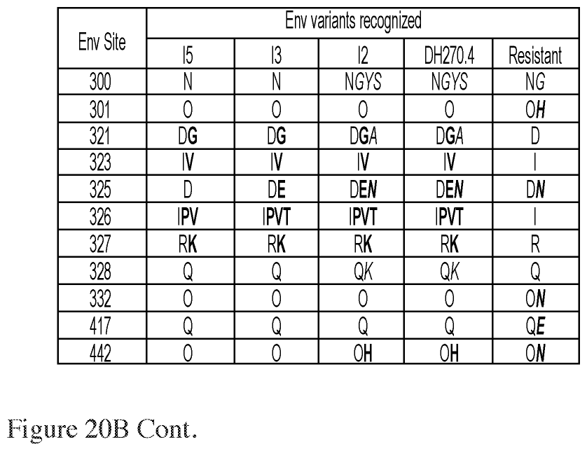

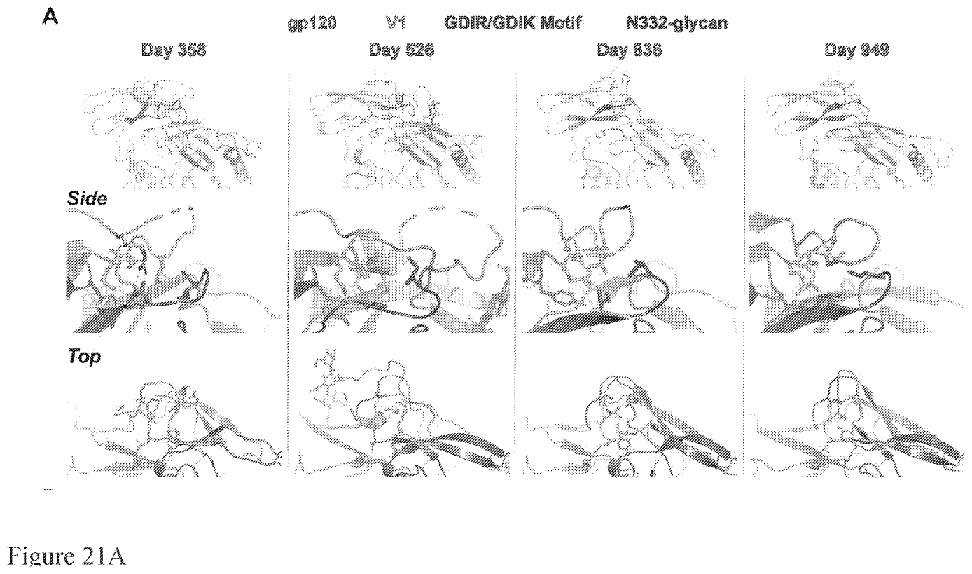

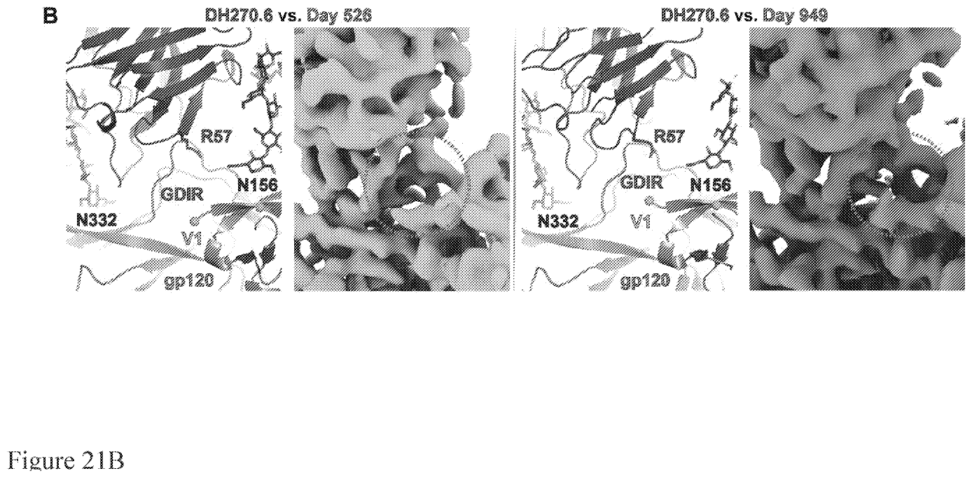

The evolution of HIV-1 neutralization breadth in the DH270 lineage occurred in the backdrop of co-evolution with the virus, with key changes in the Env protein driving antibody maturation, and vice versa (Bonsignori et al., (2017) Sci Transl Med 9). We first examined how the DH270 epitope evolved during infection in the CH848 HIV-1 infected individual (FIG. 17). The first DH270 lineage antibodies appeared between 793- and 1304-days post infection coinciding with a reduction in Env V1 loop length by 18-19 amino acids and a G300N substitution in Env at a location proximal to the DH270 epitope that appeared at day 699 post infection (FIG. 18A). The early I5 and I3 intermediates recognize only the Envs that harbor these shorter V1 loops and the G300N mutation. Ability of the DH270 lineage to neutralize viruses with both longer V1 loop lengths and G300 appears at the 12 intermediate (FIG. 18A). Based on these two early intermediate resistance features, we selected Envs from days 358 and 526, having both G300 and long V1 loops of 18 and 28 amino acids, respectively, and Envs from days 836 and 949, having both N300 and short V1 loops of 11 amino acids. Despite poor sequence similarity, examination of the V1 loop regions of each demonstrated a structurally conserved hydrophobic core and variable hydrogen bonding and/or salt bridge formation that ensures coupling of the V1 loop to the GDIR/K motif. The day 358 Env V1 displayed considerable disorder in a portion of the loop with flanking hydrophobic and salt bridge interactions ensuring the loop remained closed over the GDIR/K motif (FIGS. 18B and 19A). The day 526 Env contained a similar hydrophobic core and displayed more order despite its longer length. This was due to direct V1 loop interaction with the nearby N332-glycan (FIGS. 18B and 19B). Comparison of autologous viruses sensitive to progressively matured DH270 Abs shows that longer V1 loop lengths reemerge, consistent with selection by the later DH270 lineage antibodies (FIG. 20). Longer V1 loop recognition by later stage lineage antibodies is mirrored for heterologous viruses (Bonsignori et al., (2017) Sci Transl Med 9). The day 836 and day 949 Env V1 were markedly shorter but still retain the hydrophobic core and closed state position of the earlier viruses (FIGS. 18B, 19C and 19D). The day 836 loop displayed a kink in the loop near the GDIR/K motif leading to somewhat greater exposure of the motif (FIGS. 18B and 19C). Together, these results indicate a shared protective role of the V1 for the neutralization sensitive GDIR/K motif that is mediated by a conserved hydrophobic core.

We next examined the effect on the DH270 epitope of the Env G300N substitution that is critical for lineage initiation. The GDIR/K motif is proximal to position 300, forming a paired, broken beta sheet secondary structure. Visual inspection of the GDIR/K motif in the G300 containing day 358 and 526 maps suggested greater mobility in the broken sheet structure compared to that of the N300 containing day 836 and 949 maps (FIG. 18C). N300 formed a hydrogen bonding contact with the 1326 backbone of the GDIK motif (SEQ ID NO: 1) in the unbound state. This interaction is retained in the bound state structures for the early intermediates with little change in the GDIK motif (SEQ ID NO: 1) conformation (FIG. 18C). These results suggest the G300N stabilized the DH270 epitope allowing the UCA and early intermediates to bind and neutralize the virus. Mutations in the N300 begin to occur at day 1431 days post infection (FIG. 17) at which point Glycine and Tyrosine variants co-circulate with Asparagine variants. This occurs around the time that the LCDR3 mutations proximal the Env residue 300 occur in 12, DH270.1, and DH270.3, suggesting the evolving virus selected for these mutations in the antibody lineage. Comparison of the Env-bound 13 and 12 structures indicated little overall change in their epitope configuration (FIG. 18D). Additionally, structures of DH270.6 bound to day 526 Env containing G300 and day 949 Env containing N300 show few changes in the overall epitope conformation (FIG. 18E). As the principal effect of the LCDR3 mutations was rigidification of the LCDR3 and closer contact with the N301-glycan base, with little change to the bound state configuration in the unliganded or bound states, these mutants likely enhanced the interaction by compensating for increased epitope flexibility. Additional coevolving Env sites outside the V1 loop and position 300 included positions 325 to 327 in the ³²⁴GDIR/K³²⁷ motif and position 328 (FIG. 17, right). Mutations also occurred that eliminated the N301, N332, and N442 glycan sequons beginning 1304 days post infection. Autologous viruses that were resistant to the mature DH270.4 were predominantly enriched for the D325N substitution, but also had loss of glycans at 301, 332 and/or 442. A clear structural impact for D325N was not observed while the close contacts in all structures with the glycans suggest their loss would indeed have major impacts on DH270 antibody binding. Together, these results show that virus evolution initiated the DH270 lineage evolution by stabilizing the GDIR/K epitope through G300N and removing the protection of the conserved V3 loop GDIR/K motif by the longer V1 loops. The return of these protective features then spurred maturation of the lineage toward stabilization of the paratope to combat enhanced flexibility, resulting in greater neutralization breadth.

Germline antibody-antigen affinities are rarely sufficient to enable pathogen neutralization. Thus, affinity maturation of antibodies is required to improve their interaction with antigen. This is particularly true for neutralizing antibodies that target highly sequence variable viruses such as influenza, HIV-1 and SARS-CoV-2. To better understand this affinity maturation process, we obtained structures for the complete HIV-1 targeting DH270 bnAb lineage to learn how the mutations acquired by the lineage altered the Env-antibody binding interface along the differing paths that the lineage took during its evolution within the HIV-1 infected individual. Our results provide a window into this fraught process revealing that, through the hallmark stochasticity of the affinity maturation process, distinct sites of affinity gain on the antibody are sequentially targeted for optimization as the lineage developed. A recent study of a broadly neutralizing influenza hemagglutinin targeting antibody lineage suggested maturation involves developing a diverse pool of a potential solutions from which further optimization can induce a broad response (McCarthy et al., (2019) Proceedings of the National Academy of Sciences 116, 26745-26751). Consistent with this study, we find that differing mutations solve similar problems presented by the pathogen. However, these solutions are not necessarily equally effective and the developing lineage appears to recognize this, shifting toward the optimal solution even after starting down a less optimal path. What supports the antibody “pluripotency” without being bound by theory developed based on an influenza bnAb creates a vexing problem for vaccine design. As specific endpoints in lineages are desired for their breadth and potency properties, careful steering of developing responses will be required towards these endpoints and away from less productive solutions.

Our results indicate that solutions to particular epitope-paratope pairing issues, rather than occurring together, tend to result in branching. Seen from the perspective of the entire DH270 lineage, failure to acquire an optimal solution results in continued optimization and limits to further development. With a view to vaccine immunogen design, this suggests that failing to acquire specific problem site solutions early is likely to hinder favorable development of neutralization breadth. In the context of the DH270 lineage, vaccination that fails to induce selection of the I5 G57R and S27Y mutations may limit acquisition of the I3 R98T and L48Y mutations. Further, our results suggest off-track mutations can lead lineage development to suboptimal breadth. These off-target responses are likely unavoidable. However, knowing which off-target responses are likely to occur and which portions of the epitope may be involved in selecting for mutations that lead the lineage off-track allows us to select immunogen modifications with greater precision, ensuring modifications produce a positive differential affinity gain favoring the desired mutation(s). Where selection of specific mutations through I3 is challenging from an immunogen development perspective, the considerable number of I2 mutations acquired in concert to achieve a stable LCDR3 interaction will likely require not only rational immunogen design by careful analyses of longitudinal Env-bNAb coevolution but also thinking beyond immunogen properties to issues related to adjuvant, vaccination timing, and the number and identity of boosting immunogens. These issues are further complicated due to the substantial impact of contacts with glycans that cannot be easily controlled in most immunogen design and presentation strategies. Beyond the DH270 lineage, these concerns provide a roadmap to precision lineage-based immunogen development. By isolating distinct structural elements at the antibody-antigen interface, sites of gain can be established, and immunogens can be developed to sequentially solve structural problems associated with each site individually. By shifting focus to specific, manageable steps, rational affinity design can proceed through a series of targets rather than attempting a grand solution to all.

### Methods

### Recombinant Antibody Production

Expi293 cells (ThermoFisher Cat No. A14527) were diluted to a final volume of 0.5 L at a concentration of 2.5×10⁶ cells mL⁻¹ in Expi293 media. Four hundred micrograms of heavy chain and light chain plasmid were complexed with Expifectamine (ThermoFisher Cat No. A14526) and added to the Expi293 cells. On day five, cells were cleared from transfection cell culture media by centrifugation and 0.8 μM filtration. The supernatant containing recombinant antibody was incubated with protein A resin (ThermoFisher) overnight at 4° C. The protein A resin was collected by centrifugation and culture supernatant was removed. The resin was washed with 25 mL of phosphate-buffered saline (PBS) containing a total of 340 mM NaCl. Thirty mL of 10 mM glycine pH 2.4, 150 mM NaCl were used to elute the antibody off of the protein A resin. The pH of the eluted antibody solution was increased to approximately 7 by the addition of 1M Tris pH8.0. The antibody solution was buffer exchanged into PBS with successive rounds of centrifugation, filtered, and stored at −80° C.

### Recombinant HIV-1 Envelope SOSIP Gp140 Production

CH848 SOSIP gp140 envelope production was performed with Freestyle293 cells (ThermoFisher Cat No. R79007). On the day of transfection, Freestyle293 were diluted to 1.25×10⁶ cells/mL with fresh Freestyle293 media up to 1 L total volume. The cells were co-transfected with 650 μg of SOSIP expressing plasmid DNA and 150 μg of furin expressing plasmid DNA complexed with 293Fectin (ThermoFisher Cat No. 12347019). On day 6 cell culture supernatants were harvested by centrifugation of the cell culture for 30 min at 3500 rpm. The cell-free supernatant was filtered through a 0.8 μm filter and concentrated to less than 100 mL with a single-use tangential flow filtration cassette and 0.8 μm filtered again. Trimeric Env protein was purified with monoclonal antibody PGT145 affinity chromatography. PGT145-coupled resin was packed into Tricorn column (GE Healthcare) and stored in PBS supplemented with 0.05% sodium azide. Cell-free supernatant was applied to the column at 2 mL/min using an AKTA Pure (GE Healthcare), washed, and protein was eluted off of the column with 3M MgCl₂. The eluate was immediately diluted in 10 mM Tris pH8, 0.2 μm filtered, and concentrated down to 2 mL for size exclusion chromatography. Size exclusion chromatography was performed with a Superose6 16/600 column (GE Healthcare) in 10 mM Tris pH8, 500 mM NaCl. Fractions containing trimeric HIV-1 Env protein were pooled together, sterile-filtered, snap frozen, and stored at −80° C.

### Cryo-EM Sample Preparation

The CH848 SOSIP trimer complexes were prepared using a stock solution of 2 mg/ml trimer incubated with a six-fold molar excess of Fab. To prevent interaction of the trimer complexes with the air-water interface during vitrification, the samples were incubated in 0.085 mM n-dodecyl β-D-maltoside (DDM). Samples were applied to plasma-cleaned QUANTIFOIL holey carbon grids (EMS, R1.2/1.3 Cu 300 mesh) followed by a 30 second adsorption period and blotting with filter paper. The grid was then plunge frozen in liquid ethane using an EM GP2 plunge freezer (Leica, 90-95% relative humidity).

### Cryo-EM Data Collection

Cryo-EM imaging was performed on a FEI Titan Krios microscope (Thermo Fisher Scientific) operated at 300 kV. Data collection images were acquired with a Falcon 3EC Direct Electron Detector operated in counting mode with a calibrated physical pixel size of 1.08 Å with a defocus range between −1.0 and −3.5 μm using the EPU software (Thermo Fisher Scientific). No energy filter or Cₛ corrector was installed on the microscope. The dose rate used was ~0.8 e⁻/Å²·s to ensure operation in the linear range of the detector. The total exposure time was 60 s, and intermediate frames were recorded every 2 s giving an accumulated dose of ~42 e⁻/Å² and a total of 30 frames per image.

### Data Processing

Cryo-EM image quality was monitored on-the-fly during data collection using automated processing routines. For datasets DH270.I3, DH270.12, DH270.6, DH270.I4 and DH270.2, data processing was performed within cryoSPARC (Swanson et al., (2021) Cell Reports 36, 109561) including particle picking, multiple rounds of 2D classification, ab initio reconstruction, homogeneous map refinement and non-uniform map refinement. The rest of the datasets were further processed outside of cryoSPARC to improve map quality as described next. Movie frame alignment was carried out using UNBLUR (Fera et al., (2018) Nature Communications 9, 1111), and CTF determination using CTFFIND4 (Punjani et al., (2017) Nature Methods 14, 290). Particles were picked automatically using a Gaussian disk of 80 Å in radius as the search template. All the picked particles were extracted with box size 384 pixels and down scaled to 192 pixels (binning 2) and subjected to 8 rounds of refinement in cisTEM (Grant & Grigorieff (2015) eLife 4, e06980), using an ab-initio model generated with cryoSPARC (Swanson et al., (2021) Cell Reports 36, 109561). The distribution of scores assigned to each particle by cisTEM showed a clear bi-modal distribution and only particles in the group containing the higher scores were selected for further processing. The clean subset particles were re-extracted without binning and subjected to 10 iterations of local refinement followed by 5 additional iterations using a shape mask generated with EMAN2 (Rohou & Grigorieff (2015) Journal of Structural Biology 192, 216-221). Per-particle CTF refinement was then conducted until no further improvement in the FSC curve was observed. At this point, particle frames were re-extracted from the raw data and subjected to per-particle motion correction using all movie frames and applying a data-driven dose weighting scheme (Grant, Rohou & Grigorieff (2018) eLife 7, e35383). The new local aligned particles were exported to cryoSPARC and conduct additional round of homogeneous refinement and non-uniform refinement.

For elbow angle analysis with DH270.I3, I4, and DH270.5, local focus refinement with the clean particles after symmetry expansion in cryoSPARC. Then the particles together with refinement parameters were exported to RELION (J. Zivanov, T. Nakane, B. O. Forsberg, D. Kimanius, W. J. H. Hagen, E. Lindahl, et al., New tools for automated high-resolution cryo-EM structure determination in RELION-3 eLife 2018 Vol. 7 Pages e42166) to perform 3D classification without alignment using T=25.

### Cryo-EM Structure Fitting

Structures of each antibody were prepared using Modeller (B. Webb, A. Sali. Comparative Protein Structure Modeling Using Modeller. Current Protocols in Bioinformatics 54, John Wiley & Sons, Inc., 5.6.1-5.6.37, 2016.) and available Fab crystal structures (Bonsignori et al., (2017) Sci Transl Med 9; Fera et al., (2018) Nature Communications 9, 1111). The SOSIP Env structures were prepared using modeler and the DH270.6 bound CH848 day 949 trimer (PDB ID 6UM6 chains A and B) (Saunders et al., (2019) Science 366, eaay719). Coordinates for VRC01 from PDB ID 3NGB (Zhou et al., (2010) Science 329, 811) (chains H and L). Structure fitting of the cryo-EM maps was performed in ChimeraX (Pettersen et al., (2021) Protein science: a publication of the Protein Society 30, 70-82) using the Isolde application (Croll (2018) Acta Crystallographica Section D 74, 519-530). Structure fit and map quality statistics were determined using MolProbity (Chen et al., (2010) Acta Crystallogr D Biol Crystallogr 66, 12-21) and EMRinger (Barad et al., (2015) Nature Methods 12, 943), respectively. Glycans were fit using a combination of the sharpened and gaussian filtered maps (1.0-1.5 standard deviation). Structure and map analyses were performed using a combination of PyMol (Schrodinger, L. The PyMOL Molecular Graphic System (2015)) and ChimeraX with theta and phi angles calculated using VMD (Humphrey, Dalke & Schulten (1996) Journal of Molecular Graphics 14, 33-38).

### REFERENCES

1 Vajda, S., Porter, K. A. & Kozakov, D. Progress toward improved understanding of antibody maturation. Current Opinion in Structural Biology 67, 226-231, doi:https://doi.org/10.1016/j.sbi.2020.11.008 (2021).
2 Mishra, A. K. & Mariuzza, R. A. Insights into the Structural Basis of Antibody Affinity Maturation from Next-Generation Sequencing. Front Immunol 9, 117, doi:10.3389/fimmu.2018.00117 (2018).
3 Wardemann, H. & Busse, C. E. Novel Approaches to Analyze Immunoglobulin Repertoires. Trends Immunol 38, 471-482, doi:10.1016/j.it.2017.05.003 (2017).
4 McCarthy Kevin, R., Raymond Donald, D., Do Khoi, T., Schmidt Aaron, G. & Harrison Stephen, C. Affinity maturation in a human humoral response to influenza hemagglutinin. Proceedings of the National Academy of Sciences 116, 26745-26751, doi:10.1073/pnas.1915620116 (2019).
5 Schmidt, A. G. et al. Preconfiguration of the antigen-binding site during affinity maturation of a broadly neutralizing influenza virus antibody. Proceedings of the National Academy of Sciences 110, 264, doi:10.1073/pnas.1218256109 (2013).
6 Henderson, R. et al. Selection of immunoglobulin elbow region mutations impacts interdomain conformational flexibility in HIV-1 broadly neutralizing antibodies. Nature Communications 10, 654, doi:10.1038/s41467-019-08415-7 (2019).
7 Bonsignori, M. et al. Maturation Pathway from Germline to Broad HIV-1 Neutralizer of a CD4-Mimic Antibody. Cell 165, 449-463, doi:10.1016/j.cell.2016.02.022 (2016).
8 Zhou, J. O., Zaidi, H. A., Ton, T. & Fera, D. The Effects of Framework Mutations at the Variable Domain Interface on Antibody Affinity Maturation in an HIV-1 Broadly Neutralizing Antibody Lineage. Front Immunol 11, 1529, doi:10.3389/fimmu.2020.01529 (2020).
9 Eisen, H. N. Affinity enhancement of antibodies: how low-affinity antibodies produced early in immune responses are followed by high-affinity antibodies later and in memory B-cell responses. Cancer Immunol Res 2, 381-392, doi:10.1158/2326-6066.Cir-14-0029 (2014).
10 Stephenson, K. E., Wagh, K., Korber, B. & Barouch, D. H. Vaccines and Broadly Neutralizing Antibodies for HIV-1 Prevention. Annual Review of Immunolog 38, 673-703, doi:10.1146/annurev-immunol-080219-023629 (2020).
11 Laursen, N. S. & Wilson, I. A. Broadly neutralizing antibodies against influenza viruses. Antiviral Research 98, 476-483, doi:https://doi.org/10.1016/j.antiviral.2013.03.021 (2013).
12 Saunders, K. O. et al. Neutralizing antibody vaccine for pandemic and pre-emergent coronaviruses. Nature 594, 553-559, doi:10.1038/s41586-021-03594-0 (2021).
13 Mu, Z., Haynes, B. F. & Cain, D. W. Strategies for eliciting multiple lineages of broadly neutralizing antibodies to HIV by vaccination. Current Opinion in Virology 51, 172-178, doi:https://doi.org/10.1016/j.coviro.2021.09.015 (2021).
14 Haynes, B. F., Kelsoe, G., Harrison, S. C. & Kepler, T. B. B-cell-lineage immunogen design in vaccine development with HIV-1 as a case study. Nat Biotech 30, 423-433, doi:10.1038/nbt.2197 (2012).
15 Liao, H. X. et al. Co-evolution of a broadly neutralizing HIV-1 antibody and founder virus. Nature 496, 469-476, doi:10.1038/nature12053 (2013).
16 Bonsignori, M. et al. Maturation Pathway from Germline to Broad HIV-1 Neutralizer of a CD4-Mimic Antibody. Cell 165, 449-463, doi:10.1016/j.cell.2016.02.022 (2016).
17 Bonsignori, M. et al. Staged induction of HIV-1 glycan-dependent broadly neutralizing antibodies. Sci Transl Med 9, doi:10.1126/scitranslmed.aai7514 (2017).
18 Saunders, K. O. et al. Targeted selection of HIV-specific antibody mutations by engineering B cell maturation. Science 366, eaay7199, doi:10.1126/science.aay7199 (2019).
19 Ward, A. B. & Wilson, I. A. Insights Into the Trimeric HIV-1 Envelope Glycoprotein Structure. Trends in biochemical sciences 40, 101-107, doi:10.1016/j.tibs.2014.12.006 (2015).
20 Steichen, J. M. et al. A generalized HIV vaccine design strategy for priming of broadly neutralizing antibody responses. Science (New York, N.Y.) 366, eaax4380, doi:10.1126/science.aax4380 (2019).
21 Torrents de la Peña, A. et al. Improving the Immunogenicity of Native-like HIV-1 Envelope Trimers by Hyperstabilization. Cell reports 20, 1805-1817, doi:10.1016/j.celrep.2017.07.077 (2017).
22 Moyo, T., Kitchin, D. & Moore, P. L. Targeting the N332-supersite of the HIV-1 envelope for vaccine design. Expert Opinion on Therapeutic Targets 24, 499-509, doi:10.1080/14728222.2020.1752183 (2020).
23 Griffith, S. A. & McCoy, L. E. To bnAb or Not to bnAb: Defining Broadly Neutralising Antibodies Against HIV-1. Frontiers in Immunology 12 (2021).
24 Anthony, C. et al. Cooperation between Strain-Specific and Broadly Neutralizing Responses Limited Viral Escape and Prolonged the Exposure of the Broadly Neutralizing Epitope. J Virol 91, doi:10.1128/jvi.00828-17 (2017).
25 Wiehe, K. et al. Functional Relevance of Improbable Antibody Mutations for HIV Broadly Neutralizing Antibody Development. Cell host & microbe 23, 759-765.e756, doi:10.1016/j.chom.2018.04.018 (2018).
26 Swanson, O. et al. Rapid selection of HIV envelopes that bind to neutralizing antibody B cell lineage members with functional improbable mutations. Cell Reports 36, 109561, doi:https://doi.org/10.1016/j.celrep.2021.109561 (2021).
27 Fera, D. et al. HIV envelope V3 region mimic embodies key features of a broadly neutralizing antibody lineage epitope. Nature Communications 9, 1111, doi:10.1038/s41467-018-03565-6 (2018).
28 Punjani, A., Rubinstein, J. L., Fleet, D. J. & Brubaker, M. A. cryoSPARC: algorithms for rapid unsupervised cryo-EM structure determination. Nature Methods 14, 290, doi:10.1038/nmeth.4169 https://www.nature.com/articles/nmeth.4169 #supplementary-information (2017).
29 Grant, T. & Grigorieff, N. Measuring the optimal exposure for single particle cryo-EM using a 2.6 Å reconstruction of rotavirus VP6. eLife 4, e06980, doi:10.7554/eLife.06980 (2015).
30 Rohou, A. & Grigorieff, N. CTFFIND4: Fast and accurate defocus estimation from electron micrographs. Journal of Structural Biology 192, 216-221, doi:https://doi.org/10.1016/j.jsb.2015.08.008 (2015).
31 Grant, T., Rohou, A. & Grigorieff, N. cisTEM, user-friendly software for single-particle image processing. eLife 7, e35383, doi:10.7554/eLife.35383 (2018).
32 Tang, G. et al. EMAN2: An extensible image processing suite for electron microscopy. Journal of Structural Biology 157, 38-46, doi:http://dx.doi.org/10.1016/j.jsb.2006.05.009 (2007).
33 Bartesaghi, A. et al. Atomic Resolution Cryo-EM Structure of β-Galactosidase. Structure 26, 848-856.e843, doi:https://doi.org/10.1016/j.str.2018.04.004 (2018).
34 B. Webb, A. Sali. Comparative Protein Structure Modeling Using Modeller. Current Protocols in Bioinformatics 54, John Wiley & Sons, Inc., 5.6.1-5.6.37, 2016.
35 Zhou, T. et al. Structural Basis for Broad and Potent Neutralization of HIV-1 by Antibody VRC01. Science 329, 811 (2010).
36 Pettersen, E. F. et al. UCSF ChimeraX: Structure visualization for researchers, educators, and developers. Protein science: a publication of the Protein Society 30, 70-82, doi:10.1002/pro.3943 (2021).
37 Croll, T. ISOLDE: a physically realistic environment for model building into low-resolution electron-density maps. Acta Crystallographica Section D 74, 519-530, doi:10.1107/S2059798318002425 (2018).
38 Chen, V. B. et al. MolProbity: all-atom structure validation for macromolecular crystallography. Acta Crystallogr D Biol Crystallogr 66, 12-21, doi:10.1107/S0907444909042073 (2010).
39 Barad, B. A. et al. EMRinger: side chain-directed model and map validation for 3D cryo-electron microscopy. Nature Methods 12, 943, doi:10.1038/nmeth.3541 https://www.nature.com/articles/nmeth.3541 #supplementary-information (2015).
40 Schrodinger, L. The PyMOL Molecular Graphics System. (2015).
41 Humphrey, W., Dalke, A. & Schulten, K. VMD: Visual molecular dynamics. Journal of Molecular Graphics 14, 33-38, doi: https://doi.org/10.1016/0263-7855(96)00018-5 (1996).

### Example 2

This example describes animal studies with HIV-1 envelopes designed to prime and boost V3 glycan antibodies lineages.

The envelopes described in Table 1 (and optionally comprising any combinations of additional modifications, such as the modifications described in Tables 2A-B) or Table 2B, expressed as recombinant proteins or modified mRNA formulated in LNP, are analyzed in animal studies including mouse and NHP animal models. The mouse animal model could be any model, including an animal model comprising a DH270UCA transgene.

Any suitable adjuvant will be used. The number and time interval between boost can be determined experimentally.

The envelopes in Table 1 (and optionally comprising any combinations of additional modifications, such as the modifications described in Tables 2A-B) or Table 2B will be produced under cGMP conditions as a recombinant protein and/or mRNA formulated in LNP for use in Phase I clinical trial.

### Example 3

Despite the lengths of d0358.80.06 V1 loop (18aa) and d1432.5.41 V1 loop (24aa), their coverage of the V3-glycan site is similar to the shorter V1 loop (11aa) in d0949.10.17, leaving most of the site exposed. However, d949 Env was shown as neutralizable by the early DH270 clones, while d358 was resistant and only neutralized by the matured bnAb. Intriguingly, a novel H-bond between R327 on V3 and the N332-GlcNAc-2 was observed in d358 where N332 acted as a protection layer. In contrast, the density of N332-glycan was less clear in d949 Env with a R327K mutation. This interaction was also not seen in d1432 R327 because of the I326P mutation that twisted the R327 sidechain away from the N332-glycan. (FIGS. 50A-C) Therefore, GDIR (SEQ ID NO: 2) is potentially a better motif than GDIK/GDPR for filing the glycan hole and immunogen that can elicit broad neutralization.

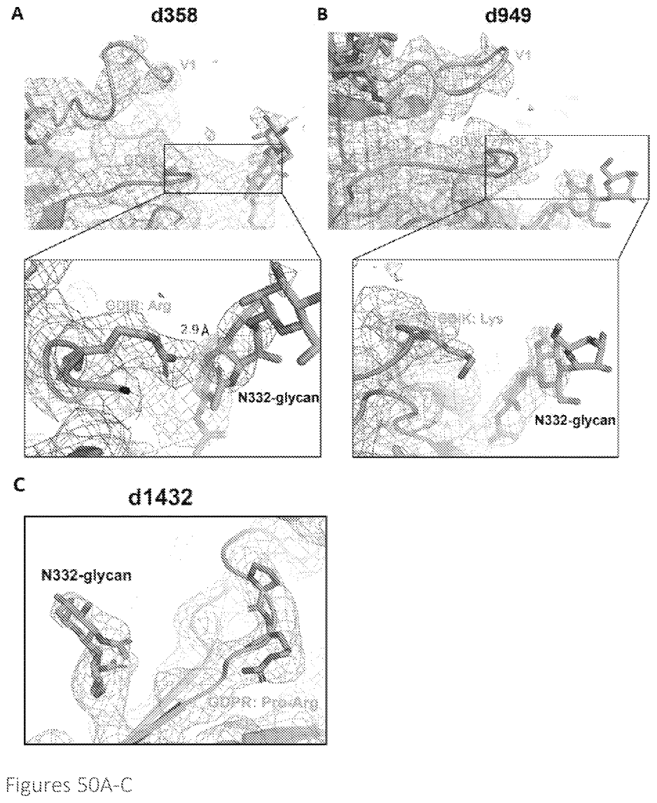

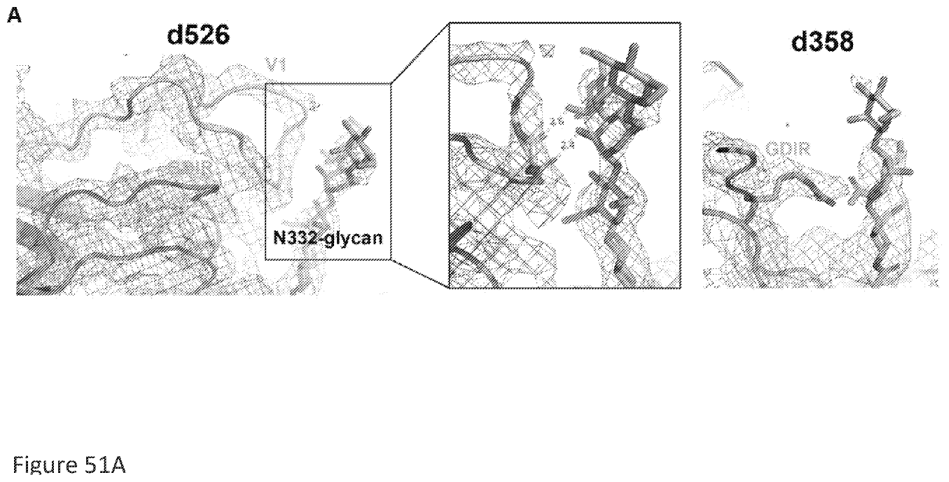

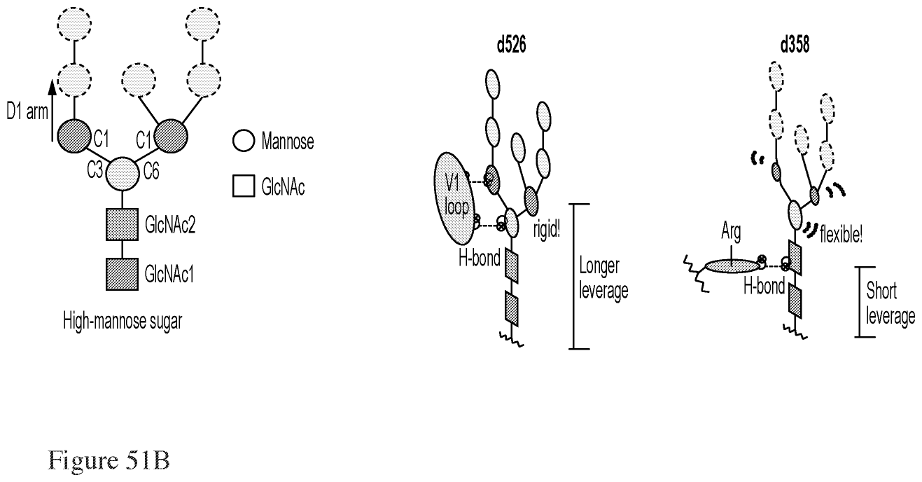

D0526.25.02 Env has an extremely long V1 loop (28aa) that binds the N332-glycan-D1-arm. Therefore, the V1 loop in d526 held the N332-glycan tighter than the V3 loop in d358 by a more optimal leverage. (FIGS. 51A-B) This difference of leverage corresponded to a split along the DH270 phylogenetic tree: the HCDR3 of each branch mutated at the site interacting with one of the two parts on the N332-glycan, hence providing a rationale for the preference of the DH270.6 bnAb path. Specifically, DH270.14 carries the mutations Y106V and S108Y that H-bond with N332-GlcNAc2, while R98T in DH270.I3 facilitate another residue to interact with N332-D1-arm. This is consistent with our observation of the N332-glycan leverage, where DH270.I3 path that breaks at the longer leverage achieved the optimal neutralization DH270.6 in the co-evolution. Therefore, including the sequence of d526 V1 loop may improve the breadth of induced response.

FIG. 52 shows mutated d926 and d1432 Envs with V1 loop substitution of d526 Env.

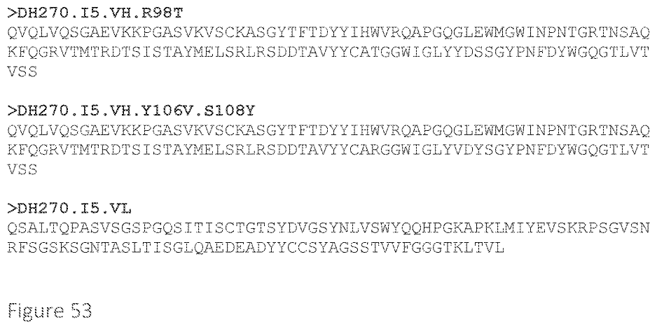

TABLE 5
Proposed mutations

| | Mutation | | |
|---|---|---|---|
| | introduced | | |
| | (HXB2 | | Expected |
| Construct | numbering) | Purpose | results |
| CH848.3.D0949.10.17chim.6R.DS.SOSIP.664 | K327R | Test the | Expect a |
| | | hypothesis | weaker |
| | | that the R327 | binding |
| | | is | affinity to |
| | | critical for | the |
| | | N332- | DH270 |
| | | glycan | lineage |
| | | interaction | than |
| | | | 10.17 |
| | | | K327. |
| | | | Interaction |
| | | | between |
| | | | R327 and |
| | | | N332- |
| | | | glycan is |
| | | | expected |
| | | | in the |
| | | | structure. |
| CH848.3.D0949.10.17chim.6R.DS.SOSIP.664 | V1 loop | Validate the | |
| | replaced by | feasibility to | |
| | d0526.25.02 | incorporate | |
| | V1. See | d526 | |
| | FIG. 52. | long V1 loop | |
| | | to other | |
| | | strains while | |
| | | giving the | |
| | | similar | |
| | | interactions | |
| | | with | |
| | | N332-glycan. | |
| CH848.3.D1432.5.41CHIM.DS.6R.SOSIP.664 | V1 loop | Validate the | |
| | replaced by | feasibility to | |
| | d0526.25.02 | incorporate | |
| | V1. See | d526 long | |
| | FIG. 52. | V1 loop | |
| | | to other | |
| | | strains while | |
| | | giving the | |
| | | similar | |
| | | interactions | |
| | | with | |
| | | N332-glycan. | |
| DH270.I5 | R98T, See | Test the | R98T |
| | FIG. 53 | hypothesis | mutation |
| | | that the | has a |
| | | different | detectable |
| | | leverage of | binding |
| | | binding to | to d526 |
| | | N332 is | and d358 |
| | | corresponding | Envs. |
| | | to the I5 split | while the |
| | | path (I3 | double- |
| | | R98T, I4 | mutant |
| | | Y106V/S108Y) | does not. |
| DH270.I5 | Y106V/S108Y. | Test the | R98T |
| | See FIG. 53 | hypothesis | mutation |
| | | that the | has a |
| | | different | detectable |
| | | leverage of | binding |
| | | binding to | to d526 |
| | | N332 is | and d358 |
| | | corresponding | Envs |
| | | to the I5 split | while the |
| | | path (I3 R98T, | double- |
| | | I4 | mutant |
| | | Y106V/S108Y) | does not. |
| DH270.I5.VL | See FIG. 53 | Light chain for | |
| | | DH570.I5 Abs | |

What is claimed is:

1. A recombinant HIV-1 envelope selected from the envelopes listed in Table 1, FIGS. 22-28 or Table 2B, FIG. 52.

2. A composition comprising the envelope of claim 1 and a carrier, wherein the envelope is a protomer comprised in a trimer.

3. The composition of claim 2, wherein the envelope is comprised in a stable trimer.

4. A composition comprising a nanoparticle and a carrier, wherein the nanoparticle comprises any one of the envelopes of claim 1.

5. The composition of claim 4, wherein the nanoparticle is ferritin self-assembling nanoparticle.

6. A composition comprising a nanoparticle and a carrier, wherein the nanoparticle comprises any one of the trimers of claim 2 or 3.

7. The composition of claim 6, wherein the nanoparticle is ferritin self-assembling nanoparticle.

8. The composition of claim 7, wherein the nanoparticle comprises multimers of trimers.

9. The composition of claim 7, wherein the nanoparticle comprises 1-8 trimers.

10. A method of inducing an immune response in a subject comprising administering to the subject an immunogenic composition comprising any one of the recombinant envelopes of the preceding claims or any one of the compositions of the preceding claims, in an amount sufficient to induce an immune response.

11. The method of claim 10, wherein the composition is administered as a prime.

12. The method of claim 10, wherein the composition is administered as a boost.

13. A nucleic acid encoding any of the recombinant envelopes of the preceding claims.

14. A composition comprising the nucleic acid of claim 13 and a carrier.

15. A method of inducing an immune response in a subject comprising administering to the subject an immunogenic composition comprising the nucleic acid of claim 13 or the composition of claim 14.

16. A method of inducing an immune response comprising administering to a subject an immunogenic composition comprising a prime immunogen from Table 4 followed by at least one boost immunogen from Table 1 or Table 2B, wherein the boost immunogens are administered in an amount sufficient to induce an immune response.

17. The method of claim 16, further comprising administering a boost from Table 4, wherein the boost is CH848.0808.15.15 in any suitable form.

18. The method of claim 17, further comprising administering a boost from Table 4, wherein the boost is CH848.0358.80.06 in any suitable form.

19. The method of claim 17, further comprising administering a boost from Table 4, wherein the boost is CH848.1432.5.41 in any suitable form.

20. The method of claim 17, further comprising administering a boost from Table 4, wherein the boost is CH848.1621.4.44 in any suitable form.

21. The method of claim 17, further comprising administering a boost from Table 4, wherein the boost is CH848.1305.10.35 in any suitable form.

22. The method of claim 17, further comprising administering a boost from Table 4, wherein the boost is P0402.c2.11 (G) in any suitable form.

23. The method of claim 17, further comprising administering a boost from Table 4, wherein the boost is ZM246F (C) in any suitable form.

24. The method of claim 16-23, wherein the prime or boost immunogen are administered as a nanoparticle.

25. The method of any one of claims 16-23, wherein the nanoparticle is a ferritin nanoparticle.

26. The method of any one of claims 16-23, wherein the prime or boost immunogen are administered as mRNA-LNP formulation.
